SEQUENCE LISTING (113 sequences)

SEQ ID NO: 1 - DNA, 1509 bp, Homo sapien
atggctttgattcctgatttggctatggaaactagattgttgttggctgtttcattggttttgttgtatttgtatggaactcattcacatggattgtttaaaaaattgggaattcctggacctactcctttgccttttttgggaaatattttgtcatatcataaaggattttgcatgtttgatatggaatgccataaaaaatatggaaaagtttggggattttatgatggacaacaacctgttttggctattactgatcctgatatgattaaaactgttttggttaaagaatgctattcagtttttactaatagaagaccttttggacctgttggatttatgaaatcagctatttcaattgctgaagatgaagaatggaaaagattgagatcattgttgtcacctacttttacttcaggaaaattgaaagaaatggttcctattattgctcaatatggagatgttttggttagaaatttgagaagagaagctgaaactggaaaacctgttactttgaaagatgtttttggagcttattcaatggatgttattacttcaacttcatttggagttaatattgattcattgaataatcctcaagatccttttgttgaaaatactaaaaaattgttgagatttgattttttggatcctttttttttgtcaattactgtttttccttttttgattcctattttggaagttttgaatatttgcgtttttcctagagaagttactaattttttgagaaaatcagttaaaagaatgaaagaatcaagattggaagatactcaaaaacatagagttgattttttgcaattgatgattgattcacaaaattcaaaagaaactgaatcacataaagctttgtcagatttggaattggttgctcaatcaattatttttatttttgctggatgcgaaactacttcatcagttttgtcatttattatgtatgaattggctactcatcctgatgttcaacaaaaattgcaagaagaaattgatgctgttttgcctaataaagctcctcctacttatgatactgttttgcaaatggaatatttggatatggttgttaatgaaactttgagattgtttcctattgctatgagattggaaagagtttgcaaaaaagatgttgaaattaatggaatgtttattcctaaaggagttgttgttatgattccttcatatgctttgcatagagatcctaaatattggactgaacctgaaaaatttttgcctgaaagattttcaaaaaaaaataaagataatattgatccttatatttatactccttttggatcaggacctagaaattgcattggaatgagatttgctttgatgaatatgaaattggctttgattagagttttgcaaaatttttcatttaaaccttgcaaagaaactcaaattcctttgaaattgtcattgggaggattgttgcaacctgaaaaacctgttgttttgaaagttgaatcaagagatggaactgtttcaggagct

SEQ ID NO: 2 - protein, 503 aa, Homo sapien
MALIPDLAMETRLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGCETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 3 - DNA, 2040 bp, Homo sapien
atgattaatatgggagattcacatgttgatacttcatcaactgtttcagaagctgttgctgaagaagtttcattgttttcaatgactgatatgattttgttttcattgattgttggattgttgacttattggtttttgtttagaaaaaaaaaagaagaagttcctgaatttactaaaattcaaactttgacttcatcagttagagaatcatcatttgttgaaaaaatgaaaaaaactggaagaaatattattgttttttatggatcacaaactggaactgctgaagaatttgctaatagattgtcaaaagatgctcatagatatggaatgagaggaatgtcagctgatcctgaagaatatgatttggctgatttgtcatcattgcctgaaattgataatgctttggttgttttttgcatggctacttatggagaaggagatcctactgataatgctcaagatttttatgattggttgcaagaaactgatgttgatttgtcaggagttaaatttgctgtttttggattgggaaataaaacttatgaacattttaatgctatgggaaaatatgttgataaaagattggaacaattgggagctcaaagaatttttgaattgggattgggagatgatgatggaaatttggaagaagattttattacttggagagaacaattttggttggctgtttgcgaacattttggagttgaagctactggagaagaatcatcaattagacaatatgaattggttgttcatactgatattgatgctgctaaagtttatatgggagaaatgggaagattgaaatcatatgaaaatcaaaaacctccttttgatgctaaaaatccttttttggctgctgttactactaatagaaaattgaatcaaggaactgaaagacatttgatgcatttggaattggatatttcagattcaaaaattagatatgaatcaggagatcatgttgctgtttatcctgctaatgattcagctttggttaatcaattgggaaaaattttgggagctgatttggatgttgttatgtcattgaataatttggatgaagaatcaaataaaaaacatccttttccttgccctacttcatatagaactgctttgacttattatttggatattactaatcctcctagaactaatgttttgtatgaattggctcaatatgcttcagaaccttcagaacaagaattgttgagaaaaatggcttcatcatcaggagaaggaaaagaattgtatttgtcatgggttgttgaagctagaagacatattttggctattttgcaagattgcccttcattgagacctcctattgatcatttgtgcgaattgttgcctagattgcaagctagatattattcaattgcttcatcatcaaaagttcatcctaattcagttcatatttgcgctgttgttgttgaatatgaaactaaagctggaagaattaataaaggagttgctactaattggttgagagctaaagaacctgttggagaaaatggaggaagagctttggttcctatgtttgttagaaaatcacaatttagattgccttttaaagctactactcctgttattatggttggacctggaactggagttgctccttttattggatttattcaagaaagagcttggttgagacaacaaggaaaagaagttggagaaactttgttgtattatggatgcagaagatcagatgaagattatttgtatagagaagaattggctcaatttcatagagatggagctttgactcaattgaatgttgctttttcaagagaacaatcacataaagtttatgttcaacatttgttgaaacaagatagagaacatttgtggaaattgattgaaggaggagctcatatttatgtttgcggagatgctagaaatatggctagagatgttcaaaatactttttatgatattgttgctgaattgggagctatggaacatgctcaagctgttgattatattaaaaaattgatgactaaaggaagatattcattggatgtttggtca

SEQ ID NO: 4 - protein, 680 aa, Homo sapien
MINMGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWLAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPVGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 5 - DNA, 1554 bp, Cannabis sativa
atgaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaattcaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcaaccacggcatcgtcattaa

SEQ ID NO: 6 - protein, 517 aa, Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNSRLAYLNYRDLDIGINDPKNPNNYTQARIWGEKYFGKNFDRLVKVKTLVDPNNFFRNEQSIPPQPRHRH

SEQ ID NO: 7 - DNA, 1374 bp, Stevia rebaudiana
atggaaaataaaactgaaactactgttagaagaagaagaagaattattttgtttcctgttccttttcaaggacatattaatcctattttgcaattggctaatgttttgtattcaaaaggattttcaattactatttttcatactaattttaataaacctaaaacttcaaattatcctcattttacttttagatttattttggataatgatcctcaagatgaaagaatttcaaatttgcctactcatggacctttggctggaatgagaattcctattattaatgaacatggagctgatgaattgagaagagaattggaattgttgatgttggcttcagaagaagatgaagaagtttcatgcttgattactgatgctttgtggtattttgctcaatcagttgctgattcattgaatttgagaagattggttttgatgacttcatcattgtttaattttcatgctcatgtttcattgcctcaatttgatgaattgggatatttggatcctgatgataaaactagattggaagaacaagcttcaggatttcctatgttgaaagttaaagatattaaatcagcttattcaaattggcaaattttgaaagaaattttgggaaaaatgattaaacaaactagagcttcatcaggagttatttggaattcatttaaagaattggaagaatcagaattggaaactgttattagagaaattcctgctccttcatttttgattcctttgcctaaacatttgactgcttcatcatcatcattgttggatcatgatagaactgtttttcaatggttggatcaacaacctccttcatcagttttgtatgtttcatttggatcaacttcagaagttgatgaaaaagattttttggaaattgctagaggattggttgattcaaaacaatcatttttgtgggttgttagacctggatttgttaaaggatcaacttgggttgaacctttgcctgatggatttttgggagaaagaggaagaattgttaaatgggttcctcaacaagaagttttggctcatggagctattggagctttttggactcattcaggatggaattcaactttggaatcagtttgcgaaggagttcctatgattttttcagattttggattggatcaacctttgaatgctagatatatgtcagatgttttgaaagttggagtttatttggaaaatggatgggaaagaggagaaattgctaatgctattagaagagttatggttgatgaagaaggagaatatattagacaaaatgctagagttttgaaacaaaaagctgatgtttcattgatgaaaggaggatcatcatatgaatcattggaatcattggtttcatatatttcatcattg

SEQ ID NO: 8 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 9 - DNA, 1965 bp, Homo sapien
atgtcatcatcaaatgttgaagtttttattcctgtttcacaaggaaatactaatggatttcctgctactgcttcaaatgatttgaaagcttttactgaaggagctgttttgtcatttcataatatttgctatagagttaaattgaaatcaggatttttgccttgcagaaaacctgttgaaaaagaaattttgtcaaatattaatggaattatgaaacctggattgaatgctattttgggacctactggaggaggaaaatcatcattgttggatgttttggctgctagaaaagatccttcaggattgtcaggagatgttttgattaatggagctcctagacctgctaattttaaatgcaattcaggatatgttgttcaagatgatgttgttatgggaactttgactgttagagaaaatttgcaattttcagctgctttgagattggctactactatgactaatcatgaaaaaaatgaaagaattaatagagttattcaagaattgggattggataaagttgctgattcaaaagttggaactcaatttattagaggagtttcaggaggagaaagaaaaagaacttcaattggaatggaattgattactgatccttcaattttgtttttggatgaacctactactggattggattcatcaactgctaatgctgttttgttgttgttgaaaagaatgtcaaaacaaggaagaactattattttttcaattcatcaacctagatattcaatttttaaattgtttgattcattgactttgttggcttcaggaagattgatgtttcatggacctgctcaagaagctttgggatattttgaatcagctggatatcattgcgaagcttataataatcctgctgatttttttttggatattattaatggagattcaactgctgttgctttgaatagagaagaagattttaaagctactgaaattattgaaccttcaaaacaagataaacctttgattgaaaaattggctgaaatttatgttaattcatcattttataaagaaactaaagctgaattgcatcaattgtcaggaggagaaaaaaaaaaaaaaattactgtttttaaagaaatttcatatactacttcattttgccatcaattgagatgggtttcaaaaagatcatttaaaaatttgttgggaaatcctcaagcttcaattgctcaaattattgttactgttgttttgggattggttattggagctatttattttggattgaaaaatgattcaactggaattcaaaatagagctggagttttgttttttttgactactaatcaatgcttttcatcagtttcagctgttgaattgtttgttgttgaaaaaaaattgtttattcatgaatatatttcaggatattatagagtttcatcatattttttgggaaaattgttgtcagatttgttgcctatgagaatgttgccttcaattatttttacttgcattgtttattttatgttgggattgaaagctaaagctgatgctttttttgttatgatgtttactttgatgatggttgcttattcagcttcatcaatggctttggctattgctgctggacaatcagttgtttcagttgctactttgttgatgactatttgctttgtttttatgatgattttttcaggattgttggttaatttgactactattgcttcatggttgtcatggttgcaatatttttcaattcctagatatggatttactgctttgcaacataatgaatttttgggacaaaatttttgccctggattgaatgctactggaaataatccttgcaattatgctacttgcactggagaagaatatttggttaaacaaggaattgatttgtcaccttggggattgtggaaaaatcatgttgctttggcttgcatgattgttatttttttgactattgcttatttgaaattgttgtttttgaaaaaatattca

SEQ ID NO: 10 - protein, 655 aa, Homo sapien [sequence omitted]

SEQ ID NO: 11 - DNA, 1074 bp, Cannabis
atgaagaagaacaaatcaactagtaataataagaacaacaacagtaataatatcatcaaaaacgacatcgtatcatcatcatcatcaacaacaacaacatcatcaacaactacagcaacatcatcatttcataatgagaaagttactgtcagtactgatcatattattaatcttgatgataagcagaaacgacaattatgtcgttgtcgtttagaaaaagaagaagaagaagaaggaagtggtggttgtggtgagacagtagtaatgatgctagggtcagtatctcctgctgctgctactgctgctgcagctgggggctcatcaagttgtgatgaagacatgttgggtggtcatgatcaactgttgttgttgtgttgttctgagaaaaaaacgacagaaatttcatcagtggtgaactttaataataataataataataataaggaaaatggtgacgaagtttcaggaccgtacgattatcatcatcataaagaagaggaagaagaagaagaagaagatgaagcatctgcatcagtagcagctgttgatgaagggatgttgttgtgctttgatgacataatagatagccacttgctaaatccaaatgaggttttgactttaagagaagatagccataatgaaggtggggcagctgatcagattgacaagactacttgtaataatactactattactactaatgatgattataacaataacttgatgatgttgagctgcaataataacggagattatgttattagtgatgatcatgatgatcagtactggatagacgacgtcgttggagttgacttttggagttgggagagttcgactactactgttattacccaagaacaagaacaagaacaagatcaagttcaagaacagaagaatatgtgggataatgagaaagagaaactgttgtctttgctatgggataatagtgataacagcagcagttgggagttacaagataaaagcaataataataataataataatgttcctaacaaatgtcaagagattacctctgataaagaaaatgctatggttgcatggcttctctcctga

SEQ ID NO: 12 - protein, 357 aa, Cannabis
MKKNKSTSNNKNNNSNNIIKNDIVSSSSSTTTTSSTTTATSSFHNEKVTVSTDHIINLDDKQKRQLCRCRLEKEEEEEGSGGCGETVVMMLGSVSPAAATAAAAGGSSSCDEDMLGGHDQLLLLCCSEKKTTEISSVVNFNNNNNNNKENGDEVSGPYDYHHHKEEEEEEEEDEASASVAAVDEGMLLCFDDIIDSHLLNPNEVLTLREDSHNEGGAADQIDKTTCNNTTITTNDDYNNNLMMLSCNNNGDYVISDDHDDQYWIDDVVGVDFWSWESSTTTVITQEQEQEQDQVQEQKNMWDNEKEKLLSLLWDNSDNSSSWELQDKSNNNNNNNVPNKCQEITSDKENAMVAWLLS

SEQ ID NO: 13 - DNA, 1476 bp, Arabidopsis thaliana
atggatccttataaatatagacctgcttcatcatataattcacctttttttactactaattcaggagctcctgtttggaataataattcatcaatgactgttggacctagaggattgattttgttggaagattatcatttggttgaaaaattggctaattttgatagagaaagaattcctgaaagagttgttcatgctagaggagcttcagctaaaggattttttgaagttactcatgatatttcaaatttgacttgcgctgattttttgagagctcctggagttcaaactcctgttattgttagattttcaactgttattcatgctagaggatcacctgaaactttgagagatcctagaggatttgctgttaaattttatactagagaaggaaattttgatttggttggaaataattttcctgttttttttattagagatggaatgaaatttcctgatattgttcatgctttgaaacctaatcctaaatcacatattcaagaaaattggagaattttggattttttttcacatcatcctgaatcattgaatatgtttacttttttgtttgatgatattggaattcctcaagattatagacatatggatggatcaggagttaatacttatatgttgattaataaagctggaaaagctcattatgttaaatttcattggaaacctacttgcggagttaaatcattgttggaagaagatgctattagattgggaggaactaatcattcacatgctactcaagatttgtatgattcaattgctgctggaaattatcctgaatggaaattgtttattcaaattattgatcctgctgatgaagataaatttgattttgatcctttggatgttactaaaacttggcctgaagatattttgcctttgcaacctgttggaagaatggttttgaataaaaatattgataatttttttgctgaaaatgaacaattggctttttgccctgctattattgttcctggaattcattattcagatgataaattgttgcaaactagagttttttcatatgctgatactcaaagacatagattgggacctaattatttgcaattgcctgttaatgctcctaaatgcgctcatcataataatcatcatgaaggatttatgaattttatgcatagagatgaagaagttaattattttccttcaagatatgatcaagttagacatgctgaaaaatatcctactcctcctgctgtttgctcaggaaaaagagaaagatgcattattgaaaaagaaaataattttaaagaacctggagaaagatatagaacttttactcctgaaagacaagaaagatttattcaaagatggattgatgctttgtcagatcctagaattactcatgaaattagatcaatttggatttcatattggtcacaagctgataaatcattgggacaaaaattggcttcaagattgaatgttagaccttcaatt

SEQ ID NO: 14 - protein, 492 aa, Arabidopsis thaliana
MDPYKYRPASSYNSPFFTTNSGAPVWNNNSSMTVGPRGLILLEDYHLVEKLANFDRERIPERVVHARGASAKGFFEVTHDISNLTCADFLRAPGVQTPVIVRFSTVIHARGSPETLRDPRGFAVKFYTREGNFDLVGNNFPVFFIRDGMKFPDIVHALKPNPKSHIQENWRILDFFSHHPESLNMFTFLFDDIGIPQDYRHMDGSGVNTYMLINKAGKAHYVKFHWKPTCGVKSLLEEDAIRLGGTNHSHATQDLYDSIAAGNYPEWKLFIQIIDPADEDKFDFDPLDVTKTWPEDILPLQPVGRMVLNKNIDNFFAENEQLAFCPAIIVPGIHYSDDKLLQTRVFSYADTQRHRLGPNYLQLPVNAPKCAHHNNHHEGFMNFMHRDEEVNYFPSRYDQVRHAEKYPTPPAVCSGKRERCIIEKENNFKEPGERYRTFTPERQERFIQRWIDALSDPRITHEIRSIWISYWSQADKSLGQKLASRLNVRPSI

SEQ ID NO: 15 - DNA, 2262 bp, Escherichia coli
atgtcgcaacataacgaaaagaacccacatcagcaccagtcaccactacacgattccagcgaagcgaaaccggggatggactcactggcacctgaggacggctctcatcgtccagcggctgaaccaacaccgccaggtgcacaacctaccgccccagggagcctgaaagcccctgatacgcgtaacgaaaaacttaattctctggaagacgtacgcaaaggcagtgaaaattatgcgctgaccactaatcagggcgtgcgcatcgccgacgatcaaaactcactgcgtgccggtagccgtggtccaacgctgctggaagattttattctgcgcgagaaaatcacccactttgaccatgagcgcattccggaacgtattgttcatgcacgcggatcagccgctcacggttatttccagccatataaaagcttaagcgatattaccaaagcggatttcctctcagatccgaacaaaatcaccccagtatttgtacgtttctctaccgttcagggtggtgctggctctgctgataccgtgcgtgatatccgtggctttgccaccaagttctataccgaagagggtatttttgacctcgttggcaataacacgccaatcttctttatccaggatgcgcataaattccccgattttgttcatgcggtaaaaccagaaccgcactgggcaattccacaagggcaaagtgcccacgatactttctgggattatgtttctctgcaacctgaaactctgcacaacgtgatgtgggcgatgtcggatcgcggcatcccccgcagttaccgcaccatggaaggcttcggtattcacaccttccgcctgattaatgccgaagggaaggcaacgtttgtacgtttccactggaaaccactggcaggtaaagcctcactcgtttgggatgaagcacaaaaactcaccggacgtgacccggacttccaccgccgcgagttgtgggaagccattgaagcaggcgattttccggaatacgaactgggcttccagttgattcctgaagaagatgaattcaagttcgacttcgatcttctcgatccaaccaaacttatcccggaagaactggtgcccgttcagcgtgtcggcaaaatggtgctcaatcgcaacccggataacttctttgctgaaaacgaacaggcggctttccatcctgggcatatcgtgccgggactggacttcaccaacgatccgctgttgcagggacgtttgttctcctataccgatacacaaatcagtcgtcttggtgggccgaatttccatgagattccgattaaccgtccgacctgcccttaccataatttccagcgtgacggcatgcatcgcatggggatcgacactaacccggcgaattacgaaccgaactcgattaacgataactggccgcgcgaaacaccgccggggccgaaacgcggcggttttgaatcataccaggagcgcgtggaaggcaataaagttcgcgagcgcagcccatcgtttggcgaatattattcccatccgcgtctgttctggctaagtcagacgccatttgagcagcgccatattgtcgatggtttcagttttgagttaagcaaagtcgttcgtccgtatattcgtgagcgcgttgttgaccagctggcgcatattgatctcactctggcccaggcggtggcgaaaaatctcggtatcgaactgactgacgaccagctgaatatcaccccacctccggacgtcaacggtctgaaaaaggatccatccttaagtttgtacgccattcctgacggtgatgtgaaaggtcgcgtggtagcgattttacttaatgatgaagtgagatcggcagaccttctggccattctcaaggcgctgaaggccaaaggcgttcatgccaaactgctctactcccgaatgggtgaagtgactgcggatgacggtacggtgttgcctatagccgctacctttgccggtgcaccttcgctgacggtcgatgcggtcattgtcccttgcggcaatatcgcggatatcgctgacaacggcgatgccaactactacctgatggaagcctacaaacaccttaaaccgattgcgctggcgggtgacgcgcgcaagtttaaagcaacaatcaagatcgctgaccagggtgaagaagggattgtggaagctgacagcgctgacggtagttttatggatgaactgctaacgctgatggcagcacaccgcgtgtggtcacgcattcctaagattgacaaaattcctgcctga

SEQ ID NO: 16 - protein, 753 aa, Escherichia coli
MSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 17 - DNA, 1635 bp, Cannabis
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctctaccacggcatcgtcattaa

SEQ ID NO: 18 - protein, 544 aa, Cannabis
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 19 - DNA, 1467 bp, Stevia rebaudiana
atgaagtgctcaacattctccttttggtttgtttgcaagataatatttttctttttctcattcaatatccaaacttccattgctaatcctcgagaaaataaaactgaaactactgttagaagaagaagaagaattattttgtttcctgttccttttcaaggacatattaatcctattttgcaattggctaatgttttgtattcaaaaggattttcaattactatttttcatactaattttaataaacctaaaacttcaaattatcctcattttacttttagatttattttggataatgatcctcaagatgaaagaatttcaaatttgcctactcatggacctttggctggaatgagaattcctattattaatgaacatggagctgatgaattgagaagagaattggaattgttgatgttggcttcagaagaagatgaagaagtttcatgcttgattactgatgctttgtggtattttgctcaatcagttgctgattcattgaatttgagaagattggttttgatgacttcatcattgtttaattttcatgctcatgtttcattgcctcaatttgatgaattgggatatttggatcctgatgataaaactagattggaagaacaagcttcaggatttcctatgttgaaagttaaagatattaaatcagcttattcaaattggcaaattttgaaagaaattttgggaaaaatgattaaacaaactagagcttcatcaggagttatttggaattcatttaaagaattggaagaatcagaattggaaactgttattagagaaattcctgctccttcatttttgattcctttgcctaaacatttgactgcttcatcatcatcattgttggatcatgatagaactgtttttcaatggttggatcaacaacctccttcatcagttttgtatgtttcatttggatcaacttcagaagttgatgaaaaagattttttggaaattgctagaggattggttgattcaaaacaatcatttttgtgggttgttagacctggatttgttaaaggatcaacttgggttgaacctttgcctgatggatttttgggagaaagaggaagaattgttaaatgggttcctcaacaagaagttttggctcatggagctattggagctttttggactcattcaggatggaattcaactttggaatcagtttgcgaaggagttcctatgattttttcagattttggattggatcaacctttgaatgctagatatatgtcagatgttttgaaagttggagtttatttggaaaatggatgggaaagaggagaaattgctaatgctattagaagagttatggttgatgaagaaggagaatatattagacaaaatgctagagttttgaaacaaaaagctgatgtttcattgatgaaaggaggatcatcatatgaatcattggaatcattggtttcatatatttcatcattgtaa

SEQ ID NO: 20 - protein, 488 aa, Stevia rebaudiana
MKCSTFSFWFVCKIIFFFFSFNIQTSIANPRENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 21 - DNA, 1022 bp, Arabidopsis thaliana
atggaggtccatggctccggattccgtcgaattctgttgttggcgttgtgtatctccgggatctggtccgcctacatctaccaaggcgttcttcaagagactctgtccacgaagagatttggtccagatgagaagaggttcgagcatcttgcattcttgaacttagctcaaagtgtagtctgcttgatctggtcttatataatgatcaagctctggtcaaatgctggtaacggtggagcaccatggtggacgtattggagtgcaggcattactaatacaattggtcctgccatgggaattgaagccttgaagtatatcagttatccagctcaggttttggcaaaatcgtcaaaaatgattccagttatgctaatgggaactttagtttacggaataagatacactttccctgaatacatgtgcacctttcttgtcgctggaggagtatccatctttgctcttcttaagacaagctctaagacaattagcaagctagcacatccaaatgctcccctcggttacgcactttgttccttaaacctcgcctttgacggattcacaaatgccacacaagactccattgcctcaaggtacccaaaaaccgaagcgtgggacataatgctgggaatgaacttatggggcacaatatacaacattatctacatgtttggcttgccacaagggatggattcgaagcaattcagttctgtaagctacacccggaagcggcatgggacattctaaagtattgtatatgcggtgccgtgggacaaaacttcatcttcatgacaataagtaacttcgggtcactagctaacacgaccataaccacgaccaggaagtttgttagcattgttgtatcatcagtaatgagcggaaatccattgtcgttgaagcaatggggatgtgtttcgatggtctttggtggtttggcatatcaaatttatcttaaatggaagaaattgcagagagtggagtgctccataatgaacttaatgtgtgggtctacctgcgccgcttga

SEQ ID NO: 22 - DNA, 1554 bp, Cannabis sativa
atgaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctctaccacggcatcgtcattaa

SEQ ID NO: 23 - protein, 517 aa, Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 24 - DNA, 1377 bp, Stevia rebaudiana
atggaaaataaaaccgaaaccaccgtccgccgtcgtcgccgtatcattctgttcccggtcccgttccagggccacatcaacccgattctgcaactggcgaacgtgctgtattcgaaaggtttcagcatcaccatcttccatacgaacttcaacaagccgaagaccagcaattacccgcactttacgttccgttttattctggataacgacccgcaggatgaacgcatctctaatctgccgacccacggcccgctggcgggtatgcgtattccgattatcaacgaacacggcgcagatgaactgcgtcgcgaactggaactgctgatgctggccagcgaagaagatgaagaagtttcttgcctgatcaccgacgcactgtggtattttgcccagtctgttgcagatagtctgaacctgcgtcgcctggtcctgatgaccagcagcctgttcaattttcatgcccacgttagtctgccgcagttcgatgaactgggttatctggacccggatgacaaaacccgcctggaagaacaggcgagcggctttccgatgctgaaagtcaaggatattaagtcagcgtactcgaactggcagattctgaaagaaatcctgggtaaaatgattaagcaaaccaaagcaagttccggcgtcatctggaatagtttcaaagaactggaagaatccgaactggaaacggtgattcgtgaaatcccggctccgagttttctgattccgctgccgaagcatctgaccgcgagcagcagcagcctgctggatcacgaccgcacggtgtttcagtggctggatcagcaaccgccgagttccgtgctgtatgttagcttcggtagtacctcggaagtggatgaaaaggactttctggaaatcgctcgtggcctggttgatagcaaacaatctttcctgtgggtggttcgcccgggttttgtgaagggctctacgtgggttgaaccgctgccggacggcttcctgggtgaacgtggccgcattgtcaaatgggtgccgcagcaagaagtgctggcgcatggcgcgattggcgcgttttggacccactccggttggaactcaacgctggaatcggtttgtgaaggtgtcccgatgattttctcagattttggcctggaccagccgctgaatgcacgttatatgtcggatgttctgaaagtcggtgtgtacctggaaaacggttgggaacgcggcgaaattgcgaatgccatccgtcgcgttatggtcgatgaagaaggcgaatacattcgtcagaatgctcgcgtcctgaaacaaaaggcggacgtgagcctgatgaaaggcggttcatcgtatgaaagtctggaatccctggtttcatacatcagctctctgtaa

SEQ ID NO: 25 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 26 - protein, 485 aa, Nicotiana tabacum
MGSIGAELTKPHAVCIPYPAQGHINPMLKLAKILHHKGFHITFVNTEFNHRRLLKSRGPDSLKGLSSFRFETIPDGLPPCEADATQDIPSLCESTTNTCLAPFRDLLAKLNDTNTSNVPPVSCIVSDGVMSFTLAAAQELGVPEVLFWTTSACGFLGYMHYCKVIEKGYAPLKDASDLTNGYLETTLDFIPGMKDVRLRDLPSFLRTTNPDEFMIKFVLQETERARKASAIILNTFETLEAEVLESLRNLLPPVYPIGPLHFLVKHVDDENLKGLRSSLWKEEPECIQWLDTKEPNSVVYVNFGSITVMTPNQLIEFAWGLANSQQTFLWIIRPDIVSGDASILPPEFVEETKNRGMLASWCSQEEVLSHPAIVGFLTHSGWNSTLESISSGVPMICWPFFAEQQTNCWFSVTKWDVGMEIDSDVKRDEVESLVRELMVGGKGKKMKKKAMEWKELAEASAKEHSGSSYVNIEKLVNDILLSSKH

SEQ ID NO: 27 - DNA, 1458 bp, Nicotiana tabacum
atgggttccattggtgctgaattaacaaagccacatgcagtttgcataccatatcccgcccaaggccatattaaccccatgttaaagctagccaaaatccttcatcacaaaggctttcacatcacttttgtcaatactgaatttaaccaccgacgtctccttaaatctcgtggccctgattctctcaagggtctttcttctttccgttttgagaccattcctgatggacttccgccatgtgaggcagatgccacacaagatataccttctttgtgtgaatctacaaccaatacttgcttggctccttttagggatcttcttgcgaaactcaatgatactaacacatctaacgtgccacccgtttcgtgcatcgtctcggatggtgtcatgagcttcaccttagccgctgcacaagaattgggagtccctgaagttctgttttggaccactagtgcttgtggtttcttaggttacatgcattactgcaaggttattgaaaaaggatatgctccacttaaagatgcgagtgacttgacaaatggatacctagagacaacattggattttataccaggcatgaaagacgtacgtttaagggatcttccaagtttcttgagaactacaaatccagatgaattcatgatcaaatttgtcctccaagaaacagagagagcaagaaaggcttctgcaattatcctcaacacatttgaaacactagaggctgaagttcttgaatcgctccgaaatcttcttcctccagtctaccccatagggcccttgcattttctagtgaaacatgttgatgatgagaatttgaagggacttagatccagcctttggaaagaggaaccagagtgtatacaatggcttgataccaaagaaccaaattctgttgtttatgttaactttggaagcattactgttatgactcctaatcagcttattgagtttgcttggggacttgcaaacagccagcaaacattcttatggatcataagacctgatattgtttcaggtgatgcatcgattcttccacccgaattcgtggaagaaacgaagaacagaggtatgcttgctagttggtgttcacaagaagaagtacttagtcaccctgcaatagtaggattcttgactcacagtggatggaattcgacactcgaaagtataagcagtggggtgcctatgatttgctggccatttttcgctgaacagcaaacaaattgttggttttccgtcactaaatgggatgttggaatggagattgacagtgatgtgaagagagatgaagtggaaagccttgtaagggaattgatggttgggggaaaaggcaaaaagatgaagaaaaaggcaatggaatggaaggaattggctgaagcatctgctaaagaacattcagggtcatcttatgtgaacattgaaaagttggtcaatgatattcttctttcatccaaacattaa

SEQ ID NO: 28 - protein, 485 aa, Nicotiana tabacum
MGSIGAEFTKPHAVCIPYPAQGHINPMLKLAKILHHKGFHITFVNTEFNHRRLLKSRGPDSLKGLSSFRFETIPDGLPPCDADATQDIPSLCESTTNTCLGPFRDLLAKLNDTNTSNVPPVSCIISDGVMSFTLAAAQELGVPEVLFWTTSACGFLGYMHYYKVIEKGYAPLKDASDLTNGYLETTLDFIPCMKDVRLRDLPSFLRTTNPDEFMIKFVLQETERARKASAIILNTYETLEAEVLESLRNLLPPVYPIGPLHFLVKHVDDENLKGLRSSLWKEEPECIQWLDTKEPNSVVYVNFGSITVMTPNQLIEFAWGLANSQQSFLWIIRPDIVSGDASILPPEFVEETKKRGMLASWCSQEEVLSHPAIGGFLTHSGWNSTLESISSGVPMICWPFFAEQQTNCWFSVTKWDVGMEIDCDVKRDEVESLVRELMVGGKGKKMKKKAMEWKELAEASAKEHSGSSYVNIEKVVNDILLSSKH

SEQ ID NO: 29 - DNA, 1458 bp, Nicotiana tabacum
atgggttccattggtgctgaatttacaaagccacatgcagtttgcataccatatcccgcccaaggccatattaaccccatgttaaagctagccaaaatccttcatcacaaaggctttcacatcacttttgtcaatactgaatttaaccacagacgtctgcttaaatctcgtggccctgattctctcaagggtctttcttctttccgttttgagacaattcctgatggacttccgccatgtgatgcagatgccacacaagatataccttctttgtgtgaatctacaaccaatacttgcttgggtccttttagggatcttcttgcgaaactcaatgatactaacacatctaacgtgccacccgtttcgtgcatcatctcagatggtgtcatgagcttcaccttagccgctgcacaagaattgggagtccctgaagttctgttttggaccactagtgcttgtggtttcttaggttacatgcattattacaaggttattgaaaaaggatacgctccacttaaagatgcgagtgacttgacaaatggatacctagagacaacattggattttataccatgcatgaaagacgtacgtttaagggatcttccaagtttcttgagaactacaaatccagatgaattcatgatcaaatttgtcctccaagaaacagagagagcaagaaaggcttctgcaattatcctcaacacatatgaaacactagaggctgaagttcttgaatcgctccgaaatcttcttcctccagtctaccccattgggcccttgcattttctagtgaaacatgttgatgatgagaatttgaagggacttagatccagcctttggaaagaggaaccagagtgtatacaatggcttgataccaaagaaccaaattctgttgtttatgttaactttggaagcattactgttatgactcctaatcaacttattgaatttgcttggggacttgcaaacagccaacaatcattcttatggatcataagacctgatattgtttcaggtgatgcatcgattcttccccccgaattcgtggaagaaacgaagaagagaggtatgcttgctagttggtgttcacaagaagaagtacttagtcaccctgcaataggaggattcttgactcacagtggatggaattcgacactcgaaagtataagcagtggggtgcctatgatttgctggccatttttcgctgaacagcaaacaaattgttggttttccgtcactaaatgggatgttggaatggagattgactgtgatgtgaagagggatgaagtggaaagccttgtaagggaattgatggttgggggaaaaggcaaaaagatgaagaaaaaggcaatggaatggaaggaattggctgaagcatctgctaaagaacattcagggtcatcttatgtgaacattgagaaggtggtcaatgatattcttctttcgtccaaacattaa

SEQ ID NO: 30 - protein, 496 aa, Nicotiana tabacum
MATQVHKLHFILFPLMAPGHMIPMIDIAKLLANRGVITTIITTPVNANRFSSTITRAIKSGLRIQILTLKFPSVEVGLPEGCENIDMLPSLDLASKFFAAISMLKQQVENLLEGINPSPSCVISDMGFPWTTQIAQNFNIPRIVFHGTCCFSLLCSYKILSSNILENITSDSEYFVVPDLPDRVELTKAQVSGSTKNTTSVSSSVLKEVTEQIRLAEESSYGVIVNSFEELEQVYEKEYRKARGKKVWCVGPVSLCNKEIEDLVTRGNKTAIDNQDCLKWLDNFETESVVYASLGSLSRLTLLQMVELGLGLEESNRPFVWVLGGGDKLNDLEKWILENGFEQRIKERGVLIRGWAPQVLILSHPAIGGVLTHCGWNSTLEGISAGLPMVTWPLFAEQFCNEKLVVQVLKIGVSLGVKVPVKWGDEENVGVLVKKDDVKKALDKLMDEGEEGQVRRTKAKELGELAKKAFGEGGSSYVNLTSLIEDIIEQQNHKEK

SEQ ID NO: 31 - DNA, 1491 bp, Nicotiana tabacum
atggcaactcaagtgcacaaacttcatttcatactattccctttaatggctccaggccacatgattcctatgatagacatagctaaacttctagcaaatcgcggtgtcattaccactatcatcaccactccagtaaacgccaatcgtttcagttcaacaattactcgtgccataaaatccggtctaagaatccaaattcttacactcaaatttccaagtgtagaagtaggattaccagaaggttgcgaaaatattgacatgcttccttctcttgacttggcttcaaagttttttgctgcaattagtatgctgaaacaacaagttgaaaatctcttagaaggaataaatccaagtccaagttgtgttatttcagatatgggatttccttggactactcaaattgcacaaaattttaatatcccaagaattgtttttcatggtacttgttgtttctcacttttatgttcctataaaatactttcctccaacattcttgaaaatataacctcagattcagagtattttgttgttcctgatttacccgatagagttgaactaacgaaagctcaggtttcaggatcgacgaaaaatactacttctgttagttcttctgtattgaaagaagttactgagcaaatcagattagccgaggaatcatcatatggtgtaattgttaatagttttgaggagttggagcaagtgtatgagaaagaatataggaaagctagagggaaaaaagtttggtgtgttggtcctgtttctttgtgtaataaggaaattgaagatttggttacaaggggtaataaaactgcaattgataatcaagattgcttgaaatggttagataattttgaaacagaatctgtggtttatgcaagtcttggaagtttatctcgtttgacattattgcaaatggtggaacttggtcttggtttagaagagtcaaataggccttttgtatgggtattaggaggaggtgataaattaaatgatttagagaaatggattcttgagaatggatttgagcaaagaattaaagaaagaggagttttgattagaggatgggctcctcaagtgcttatactttcacaccctgcaattggtggagtattgactcattgcggatggaattctacattggaaggtatttcagcaggattaccaatggtaacatggccactatttgctgagcaattttgcaatgagaagttagtagtccaagtgctaaaaattggagtgagcctaggtgtgaaggtgcctgtcaaatggggagatgaggaaaatgttggagttttggtaaaaaaggatgatgttaagaaagcattagacaaactaatggatgaaggagaagaaggacaagtaagaagaacaaaagcaaaagagttaggagaattggctaaaaaggcatttggagaaggtggttcttcttatgttaacttaacatctctgattgaagacatcattgagcaacaaaatcacaaggaaaaatag

SEQ ID NO: 32 - protein, 479 aa, Nicotiana tabacum
MKTAELVFIPAPGMGHLVPTVEVAKQLVDRHEQLSITVLIMTIPLETNIPSYTKSLSSDYSSRITLLPLSQPETSVTMSSFNAINFFEYISSYKGRVKDAVSETSFSSSNSVKLAGFVIDMFCTAMIDVANEFGIPSYVFYTSSAAMLGLQLHFQSLSIECSPKVHNYVEPESEVLISTYMNPVPVKCLPGIILVNDESSTMFVNHARRFRETKGIMVNTFTELESHALKALSDDEKIPPIYPVGPILNLENGNEDHNQEYDAIMKWLDEKPNSSVVFLCFGSKGSFEEDQVKEIANALESSGYHFLWSLRRPPPKDKLQFPSEFENPEEVLPEGFFQRTKGRGKVIGWAPQLAILSHPSVGGFVSHCGWNSTLESVRSGVPIATWPLYAEQQSNAFQLVKDLGMAVEIKMDYREDFNTRNPPLVKAEEIEDGIRKLMDSENKIRAKVTEMKDKSRAALLEGGSSYVALGHFVETVMKN

SEQ ID NO: 33 - DNA, 1440 bp, Nicotiana tabacum
atgaagacagcagagttagtattcattcctgctcctgggatgggtcaccttgtaccaactgtggaggtggcaaagcaactagtcgacagacacgagcagctttcgatcacagttctaatcatgacaattcctttggaaacaaatattccatcatatactaaatcactgtcctcagactacagttctcgtataacgctgcttccactctctcaacctgagacctctgttactatgagcagttttaatgccatcaatttttttgagtacatctccagctacaagggtcgtgtcaaagatgctgttagtgaaacctcctttagttcgtcaaattctgtgaaacttgcaggatttgtaatagacatgttctgcactgcgatgattgatgtagcgaacgagtttggaatcccaagttatgtgttctacacttctagtgcagctatgcttggactacaactgcattttcaaagtcttagcattgaatgcagtccgaaagttcataactacgttgaacctgaatcagaagttctgatctcaacttacatgaatccggttccagtcaaatgtttgcccggaattatactagtaaatgatgaaagtagcaccatgtttgtcaatcatgcacgaagattcagggagacgaaaggaattatggtgaacacgttcactgagcttgaatcacacgctttgaaagccctttccgatgatgaaaaaatcccaccaatctacccagttggacctatacttaaccttgaaaatgggaatgaagatcacaatcaagaatatgatgcgattatgaagtggcttgacgagaagcctaattcatcagtggtgttcttatgctttggaagcaaggggtctttcgaagaagatcaggtgaaggaaatagcaaatgctctagagagcagtggctaccacttcttgtggtcgctaaggcgaccgccaccaaaagacaagctacaattcccaagcgaattcgagaatccagaggaagtcttaccagagggattctttcaaaggactaaaggaagaggaaaggtgataggatgggcaccccagttggctattttgtctcatccttcagtaggaggattcgtgtcgcattgtgggtggaattcaactctggagagcgttcgaagtggagtgccgatagcaacatggccattgtatgcagagcaacagagcaatgcatttcaactggtgaaggatttgggtatggcagtagagattaagatggattacagggaagattttaatacgagaaatccaccactggttaaagctgaggagatagaagatggaattaggaagctgatggattcagagaataaaatcagggctaaggtgacggagatgaaggacaaaagtagagcagcactgctggagggcggatcatcatatgtagctcttgggcattttgttgagactgtcatgaaaaactag

SEQ ID NO: 34 - protein, 478 aa, Nicotiana tabacum
MKTTELVFIPAPGMGHLVPTVEVAKQLVDRDEQLSITVLIMTLPLETNIPSYTKSLSSDYSSRITLLQLSQPETSVSMSSFNAINFFEYISSYKDRVKDAVNETFSSSSSVKLKGFVIDMFCTAMIDVANEFGIPSYVFYTSNAAMLGLQLHFQSLSIEYSPKVHNYLDPESEVAISTYINPIPVKCLPGIILDNDKSGTMFVNHARRFRETKGIMVNTFAELESHALKALSDDEKIPPIYPVGPILNLGDGNEDHNQEYDMIMKWLDEQPHSSVVFLCFGSKGSFEEDQVKEIANALERSGNRFLWSLRRPPPKDTLQFPSEFENPEEVLPVGFFQRTKGRGKVIGWAPQLAILSHPAVGGFVSHCGWNSTLESVRSGVPIATWPLYAEQQSNAFQLVKDLGMAVEIKMDYREDFNKTNPPLVKAEEIEDGIRKLMDSENKIRAKVMEMKDKSRAALLEGGSSYVALGHFVETVMKN

SEQ ID NO: 35 - DNA, 1437 bp, Nicotiana tabacum
atgaagacaacagagttagtattcattcctgctcctggcatgggtcaccttgtacccactgtggaggtggcaaagcaactagtcgacagagacgaacagctttcaatcacagttctcatcatgacgcttcctttggaaacaaatattccatcatatactaaatcactgtcctcagactacagttctcgtataacgctgcttcaactttctcaacctgagacctctgttagtatgagcagttttaatgccatcaatttttttgagtacatctccagctacaaggatcgtgtcaaagatgctgttaatgaaacctttagttcgtcaagttctgtgaaactcaaaggatttgtaatagacatgttctgcactgcgatgattgatgtggcgaacgagtttggaatcccaagttatgtcttctacacttctaatgcagctatgcttggactccaactccattttcaaagtcttagtattgaatacagtccgaaagttcataattacctagaccctgaatcagaagtagcgatctcaacttacattaatccgattccagtcaaatgtttgcccgggattatactagacaatgataaaagtggcaccatgttcgtcaatcatgcacgaagattcagggagacgaaaggaattatggtgaacacattcgctgagcttgaatcacacgctttgaaagccctttccgatgatgagaaaatcccaccaatctacccagttgggcctatacttaaccttggagatgggaatgaagatcacaatcaagaatatgatatgattatgaagtggctcgacgagcagcctcattcatcagtggtgttcctatgctttggaagcaagggatctttcgaagaagatcaagtgaaggaaatagcaaatgctctagagagaagtggtaaccggttcttgtggtcgctaagacgaccgccaccaaaagacacgctacaattcccaagcgaattcgagaatccagaggaagtcttgccggtgggattctttcaaaggactaaaggaagaggaaaggtgataggatgggcaccccagttggctattttgtctcatcctgcagtaggaggattcgtgtcgcattgtgggtggaattcaactttggagagtgttcgtagtggagtaccgatagcaacatggccattgtatgcagagcaacagagcaatgcatttcaactggtgaaggatttggggatggcagtggagattaagatggattacagggaagattttaataagacaaatccaccactggttaaagctgaggagatagaagatggaattaggaagctgatggattcagagaataaaatcagggctaaggtgatggagatgaaggacaaaagtagagcagcgttattagaaggcggatcatcatatgtagctctcgggcattttgttgagactgtcatgaaaaactaa

SEQ ID NO: 36 - protein, 482 aa, Nicotiana tabacum
MKETKKIELVFIPSPGIGHLVSTVEMAKLLIAREEQLSITVLIIQWPNDKKLDSYIQSVANFSSRLKFIRLPQDDSIMQLLKSNIFTTFIASHKPAVRDAVADILKSESNNTLAGIVIDLFCTSMIDVANEFELPTYVFYTSGAATLGLHYHIQNLRDEFNKDITKYKDEPEEKLSIATYLNPFPAKCLPSVALDKEGGSTMFLDLAKRFRETKGIMINTFLELESYALNSLSRDKNLPPIYPVGPVLNLNNVEGDNLGSSDQNTMKWLDDQPASSVVFLCFGSGGSFEKHQVKEIAYALESSGCRFLWSLRRPPTEDARFPSNYENLEEILPEGFLERTKGIGKVIGWAPQLAILSHKSTGGFVSHCGWNSTLESTYFGVPIATWPMYAEQQANAFQLVKDLRMGVEIKMDYRKDMKVMGKEVIVKAEEIEKAIREIMDSESEIRVKVKEMKEKSRAAQMEGGSSYTSIGGFIQIIMENSQ

SEQ ID NO: 37 - DNA, 1449 bp, Nicotiana tabacum
atgaaagaaaccaagaaaatagagttagtcttcattccttcaccaggaattggccatttagtatccacagttgaaatggcaaagcttcttatagctagagaagagcagctatctatcacagtcctcatcatccaatggcctaacgacaagaagctcgattcttatatccaatcagtcgccaatttcagctcgcgtttgaaattcattcgactccctcaggatgattccattatgcagctactcaaaagcaacattttcaccacgtttattgccagtcataagcctgcagttagagatgctgttgctgatattctcaagtcagaatcaaataatacgctagcaggtattgttatcgacttgttctgcacctcaatgatagacgtggccaatgagttcgagctaccaacctatgttttctacacgtctggtgcagcaacccttggtcttcattatcatatacagaatctcagggatgaatttaacaaagatattaccaagtacaaagacgaacctgaagaaaaactctctatagcaacatatctcaatccatttccagcaaaatgtttgccgtctgtagccttagacaaagaaggtggttcaacaatgtttcttgatctcgcaaaaaggtttcgagaaaccaaaggtattatgataaacacatttctagagctcgaatcctatgcattaaactcgctctcacgagacaagaatcttccacctatataccctgtcggaccagtattgaaccttaacaatgttgaaggtgacaacttaggttcatctgaccagaatactatgaaatggttagatgatcagcccgcttcatctgtagtgttcctttgttttggtagtggtggaagctttgaaaaacatcaagttaaggaaatagcctatgctctggagagcagtgggtgtcggtttttgtggtcgttaaggcgaccaccaaccgaagatgcaagatttccaagcaactatgaaaatcttgaagaaattttgccagaaggattcttggaaagaacaaaagggattggaaaagtgataggatgggcacctcagttggcgattttgtcacataaatcgacggggggatttgtgtcgcactgtggatggaattcgactttggaaagtacatattttggagtgccaatagcaacctggccaatgtacgcggagcaacaagcgaatgcatttcaattggttaaggatttgagaatgggagttgagattaagatggattataggaaggatatgaaagtgatgggcaaagaagttatagtgaaagctgaggagattgagaaagcaataagagaaattatggattccgagagtgaaattcgggtgaaggtgaaagagatgaaggagaagagcagagcagcacaaatggaaggtggctcttcttacacttctattggaggtttcatccaaattatcatggagaattctcaataa

SEQ ID NO: 38 - protein, 470 aa, Nicotiana tabacum
MVQPHVLLVTFPAQGHINPCLQFAKRLIRMGIEVTFATSVFAHRRMAKTTTSTLSKGLNFAAFSDGYDDGFKADEHDSQHYMSEIKSRGSKTLKDIILKSSDEGRPVTSLVYSLLLPWAAKVAREFHIPCALLWIQPATVLDIYYYYFNGYEDAIKGSTNDPNWCIQLPRLPLLKSQDLPSFLLSSSNEEKYSFALPTFKEQLDTLDVEENPKVLVNTFDALEPKELKAIEKYNLIGIGPLIPSTFLDGKDPLDSSFGGDLFQKSNDYIEWLNSKANSSVVYISFGSLLNLSKNQKEEIAKGLIEIKKPFLWVIRDQENGKGDEKEEKLSCMMELEKQGKIVPWCSQLEVLTHPSIGCFVSHCGWNSTLESLSSGVSVVAFPHWTDQGTNAKLIEDVWKTGVRLKKNEDGVVESEEIKRCIEMVMDGGEKGEEMRRNAQKWKELAREAVKEGGSSEMNLKAFVQEVGKGC

SEQ ID NO: 39 - DNA, 1413 bp, Nicotiana tabacum
atggtgcaaccccatgtcctcttggtgacttttccagcacaaggccatattaatccatgtctccaatttgccaagaggctaattagaatgggcattgaggtaacttttgccacgagcgttttcgcccatcgtcgtatggcaaaaactacgacttccactctatccaagggcttaaattttgcggcattctctgatgggtacgacgatggtttcaaggccgatgagcatgattctcaacattacatgtcggagataaaaagtcgcggttctaaaaccctaaaagatatcattttgaagagctcagacgagggacgtcctgtgacatccctcgtctattctcttttgcttccatgggctgcaaaggtagcgcgtgaatttcacataccgtgcgcgttactatggattcaaccagcaactgtgctagacatatattattattacttcaatggctatgaggatgccataaaaggtagcaccaatgatccaaattggtgtattcaattgcctaggcttccactactaaaaagccaagatcttccttcttttttactttcttctagtaatgaagaaaaatatagctttgctctaccaacatttaaagagcaacttgacacattagatgttgaagaaaatcctaaagtacttgtgaacacatttgatgcattagagccaaaggaactcaaagctattgaaaagtacaatttaattgggattggaccattgattccttcaacatttttggacggaaaagaccctttggattcttcctttggtggtgatctttttcaaaagtctaatgactatattgaatggttgaactcaaaggctaactcatctgtggtttatatctcatttgggagtctcttgaatttgtcaaaaaatcaaaaggaggagattgcaaaagggttgatagagattaaaaagccattcttgtgggtaataagagatcaagaaaatggtaagggagatgaaaaagaagagaaattaagttgtatgatggagttggaaaagcaagggaaaatagtaccatggtgttcacaacttgaagtcttaacacatccatctataggatgtttcgtgtcacattgtggatggaattcgactctggaaagtttatcgtcaggcgtgtcagtagtggcatttcctcattggacggatcaagggacaaatgctaaactaattgaagatgtttggaagacaggtgtaaggttgaaaaagaatgaagatggtgtggttgagagtgaagagataaaaaggtgcatagaaatggtaatggatggtggagagaaaggagaagaaatgagaagaaatgctcaaaaatggaaagaattggcaagggaagctgtaaaagaaggcggatcttcggaaatgaatctaaaagcttttgttcaagaagttggcaaaggttgctga

SEQ ID NO: 40 - protein, 28 aa, Cannabis
MNCSAFSFWFVCKIIFFFLSFHIQISIA

SEQ ID NO: 41 - protein, 28 aa, Cannabis
MKCSTFSFWFVCKIIFFFFSFNIQTSIA

SEQ ID NO: 42 - protein, 545 aa, Cannabis
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVDVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 43 - protein, 462 aa, Humulus lupulus
MGRAPCCEKVGLKKGRWTSEEDEILTKYIQSNGEGCWRSLPKNAGLLRCGKSCRLRWINYLRADLKRGNISSEEEDIIIKLHSTLGNRWSLIASHLPGRTDNEIKNYWNSHLSRKIHTFRRCNNTTTHHHHLPNLVTVTKVNLPIPKRKGGRTSRLAMKKNKSSTSNQNSSVIKNDVGSSSSTTTTSVHQRTTTTTPTMDDQQKRQLSRCRLEEKEDQDGASTGTVVMMLGQAAAVGSSCDEDMLGHDQLSFLCCSEEKTTENSMTNLKENGDHEVSGPYDYDHRYEKETSVDEGMLLCFNDIIDSNLLNPNEVLTLSEESLNLGGALMDTTTSTTTNNNNYSLSYNNNGDCVISDDHDQYWLDDVVGVDFWSWESSTTVTQEQEQEQEQEQEQEQEQEQEQEHHHQQDQKKNTWDNEKEKMLALLWDSDNSNWELQDNNNYHKCQEITSDKENAMVAWLLS

SEQ ID NO: 44 - protein, 371 aa, Arabidopsis thaliana
MGRAPCCEKVGIKRGRWTAEEDQILSNYIQSNGEGSWRSLPKNAGLKRCGKSCRLRWINYLRSDLKRGNITPEEEELVVKLHSTLGNRWSLIAGHLPGRTDNEIKNYWNSHLSRKLHNFIRKPSISQDVSAVIMTNASSAPPPPQAKRRLGRTSRSAMKPKIHRTKTRKTKKTSAPPEPNADVAGADKEALMVESSGAEAELGRPCDYYGDDCNKNLMSINGDNGVLTFDDDIIDLLLDESDPGHLYTNTTCGGDGELHNIRDSEGARGFSDTWNQGNLDCLLQSCPSVESFLNYDHQVNDASTDEFIDWDCVWQEGSDNNLWHEKENPDSMVSWLLDGDDEATIGNSNCENFGEPLDHDDESALVAWLLS

SEQ ID NO: 45 - protein, 243 aa, Arabidopsis thaliana
MNISRTEFANCKTLINHKEEVEEVEKKMEIEIRRGPWTVEEDMKLVSYISLHGEGRWNSLSRSAGLNRTGKSCRLRWLNYLRPDIRRGDISLQEQFIILELHSRWGNRWSKIAQHLPGRTDNEIKNYWRTRVQKHAKLLKCDVNSKQFKDTIKHLWMPRLIERIAATQSVQFTSNHYSPENSSVATATSSTSSSEAVRSSFYGGDQVEFGTLDHMTNGGYWFNGGDTFETLCSFDELNKWLIQ

SEQ ID NO: 46 - protein, 517 aa, Cannabis
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVDVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 47 - protein, 520 aa, Arabidopsis thaliana
MNCSAFSFWFVCKIIFFFLSFHIQISIAMDPYKYRPASSYNSPFFTTNSGAPVWNNNSSMTVGPRGLILLEDYHLVEKLANFDRERIPERVVHARGASAKGFFEVTHDISNLTCADFLRAPGVQTPVIVRFSTVIHARGSPETLRDPRGFAVKFYTREGNFDLVGNNFPVFFIRDGMKFPDIVHALKPNPKSHIQENWRILDFFSHHPESLNMFTFLFDDIGIPQDYRHMDGSGVNTYMLINKAGKAHYVKFHWKPTCGVKSLLEEDAIRLGGTNHSHATQDLYDSIAAGNYPEWKLFIQIIDPADEDKFDFDPLDVTKTWPEDILPLQPVGRMVLNKNIDNFFAENEQLAFCPAIIVPGIHYSDDKLLQTRVFSYADTQRHRLGPNYLQLPVNAPKCAHHNNHHEGFMNFMHRDEEVNYFPSRYDQVRHAEKYPTPPAVCSGKRERCIIEKENNFKEPGERYRTFTPERQERFIQRWIDALSDPRITHEIRSIWISYWSQADKSLGQKLASRLNVRPSI

SEQ ID NO: 48 - protein, 520 aa, Arabidopsis thaliana
MKCSTFSFWFVCKIIFFFFSFNIQTSIAMDPYKYRPASSYNSPFFTTNSGAPVWNNNSSMTVGPRGLILLEDYHLVEKLANFDRERIPERVVHARGASAKGFFEVTHDISNLTCADFLRAPGVQTPVIVRFSTVIHARGSPETLRDPRGFAVKFYTREGNFDLVGNNFPVFFIRDGMKFPDIVHALKPNPKSHIQENWRILDFFSHHPESLNMFTFLFDDIGIPQDYRHMDGSGVNTYMLINKAGKAHYVKFHWKPTCGVKSLLEEDAIRLGGTNHSHATQDLYDSIAAGNYPEWKLFIQIIDPADEDKFDFDPLDVTKTWPEDILPLQPVGRMVLNKNIDNFFAENEQLAFCPAIIVPGIHYSDDKLLQTRVFSYADTQRHRLGPNYLQLPVNAPKCAHHNNHHEGFMNFMHRDEEVNYFPSRYDQVRHAEKYPTPPAVCSGKRERCIIEKENNFKEPGERYRTFTPERQERFIQRWIDALSDPRITHEIRSIWISYWSQADKSLGQKLASRLNVRPSI

SEQ ID NO: 49 - protein, 781 aa, Escherichia coli
MNCSAFSFWFVCKIIFFFLSFHIQISIAMSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 50 - protein, 781 aa, Escherichia coli
MKCSTFSFWFVCKIIFFFFSFNIQTSIAMSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 51 - DNA, 1437 bp, Nicotiana tabacum
atgaaaacaacagaacttgtcttcatacccgcccccggtatgggtcaccttgtacccacagtcgaagtcgccaaacaactagttgatagagacgaacagttgtctattaccgtcttgataatgacgttacccctggagactaatatcccaagttacaccaagagtttgtcctctgactattcatcccgtatcacgttgttacaactaagtcaacctgagacgagtgtctcaatgagtagttttaacgccataaacttcttcgaatacattagttcctataaggatcgtgttaaagatgccgtaaacgagacattctcctcttcatcctccgtcaaacttaaaggatttgtaatcgacatgttttgcacggcaatgatagacgtggccaacgagttcggtattccatcttatgtattctacacgtccaacgctgccatgctaggcctacaacttcacttccaatccttgtccatcgaatattcacctaaggttcataattatttagaccctgaatctgaggtagctatatcaacgtacattaacccaataccagtaaaatgcttacccggtataattcttgacaatgataagagtggcactatgttcgtaaaccatgccaggagattccgtgaaacaaagggtataatggtaaatacttttgcagaattagaaagtcacgccctaaaggcacttagtgacgatgagaaaattcctccaatctatcccgtcggacccattctaaacttgggtgatggtaatgaggatcataaccaagagtacgacatgataatgaaatggctggatgaacaaccacacagttcagtggttttcctgtgcttcggttccaaaggttcatttgaagaagaccaggttaaagagatagcaaatgctttagagagatcaggcaataggttcctgtggagtttaagacgtccccctcccaaggatactcttcaattcccttccgaatttgaaaaccccgaggaagtgctacctgtaggattttttcaaagaaccaaaggcagaggaaaagtcatcggatgggcaccacagcttgcaattctatctcaccctgccgtcggtggattcgtttcccactgcggctggaatagtactttggaatcagttagatcaggtgtacccatagcaacatggcctctttatgcagagcagcagtccaatgcatttcaattggtcaaggatctaggtatggccgtcgaaattaaaatggattaccgtgaggactttaacaagactaatcctccattggtaaaggcagaggaaatagaagacggcattaggaagttgatggactccgagaataagattagggcaaaggtgatggaaatgaaagataagtccagagctgcattactggaaggaggatcctcctatgttgcactgggtcacttcgtggagaccgtaatgaagaactaa

SEQ ID NO: 52 - protein, 478 aa, Nicotiana tabacum; identical to SEQ ID NO: 34

SEQ ID NO: 53 - DNA, 1413 bp, Nicotiana tabacum
atggttcaaccacacgtcttactggttacttttccagcacaaggccatatcaacccttgcctacaattcgccaaaagactaataaggatgggcatcgaagtaacttttgccacgagtgtattcgcacataggcgtatggctaaaactacgacatcaactttgtccaaaggactaaacttcgccgccttcagtgatggctatgacgatggattcaaagccgacgaacatgacagtcaacactacatgagtgaaataaagtcccgtggatctaaaacacttaaggatattatacttaaatcctccgatgagggaagacccgttacctctttagtttattcactgttactgccctgggctgcaaaagtcgccagagagtttcatattccttgcgctttattgtggatccaaccagctacggtattagacatctactattactacttcaatggatacgaggatgcaataaagggatcaacaaacgaccccaactggtgtattcaactgcctagacttcctctattaaaaagtcaggacttacctagttttttactgtcatccagtaacgaagaaaaatattcattcgctttacccaccttcaaagagcagcttgacactttggatgttgaagagaaccccaaggttttggtcaatacttttgacgctttggagccaaaagagctaaaggctattgaaaaatataaccttatcggcataggacctttaatcccctctactttcttagatggcaaagaccctctagattcaagtttcggaggtgatttgtttcaaaagagtaacgattatatcgagtggctaaatagtaaagccaactccagtgtggtctacatttctttcggaagtcttctgaatttatcaaaaaaccaaaaggaagagatcgcaaaaggactgatagagataaaaaaacctttcttatgggtgatcagagaccaggaaaacggtaaaggcgatgagaaggaggaaaaactgtcctgtatgatggagctagagaaacaaggaaaaatcgttccctggtgttcacagttagaagtgttaacccatccatccataggttgcttcgtatcacattgtggttggaatagtacacttgaaagtctttcatcaggcgtctctgtcgtcgcattcccccactggacggaccagggcacaaacgccaaactgatcgaagatgtatggaagacgggcgtcaggctaaaaaaaaatgaggatggcgtggtagagagtgaagagataaagcgttgcatagaaatggtcatggatggcggtgaaaagggagaggaaatgaggcgtaacgcacaaaagtggaaggaactagcccgtgaagcagtgaaagaaggaggttctagtgagatgaatttaaaagctttcgtgcaggaagttggaaaaggctgctga

SEQ ID NO: 54 - protein, 470 aa, Nicotiana tabacum; identical to SEQ ID NO: 38

SEQ ID NO: 55 - DNA, 1449 bp, Nicotiana tabacum
atgaaagagactaaaaaaattgagttagtttttatccccagtcctggtataggacacttagtctcaactgtggagatggccaaactgttgatagcccgtgaagagcaactttctattactgtcctgattatacaatggcctaatgataaaaagctagacagttatatccagtccgtcgcaaactttagttctagactgaagtttatacgtctgccccaagatgactcaatcatgcaacttttgaaatcaaacattttcacgacattcatcgcctctcacaagccagctgtaagagacgccgttgctgacatactaaagagtgaaagtaataacacattggcaggcattgtaatcgatcttttctgcacatccatgatcgatgtagccaatgagtttgagctgcctacttatgtgttttacactagtggcgcagccacgttgggtctgcactaccatattcaaaatctgcgtgatgagtttaataaagacattaccaaatataaggatgagccagaagaaaaattaagtatagccacgtaccttaacccattccctgctaagtgtctaccctccgtggcattggataaggaaggaggatcaacgatgttcctagacttagctaagaggttcagggagaccaaaggcataatgattaacacttttcttgagctggaatcatacgctctaaactcattgtctagagataaaaacttgccccctatataccctgtaggccctgttttgaacttgaacaacgttgagggtgataacttgggctctagtgatcaaaataccatgaaatggctggacgaccagccagcttcttccgttgtgttcctatgttttggctcaggaggaagtttcgaaaaacaccaagtcaaagaaatagcttatgccttagaatcttccggatgcaggttcttgtggagtttgcgtagaccccccacggaagatgctaggttcccttctaattacgaaaacttagaggaaattttaccagagggatttctggaaagaacgaaaggcattggtaaggtcattggatgggccccacagttagcaatcttgtctcacaagtccacaggaggattcgtgtctcattgcggatggaactctacccttgaaagtacctatttcggcgttcctattgctacttggccaatgtatgctgaacaacaggccaacgcttttcaacttgttaaagatttgaggatgggtgttgagatcaaaatggattataggaaggatatgaaggtaatgggcaaggaggttatcgttaaggcagaagaaattgaaaaggccataagggaaatcatggactcagaatcagaaatcagggtcaaggtcaaagagatgaaggagaaaagtcgtgcagcccaaatggaaggaggatcatcatatacctctatcggcggcttcattcaaataatcatggagaactcacagtaa

SEQ ID NO: 56 - protein, 482 aa, Nicotiana tabacum; identical to SEQ ID NO: 36

SEQ ID NO: 57 - DNA, 1491 bp, Nicotiana tabacum
atggctactcaggtgcataaattgcatttcattctgttcccactgatggctcccggtcacatgatccctatgatagacatcgcaaaactattggctaaccgtggcgtgataactaccataataactacgcccgttaacgccaatcgtttttcctctacgatcactagggccattaaatcaggcctaagaatccagattttaaccttaaaattcccatcagttgaggtaggcctgcctgaaggatgtgaaaacatcgacatgttgccatctttggacttagcctctaaattctttgctgctatttctatgcttaaacaacaagtggagaacttgctagagggtattaaccctagtccctcatgcgttatttctgacatgggcttcccatggacgacacagatcgctcaaaatttcaatattcctcgtatcgtatttcatggcacgtgttgcttttctcttctttgttcttacaaaatcctgtcatccaatatcttagagaacattactagtgactcagagtattttgtcgtgccagatctgccagaccgtgtcgagctaactaaggcccaagtctctggatctacaaagaatactacatcagtaagtagttcagtactgaaggaggttacagagcagatcaggcttgcagaggaatcatcctacggtgtgatagttaattccttcgaagaactggaacaggtgtatgaaaaagagtacagaaaagccaggggcaaaaaggtctggtgcgtgggtcctgtctctttgtgcaacaaggagattgaagatcttgttactagaggaaacaaaaccgctatagacaatcaggattgtcttaagtggttagacaacttcgagactgaatccgtcgtctatgcaagtttaggctcactaagtaggcttacgttactgcaaatggttgagctgggattgggactggaggagagtaataggccatttgtatgggttctgggaggaggagacaaactaaatgatcttgagaaatggatattggagaatggctttgaacagcgtataaaggagagaggtgtcctgatacgtggctgggcacctcaagtattgattttaagtcaccccgcaattggaggagttttaacgcattgtggatggaactctacattagagggcatttcagccggactacccatggtcacctggccactatttgccgaacagttctgtaacgaaaaattagtagtgcaggttcttaaaatcggtgtctcacttggagtgaaggtccctgttaagtggggtgacgaagagaacgtaggtgtcttagtgaaaaaggatgacgttaaaaaagcactggataagctaatggatgagggtgaggagggccaggttaggaggaccaaagccaaagagcttggtgagttagctaaaaaagcctttggagagggcggatcatcctacgtgaacctaacgtccctaattgaagatataatcgagcagcagaaccataaggagaagtag

SEQ ID NO: 58 - protein, 496 aa, Nicotiana tabacum; identical to SEQ ID NO: 30

SEQ ID NO: 59 - DNA, 1458 bp, Nicotiana tabacum
atgggctctatcggtgcagaactaaccaagccacacgccgtatgcattccctatcccgcccagggacacataaatcctatgctgaagttagctaagatactgcatcacaagggcttccatataaccttcgtaaatacggaatttaatcacaggcgtctgctgaagtccagaggtcctgactccctgaaaggtctttcaagtttcaggttcgagacgatacctgacggactgcccccatgcgaagctgacgctacacaggacattccttcactgtgtgaatccacgactaatacatgtctagctccttttagagacctacttgctaagctaaatgatacgaatacttctaacgtccctcccgtaagttgtattgtcagtgacggagtgatgtcatttacccttgcagctgcacaggaactgggtgtcccagaggttttattttggactacatctgcttgtggattcttaggttacatgcactattgcaaagtcattgaaaaaggatatgctccattaaaagacgcatcagacctgacgaatggctatcttgagacaaccttggacttcatccccggcatgaaggacgtcaggctgagagacttaccttcctttcttaggaccaccaatccagacgaatttatgattaagtttgtactacaggaaactgagcgtgctcgtaaggccagtgccataatacttaatacctttgaaaccttagaggcagaggtattagaatcattaaggaaccttctaccccccgtctatccaatcggccccttgcatttccttgtcaaacacgtagacgatgagaacctaaaaggtctacgttcctcactttggaaggaggaacctgaatgtattcaatggttagacaccaaagaacctaactctgtcgtgtacgtgaatttcggatccattactgtgatgactcccaatcaattaatagagttcgcttggggactggcaaactctcaacagaccttcctttggatcataaggcctgacatcgtaagtggtgatgcttccatattacctcccgagtttgttgaggagactaagaacagaggcatgcttgcctcctggtgctctcaggaggaggtactatcccatcccgcaatagtgggatttttgacgcactctggttggaactcaactttagaatcaatttctagtggcgtccccatgatctgttggcctttctttgctgagcagcaaacgaactgctggttttcagtgacgaagtgggacgttggaatggaaattgattcagatgtgaagagagatgaagtagagagtttagtaagagagttaatggtgggtggtaaaggcaagaagatgaagaagaaggcaatggagtggaaggaactggccgaggcttcagcaaaagaacactctggctcctcttacgtcaatatcgagaagttggttaacgatatattactatctagtaagcactaa

SEQ ID NO: 60 - protein, 485 aa, Nicotiana tabacum; identical to SEQ ID NO: 26

SEQ ID NO: 61 - DNA, 1377 bp, Stevia rebaudiana
atggagaacaaaaccgagacaaccgttaggcgtagacgtaggataatattgtttcccgtgccctttcaaggccatataaacccaatcctgcagctagccaacgtattgtactcaaagggcttcagtataacgatcttccacaccaactttaataagccaaaaacgtctaattatccacacttcacatttagatttatacttgataacgacccacaggatgaaagaatatcaaacttgcccacgcacggcccactagccggaatgagaataccaataatcaatgagcatggcgccgacgagttgcgtagagagctggaattgttgatgctagccagtgaggaagacgaagaggtgtcctgcttaataacggatgcactttggtattttgctcaatctgtggccgactcccttaacctgaggcgtcttgtccttatgacctccagtctattcaactttcatgcccatgtctcattgccccaatttgatgagcttggctatttggatcctgatgacaaaactaggctggaggaacaggcttccggttttcccatgctaaaggttaaggacatcaaatccgcctactcaaactggcagatccttaaggaaattcttggcaaaatgatcaaacagacgagggcatccagtggcgtcatctggaactcctttaaggaacttgaagaatcagaacttgaaacagtaatcagagaaatacctgccccaagtttcttgatccctctacctaagcaccttacggcttctagttcttctttgttggaccacgatcgtactgtctttcaatggttagatcagcaacccccctcatcagtgctatatgtgtcattcggtagtacatcagaagtggacgaaaaggatttccttgagatagcccgtggattggtggactctaaacagtcctttttatgggttgtgagacctggatttgtaaagggatccacgtgggtcgaacccttgcccgatggtttcctgggtgaaagaggaaggatagtgaagtgggtccctcagcaagaggtactggcccatggtgctataggtgctttctggacccactccggctggaatagtacactagaatccgtttgcgagggtgtccctatgattttttctgattttggtttagatcaacccctgaatgctaggtacatgtcagacgtccttaaagtcggcgtctacctagaaaatggctgggagaggggtgagatagcaaacgctatcagacgtgttatggtagacgaagagggagagtacataaggcaaaacgccagggtcctgaaacaaaaagccgatgtgtccttgatgaagggcggctcttcatacgaaagtctagaaagtcttgtttcttatatttcctcactataa

SEQ ID NO: 62 - protein, 458 aa, Stevia rebaudiana; identical to SEQ ID NO: 8

SEQ ID NO: 63 - DNA, 1434 bp, Lycium barbarum
atgggtcaattgcatttttttttgtttccaatgatggctcaaggtcatatgattccaactttggatatggctaagttgattgcttctagaggtgttaaggctactattattactactccattgaacgaatctgttttttctaaggctattcaaagaaacaagcaattgggtattgaaattgaaattgaaattagattgattaagtttccagctttggaaaacgatttgccagaagattgtgaaagattggatttgattccaactgaagctcatttgccaaacttttttaaggctgctgctatgatgcaagaaccattggaacaattgattcaagaatgtagaccagattgtttggtttctgatatgtttttgccatggactactgatactgctgctaagtttaacattccaagaattgtttttcatggtactaactactttgctttgtgtgttggtgattctatgagaagaaacaagccatttaagaacgtttcttctgattctgaaacttttgttgttccaaacttgccacatgaaattaagttgactagaactcaagtttctccatttgaacaatctgatgaagaatctgttatgtctagagttttgaaggaagttagagaatctgatttgaagtcttacggtgttatttttaactctttttacgaattggaaccagattacgttgaacattacactaaggttatgggtagaaagtcttgggctattggtccattgtctttgtgtaacagagatgttgaagataaggctgaaagaggtaagaagtcttctattgataagcatgaatgtttggaatggttggattctaagaagccatcttctattgtttacgtttgttttggttctgttgctaactttactgttactcaaatgagagaattggctttgggtttggaagcttctggtttggattttatttgggctgttagagctgataacgaagattggttgccagaaggttttgaagaaagaactaaggaaaagggtttgattattagaggttgggctccacaagttttgattttggatcatgaatctgttggtgcttttgttactcattgtggttggaactctactttggaaggtatttctgctggtgttccaatggttacttggccagtttttgctgaacaattttttaacgaaaagttggttactcaagttatgagaactggtgctggtgttggttctgttcaatggaagagatctgcttctgaaggtgttgaaaaggaagctattgctaaggctattaagagagttatggtttctgaagaagctgaaggttttagaaacagagctagagcttacaaggaaatggctagacaagctattgaagaaggtggttcttcttacactggtttgactactttgttggaagatatttcttcttacgaatctttgtcttctgattaa

SEQ ID NO: 64 - protein, 477 aa, Lycium barbarum
MGQLHFFLFPMMAQGHMIPTLDMAKLIASRGVKATIITTPLNESVFSKAIQRNKQLGIEIEIEIRLIKFPALENDLPEDCERLDLIPTEAHLPNFFKAAAMMQEPLEQLIQECRPDCLVSDMFLPWTTDTAAKFNIPRIVFHGTNYFALCVGDSMRRNKPFKNVSSDSETFVVPNLPHEIKLTRTQVSPFEQSDEESVMSRVLKEVRESDLKSYGVIFNSFYELEPDYVEHYTKVMGRKSWAIGPLSLCNRDVEDKAERGKKSSIDKHECLEWLDSKKPSSIVYVCFGSVANFTVTQMRELALGLEASGLDFIWAVRADNEDWLPEGFEERTKEKGLIIRGWAPQVLILDHESVGAFVTHCGWNSTLEGISAGVPMVTWPVFAEQFFNEKLVTQVMRTGAGVGSVQWKRSASEGVEKEAIAKAIKRVMVSEEAEGFRNRARAYKEMARQAIEEGGSSYTGLTTLLEDISSYESLSSD

SEQ ID NO: 65 - DNA, 2262 bp, Escherichia coli
atgtctcaacataacgagaaaaacccacatcagcatcaatcaccactacatgactcctctgaagcaaagccaggaatggactccctggctcctgaagatggctctcaccgtcccgctgccgaacctacgccacccggcgcacagccaactgcccccggttccctaaaggcccctgacacaagaaatgaaaagttaaattctcttgaagacgtgcgtaaaggcagtgaaaattacgctcttaccactaatcaaggcgtaaggatagctgacgaccaaaactccctgcgtgctggctctagaggccctacccttcttgaggattttatccttcgtgaaaagattactcacttcgatcacgaaaggattcctgagaggatcgtccatgctagaggttctgctgctcacggttattttcagccctataaatccctttccgacataacgaaggcagattttttgagtgatcctaataaaataacgcctgtatttgttagattttctactgtccaaggtggtgctggatcagctgacactgttagagacatcaggggatttgctacgaagttttacactgaagagggcatcttcgacttggttggtaataatacaccaatattctttatccaagacgcacacaaattcccagactttgtgcatgctgtcaaacccgagccacattgggctattccacagggccagtctgcccatgacacgttctgggattacgtttctctgcaacctgagacgctgcacaacgttatgtgggcaatgtcagatcgtggaatacctagatcttacaggacaatggaaggctttggcatacatactttcaggttaataaatgccgaaggaaaggccacattcgtcaggtttcattggaagcccttagcaggtaaggcctctctagtatgggacgaagctcaaaaacttactggtagagatccagactttcataggcgtgaattgtgggaagcaatcgaagccggcgactttcctgagtatgagctgggcttccagttgatcccagaagaggacgaatttaaatttgatttcgacttacttgatccaacgaaactgattcccgaggagttggtccctgtccaacgtgtcggtaaaatggtgttgaacaggaaccctgacaatttctttgcagaaaacgaacaagccgccttccatccaggccatatagtaccaggcttagacttcactaatgacccactgctgcaaggtagactgtttagttacactgatacacagatatccagactaggtggtccaaacttccatgaaatccccatcaacaggcccacgtgcccctatcacaatttccagcgtgatggcatgcatagaatgggtattgacacgaatcccgctaattatgagccaaactctataaacgataactggcctagagagacgccaccaggccctaagcgtggtggttttgaatcctatcaagagcgtgtcgaaggtaataaagtaagggagagatcaccctctttcggcgaatattatagtcatccccgtttgttttggttatcacagacgcctttcgaacaacgtcacatagttgatggattctcttttgagctttcaaaagtggttcgtccctatatcagggaaagggttgtcgaccagcttgcccatattgatttaacacttgcacaagctgttgccaaaaacctaggaatagagctgacagacgatcaactaaatatcaccccacctcctgatgtcaacggcttaaagaaggatccatctttaagtctatacgcaattcccgacggtgatgttaaaggtagagtggtagcaattttgctaaacgatgaagtgcgtagtgctgacctactagccatcttaaaggccttgaaagcaaagggagtgcacgcaaagttactgtacagtcgtatgggagaggttactgctgacgacggtacggtactacctatcgccgcaacatttgccggagccccaagtttgacagtcgatgccgttatcgtaccttgtggtaatatcgccgatattgccgacaacggagacgctaattactacttaatggaggcctataagcacttgaagcccatagcactggctggagacgctcgtaaatttaaggctactatcaagattgcagatcagggcgaggagggtattgttgaggcagacagtgcagatggatctttcatggatgagcttctaacactaatggcagcacatagagtatggtctcgtatccccaagatcgacaaaatccctgcgtaa

SEQ ID NO: 66 - protein, 753 aa, Escherichia coli; identical to SEQ ID NO: 16

SEQ ID NO: 67 - DNA, 1974 bp, Mus musculus
atgtcttcttctaacgatcatgttttggttccaatgtctcaaagaaacaacaacggtttgccaagaatgaactctagagctgttagaactttggctgaaggtgatgttttgtcttttcatcatattacttacagagttaaggttaagtctggttttttggttagaaagactgttgaaaaggaaattttgtctgatattaacggtattatgaagccaggtttgaacgctattttgggtccaactggtggtggtaagtcttctttgttggatgttttggctgctagaaaggatccaaagggtttgtctggtgatgttttgattaacggtgctccacaaccagctcattttaagtgttgttctggttacgttgttcaagatgatgttgttatgggtactttgactgttagagaaaacttgcaattttctgctgctttgagattgccaactactatgaagaaccatgaaaagaacgaaagaattaacactattattaaggaattgggtttggaaaaggttgctgattctaaggttggtactcaatttattagaggtatttctggtggtgaaagaaagagaacttctattggtatggaattgattactgatccatctattttgtttttggatgaaccaactactggtttggattcttctactgctaacgctgttttgttgttgttgaagagaatgtctaagcaaggtagaactattattttttctattcatcaaccaagatactctatttttaagttgtttgattctttgactttgttggcttctggtaagttggtttttcatggtccagctcaaaaggctttggaatactttgcttctgctggttaccattgtgaaccatacaacaacccagctgatttttttttggatgttattaacggtgattcttctgctgttatgttgaacagagaagaacaagataacgaagctaacaagactgaagaaccatctaagggtgaaaagccagttattgaaaacttgtctgaattttacattaactctgctatttacggtgaaactaaggctgaattggatcaattgccaggtgctcaagaaaagaagggtacttctgcttttaaggaaccagtttacgttacttctttttgtcatcaattgagatggattgctagaagatcttttaagaacttgttgggtaacccacaagcttctgttgctcaattgattgttactgttattttgggtttgattattggtgctatttactttgatttgaagtacgatgctgctggtatgcaaaacagagctggtgttttgttttttttgactactaaccaatgtttttcttctgtttctgctgttgaattgtttgttgttgaaaagaagttgtttattcatgaatacatttctggttactacagagtttcttcttacttttttggtaaggttatgtctgatttgttgccaatgagatttttgccatctgttatttttacttgtattttgtactttatgttgggtttgaagaagactgttgatgctttttttattatgatgtttactttgattatggttgcttacactgcttcttctatggctttggctattgctactggtcaatctgttgtttctgttgctactttgttgatgactattgcttttgtttttatgatgttgttttctggtttgttggttaacttgagaactattggtccatggttgtcttggttgcaatacttttctattccaagatacggttttactgctttgcaatacaacgaatttttgggtcaagaattttgtccaggttttaacgttactgataactctacttgtgttaactcttacgctatttgtactggtaacgaatacttgattaaccaaggtattgaattgtctccatggggtttgtggaagaaccatgttgctttggcttgtatgattattatttttttgactattgcttacttgaagttgttgtttttgaagaagtactcttaa

SEQ ID NO: 68 - protein, 657 aa, Mus musculus
MSSSNDHVLVPMSQRNNNGLPRMNSRAVRTLAEGDVLSFHHITYRVKVKSGFLVRKTVEKEILSDINGIMKPGLNAILGPTGGGKSSLLDVLAARKDPKGLSGDVLINGAPQPAHFKCCSGYVVQDDVVMGTLTVRENLQFSAALRLPTTMKNHEKNERINTIIKELGLEKVADSKVGTQFIRGISGGERKRTSIGMELITDPSILFLDEPTTGLDSSTANAVLLLLKRMSKQGRTIIFSIHQPRYSIFKLFDSLTLLASGKLVFHGPAQKALEYFASAGYHCEPYNNPADFFLDVINGDSSAVMLNREEQDNEANKTEEPSKGEKPVIENLSEFYINSAIYGETKAELDQLPGAQEKKGTSAFKEPVYVTSFCHQLRWIARRSFKNLLGNPQASVAQLIVTVILGLIIGAIYFDLKYDAAGMQNRAGVLFFLTTNQCFSSVSAVELFVVEKKLFIHEYISGYYRVSSYFFGKVMSDLLPMRFLPSVIFTCILYFMLGLKKTVDAFFIMMFTLIMVAYTASSMALAIATGQSVVSVATLLMTIAFVFMMLFSGLLVNLRTIGPWLSWLQYFSIPRYGFTALQYNEFLGQEFCPGFNVTDNSTCVNSYAICTGNEYLINQGIELSPWGLWKNHVALACMIIIFLTIAYLKLLFLKKYS

SEQ ID NO: 69 - DNA, 1515 bp, Mus musculus
atgaacttgttttctgctttgtctttggatactttggttttgttggctattattttggttttgttgtacagatacggtactagaactcatggtttgtttaagaagcaaggtattccaggtccaaagccattgccatttttgggtactgttttgaactactacactggtatttggaagtttgatatggaatgttacgaaaagtacggtaagacttggggtttgtttgatggtcaaactccattgttggttattactgatccagaaactattaagaacgttttggttaaggattgtttgtctgtttttactaacagaagagaatttggtccagttggtattatgtctaaggctatttctatttctaaggatgaagaatggaagagatacagagctttgttgtctccaacttttacttctggtagattgaaggaaatgtttccagttattgaacaatacggtgatattttggttaagtacttgagacaagaagctgaaaagggtatgccagttgctatgaaggatgttttgggtgcttactctatggatgttattacttctacttcttttggtgttaacgttgattctttgaacaacccagaagatccatttgttgaagaagctaagaagtttttgagagttgatttttttgatccattgttgttttctgttgttttgtttccattgttgactccagtttacgaaatgttgaacatttgtatgtttccaaacgattctattgaattttttaagaagtttgttgatagaatgcaagaatctagattggattctaaccaaaagcatagagttgattttttgcaattgatgatgaactctcataacaactctaaggataaggattctcataaggctttttctaacatggaaattactgttcaatctattatttttatttctgctggttacgaaactacttcttctactttgtcttttactttgtactgtttggctactcatccagatattcaaaagaagttgcaagctgaaattgataaggctttgccaaacaaggctactccaacttgtgatactgttatggaaatggaatacttggatatggttttgaacgaaactttgagattgtacccaattgttactagattggaaagagtttgtaagaaggatgttgaattgaacggtgtttacattccaaagggttctatggttatgattccatcttacgctttgcatcatgatccacaacattggccagatccagaagaatttcaaccagaaagattttctaaggaaaacaagggttctattgatccatacgtttacttgccatttggtattggtccaagaaactgtattggtatgagatttgctttgatgaacatgaagttggctgttactaaggttttgcaaaacttttcttttcaaccatgtcaagaaactcaaattccattgaagttgtctagacaaggtattttgcaaccagaaaagccaattgttttgaaggttgttccaagagatgctgttattactggtgcttaa

SEQ ID NO: 70 - protein, 504 aa, Mus musculus
MNLFSALSLDTLVLLAIILVLLYRYGTRTHGLFKKQGIPGPKPLPFLGTVLNYYTGIWKFDMECYEKYGKTWGLFDGQTPLLVITDPETIKNVLVKDCLSVFTNRREFGPVGIMSKAISISKDEEWKRYRALLSPTFTSGRLKEMFPVIEQYGDILVKYLRQEAEKGMPVAMKDVLGAYSMDVITSTSFGVNVDSLNNPEDPFVEEAKKFLRVDFFDPLLFSVVLFPLLTPVYEMLNICMFPNDSIEFFKKFVDRMQESRLDSNQKHRVDFLQLMMNSHNNSKDKDSHKAFSNMEITVQSIIFISAGYETTSSTLSFTLYCLATHPDIQKKLQAEIDKALPNKATPTCDTVMEMEYLDMVLNETLRLYPIVTRLERVCKKDVELNGVYIPKGSMVMIPSYALHHDPQHWPDPEEFQPERFSKENKGSIDPYVYLPFGIGPRNCIGMRFALMNMKLAVTKVLQNFSFQPCQETQIPLKLSRQGILQPEKPIVLKVVPRDAVITGA

SEQ ID NO: 71 - DNA, 2037 bp, Mus musculus
atgggtgattctcatgaagatacttctgctactgttccagaagctgttgctgaagaagtttctttgttttctactactgatattgttttgttttctttgattgttggtgttttgacttactggtttatttttaagaagaagaaggaagaaattccagaattttctaagattcaaactactgctccaccagttaaggaatcttcttttgttgaaaagatgaagaagactggtagaaacattattgttttttacggttctcaaactggtactgctgaagaatttgctaacagattgtctaaggatgctcatagatacggtatgagaggtatgtctgctgatccagaagaatacgatttggctgatttgtcttctttgccagaaattgataagtctttggttgttttttgtatggctacttacggtgaaggtgatccaactgataacgctcaagatttttacgattggttgcaagaaactgatgttgatttgactggtgttaagtttgctgtttttggtttgggtaacaagacttacgaacattttaacgctatgggtaagtacgttgatcaaagattggaacaattgggtgctcaaagaatttttgaattgggtttgggtgatgatgatggtaacttggaagaagattttattacttggagagaacaattttggccagctgtttgtgaattttttggtgttgaagctactggtgaagaatcttctattagacaatacgaattggttgttcatgaagatatggatactgctaaggtttacactggtgaaatgggtagattgaagtcttacgaaaaccaaaagccaccatttgatgctaagaacccatttttggctgctgttactactaacagaaagttgaaccaaggtactgaaagacatttgatgcatttggaattggatatttctgattctaagattagatacgaatctggtgatcatgttgctgtttacccagctaacgattctactttggttaaccaaattggtgaaattttgggtgctgatttggatgttattatgtctttgaacaacttggatgaagaatctaacaagaagcatccatttccatgtccaactacttacagaactgctttgacttactacttggatattactaacccaccaagaactaacgttttgtacgaattggctcaatacgcttctgaaccatctgaacaagaacatttgcataagatggcttcttcttctggtgaaggtaaggaattgtacttgtcttgggttgttgaagctagaagacatattttggctattttgcaagattacccatctttgagaccaccaattgatcatttgtgtgaattgttgccaagattgcaagctagatactactctattgcttcttcttctaaggttcatccaaactctgttcatatttgtgctgttgctgttgaatacgaagctaagtctggtagagttaacaagggtgttgctacttcttggttgagaactaaggaaccagctggtgaaaacggtagaagagctttggttccaatgtttgttagaaagtctcaatttagattgccatttaagccaactactccagttattatggttggtccaggtactggtgttgctccatttatgggttttattcaagaaagagcttggttgagagaacaaggtaaggaagttggtgaaactttgttgtactacggttgtagaagatctgatgaagattacttgtacagagaagaattggctagatttcataaggatggtgctttgactcaattgaacgttgctttttctagagaacaagctcataaggtttacgttcaacatttgttgaagagagataaggaacatttgtggaagttgattcatgaaggtggtgctcatatttacgtttgtggtgatgctagaaacatggctaaggatgttcaaaacactttttacgatattgttgctgaatttggtccaatggaacatactcaagctgttgattacgttaagaagttgatgactaagggtagatactctttggatgtttggtcttaa

SEQ ID NO: 72 - protein, 678 aa, Mus musculus
MGDSHEDTSATVPEAVAEEVSLFSTTDIVLFSLIVGVLTYWFIFKKKKEEIPEFSKIQTTAPPVKESSFVEKMKKTGRNIIVFYGSQTGTAEEFANRLSKDAHRYGMRGMSADPEEYDLADLSSLPEIDKSLVVFCMATYGEGDPTDNAQDFYDWLQETDVDLTGVKFAVFGLGNKTYEHFNAMGKYVDQRLEQLGAQRIFELGLGDDDGNLEEDFITWREQFWPAVCEFFGVEATGEESSIRQYELVVHEDMDTAKVYTGEMGRLKSYENQKPPFDAKNPFLAAVTTNRKLNQGTERHLMHLELDISDSKIRYESGDHVAVYPANDSTLVNQIGEILGADLDVIMSLNNLDEESNKKHPFPCPTTYRTALTYYLDITNPPRTNVLYELAQYASEPSEQEHLHKMASSSGEGKELYLSWVVEARRHILAILQDYPSLRPPIDHLCELLPRLQARYYSIASSSKVHPNSVHICAVAVEYEAKSGRVNKGVATSWLRTKEPAGENGRRALVPMFVRKSQFRLPFKPTTPVIMVGPGTGVAPFMGFIQERAWLREQGKEVGETLLYYGCRRSDEDYLYREELARFHKDGALTQLNVAFSREQAHKVYVQHLLKRDKEHLWKLIHEGGAHIYVCGDARNMAKDVQNTFYDIVAEFGPMEHTQAVDYVKKLMTKGRYSLDVWS

SEQ ID NO: 73 - protein, 492 aa, Arabidopsis thaliana
MDPYRVRPSSAHDSPFFTTNSGAPVWNNNSSLTVGTRGPILLEDYHLLEKLANFDRERIPERVVHARGASAKGFFEVTHDITQLTSADFLRGPGVQTPVIVRFSTVIHERGSPETLRDPRGFAVKFYTREGNFDLVGNNFPVFFVRDGMKFPDMVHALKPNPKSHIQENWRILDFFSHHPESLHMFSFLFDDLGIPQDYRHMEGAGVNTYMLINKAGKAHYVKFHWKPTCGIKCLSDEEAIRVGGANHSHATKDLYDSIAAGNYPQWNLFVQVMDPAHEDKFDFDPLDVTKIWPEDILPLQPVGRLVLNKNIDNFFNENEQIAFCPALVVPGIHYSDDKLLQTRIFSYADSQRHRLGPNYLQLPVNAPKCAHHNNHHDGFMNFMHRDEEVNYFPSRLDPVRHAEKYPTTPIVCSGNREKCFIGKENNFKQPGERYRSWDSDRQERFVKRFVEALSEPRVTHEIRSIWISYWSQADKSLGQKLATRLNVRPNF

SEQ ID NO: 74 - protein, 492 aa, Arabidopsis thaliana
MDPYKYRPASSYNSPFFTTNSGAPVWNNNSSMTVGPRGPILLEDYHLVEKLANFDRERIPERVVHARGASAKGFFEVTHDISNLTCADFLRAPGVQTPVIVRFSTVIHERGSPETLRDPRGFAVKFYTREGNFDLVGNNFPVFFIRDGMKFPDMVHALKPNPKSHIQENWRILDFFSHHPESLNMFTFLFDDIGIPQDYRHMDGSGVNTYMLINKAGKAHYVKFHWKPTCGVKSLLEEDAIRVGGTNHSHATQDLYDSIAAGNYPEWKLFIQIIDPADEDKFDFDPLDVTKTWPEDILPLQPVGRMVLNKNIDNFFAENEQLAFCPAIIVPGIHYSDDKLLQTRVFSYADTQRHRLGPNYLQLPVNAPKCAHHNNHHEGFMNFMHRDEEVNYFPSRYDQVRHAEKYPTPPAVCSGKRERCIIEKENNFKEPGERYRTFTPERQERFIQRWIDALSDPRITHEIRSIWISYWSQADKSLGQKLASRLNVRPSI

SEQ ID NO: 75 - protein, 492 aa, Arabidopsis thaliana
MDPYKYRPSSAYNAPFYTTNGGAPVSNNISSLTIGERGPVLLEDYHLIEKVANFTRERIPERVVHARGISAKGFFEVTHDISNLTCADFLRAPGVQTPVIVRFSTVVHERASPETMRDIRGFAVKFYTREGNFDLVGNNTPVFFIRDGIQFPDVVHALKPNPKTNIQEYWRILDYMSHLPESLLTWCWMFDDVGIPQDYRHMEGFGVHTYTLIAKSGKVLFVKFHWKPTCGIKNLTDEEAKVVGGANHSHATKDLHDAIASGNYPEWKLFIQTMDPADEDKFDFDPLDVTKIWPEDILPLQPVGRLVLNRTIDNFFNETEQLAFNPGLVVPGIYYSDDKLLQCRIFAYGDTQRHRLGPNYLQLPVNAPKCAHHNNHHEGFMNFMHRDEEINYYPSKFDPVRCAEKVPTPTNSYTGIRTKCVIKKENNFKQAGDRYRSWAPDRQDRFVKRWVEILSEPRLTHEIRGIWISYWSQADRSLGQKLASRLNVRPSI

SEQ ID NO: 76 - DNA, 23 bp, Artificial Sequence
  Forward primer of CYP3A4
tgcctaataaagctcctcctact

SEQ ID NO: 77 - DNA, 22 bp, Artificial Sequence
  Reverse primer of CYP3A4
gctcctgaaacagttccatctc

SEQ ID NO: 78 - DNA, 22 bp, Artificial Sequence
  Forward primer of P450 oxidoreductase
ggaagagctttggttcctatgt

SEQ ID NO: 79 - DNA, 23 bp, Artificial Sequence
  Reverse primer of P450 oxidoreductase
gctcccaattcagcaacaatatc

SEQ ID NO: 80 - DNA, 29 bp, Artificial Sequence
  Forward primer of CBDA synthase
acatcacaatcacacaaaactaacaaaag

SEQ ID NO: 81 - DNA, 22 bp, Artificial Sequence
  Reverse primer of CBDA synthase
ggccatagtttctcatcaatgg

SEQ ID NO: 82 - DNA, 28 bp, Artificial Sequence
  Forward primer of UGT76G1
gattggaagaacaagcttcaggatttcc

SEQ ID NO: 83 - DNA, 26 bp, Artificial Sequence
  Reverse primer of UGT76G1
ccatcctgaatgagtccaaaaagctc

SEQ ID NO: 84 - DNA, 22 bp, Artificial Sequence
  Forward primer of ABCG2
ccttcaggattgtcaggagatg

SEQ ID NO: 85 - DNA, 22 bp, Artificial Sequence
  Reverse primer of ABCG2
gcaggtccatgaaacatcaatc

SEQ ID NO: 86 - DNA, 27 bp, Artificial Sequence
  Forward primer of Trichomoe-targeted CBDAs
aaagatcaaaagcaagttcttcactgt

SEQ ID NO: 87 - DNA, 25 bp, Artificial Sequence
  Reverse primer of Trichome-targeted CBDAs
ccatgcagtttggctatgaacatct

SEQ ID NO: 88 - DNA, 25 bp, Artificial Sequence
  Forward primer of Trichome-targeted UGT
agtgctcaacattctccttttggtt

SEQ ID NO: 89 - DNA, 25 bp, Artificial Sequence
  Reverse primer of Trichome-targeted UGT
tctgaagccaacatcaacaattcca

SEQ ID NO: 90 - DNA, 25 bp, Artificial Sequence
  Forward primer of Plasma membrane-targeted UTRI
ttgttccttaaacctcgcctttgac

SEQ ID NO: 91 - DNA, 25 bp, Artificial Sequence
  Reverse primer of Plasma membrane-targeted UTRI
tcattatggagcactccactctctg

SEQ ID NO: 92 - DNA, 27 bp, Artificial Sequence
  Forward primer of Cytosolic-targeted CBDA synthase
aaagatcaaaagcaagttcttcactgt

SEQ ID NO: 93 - DNA, 25 bp, Artificial Sequence
  Reverse primer of Cytosolic-targeted CBDA synthase
ataaacttctccaagggtagctccg

SEQ ID NO: 94 - DNA, 25 bp, Artificial Sequence
  Forward primer of Cytosolic-targeted UGT
agaactggaagaatccgaactggaa

SEQ ID NO: 95 - DNA, 25 bp, Artificial Sequence
  Reverse primer of Cytosolic-targeted UGT
aaatcatcgggacaccttcacaaac

SEQ ID NO: 96 - DNA, 25 bp, Artificial Sequence
  Forward primer of NtGT1
atgaaaacaacagaacttgtcttca

SEQ ID NO: 97 - DNA, 22 bp, Artificial Sequence
  Reverse primer of NtGT1
tgaagttgtaggcctagcatgg

SEQ ID NO: 98 - DNA, 25 bp, Artificial Sequence
  Forward primer of NtGT2
atggttcaaccacacgtcttactgg

SEQ ID NO: 99 - DNA, 22 bp, Artificial Sequence
  Reverse primer of NtGT2
ttgaatacaccagttggggtcg

SEQ ID NO: 100 - DNA, 25 bp, Artificial Sequence
  Forward primer of NtGT3
atgaaagagactaaaaaaattgagt

SEQ ID NO: 101 - DNA, 23 bp, Artificial Sequence
  Reverse primer of NtGT3
catcacgcagattttgaatatgg

SEQ ID NO: 102 - DNA, 25 bp, Artificial Sequence
  Forward primer of NtGT4
atggctactcaggtgcataaattgc

SEQ ID NO: 103 - DNA, 22 bp, Artificial Sequence
  Reverse primer of NtGT4
ggccttagttagctcgacacgg

SEQ ID NO: 104 - DNA, 25 bp, Artificial Sequence
  Forward primer of NtGT5
atgggctctatcggtgcagaactaa

SEQ ID NO: 105 - DNA, 22 bp, Artificial Sequence
  Reverse primer of NtGT5
cggggatgaagtccaaggttgt

SEQ ID NO: 106 - DNA, 25 bp, Artificial Sequence
  Forward primer of Kat-E
atgtctcaacataacgagaaaaacc

SEQ ID NO: 107 - DNA, 22 bp, Artificial Sequence
  Reverse primer of Kat-E
cgtagcaaatcccctgatgtct

SEQ ID NO: 108 - DNA, 25 bp, Artificial Sequence
  Forward primer of UGT76G1
atggagaacaaaaccgagacaaccg

SEQ ID NO: 109 - DNA, 22 bp, Artificial Sequence
  Reverse primer of UGT76G1
cctttagcatgggaaaaccgga

SEQ ID NO: 110 - DNA, 28 bp, Artificial Sequence
  Forward primer of UGT76G1 (for tobacco BY2 cells)
gattggaagaacaagcttcaggatttcc

SEQ ID NO: 111 - DNA, 26 bp, Artificial Sequence
  Reverse primer of UGT76G1 (for tobacco BY2 cells)
ccatcctgaatgagtccaaaaagctc

SEQ ID NO: 112 - DNA, 22 bp, Artificial Sequence
  Forward primer of ABCG2 (for tobacco BY2 cells)
ccttcaggattgtcaggagatg

SEQ ID NO: 113 - DNA, 22 bp, Artificial Sequence
  Reverse primer of ABCG2 (for tobacco BY2 cells)
gcaggtccatgaaacatcaatc

(19) United States
(12) Patent Application Publication
(10) Pub. No.: US 2020/0046639 A1
(43) Pub. Date: Feb. 13, 2020

(54) CONSUMABLE WATER-SOLUBLE CANNABINOID FOOD AND BEVERAGE ADDITIVE HAVING ENHANCED STABILITY

(71) Applicant: Trait Biosciences Inc., Los Alamos, NM (US)

(72) Inventors: Richard T. Sayre, Los Alamos, NM (US); Elton Carvalho Gonçalves, Los Alamos, NM (US); Tawanda Zidenga, White Rock, NM (US); Stephanie Willette, Santa Fe, NM (US)

(21) Appl. No.: 16/425,744

(22) Filed: May 29, 2019

Related U.S. Application Data
(63) Continuation-in-part of application No. 16/110,954, filed on Aug. 23, 2018.
Application No. 16/110,954 is a continuation-in-part of application No. PCT/US18/41710, filed on Jul. 11, 2018.
Application No. PCT/US18/41710 is a continuation-in-part of application No. PCT/US18/24409, filed on Mar. 26, 2018.
(60) Provisional application No. 62/531,123, filed on Jul. 11, 2017.
(60) Provisional application No. 62/588,662, filed on Nov. 20, 2017.
(60) Provisional application No. 62/621,166, filed on Jan. 24, 2018.

(51) Int. Cl. A61K 9/00; A61K 31/7034; A61K 9/08; A61K 47/36; A61K 47/26; A23L 33/105; A23L 33/125; C12G 3/04

(52) U.S. Cl. CPC A61K 9/0095; A61K 31/7034; A61K 9/08; A61K 47/36; A23V 2002/00; A23L 33/105; A23L 33/125; C12G 3/04; A61K 47/26

(57) ABSTRACT

The present invention includes systems, methods and compositions related to the production and use of chemically modified cannabinoid food and beverage additives having enhanced water solubility and stability.

### CROSS REFERENCE TO RELATED APPLICATIONS

This application is a Continuation-in-Part of U.S. application Ser. No. 16/110,954, filed Aug. 23, 2018; which is a Continuation-in-Part of International Application No. PCT/US18/41710, filed Jul. 11, 2018; which claims the benefit of and priority to U.S. Provisional Application No. 62/531,123, filed Jul. 11, 2017. International Application No. PCT/US18/41710, filed Jul. 11, 2018 is a Continuation-in-Part of International Application No. PCT/US18/24409, filed Mar. 26, 2018; which claims the benefit of and priority to U.S. Provisional Application Nos. 62/588,662, filed Nov. 20, 2017 and 62/621,166, filed Jan. 24, 2018.

### SEQUENCE LISTING

The instant application contains a Sequence Listing which has been submitted electronically in ASCII format and is hereby incorporated by reference in its entirety.

### TECHNICAL FIELD

The field of the present invention also relates generally to consumable compositions of matter that may contain one or more water-soluble cannabinoids.

### BACKGROUND

Cannabinoids are a class of specialized compounds synthesized by Cannabis. They are formed by condensation of terpene and phenol precursors. They include these more abundant forms: Delta-9-tetrahydrocannabinol (THC), cannabidiol (CBD), cannabichromene (CBC), and cannabigerol (CBG). Another cannabinoid, cannabinol (CBN), is formed from THC as a degradation product and can be detected in some plant strains. Typically, THC, CBD, CBC, and CBG occur together in different ratios in the various plant strains.

Cannabinoids are generally classified into two types, neutral cannabinoids and cannabinoid acids, based on whether they contain a carboxyl group or not. It is known that, in fresh plants, the concentrations of neutral cannabinoids are much lower than those of cannabinoid acids. One strain Cannabis sativa contains approximately 61 compounds belonging to the general class of cannabinoids. These cannabinoids are generally lipophilic, nitrogen-free, mostly phenolic compounds, and are derived biogenetically from a monoterpene and phenol, the acid cannabinoids from a monoterpene and phenol carboxylic acid, and have a C21 to base material.

Cannabinoids also find their corresponding carboxylic acids in plant products. In general, the carboxylic acids have the function of a biosynthetic precursor. For example, these compounds arise in vivo from the THC carboxylic acids by decarboxylation the tetrahydrocannabinols Δ9- and Δ8-THC and CBD from the associated cannabidiol. As generally shown in FIG. 28, THC and CBD may be derived artificially from their acidic precursor's tetrahydrocannabinolic acid (THCA) and cannabidiolic acid (CBDA) by non-enzymatic decarboxylation.

Cannabinoids are widely consumed, in a variety of forms around the world. Cannabinoid-rich preparations of Cannabis, either in herb (i.e. marijuana) or resin form (i.e., hash oil), are used by an estimated 2.6-5.0% of the world population (UNODC, 2012). Cannabinoid containing pharmaceutical products, either containing natural cannabis extracts (Sativex®) or the synthetic cannabinoids dronabinol or nabilone, are available for medical use in several countries

As noted above, Δ-9-tetrahydrocannabinol (also known as THC) is one of the main biologically active components in the Cannabis plant which has been approved by the Food and Drug Administration (FDA) for the control of nausea and vomiting associated with chemotherapy and, more recently, for appetite stimulation of AIDS patients suffering from wasting syndrome. The drug, however, shows other biological activities which lend themselves to possible therapeutic applications, such as in the treatment of glaucoma, migraine headaches, spasticity, anxiety, and as an analgesic.

Indeed, it is well documented that agents, such as cannabinoids and endocannabinoids that activate cannabinoid receptors in the body modulate appetite, and alleviate nausea, vomiting, and pain (Martin B. R. and Wiley, J. L, Mechanism of action of cannabinoids: how it may lead to treatment of cachexia, emesis and pain, Journal of Supportive Oncology 2: 1-10, 2004), multiple sclerosis (Pertwee, R. G., Cannabinoids and multiple sclerosis, Pharmacol. Ther. 95, 165-174, 2002), and epilepsy (Wallace, M. J., Blair, R. E., Falenski, K. WW., Martin, B. R., and DeLorenzo, R. J. Journal Pharmacology and Experimental Therapeutics, 307: 129-137, 2003). In addition, CB2 receptor agonists have been shown to be effective in treating pain (Clayton N., Marshall F. H., Bountra C., O'Shaughnessy C. T., 2002. CB1 and CB2 cannabinoid receptors are implicated in inflammatory pain. 96, 253-260; Malan T. P., Ibrahim M. M., Vanderah T. W., Makriyannis A., Porreca F., 2002. Inhibition of pain responses by activation of CB(2) cannabinoid receptors. Chemistry and Physics of Lipids 121, 191-200; Malan T. P., Jr., Ibrahim M. M., Deng H., Liu Q., Mata H. P., Vanderah T., Porreca F., Makriyannis A., 2001. CB2 cannabinoid receptor-mediated peripheral antinociception. 93, 239-245; Quartilho A., Mata H. P., Ibrahim M. M., Vanderah T. W., Porreca F., Makriyannis A., Malan T. P., Jr., 2003. Inhibition of inflammatory hyperalgesia by activation of peripheral CB2 cannabinoid receptors. Anesthesiology 99, 955-960) and multiple sclerosis (Pertwee, R. G., Cannabinoids and multiple sclerosis, Pharmacol. Ther. 95, 165-174, 2002) in animal models.

More recently, several states have approved use of Cannabis and cannabinoid infused products for both recreational and medical uses. As these new medical and commercial markets have developed, there has grown a need to develop more efficient production and isolation of cannabinoid compounds. Traditional methods of cannabinoid production typically focus on extraction and purification of cannabinoids from raw harvested Cannabis. However, traditional cannabinoid extraction and purification methods have a number of technical and practical problems that limits its usefulness.

Limitations of Traditional Cannabinoid Production and Extraction Methods

For example, in U.S. Pat. No. 6,403,126 (Webster et al.), cannabinoids, and other related compounds are isolated from raw harvested Cannabis and treated with an organic solvent, typically a petroleum derived hydrocarbon, or a low molecular-weight alcohol to solubilize the cannabinoids for later isolation. This traditional method is limited in that it relies on naturally grown plant matter that may have been exposed to various toxic pesticides, herbicides and the like. In addition, such traditional extraction methods are imprecise resulting in unreliable and varied concentrations of extracted THC. In addition, many Cannabis strains are grown in hydroponic environments which are also not regulated and can results in the widespread contamination of such strains with chemical and other undesired compounds.

In another example, US Pat. App. No. 20160326130 (Lekhram et al.), cannabinoids, and other related compounds are isolated from raw harvested Cannabis using, again, a series of organic solvents to convert the cannabinoids cannabinoids into a salt, and then back to its original carboxylic acid form. Similar to Webster, this traditional method is limited in that is relies on naturally grown plant matter that may have been exposed to various toxic pesticides, herbicides and the like. In addition, the multiple organic solvents used in this traditional process must be recovered and either recycled and/or properly disposed of.

Another traditional method of cannabinoid extraction involves the generation of hash oils utilizing supercritical carbon-dioxide (sCO₂). Under this traditional method, again the dried plant matter is ground and subjected to a sCO₂ extraction environment. The primary extract being initially obtained and further separated. For example, as generally described by CA2424356 (Muller et al.) cannabinoids are extracted with the aid of sCO₂ under supercritical pressure and temperature conditions and by the addition of accessory solvents (modifiers) such as alcohols. Under this process, this supercritical CO₂ evaporates and dissolves into the cannabinoids. However, this traditional process also has certain limiting disadvantages. For example, due to the low solubility in supercritical sCO₂, recovery of the cannabinoids of interest is inconsistent. Additionally, any solvents used must be recycled and pumped back to the extractor, in order to minimize operating costs.

Another method utilizes butane to extract cannabinoids, in particular high concentrations of THC, from raw harvested Cannabis. Because butane is non-polar, this process does not extract water soluble by-products such as chlorophyll and plant alkaloids. That said, this process may take up to 48 hours and as such is limited in its ability to scale-up for maximum commercial viability. The other major drawback of traditional butane-based extraction processes is the potential dangers of using flammable solvents, as well as the need to ensure all of the butane is fully removed from the extracted cannabinoids.

Another limiting factor in the viability of these traditional methods of cannabinoid extraction methods is the inability to maintain Cannabis strain integrity. For example, cannabinoids used in medical and research applications, or that are subject to controlled clinical trials, are tightly regulated by various government agencies in the United States and elsewhere. These regulatory agencies require that the Cannabis strains remain chemically consistent over time. Unfortunately, the genetic/chemical compositions of the Cannabis strains change over generations such that they cannot satisfy regulatory mandates present in most clinical trials or certified for use in other pharmaceutical applications.

Several attempts have been made to address these concerns. For example, efforts have been made to produce cannabinoids in genetically engineered organisms. For example, in U.S. patent application Ser. No. 14/795,816 (Poulos, et al.) Here, the applicant claims to have generated a genetically modified strain of yeast capable of producing a cannabinoid by inserting genes that produce the appropriate enzymes for its metabolic production. However, such application is limited in its ability to produce only a single or very limited number of cannabinoid compounds. This limitation is clinically significant. Recent clinical studies have found that the use of a single isolated cannabinoid as a therapeutic agent is not as effective as treatment with the naturally-occurring “entourage” of primary and secondary cannabinoids associated with various select strains. The system in Poulos is further limited in the ability to account for toxic by-products of cannabinoid synthesis, as well as the directly toxic effects of the insoluble, and/or only lipid-soluble, cannabinoid compounds themselves.

Additional attempts have been made to chemically synthesize cannabinoids, such as THC. However, the chemical synthesis of various cannabinoids is a costly process when compared to the extraction of cannabinoids from naturally occurring plants. The chemical synthesis of cannabinoids also involves the use of chemicals that are not environmentally friendly, which can be considered as an additional cost to their production. Furthermore, the synthetic chemical production of various cannabinoids has been classified as less pharmacologically active as those extracted from plants such as Cannabis sativa.

Efforts to generate large-scale Cannabis cell cultures have also raised a number of technical problems. Chief among them is the fact that cannabinoids are cytotoxic. Under natural conditions cannabinoids are generated and then stored extracellularly in small glandular structures called trichomes. Trichomes can be visualized as small hairs or other outgrowths from the epidermis of a Cannabis plant. As a result, in Cannabis cell cultures, the inability to store cannabinoids extracellularly means any accumulation of cannabinoids would be toxic to the cultured cells. Such limitations impair the ability of Cannabis cell cultures to be scaled-up for industrial levels of production.

Cannabinoid Biosynthesis Toxicity Limits In Vivo Production Systems

Efforts to generate Cannabis strains/cell cultures that produce or accumulate high-levels of cannabinoids have raised a number of technical problems. Chief among them is the fact that cannabinoid synthesis produces toxic by-products. Notably, both CBDA and THCA synthases require molecular oxygen, in conjunction with a molecule of FAD, to oxidize Cannabigerolic acid (CBGA). Specifically, as shown in FIG. 29, two electrons from the substrate are accepted by an enzyme-bound FAD, and then transferred to molecular oxygen to re-oxidize FAD. CBDA and THCA are synthesized from the ionic intermediates via stereoselective cyclization by the enzymes. The hydride ion is transferred from the reduced flavin to molecular oxygen, resulting in the formation of hydrogen peroxide and re-activation of the flavin for the next cycle. As a result, in addition to producing CBDA and THCA respectively, this reaction produces hydrogen peroxide (H₂O₂) which is naturally toxic to the host cell. Due to this production of a toxic hydrogen peroxide byproduct, cannabinoid synthesis generates a self-limiting feed-back loop preventing high-level production and/or accumulation of cannabinoids in in vivo systems. One way that Cannabis plants deal with these cellular cytotoxic effects is through the use of trichomes for Cannabinoid production and accumulations.

Cannabis plants deal with this toxicity by sequestering cannabinoid biosynthesis and storage extracellularly in small glandular structures called trichomes as note above. For example, THCA synthase is a water soluble enzyme that is responsible for the production of THC. For example, THC biosynthesis occurs in glandular trichomes and begins with condensation of geranyl pyrophosphate with olivetolic acid to produce cannabigerolic acid (CBGA); the reaction is catalyzed by an enzyme called geranylpyrophosphate:olivatolate geranyltransferase. CBGA then undergoes oxidative cyclization to generate tetrahydrocannabinolic acid (THCA) in the presence of THCA synthase. THCA is then transformed into THC by non-enzymatic decarboxylation. Sub-cellular localization studies using RT-PCR and enzymatic activity analyses demonstrate that THCA synthase is expressed in the secretory cells of glandular trichomes, and then is translocated into the secretory cavity where the end product THCA accumulates. THCA synthase present in the secretory cavity is functional, indicating that the storage cavity is the site for THCA biosynthesis and storage. In this way, the Cannabis is able to produce cannabinoids extracellularly and thereby avoid the cytotoxic effects of these compounds. However, as a result, the ability to access and chemically alter cannabinoids in vivo is impeded by this cellular compartmentalization.

To address these concerns, some have proposed chemically modifying cannabinoid compounds to reduce their cytotoxic effects. For example, Zipp, et al., have proposed utilizing an in vitro method to produce cannabinoid glycosides. However, this application is limited to in vitro systems only. Specifically, as noted above, cannabinoid synthase enzymes, such as THCA synthase, are water soluble proteins that are exported out of the basal trichome cells into the storage compartment where it is active and catalyzes the synthesis of THCA. Specifically, in order to effectively mediate the cellular export of such cannabinoid synthase, this enzyme contains a 28 amino acid signal peptide that directs its export out of the cell and into the extracellular trichrome where cannabinoid synthesis occurs.

The foregoing problems regarding the production, detoxification and isolation of cannabinoids may represent a long-felt need for an effective—and economical—solution to the same. While implementing elements may have been available, actual attempts to meet this need may have been lacking to some degree. This may have been due to a failure of those having ordinary skill in the art to fully appreciate or understand the nature of the problems and challenges involved.

As a result of this lack of understanding, attempts to meet these long-felt needs may have failed to effectively solve one or more of the problems or challenges here identified. These attempts may even have led away from the technical directions taken by the present inventive technology and may even result in the achievements of the present inventive technology being considered to some degree an unexpected result of the approach taken by some in the field.

As will be discussed in more detail below, the current inventive technology overcomes the limitations of traditional cannabinoid production systems while meeting the objectives of a truly effective and scalable cannabinoid production, modification and isolation system.

### SUMMARY OF THE INVENTION(S)

Generally, the inventive technology relates to the field of chemical modification and isolation in yeast suspension cultures. The present inventive technology further relates to improved systems and methods for the modification and isolation of pharmaceutically active components from plant materials. In one embodiment, the inventive technology may encompass a novel system for the generation of chemically modified-cannabinoid compounds in a yeast suspension culture. The inventive technology may include systems and methods for high-efficiency chemical modification and isolation of cannabinoid compounds from yeast suspension cultures. In this embodiment, various select cannabinoid compounds may be chemically modified into soluble and non-toxic configurations.

One aim of the current inventive technology includes improved systems and methods for the modification of cannabinoids in a sterile yeast and/or plant culture system. In one embodiment, the inventive technology may include the production of a sterile yeast and/or plant cell suspension culture. The inventive technology may allow for certain transgenes to be introduced into these yeast strains and/or plant to transiently modify the chemical structure of the cannabinoid compounds. This transient modification may render the cannabinoids soluble in water. Such modifications may also alter the rate at which the cannabinoids are metabolized generating a modified cannabinoid with enhanced kinetics that may be used in certain therapeutic applications or as a prodrug. These transiently modified cannabinoids, aided by their modified chemical structure, may be allowed to accumulate at higher than native levels without having a deleterious effect on the cultured yeast and/or plant cells. Being soluble, they may also be secreted through endogenous and/or exogenous ABC or other trans-membrane protein transporters into the culture medium for later harvesting and isolation. It is noted that naturally occurring cannabinoids are strong inhibitors of ABC transporters. These transiently modified cannabinoids may be harvested and isolated from the aforementioned culture systems, and then enzymatically restored to their original chemical structure. Other embodiments may allow for the regulation of cannabinoid modification and isolation. In such embodiment, discreet and known amounts of cannabinoids may be introduced into a yeast and/or plant suspension culture and transiently modified. Later, the modified cannabinoids may be extracted from the cell culture and isolated such that the quantity and relative ratios of the various cannabinoids is known and quantifiable. In this manner the isolated cannabinoid extract may be chemically consistent and as such, easily dosable for both pharmaceutical and/or recreational applications.

Additional aims of the inventive technology may include the transient modification of cannabinoid compounds to render them water-soluble in yeast cell culture systems. In a preferred embodiment, such soluble cannabinoids may have reduced cytotoxicity to yeast cells in culture and may further be actively transported out of the cell and allowed to accumulate at levels that would normally have a deleterious effect on the cell culture. Additional embodiments may include the isolation of these transiently modified cannabinoids followed by enzymatic conversion or reconstitution to their original and/or partially modified structure.

Another aim of the current invention may include the systems, methods and compositions for the generation of water-soluble cannabinoid compounds. Another aim of the current inventive technology includes the generation of various compositions of matter containing water-soluble cannabinoids. In one preferred embodiment, such compositions of matter may contain water-soluble cannabinoids generated in an in vitro and/or in vivo system.

Additional aims of the invention may include delivery systems and compositions that include water-soluble cannabinoids, preferably glycosylated and/or acetylated cannabinoids. Additional embodiments may further include methods and systems for the production of compositions that include water-soluble cannabinoids, preferably glycosylated and/or acetylated cannabinoids.

Another aim of the current invention may include systems, methods and compositions for the delivery of water-soluble cannabinoids, preferably glycosylated and/or acetylated cannabinoids as a prodrug. Included in this invention may include novel prodrug compositions.

One aim of the invention may include systems, methods and compositions for the in vivo production, modification and isolation of cannabinoid compounds from Cannabis plants. In particular, the invention provides systems and methods for high level in vivo biosynthesis of water-soluble cannabinoids in yeast. In one preferred embodiment, the suspension culture may include the biotransformation of one or more cannabinoids in yeast, or other plant cells into a water-soluble form.

One aim of the invention may include systems, methods and compositions for the in vivo production, modification and isolation of cannabinoid compounds from Cannabis plants. In particular, the invention provides systems and methods for high level in vivo biosynthesis of water-soluble cannabinoids in cell suspension cultures. In one preferred embodiment, the suspension culture may include a yeast suspension culture, a tobacco or other plant cell suspension culture or a cannabis plant cell suspension culture.

The current inventive technology includes systems and methods for enhanced production and/or accumulation of cannabinoids. In one embodiment, the invention may include systems and methods for enhanced production and/or accumulation of cannabinoids in an in vivo system, such as a yeast, or plant cell suspension culture.

Another aim of the current invention may include the generation of genetically modified plants cells that may further be in a suspension culture that may overexpress certain endogenous/exogenous genes that result in the over-production and/or accumulation of cannabinoids above wild-type levels. In one preferred embodiment, such transgenic plant cell cultures may exhibit enhanced production and accumulation of cannabinoid precursor compounds, such as THCA (tetrahydrocannabinolic acid), CBCA (cannabichromenic acid), and CBDA (cannabidiolic acid). Such transgenic plant cells in culture may additionally exhibit enhanced production and localized accumulation of cannabinoids, such as THCs, CBCs and CBDs.

An additional aim of the current invention may include the generation of genetically modified plant cells in culture expressing certain endogenous/exogenous that result in the enhanced biomodification of cannabinoids. In one preferred embodiment, such cultured transgenic plant cells may exhibit enhanced modification of cannabinoids including hydroxylation, and/or acetylation, and/or glycosylation. In additional preferred embodiments, such transgenic plants may exhibit enhanced modification of cannabinoids including acetylation and glycosylation, such as an O acetylated glycoside form. For example, acetylation adds an acetyl group (—CH₃OOH) to a cannabinoid such that the carboxylate group is acidic and charged at neutral pH making it highly water-soluble.

Another aim of the current invention may include the generation of genetically modified yeast strains overexpressing certain endogenous/exogenous genes that result in the over-production and/or accumulation of cannabinoids above wild-type levels. In one preferred embodiment, such transgenic yeast may exhibit enhanced production and localized accumulation of cannabinoid precursor compounds, such as THCA (tetrahydrocannabinolic acid), CBCA (cannabichromenic acid), and CBDA (cannabidiolic acid). Such transgenic plants may additionally exhibit enhanced production and localized accumulation of cannabinoids, such as THCs, CBCs and CBDs.

An additional aim of the current invention may include the generation of genetically modified plants expressing certain genes that result in the modification of cannabinoids into water-soluble forms. In one preferred embodiment, such transgenic yeast may exhibit enhanced modification of cannabinoids including hydroxylation, and/or acetylation, and/or glycosylation. In additional preferred embodiments, such transgenic plants may exhibit enhanced modification of cannabinoids including acetylation and glycosylation, such as an O acetyl glycoside form. For example, acetylation adds an acetate group (—CH₃COOH) to a cannabinoid such that the carboxylate group is acidic and charged at neutral pH making it highly water-soluble.

One aim of the current inventive technology may be to generate genetically modified, or transgenic plant cells in a suspension culture that overexpresses one or more transcription factors, such as myb, that enhance metabolite flux through the cannabinoid biosynthetic pathway. In one preferred embodiment, these transcription factors may include various analogues. In certain preferred embodiments, one or more of these transgenes may be operably-linked to one or more promoters.

One aim of the current inventive technology may be to generate genetically modified or transgenic Cannabis plant cells in a suspension culture that overexpresses one or more transcription factors, such as myb, that enhance metabolite flux through the cannabinoid biosynthetic pathway. In one preferred embodiment, these transcription factors may include various analogues. In certain preferred embodiment, one or more of these transgenes may be operably-linked to one or more promoters.

Another aim of the current inventive technology may be to generate a genetically modified or transgenic tobacco cell culture that overexpresses one or more transcription factors that enhance metabolite flux through the cannabinoid biosynthetic pathway. In one preferred embodiment, these transgenes may be operably linked to one or more promoters.

Yet, another aim of the current inventive technology may be to generate a genetically modified or transgenic plant cell that expresses an enzyme that is configured to be capable of reducing hydrogen peroxide (H₂O₂) levels that may be generated during cannabinoid synthesis. In one preferred embodiment, the current inventive technology may be to generate a genetically modified or transgenic tobacco and/or Cannabis plant cell in a suspension culture that expresses a catalase protein. In this embodiment, this catalase protein may reduce hydrogen peroxide (H₂O₂) levels generated during cannabinoid synthesis.

Yet, another aim of the current inventive technology may be to generate genetically modified plants, plant cells and/or yeast cells that expresses an enzyme that is configured to be capable of reducing hydrogen peroxide (H₂O₂) levels that may be generated during cannabinoid synthesis. In one preferred embodiment, the current inventive technology may be to generate a genetically modified or transgenic yeast cell in a suspension culture that expresses a catalase protein. In this embodiment, this catalase protein may reduce hydrogen peroxide (H₂O₂) levels generated during cannabinoid synthesis.

Another aim of the current invention may include the introduction of one or more compounds to facilitate the chemical decomposition of hydrogen peroxide resulting from cannabinoids biosynthesis. In one preferred embodiment, one or more chemicals, metal ions, and/or catalysts may be introduced into a growth media to detoxify hydrogen peroxide (H₂O₂) in both yeast and plant cell cultures. It should be noted that additional cell cultures and cell lines may be contemplated in the invention. For example, CHO cells, HeLa cells and insect cell lines, like SF-9 cells may be genetically modified as generally described herein to generate water-soluble cannabinoids.

Additional embodiments of the inventive technology may include the transient modification of cannabinoid compounds to reduce and/or eliminate their cytotoxicity in plants or plant cell culture systems. In a preferred embodiment, such transiently modified cannabinoids may be allowed to accumulate at levels that would normally have a deleterious effect on the cell. Additional embodiments may include the isolation of these transiently modified cannabinoids followed by enzymatic conversion or reconstitution to their original and/or partially modified structure.

Another aim of the invention may include the generation of a transgenic plant and or plant cell cultures that may over express endogenous genes that may be configured to modify cannabinoids. Additional aim may include the co-expression of heterologous transcription factors that may increase cannabinoid production. Another aim of the invention may include the co-expression of heterologous genes that detoxify the hydrogen peroxide byproducts generated through cannabinoid biosynthesis. Co-expression of such genes may be additive with the co-expression of genes configured to modify and/or localize cannabinoid biomodifications.

Another aim of the invention may include systems, methods and compositions for the generation of a yeast cannabinoid production system coupled with systems, methods and compositions for the reducing hydrogen peroxide toxicity resulting from cannabinoid synthesis. Another aim of the invention may include systems, methods and compositions for the generation of a yeast cannabinoid production system coupled with systems, methods and compositions for the biomodification of such yeast generated cannabinoids into functionalized as well as water-soluble forms as generally described herein.

Additional embodiments of the invention may include:

1. A chemically modified cannabinoid food and beverage additive having enhanced water solubility comprising:
an aqueous solution;
at least one chemically modified cannabinoid dissolved in said aqueous solution, wherein said chemically modified cannabinoid comprises an water soluble acetylated cannabinoid glycoside; and
wherein said consumable fluid composition may be introduced to a food or beverage, and wherein said water soluble acetylated cannabinoid glycoside exhibits an enhanced ability to stay in solution compared to a cannabinoid glycoside, and an unmodified cannabinoid.
   2. The consumable fluid composition of embodiment 1, wherein said one or more water soluble acetylated cannabinoid glycosides comprises a water soluble 0-acetyl cannabinoid glycoside.
   3. The consumable fluid composition of embodiment 1, wherein said water soluble acetylated cannabinoid glycoside comprises a water soluble acetylated cannabinoid glycoside generated in vivo through incubation of a cannabinoid in a tobacco cell suspension culture.
   4. The consumable fluid composition of embodiment 1, wherein said water soluble acetylated cannabinoid glycoside comprises a water soluble acetylated cannabinoid glycoside generated in vitro through incubation with a glycosyltransferase protein having glycosylation activity towards one or more cannabinoids and acetyltransferase protein having acetylation activity toward one or more cannabinoids.
   5. The consumable fluid composition of embodiment 1, wherein said glycosyltransferase protein having glycosylation activity towards one or more cannabinoids comprises a glycosyltransferase protein from tobacco selected from the group consisting of: NtGT3; NtGT4; and NtGT5, and a homolog or ortholog of NtGT3, NtGT4, and NtGT5 having glycosylation activity towards one or more cannabinoids.
   6. The consumable fluid composition of embodiment 1, wherein said aqueous solution comprises an aqueous solution selected from the group consisting of: water, saline, purified water, propylene glycol, deionized water, or combinations thereof.
   7. The consumable fluid composition of embodiment 1, wherein said composition is added to a quantity of an alcohol beverage, wherein said quantity of alcohol is selected from the group consisting of: a quantity of ethanol, a quantity of beer, a quantity of wine, and a quantity of distilled spirits.
   8. The consumable fluid composition of embodiment 1, and further comprising at least one buffer.
   9. The consumable fluid composition of embodiment 8, wherein said at least one buffer maintains said aqueous solution at a pH at or below 7.4.
   10. The consumable fluid composition of embodiment 1, wherein the cannabinoid to be chemically modified into said water soluble acetylated cannabinoid glycoside comprises a cannabinoid from hemp.
   11. The consumable fluid composition of embodiment 10, wherein said cannabinoid from hemp comprises is a non-psychoactive cannabinoid.
   12. A chemically modified cannabinoid food additive comprising:
a consumable food additive composition of embodiment comprising at least one chemically modified cannabinoid provided in a dehydrated powder or liquid form, and wherein said chemically modified cannabinoid comprises a cannabinoid glycoside, an acetylated cannabinoid glycoside, or a mixture of both; and
wherein said consumable food additive composition may be introduced to a food or beverage, and wherein said cannabinoid glycoside, and said acetylated cannabinoid glycoside exhibit enhanced solubility compared to unmodified cannabinoids.
   13. The consumable food additive composition of embodiment 12, wherein said one or more water soluble acetylated cannabinoid glycosides comprises a water soluble 0-acetyl cannabinoid glycoside.
   14. The consumable food additive composition of embodiment 12, wherein said cannabinoid glycoside comprises a cannabinoid glycoside having one, two or three sugar moieties.
   15. The consumable food additive composition of embodiment 12, wherein said water soluble acetylated cannabinoid glycoside comprises a water soluble acetylated cannabinoid glycoside generated in vivo through incubation of a cannabinoid in a tobacco cell suspension culture.
   16. The consumable food additive composition of embodiment 12, wherein said water soluble acetylated cannabinoid glycoside comprises a water soluble acetylated cannabinoid glycoside generated in vitro through incubation with a glycosyltransferase protein having glycosylation activity towards one or more cannabinoids and acetyltransferase protein having acetylation activity toward one or more cannabinoids.
   17. The consumable food additive composition of embodiment 12, wherein said glycosyltransferase protein having glycosylation activity towards one or more cannabinoids comprises a glycosyltransferase protein from tobacco selected from the group consisting of: NtGT3; NtGT4; and NtGT5, and a homolog or ortholog of NtGT3, NtGT4, and NtGT5 having glycosylation activity towards one or more cannabinoids.
   18. A consumable food additive composition of embodiment 12, and further comprising a food additive polysaccharide.
   19. A consumable food additive composition of embodiment 18, wherein said food additive polysaccharide comprises dextrin, and maltodextrin.
   20. A consumable food additive composition of embodiment 12, and further comprising an emulsifier.
   21. A consumable food additive composition of embodiment 20, wherein said emulsifier is selected from the group consisting of: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.
   22. A consumable food additive composition of embodiment 12, and further comprising a flavoring agent.
   23. A consumable food additive composition of embodiment 22, wherein said flavoring agent comprises a flavoring agent selected from the group consisting of: Sucrose (sugar), glucose, fructose, sorbitol, mannitol, corn syrup, high fructose corn syrup, saccharin, aspartame, sucralose, acesulfame potassium (acesulfame-K), and neotame.
   24. A consumable food additive composition of embodiment 12, and further comprising a coloring agent, and wherein said coloring agent comprises a coloring agent selected from the group consisting of: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate, and inosinate, or combinations thereof.
   25. A consumable food additive composition of embodiment 12, and further comprising a surfactant.
   26. A consumable food additive composition of embodiment 25, wherein said surfactant comprises a surfactant selected from the group consisting of glycerol monostearate and polysorbate 80.
   27. A consumable food additive composition of embodiment 12, and further comprising a preservative, and wherein said preservative comprises a preservative selected from the group consisting of: ascorbic acid, citric acid, sodium benzoate, calcium propionate, sodium erythorbate, sodium nitrite, calcium sorbate, potassium sorbate, BHA, BHT, EDTA, tocopherols.
   28. The consumable fluid composition of embodiment 12, wherein the cannabinoid to be chemically modified cannabinoid comprises a cannabinoid from hemp.
   29. A consumable food additive composition of embodiment 12, wherein said chemically modified cannabinoid food additive is added to an alcohol beverage, and wherein said alcohol beverage is selected from the group consisting of: ethanol, beer, wine, and distilled spirits.
   30. A consumable beverage composition comprising:
an alcohol continuing consumable beverage containing a chemically modified water soluble cannabinoid, wherein said chemically modified water soluble cannabinoid comprises a glycosylated cannabinoid glycoside, an acetylated cannabinoid glycoside, or a mixture of both.

Another aim of the invention includes compositions of novel water-soluble cannabinoids and their method or manufacture. Still other aims of the current invention include additional compositions of matter that incorporate one or more water-soluble cannabinoids.

### BRIEF DESCRIPTION OF THE FIGURES

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIG. 1. Representative Chromatographic Elution profile of CBGA Glycosides found in in vitro Assays. Chromatograms A, B, and C represent respective extracted ion chromatograms for each glycoside product. Chromatogram D is representative of the total ion chromatogram. Peak Intensities are illustrated as relative abundance to most abundant peak in each respective chromatogram.

FIG. 2. Representative Chromatographic Elution profiles of Functionalized CBGA and Glycosides found in in vitro assays. Chromatograms A, B, and C represent respective extract rated ion chromatograms for each product. Chromatogram D is representative of the total ion chromatogram. Peak Intensities are illustrated as relative abundance to most abundant peak in each respective chromatogram.

FIG. 3. Representative Chromatographic Elution profile of CBDA Glycosides profiles found in Leaf Extracts. Chromatograms A, B, C, and D represent respective extract rated ion chromatograms for each glycoside product. Chromatogram E is representative of the total ion chromatogram. Peak Intensities are illustrated as relative abundance to most abundant peak in each respective chromatogram.

FIG. 4. Chromatographic Elution of Functionalized CBDA and Functionalized Glycosides in Leaf Extracts. Chromatograms A, B, and C represent respective extract rated ion chromatograms for each product. Chromatogram D is representative of the total ion chromatogram. Peak Intensities are illustrated as relative abundance to most abundant peak in each respective chromatogram.

FIG. 5. Gene construct for expression of cytochrome P450 (CYP3A4) gene, (SEQ ID NO. 1), expressing the cytochrome P450 (CYP3A4) protein (SEQ ID NO. 2) and P450 oxidoreductase gene (oxred) (SEQ ID NO. 3) expressing the P450 oxidoreductase protein (SEQ ID NO. 4), in plants. Both genes were driven by the constitutive 35S promoter (35S) and featured 5′ untranslated regions from Arabidopsis thaliana alcohol dehydrogenase (AtADH) as translational enhancers.

FIG. 6. Confirmation of expression of CYP3A4 and P450 oxidoreductase in tobacco leaves. CB1-CB5, biological replicates of leaves infiltrated with the CYP3A4/P450 oxidoreductase; WT=wild type tobacco leaves with no infiltration. L=1 kb plus ladder (Thermo Fisher Scientific, USA). The arrows show the expected (500 bp) band indicating expression of the transgene.

FIG. 7. Enhanced glycosylation of cannabinoids in P450-over expressing N. benthamiana plants. CB1-CB5 are biological reps overexpressing CYP3A4+P450 oxidoreductase, P_control is the P19 silencing suppressor (‘empty vector’ control). Vertical axis shows relative amounts expressed as peak area per g fresh weight.

FIG. 8. Gene construct for the cytosol and suspension culture cannabinoid production system. 35S, Cauliflower mosaic 35S promoter; HSPt, HSP terminator; 35PPDK, hybrid promoter consisting of the cauliflower mosaic virus 35S enhancer fused to the maize C4PPDK basal promoter (Yoo et al. 2007); 76G1, UDP glycosyltransferase from Stevia rebaudiana; ABCG2, human multi-drug transporter.

FIG. 9. Demonstrates RT-PCR confirmation of expression of CBDA synthase (a), UDP glycosyltransferase (b) and ABCG2 (c) in tobacco leaf cells. L is the 1 kb plus ladder (Thermo Fisher Scientific, USA). Numbers on the lanes represent independent transgenic lines. The arrows point to the expected band that shows expression of the transgene.

FIG. 10. Hydroxylation and glycosylation of cannabinoids in transgenic tobacco (SUS, numbered) overexpressing CBDA synthase, UDP glycosyltransferase and ABC transporter. WTS1 and 2 are wild type fed with substrate for endogenous reactions. There was some endogenous glycosylation of CBGA, as well as evidence for enhanced transgenic glycosyltransferase activity (e.g. SUS2, SUS3 and SUS4). The data has been corrected to peak area per g fresh weight.

FIG. 11. Enhanced modification of cannabinoids in transgenic N. benthamiana plants co-infected with constructs for glycosylation, P450-mediated functionalization (hydroxylation) and detoxification of hydrogen peroxide by catalase. SUS=construct for overexpressing CBDA synthase, UDP glycosyltransferase and ABC transporter; M3 S=construct for overexpressing CBDA synthase, UDP glycosyltransferase and ABC transporter with Cannabis MYB12-like and Arabidopsis thaliana catalase.

FIG. 12. Increased glycosylation activity in transgenic N. benthamiana plants (TSA, TSB, TSC, SUS, SUS/P450) overexpressing a glycosyltransferase compared to wild type in 14-hour transient expression assays.

FIG. 13. Exemplary monooxygenase reaction, catalyzed by cytochromes P450.

FIG. 14. Gene construct 1 for the trichome cannabinoid production system. Cauliflower mosaic 35S promoter; AtADH 5′-UTR, translation enhancer element (Matsui et al. 2012); tsCBDAs, cannabidiolic acid synthase with its original trichome target sequence; HSP terminator; tsUGT76G1, UDP glycosyltransferase from Stevia rebaudiana with CBDAs trichome target sequence.

FIG. 15. Gene construct 2 for the trichome cannabinoid production system. Cauliflower mosaic 35S promoter; AtADH 5′-UTR, enhancer element; PM-UTR1, Arabidopsis thaliana UDP-glucose/galactose transporter targeted to the plasma membrane; HSP terminator.

FIG. 16. Trichome-targeted CBDA synthase RT-PCR (top), Trichome-targeted UDP glycosyltransferase (76G1) UGT RT-PCR (bottom). A, B, and C are biological replicates collected after 2 DPI.

FIG. 17. PM-UTR1 RT-PCR. A, B, and C are biological replicates collected after 2 DPI.

FIG. 18. Gene construct for the cytosolic cannabinoid production system. Cauliflower mosaic 35S promoter; AtADH 5′-UTR, enhancer element; cytCBDAs, cannabidiolic acid synthase with the trichome target sequence removed; HSP terminator; cytUGT76G1, UDP glycosyltransferase from Stevia rebaudiana.

FIG. 19. SUS-A to SUS-C are biological replicates for the cell suspension (201-SUS) transformation after 1 DPI.

FIG. 20. cytUGT RT-PCR (top), cytCBDAs RT-PCR (bottom). A, B, and C are biological replicates for cytosolic construct infiltration after 2 DPI.

FIG. 21. Cannabinoid detection in leaves infiltrated with trichome or cell suspension constructs and fed with CBGA 2.7 mM. The color code refers to the target compartment for CBDAs and UGT76G1 protein accumulation, either trichome or cell suspension cytostol. Y-axis: CBGA and CBDA expressed as parts per million (ppm). Primary, secondary, and acetylated glycosides expressed as peak area.

FIG. 22. Cannabinoid detection in leaves infiltrated with cytosolic or cell suspension construct and fed with CBGA 2.7 mM and UDP-glucose 4 mM. The color code refers to the target compartment for CBDAs and UGT76G1 protein accumulation. Y-axis: CBGA expressed as parts per million (ppm). All other cannabinoid derivatives expressed as peak area (no standards available).

FIG. 23. Extracted Ion Chromatograms of R—OH Functionalized 1× Glycosylated CBDA Analog. (A) Chromatographic trace, ion m/z, calculated elemental composition, confirming presence of trace levels of CBDA analog (B) Absence of CBDA analog in control extract (C) Absence of CBDA analog in biological duplicate control extract.

FIG. 24. Direct Infusion Mass Spectrum of Cannabis sativa extract. Spectral insets represent CBDA with a single glycosylation (519.2546 m/z), and CBDA functionalized with R—OH and a single glycosylation (535.2543 m/z). Peak Intensities are illustrated as relative abundance to most intense ion.

FIG. 25. Relative abundance of CBDA in extracts of various Cannabis sativa strains infiltrated with Agrobacterium cultures harboring CBDA synthase (CBDAs) and UGT plasmid combinations. Normalized relative abundance data is presented as the ion intensity of each compound divided by the ion intensity of the internal standard 7-hydroxycoumarin (20 ppm).

FIG. 26. Relative abundance of modified CBDA (glycosylated and/or hydroxylated) in extracts of various Cannabis sativa strains infiltrated with Agrobacterium cultures harboring CBDAs and UGT plasmid combinations. Normalized relative abundance data is presented as the ion intensity of each compound divided by the ion intensity of the internal standard 7-hydroxycoumarin (20 ppm).

FIG. 27. Gene construct used to boost cannabinoid production and mitigate toxicity. CsMYB12, predicted Cannabis sativa MYB transcription factor for enhancing flavonol biosynthesis; HSPt, efficient transcription terminator from the Arabidopsis thaliana heat shock protein 18.2 gene; 35S, constitutive promoter from cauliflower mosaic virus; Catalase, Arabidopsis thaliana catalase gene.

FIG. 28. Synthesis of THC and CBD from common precursor CBGA.

FIG. 29. Generation of hydrogen peroxide during cannabinoid biosynthesis.

FIG. 30. Hydroxylation followed by oxidation of THC by CYP2C9/FIG. 31. Transfer of a glucuronic acid component to a cannabinoid substrate by UGT.

FIG. 32. Synthesis Olivetolic Acid a precursor of CBGA

FIG. 33. Amino Acid sequence comparison of exemplary Arabidopsis catalase protein sequences. FIG. 33 also contains SEQ. ID NO. 73 which represents CAT gene 1; SEQ. ID NO. 74 which represents CAT gene 2; and SEQ. ID NO. 75 which represents CAT gene 3.

FIG. 34. Schematic diagram of increase cannabinoid production coupled with reduced oxidative damage system in one embodiment thereof.

FIG. 35A-B. Part of the pPINK-αHC (A) and pPINK-HC (B) vectors showing the α-factor secretion signal, the ADE2 gene (PpADE2) which produces phosphoribosylaminoimidazole carboxylase in Pichia pastoris, utilized for adenine biosynthesis and the multiple cloning site (MSC) for cloning genes of interest. All the genes were cloned in the MCS for both vectors.

FIG. 36A-D. CBGA Glycoside Structures with Physiochemical and Constitutional Properties. A) CBGA, B) 0 Acetyl Glycoside, C) 1× Glycoside, D) 1× Glycoside

FIG. 37A-F. CBDA Glycoside Structures with Physiochemical and Constitutional Properties. A) CBDA, B) 1× Glycoside, C) 2× Glycoside, D) 0 Acetyl Glycoside, E) 1× Glycoside, F) 2× Glycoside, the disaccharide moiety can also be located on the opposite R—OH of CBDA as illustrated with the single glycoside product found in panels B & E.

FIG. 38. Representative Chromatographic Elution Profile of CBDA Glycosides found in yeast cell extracts. Chromatograms A, and B represent respective extract rated ion chromatograms for the parent and glycoside molecules. Chromatogram C is representative of the total ion chromatogram. Peak Intensities are illustrated as relative abundance to most abundant peak in each respective chromatogram.

FIG. 39. Representative chromatographic elution profile of CBGA glycosides found in yeast cell supernatants. Chromatograms A, and B represent respective extract rated ion chromatograms for parent and glycoside molecules. Panel B also illustrates a 13C isotope of the CBDA glycoside also found in the same analysis. Chromatogram C is representative of the total ion chromatogram. Peak Intensities are illustrated as relative abundance to most abundant peak in each respective chromatogram.

FIG. 40. Representative chromatographic elution profile of CBDA glycosides found in tobacco cell extracts. Chromatograms A, B, C, and D represent respective extract rated ion chromatograms for each glycoside product. Chromatogram E is representative of the total ion chromatogram. Peak Intensities are illustrated as relative abundance to most abundant peak in each respective chromatogram.

FIG. 41. Demonstration of expression of glycosyltransferases and Kat-E in Pichia pastoris.

FIG. 42. Gene construct for intracellular expression of NtGT4 in Pichia pastoris. Expression was driven by the AOX1 promoter and terminated by the cytochrome C1 (CYC1) terminator. Other exemplary glycosyltransferases were cloned in the manner shown.

FIG. 43A-C. Post-harvest glycosylation of CBDA in yeast. Glycosides are measured in normalized arbitrary units (AU) based on LC-MS peak area. Asterisks show significant difference (a greater number of asterisks means a lower P value) from the wild type at P=0.05. (A) CBDA 1× glycosides in NtGT1, NtGT4 and NtGT5 detected in the supernatant. (B) CBDA 1× glycosides in NtGT1, NtGT4 and NtGT5 detected in the pellet. (C) CBDA 2× glycoside (NtGT5) in the supernatant. (D) CBDA 1× glycoside on a different position mainly detected in NtGT5 transgenic lines in the pellet.

FIG. 44. Representative chromatographic elution profile of CBDA 1× glycosides found in yeast cell pellets for the intracellular expression of NtGT5. Chromatogram represents extraction ion chromatograms of the 519.259 m/z 1× glycoside ion. Peak Intensities are illustrated as relative abundance to most abundant peak in each respective chromatogram.

FIG. 45A-F. Postharvest glycosylation of CBD oil in yeast. Glycosides are measured in normalized arbitrary units (AU) based on LC-MS peak area. Asterisks show significant difference (a higher number of asterisks means a lower P value) from the wild type at P=0.05. WT=wild type Pichia pastoris Strain 4, Empty vec=yeast transformed with the empty vector pPINK-HC.

FIG. 46A-B. (A) Confirmation of transgene expression in yeast from secretion expression constructs NtGT1, NtGT4 and UGT76G1. αHC-empty is the empty vector control. (B) CBDA glycosides in the supernatant of yeast cultures secreting recombinant glycosyltransferases into the media. Asterisks show significant difference from the wild type at P=0.05.

FIG. 47A-B. Time course analysis of CBDA glycosylation in transgenic yeast. Depletion of CBDA was quantified along with accumulation of CBDA glycosides in the supernatant (A) and the pellet (B).

FIG. 48A-F. Confirmation of transgene expression in BY2 cell cultures. The cell culture line 319C overexpresses the ABC transporter (ABCG2) and the glycosyltransferase UGT76G1. (B-F). Glycosylated CBDA compounds produced from wild type (WT) and transgenic (319C) BY2 cells. 319C overexpresses UGT76G1 and ABCG2. Glycosylated CBDA compounds were detected mainly in the pellet (D, E and F) and to a lesser extent in the supernatant (B and C).

FIG. 49A-B. Relative glycosylated cannabinoid yields for tobacco BY2 (319C) and yeast (NtGT4 and NtGT5) cell extracts, normalized to fresh weight. Asterisks show significant difference (a greater number of asterisks means a lower P value) from BY2 cell extracts at P=0.05.

FIG. 50A-B. Solution stability analysis for cannabinoids, cannabinoid glycosides and acetylated cannabinoid glycosides. (A) Demonstrates the percent loss of CBDA in solution over 7 day period at room temperature; and (B) Demonstrates the percent loss of 1×CBDA glycoside in solution over 7 day period at room temperature.

FIG. 51A-B. Solution stability analysis for cannabinoids, cannabinoid glycosides and acetylated cannabinoid glycosides at various pH ranges. (A) Demonstrates the 1×CBD Glycoside content in solution over at day 7; and (B) Demonstrates the percent conversion of 1×CBDA Glycoside to 1×CBD Glycoside.

### MODE(S) FOR CARRYING OUT THE INVENTION(S)

The present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

The inventive technology may include systems and methods for the chemical modification of cannabinoid compounds. In one embodiment, a suspension culture of one or more yeast strains may be established. In one preferred embodiment, culture, and more preferably a suspension culture of Saccharomyces cerevisiae and/or Pichia pastoris or other suitable yeast species may be established in a fermenter or other similar apparatus. It should be noted that the use of the above identified example in this embodiment is exemplary only, as various yeast strains, mixes of strains, hybrids of different strains or clones may be used to generate a suspension culture. For example, Pichia pastoris or any other appropriate yeast strain, including but not limited to all strains of yeast deposited with the ATCC. (The yeast strain deposit database(s) being incorporated by reference in its entirety.) In certain cases, such fermenters may include large industrial-scale fermenters allowing for a large quantity of yeast cells to be grown. In this embodiment, it may be possible to culture a large quantity of cells from a single-strain of, for example, P. pastoris or K. marxianus, which may establish a cell culture having a consistent rate of cannabinoid modification. Such cultured growth may be continuously sustained with the continual addition of nutrient and other growth factors being added to the culture. Such features may be automated or accomplished manually.

As noted above, cannabinoid producing strains of Cannabis, as well as other plants may be utilized with the inventive technology. In certain preferred embodiments, Cannabis plant material may be harvested and undergo cannabinoid extraction through one or more of the methods generally described above. These extracted cannabinoids may be introduced into a genetically modified yeast suspension cell culture to be further modified as described below.

As noted above, accumulation of high-levels of cannabinoids may be toxic for the yeast cell. As such, the inventive technology may transiently modify the cannabinoids produced in the yeast cell culture in vivo. In one preferred embodiment, cytochrome P450's (CYP) monooxygenases may be utilized to transiently modify or functionalize the chemical structure of the cannabinoids to produce water-soluble forms. CYPs constitute a major enzyme family capable of catalyzing the oxidative biotransformation of many pharmacologically active chemical compounds and other lipophilic xenobiotics. For example, the most common reaction catalyzed by cytochromes P450 is a monooxygenase reaction, e.g., insertion of one atom of oxygen into the aliphatic position of an organic substrate (RH) while the other oxygen atom is reduced to water:

RH+O₂+NADPH+H⁺→ROH+H₂O+NADP⁺

Several cannabinoids, including THC, have been shown to serve as a substrate for human CYPs (CYP2C9 and CYP3A4). Similarly, CYPs have been identified that metabolize cannabidiol (CYPs 2C19, 3A4); cannabinol (CYPs 2C9, 3A4); JWH-018 (CYPs 1A2, 2C9); and AM2201 (CYPs 1A2, 2C9). For example, as shown generally below, in one exemplary system, CYP2C9 may hydroxylate a THC molecule resulting in a hydroxyl form of THC. Further oxidation of the hydroxyl form of THC by CYP2C9 may convert it into a carboxylic acid form, which loses its psychoactive capabilities rendering it an inactive metabolite.

In one embodiment, yeast cells may be transformed with artificially created genetic constructs encoding one or more CYPs. In one preferred embodiment, genes encoding one or more non-human isoforms and/or analogs, as well as possibly other CYPs that may functionalize cannabinoids may be expressed in transgenic yeast grown in a suspension culture. Additional embodiments may include genetic control elements such as promotors and/or enhancers as well as post-transcriptional regulatory elements that may also be expressed in transgenic yeast such that the presence, quantity and activity of any CYPs present in the suspension culture may be modified and/or calibrated.

In this preferred embodiment, NADPH-cytochrome P450 oxidoreductase (CPR) may be used to assist in the activity/function of one or more of the CYPs expressed within a genetically modified yeast cell. In this embodiment, CPR may serve as an electron donor to eukaryotic CYPs facilitating their enzymatic function within the transgenic yeast strain(s) described above. In one preferred embodiment, genes encoding CPR, or one or more non-human isoforms and/or analogs of CPR that may act as an electron donor to CYPs may be expressed in transgenic yeast grown in a suspension culture. Additional embodiments may include genetic control elements such as promotors and/or enhancers as well as post-transcriptional regulatory elements that may also be expressed in transgenic yeast such that the presence, quantity and activity of CPR present in the suspension culture may be modified and/or calibrated. For example, downregulation of the expression of CPR may decrease or stop the functionalization of cannabinoids by preventing the enzymatic action of the CYPs in the yeast cell.

Additional steps may be taken to further modify the functionalized cannabinoids. In a preferred embodiment, glycosylation of functionalized cannabinoids may covert to them into a water-soluble form. In an exemplary embodiment shown below, the inventive technology may utilize one or more UDP-glucuronosyltransferases (UGT) to catalyze the glucuronosylation or glucuronidation of both primary (CBD, CBN) and secondary cannabinoids (THC, JWH-018, JWH-073). In this embodiment, glucuronidation may consist of the transfer of a glucuronic acid component of uridine diphosphate glucuronic acid to a cannabinoid substrate by any of several types of UGTs as described above. Glucuronic acid is a sugar acid derived from glucose, with its sixth carbon atom oxidized to a carboxylic acid.

The conversion of a functionalized cannabinoid, in this example a carboxylic acid form of THC, to a glycosylated form of THC may generate a transiently modified cannabinoid that may be both soluble, and non-toxic to the cells in a suspension culture. These chemical modifications may allow for greater levels of cannabinoid accumulation within a yeast cell and/or in the surrounding cell culture media without the deleterious cytotoxic effects that may be seen with unmodified cannabinoids.

The inventive technology may include the generation of transgenic yeast strains having artificial genetic constructs that that may express one or more glycosyltransferases, or other enzymes capable of glycosylating functionalized cannabinoid compounds. In one preferred embodiment, artificial genetic constructs having genes encoding one or more UDP- and/or ADP-glycosyltransferases, including non-human analogues of those described above, as well as other isoforms, may be expressed in transgenic yeast cells and grown in suspension or other cell cultures. Additional embodiments may include genetic control elements such as promotors and/or enhancers as well as post-transcriptional regulatory control elements that may also be expressed in a transgenic yeast strain such that the presence, quantity and activity of any glycosyltransferases present in the suspension culture may be regulated. Additional embodiments may include artificial genetic constructs having one or more genes encoding one or more UDP- and/or ADP-glycosyltransferases having tags that may assist in the movement of the gene product to a certain portion of the cell, such as the cellular locations were cannabinoids and/or functionalized cannabinoids may be stored, and/or excreted from the cell.

In one embodiment of the inventive technology, the water-soluble, glycosylated cannabinoids, generally being referred to as transiently modified cannabinoids, may be transported into and harvested from the yeast cell culture media. In one embodiment, transiently modified cannabinoids may accumulate within the yeast cell itself. In this example, the yeast cell culture may be allowed to grow to a desired level of cell or optical density, or in other instances until a desired level of transiently modified cannabinoids have accumulated in the cultured cells and/or media. All, or a portion of the yeast cells containing the accumulated transiently modified cannabinoids may then be harvested from the culture and/or media, which in a preferred embodiment may be an industrial-scale fermenter or other apparatus suitable for the large-scale culturing of yeast or other microorganisms. The harvested yeast cells may be lysed such that the accumulated transiently modified cannabinoids may be released to the surrounding lysate. Additional steps may include treating this lysate. Examples of such treatment may include filtering, centrifugation or screening to remove extraneous cellular material as well as chemical treatments to improve later cannabinoid yields.

The transiently modified cannabinoids may be further isolated and purified. In one preferred embodiment, the yeast lysate may be processed utilizing affinity chromatography or other purification methods. In this preferred embodiment, an affinity column having a ligand configured to bind with one or more of the transiently modified cannabinoids, for example, through association with the glucuronic acid functional group, among others, may be immobilized or coupled to a solid support. The lysate may then be passed over the column such that the transiently modified cannabinoids, having specific binding affinity to the ligand become bound and immobilized. In some embodiments, non-binding and non-specific binding proteins that may have been present in the lysate may be removed. Finally, the transiently modified cannabinoids may be eluted or displaced from the affinity column by, for example, a corresponding sugar or other compound that may displace or disrupt the cannabinoid-ligand bond. The eluted transiently modified cannabinoids may be collected and further purified or processed.

In yet another separate embodiment, the now soluble transiently modified cannabinoids may be passively and/or actively excreted from the cell. In one exemplary model, an ATP-binding cassette transporter (ABC transporters) or other similar molecular structure may recognize the glucuronic acid functional group (conjugate) on the transiently modified cannabinoid and actively transport it into the surrounding media. In this embodiment, a yeast cell culture may be allowed to grow until an output parameter is reached. In one example, an output parameter may include allowing the yeast cell culture to grow until a desired cell/optical density is reached, or a desired level of transiently modified cannabinoids is reached. In this embodiment, the culture media containing the transiently modified cannabinoid may be harvested for later cannabinoid extraction. In some embodiments, this harvested media may be treated in a manner similar to the lysate generally described above. Additionally, the transiently modified cannabinoids present in the raw and/or treated media may be isolated and purified, for example, through affinity chromatography in a manner similar to that described above.

In certain embodiments, this purified cannabinoid isolate may contain a mixture of primary and secondary glycosylated cannabinoids. As noted above, such purified glycosylated cannabinoids may be water-soluble and metabolized slower than unmodified cannabinoids providing a slow-release capability that may be desirable in certain pharmaceutical applications, such as for use in tissue-specific applications or as a prodrug. In this embodiment, purified glycosylated cannabinoids may be incorporated into a variety of pharmaceutical and/or nutraceutical applications. For example, the purified glycosylated cannabinoids may be incorporated into various solid and/or liquid delivery vectors for use in pharmaceutical applications. As noted above, absent modification, these transiently modified cannabinoids no longer possess their psychoactive component, making their application in research, therapeutic and pharmaceutical applications especially advantageous. Additional therapeutic applications may include the administration of a therapeutic dose of an “entourage” of isolated and purified transiently modified cannabinoids.

The inventive technology may also include a system to convert or reconstitute transiently modified cannabinoids. In one preferred embodiment, glycosylated cannabinoids may be converted into non-glycosylated cannabinoids through their treatment with one or more generalized or specific glycosidases. In this embodiment, these glycosidase enzymes may remove a sugar moiety. Specifically, these glycosidases may remove the glucuronic acid moiety reconstituting the cannabinoid compound to a form exhibiting psychoactive activity. This reconstitution process may generate a highly purified “entourage” of primary and secondary cannabinoids. These reconstituted cannabinoid compounds may also be incorporated into various solid and/or liquid delivery vectors for use in a variety of pharmaceutical and other commercial applications. In certain embodiments, transiently modified cannabinoids may be reconstituted through incubation with one or more generalized or specific glycosidases in an in vitro system.

As noted above, cannabinoid producing strains of Cannabis, as well as other plants may be utilized with the inventive technology. In certain preferred embodiments, Cannabis plant material may be harvested and undergo cannabinoid extraction. These traditionally extracted cannabinoids may then be modified from their native forms through the in vitro application of one or more CYP's that may generate hydroxyl and carboxylic acid forms of these cannabinoids respectively. These functionalized cannabinoids may be further modified through the in vitro application of one or more UGTs as generally described below. In this embodiment, the new transiently modified cannabinoids may be isolated and purified through a process of affinity chromatography and then applied to various commercial and other therapeutic uses. In other embodiments, the transiently modified cannabinoids may be restored and reconstituted through the in vitro application of one or more glycosidase enzymes. These restored cannabinoids may also be applied to various commercial and other therapeutic uses.

The inventive technology includes systems and methods for high-level production of cannabinoid compounds in cell culture systems. As used herein, the term “high level” in this instance may mean higher than wild-type biosynthesis or accumulation of one or more cannabinoids in a yeast or plant cell culture. In one embodiment, a suspension or hairy root or cell suspension culture of one or more plant strains may be established. In one preferred embodiment, a suspension or hairy root or cell suspension culture of a tobacco plant may be established. It should be noted that the term strain may refer to a plant strain, as well as a cell culture, or cell line derived from a plant, such as tobacco. In another preferred embodiment, a suspension or hairy root or cell suspension culture of one or more yeast strains may be established.

Another embodiment of the inventive technology may include systems and methods for high level production of modified cannabinoid compounds. In one embodiment, a suspension or hairy root culture of one or more tobacco plant strains may be established. It should be noted that the term strain may refer to a plant strain, as well as a cell culture, or cell line derived from a tobacco plant. In one preferred embodiment, a suspension or hairy root culture of BY2 tobacco cells may be established in a fermenter or other similar apparatus. In an alternative embodiment, a suspension or hairy root culture of Nicotiana tabacum and/or Nicotiana benthamiana plant may be established in a fermenter or other similar apparatus. It should be noted that the use of N. tabacum and N. benthamiana in these embodiments is exemplary only. For example, in certain other embodiments, various Nicotiana strains, mixes of strains, hybrids of different strains or clones, as well as different varieties may be used to generate a cell suspension or hairy root culture.

In certain cases, such fermenters may include large industrial-scale fermenters allowing for a large quantity of tobacco cells to be cultured. In this embodiment, harvested cannabinoids may be introduced to this suspension culture, and modified as generally described herein. Similarly, such cultured growth of tobacco cells may be continuously sustained with the continual addition of nutrient and other growth factors being added to the culture. Such features may be automated or accomplished manually.

Another embodiment of the invention may include the production of genetically modified yeast and/or tobacco cells to express varying exogenous and/or endogenous genes that may modify the chemical structure of cannabinoid compounds. Such transgenic strains may be configured to produce and/or modify large quantities of cannabinoid compounds generally, as well as targeted increases in the production of specific cannabinoids such as THC, Cannabidiol (CBD) or Cannabinol (CBN) and the like.

Additional embodiments of the inventive technology may include novel systems, methods and compositions for the production and in vivo modification of cannabinoid compounds in a plant and/or yeast suspension culture system. In certain embodiments, these in vivo modifications may lead to the production of different forms of cannabinoids with special properties, e.g. water-soluble, slow-release cannabinoids or prodrugs. In one preferred embodiment, the inventive technology may include novel systems, methods and compositions for the hydroxylation, acetylation and/or glycosylation. Modified cannabinoids can be made water-soluble, for example by glycosylation.

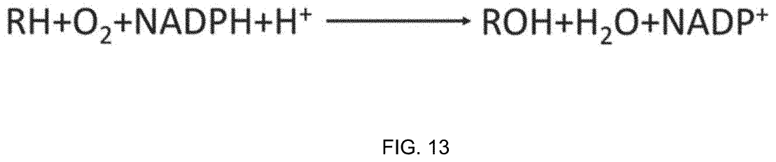

As noted above, production and/or accumulation of high-levels of cannabinoids would be toxic for a plant cell host. As such, one embodiment of the inventive technology may include systems and methods to transiently modify cannabinoids in vivo. One aim of the current invention may include the use of cytochrome P450's (CYP) monooxygenases to transiently modify or functionalize the chemical structure of the cannabinoids. CYPs constitute a major enzyme family capable of catalyzing the oxidative biotransformation of many pharmacologically active chemical compounds and other lipophilic xenobiotics. For example, as shown in FIG. 13, the most common reaction catalyzed by cytochromes P450 is a monooxygenase reaction, e.g., insertion of one atom of oxygen into the aliphatic position of an organic substrate (RH) while the other oxygen atom is reduced to water.

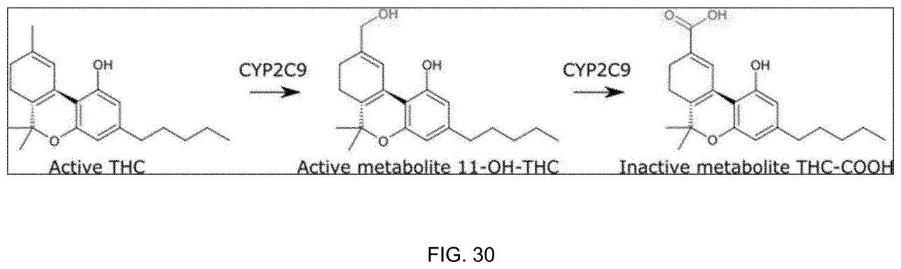

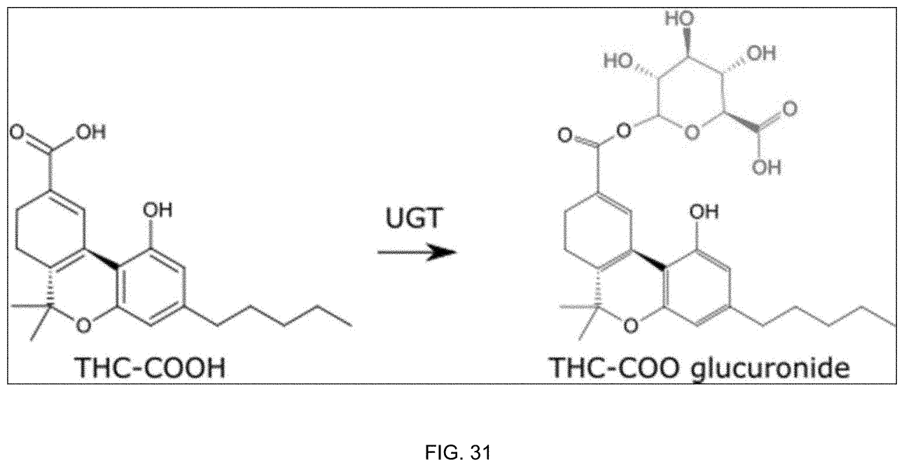

Several cannabinoids, including THC, have been shown to serve as a substrate for human CYPs (CYP2C9 and CYP3A4). Similarly, CYPs have been identified that metabolize cannabidiol (CYPs 2C19, 3A4); cannabinol (CYPs 2C9, 3A4); JWH-018 (CYPs 1A2, 2C9); and AM2201 (CYPs 1A2, 2C9). For example, as shown generally in FIG. 30, in one exemplary system, CYP2C9 may “functionalize” or hydroxylate a THC molecule resulting in a hydroxyl-form of THC. Further oxidation of the hydroxyl form of THC by CYP2C9 may convert it into a carboxylic-acid form which loses its psychoactive capabilities, rendering it an inactive metabolite.

As such, another embodiment of the invention may include the creation of a yeast or plant cell culture that may be transformed with artificially created genetic constructs encoding one or more exogenous CYPs. In one preferred embodiment, genes encoding one or more non-human isoforms and/or analogs, as well as possibly other CYPs that may functionalize cannabinoids, may be expressed in transgenic yeast or tobacco cells. In another preferred embodiment, genes encoding one or more non-human isoforms and/or analogs, as well as possibly other CYPs that may functionalize cannabinoids, may be expressed in transgenic yeast tobacco strains grown in a suspension culture. Additional embodiments may include genetic control elements such as promotors and/or enhancers as well as post-transcriptional regulatory elements that may also be expressed such that the presence, quantity and activity of any CYPs present in the suspension culture may be modified and/or calibrated.

Another embodiment of the invention may include the creation of a tobacco or yeast cells may be transformed with artificially created genetic constructs encoding one or more exogenous CYPs. In one preferred embodiment, genes encoding one or more non-human isoforms and/or analogs, as well as possibly other CYPs that may functionalize cannabinoids introduced to a transgenic tobacco cell and/or yeast suspension culture.

Another aim of the invention may be to further modify, in vivo, cannabinoids and/or already functionalized cannabinoids. In a preferred embodiment, glycosylation of cannabinoids and/or functionalized cannabinoids may covert to them into a water-soluble form. In an exemplary embodiment shown in FIG. 31, the inventive technology may utilize one or more glycosyltransferase enzymes, such as UDP-glycosyltransferase (UGT), to catalyze, in vivo the glucuronosylation or glucuronidation of cannabinoids, such as primary (CBD, CBN) and secondary cannabinoids (THC, JWH-018, JWH-073). In this embodiment, glucuronidation may consist of the transfer of a glucuronic acid component of uridine diphosphate glucuronic acid to a cannabinoid substrate by any of several types of glycosyltransferases as described herein. Glucuronic acid is a sugar acid derived from glucose, with its sixth carbon atom oxidized to a carboxylic acid.

Yet another embodiment of the current invention may include the in vivo conversion of a functionalized cannabinoid, in this example a carboxylic acid form of the cannabinoid, to a glycosylated form of cannabinoid that may be both water-soluble and non-toxic to the cell host. These chemical modifications may allow for greater levels of cannabinoid accumulation in a plant or yeast cell culture without the deleterious cytotoxic effects that would be seen with unmodified cannabinoids due to this water-solubility.

Another embodiment of the invention may include the generation of transgenic or genetically modified strains/cells of yeast and/or tobacco, having artificial genetic constructs that may express one or more genes that may increase cannabinoids solubility and/or decrease cannabinoid cytotoxicity. For example, the inventive technology may include the generation of transgenic plant and/or yeast cell lines having artificial genetic constructs that may express one or more endogenous/or exogenous glycosyltransferases or other enzymes capable of glycosylating cannabinoid compounds. For example, in one embodiment one or more exogenous glycosyltransferases from tobacco or other non-cannabis plants may be introduced into a cannabis plant or cell culture and configured to glycosylate cannabinoids in vivo.

In an additional embodiment, of the inventive technology may include the generation of artificial genetic constructs having genes encoding one or more glycosyltransferases, including non-human analogues of those described herein as well as other isoforms, that may further may be expressed in transgenic plant and/or yeast cells which may further be grown in a suspension culture. Additional embodiments may include genetic control elements such as promotors and/or enhancers as well as post-transcriptional regulatory control elements that may also be expressed in such transgenic cell systems such that the presence, quantity and activity of any glycosyltransferases present in the suspension culture may be regulated.

An additional embodiment of the invention may include artificial genetic constructs having one or more genes encoding one or more UDP- and/or ADP-glycosyltransferases having localization sequences or domains that may assist in the movement of the protein to a certain portion of the cell, such as the cellular locations were cannabinoids and/or functionalized cannabinoids may be modified, produced, stored, and/or excreted from the cell.

An additional embodiment of the invention may include artificial genetic constructs having one or more genes encoding one or more UDP- and/or ADP-glycosyltransferases being co-expressed with one or more exogenous genes that may assist in the movement of the protein to a certain portion of the cell, such as the cellular locations were cannabinoids and/or functionalized cannabinoids may be stored, and/or excreted from the cell.

One preferred embodiment of the inventive technology may include the high level in vivo production of water-soluble, glycosylated cannabinoids, generally being referred to as transiently modified cannabinoids that may be harvested from a plant and/or yeast cell culture. In one embodiment, transiently modified cannabinoids may accumulate within the cell that is part of a suspension culture. In this example, the cell culture may be allowed to grow to a desired level of cell or optical density, or in other instances until a desired level of transiently modified cannabinoids have accumulated in the cultured plant or yeast cells. Such exogenous genes may be localized, for example to the cytosol as generally described herein, and may further be co-expressed with other exogenous genes that may reduce cannabinoid biosynthesis toxicity and/or facilitate cannabinoid transport through, or out of the cell.

All or a portion of the cultured plant and/or yeast cells containing the accumulated transiently modified cannabinoids may then be harvested from the culture, which in a preferred embodiment may be an industrial-scale fermenter or other apparatus suitable for the large-scale culturing of plant cells. The harvested Cannabis cells may be lysed such that the accumulated transiently modified cannabinoids may be released to the surrounding lysate. Additional steps may include treating this lysate. Examples of such treatment may include filtering or screening this lysate to remove extraneous plant material as well as chemical treatments to improve later cannabinoid yields.

Another embodiment of inventive technology may include the high level in vivo generation of water-soluble, glycosylated cannabinoids, generally being referred to as transiently modified cannabinoids that may be harvested from a plant and/or yeast cell culture. In one embodiment, cannabinoids may be introduced to a non-cannabinoid producing plant and/or yeast cell culture, such as BY2 tobacco cells. In this preferred embodiment, the non-cannabinoid producing cell culture may be genetically modified to express one or more endogenous or exogenous genes that may modify the cannabinoids, for example through hydroxylation, acetylation and/or glycosylation. Such endogenous or exogenous genes may be localized, as generally described herein, and may further be co-expressed with other exogenous genes that may reduce cannabinoid biosynthesis toxicity and/or facilitate cannabinoid transport through, or out of the cell into a surrounding media.

This non-cannabinoid producing the cell culture may be allowed to grow to a desired level of cell or optical density, or in other instances until a desired level of transiently modified cannabinoids have accumulated in the cultured cells. In one embodiment, all or a portion of the BY2 and/or yeast cells containing the accumulated cannabinoids may then be harvested from the culture, which in a preferred embodiment may be an industrial-scale fermenter or other apparatus suitable for the large-scale culturing of cells. The harvested cells may be lysed such that the accumulated transiently modified cannabinoids may be released to the surrounding lysate. Additional steps may include treating this lysate. Examples of such treatment may include filtering or screening this lysate to remove extraneous material as well as chemical treatments to improve later cannabinoid yields.

Another embodiment of the inventive technology may include methods to isolate and purified transiently modified cannabinoids from a plant or suspension culture. In one preferred embodiment, a plant and/or yeast cell culture lysate may be generated and processed utilizing affinity chromatography or other purification methods. In this preferred embodiment, an affinity column having a ligand or protein receptor configured to bind with the transiently modified cannabinoids, for example through association with a glycosyl or glucuronic acid functional group among others, may be immobilized or coupled to a solid support. The lysate may then be passed over the column such that the transiently modified cannabinoids, having specific binding affinity to the ligand become bound and immobilized. In some embodiments, non-binding and non-specific binding proteins that may have been present in the lysate may be removed. Finally, the transiently modified cannabinoids may be eluted or displaced from the affinity column by, for example, a corresponding sugar or other compound that may displace or disrupt the cannabinoid-ligand bond. The eluted transiently modified cannabinoids may be collected and further purified or processed.

One embodiment of the invention may include the generation of transiently modified cannabinoids that may be passively and/or actively excreted from a cultured plant and/or yeast cell. In one exemplary model, an exogenous ATP-binding cassette transporter (ABC transporters) or other similar molecular structure may recognize the glycosyl or glucuronic acid functional group (conjugate) on the transiently modified cannabinoid and actively transport it across the cell wall/membrane and into the surrounding media. In this embodiment, the cell culture may be allowed to grow until an output parameter is reached. In one example, an output parameter may include allowing the cell culture to grow until a desired cell/optical density is reach, or a desired concentration of transiently modified cannabinoid is reached. In this embodiment, the culture media containing the transiently modified cannabinoids may be harvested for later cannabinoid extraction. In some embodiments, this harvested media may be treated in a manner similar to the lysate generally described above. Additionally, the transiently modified cannabinoids present in the raw and/or treated media may be isolated and purified, for example, through affinity chromatography in a manner similar to that described above.

In certain embodiments, this purified cannabinoid isolate may contain a mixture of primary and secondary glycosylated cannabinoids. As noted above, such purified glycosylated cannabinoids may be water-soluble and metabolized slower than unmodified cannabinoids providing a slow-release capability that may be desirable in certain pharmaceutical applications, such as for use in tissue-specific applications, or as a prodrug. As such, in one embodiment of the invention, isolated glycosylated cannabinoids may be incorporated into a variety of pharmaceutical and/or nutraceutical applications as well as other compositions of matter outline herein.

For example, the purified glycosylated cannabinoids may be incorporated into various solid and/or liquid delivery vectors for use in pharmaceutical applications. As noted above, these transiently modified cannabinoids may no longer possess their psychoactive component, making their application in research, therapeutic and pharmaceutical applications especially advantageous. For example, the treatment of children may be accomplished through administration of a therapeutic dose of isolated and purified transiently modified cannabinoids, without the undesired psychoactive effect. Additional therapeutic applications may include the harvesting and later administration of a therapeutic dose of an “entourage” of isolated and purified transiently modified cannabinoids.

Another embodiment of the invention may include a system to convert or reconstitute transiently modified cannabinoids. In one preferred embodiment, glycosylated cannabinoids may be converted into non-glycosylated cannabinoids through their treatment with one or more generalized or specific glycosidases. The use and availability of glycosidase enzymes would be recognized by those in the art without requiring undue experimentation. In this embodiment, these glycosidase enzymes may remove a sugar moiety. Specifically, these glycosidases may remove the glycosyl or glucuronic acid moiety reconstituting the cannabinoid compound to a form exhibiting psychoactive activity. This reconstitution process may generate a highly purified “entourage” of primary and secondary cannabinoids. These reconstituted cannabinoid compounds may also be incorporated into various solid and/or liquid delivery vectors for use in a variety of pharmaceutical and other commercial applications.

As noted above, in one embodiment of the invention, cannabinoid producing strains of Cannabis, as well as other plants may be utilized with the inventive technology. In certain preferred embodiments, in lieu of growing the target cannabinoid producing plant in a cell culture, the raw plant material may be harvested and undergo cannabinoid extraction utilizing one or more of the methods described herein. These traditionally extracted cannabinoids may then be modified from their native forms through the in vitro application of one or more CYP's that may generate hydroxyl and carboxylic acid forms of these cannabinoids respectively. These functionalized cannabinoids may be further modified through the in vitro application of one or more glycosyltransferases as generally described herein. In this embodiment, the new transiently modified cannabinoids may be isolated and purified through a process of affinity chromatography, or other extraction protocol, and then applied to various commercial and other therapeutic uses. In other embodiments, the transiently modified cannabinoids may be restored and reconstituted through the in vitro application of one or more glycosidase enzymes. These restored cannabinoids may also be applied to various commercial and other therapeutic uses.

Another embodiment of the invention may include the use of other non-cannabinoid producing plants in lieu of growing a cannabinoid producing plant in a cell culture. Here, cannabinoid may be introduced to genetically modified plants, or plant cell cultures that express one or more CYP's that may generate hydroxyl and carboxylic acid forms of these cannabinoids respectively. These functionalized cannabinoids may be further modified through the action of one or more glycosidases that may also be expressed in the non-cannabinoid producing plant or cell culture. In one preferred embodiment, a non-cannabinoid producing cell culture may include tobacco plant or tobacco cell cultures. Additional embodiments may similarly use genetically modified yeast cells grown in culture to generate biomodified cannabinoid compounds.

One embodiment of the invention may include an in vivo method of trichome-targeted cannabinoid accumulation and modification. One preferred embodiment of this in vivo system may include the creation of a recombinant protein that may allow the translocation of a CYP or glycosyltransferases to a site of extracellular cannabinoid synthesis in a whole plant. More specifically, in this preferred embodiment, one or more CYPs or glycosyltransferases may either be engineered to express all or part of the N-terminal extracellular targeting sequence as present in cannabinoid synthase protein, such as THCA synthase or CBDA synthase.

One another embodiment of the invention may include an in vivo method of high-level trichome-targeted cannabinoid biosynthesis, accumulation and/or modification. One preferred embodiment of this in vivo system may include the creation of a recombinant protein that may allow the translocation of a catalase to a site of extracellular cannabinoid synthesis in a whole plant. More specifically, in this preferred embodiment, one or more catalase enzymes may either be engineered to express all or part of the N-terminal extracellular targeting sequence as present in cannabinoid synthase protein, such as THCA synthase or CBDA synthase. In this embodiment, the catalase may be targeted to the site of cannabinoid biosynthesis allowing it to more efficiently neutralize hydrogen peroxide byproducts.

Another aim of the current invention may include the introduction of one or more compounds to facilitate the chemical decomposition of hydrogen peroxide resulting from cannabinoids biosynthesis. In one embodiment, one or more chemicals, metal ions, and/or catalysts may be introduced into a growth media to detoxify hydrogen peroxide (H₂O₂) in both yeast and plant cell cultures. Examples may include magnesium dioxide (MnO₂), permanganate ionMnO₄, and silver ion (Ag⁺), iron oxide, (Fe₂O₃), lead dioxide (PbO₂), cupric oxide (CuO), Hafnium(IV) oxide (HfO₂), ceric dioxide (CeO₂), Gadolinium trioxide (Gd₂O₃), Sodium Phosphate, Tribasic (NaPO₄), iodide ions, manganese metal, iron(III) Chloride Solution(FeCl₃). Such chemicals, ions, and/or catalyst may be added directly, or in solution to a cell culture. The amount may be dependent on the amount of hydrogen peroxide present which may be determined through a variety of established assays. As such, determinations of the optimal amounts are within the skill of those in the art.

In this preferred embodiment, this N-terminal trichome targeting sequence or domain may generally include the first 28 amino acid residues of a generalized synthase. An exemplary trichome targeting sequence for THCA synthase is identified SEQ ID NO. 40, while trichome targeting sequence for CBDA synthase is identified SEQ ID NO. 41. This extracellular targeting sequence may be recognized by the plant cell and cause the transport of the glycosyltransferase from the cytoplasm to the plant's trichrome, and in particular the storage compartment of the plant trichrome where extracellular cannabinoid glycosylation may occur. More specifically, in this preferred embodiment, one or more glycosyltransferases, such as UDP glycosyltransferase may either be engineered to express all or part of the N-terminal extracellular targeting sequence as present in an exemplary synthase enzyme.

Another embodiment of the invention may include an in vivo method of cytosolic-targeted cannabinoid production, accumulation and/or modification. One preferred embodiment of this in vivo system may include the creation of a recombinant protein that may allow the localization of cannabinoid synthases and/or glycosyltransferases to the cytosol.

More specifically, in this preferred embodiment, one or more cannabinoid synthases may be modified to remove all or part of the N-terminal extracellular targeting sequence. An exemplary trichome targeting sequence for THCA synthase is identified SEQ ID NO. 40, while trichome targeting sequence for CBDA synthase is identified SEQ ID NO. 41. Co-expression with this cytosolic-targeted synthase with a cytosolic-targeted CYP or glycosyltransferase, may allow the localization of cannabinoid synthesis, accumulation and modification to the cytosol. Such cytosolic target enzymes may be co-expressed with catalase, ABC transporter or other genes that may reduce cannabinoid biosynthesis toxicity and or facilitate transport through or out of the cell.

Another embodiment of the invention may include the generation of an expression vector comprising this polynucleotide, namely a cannabinoid synthase N-terminal extracellular targeting sequence and glycosyltransferase genes, operably linked to a promoter. A genetically altered plant or parts thereof and its progeny comprising this polynucleotide operably linked to a promoter, wherein said plant or parts thereof and its progeny produce said chimeric protein, is yet another embodiment. For example, seeds and pollen contain this polynucleotide sequence or a homologue thereof, a genetically altered plant cell comprising this polynucleotide operably linked to a promoter such that said plant cell produces said chimeric protein. Another embodiment comprises a tissue culture comprising a plurality of the genetically altered plant cells.

Another embodiment of the invention provides for a genetically altered plant or cell expressing a chimeric or fusion protein having a cannabinoid synthase N-terminal extracellular targeting sequence (see i.e., SEQ ID: 40-41; see also SEQ ID NO. 42 for full amino acid sequence of THCA synthase) coupled with a UDP glycosyltransferase genes, operably linked to a promoter. Another embodiment provides a method for constructing a genetically altered plant or part thereof having glycosylation of cannabinoids in the extracellular storage compartment of the plant's trichrome compared to a non-genetically altered plant or part thereof, the method comprising the steps of: introducing a polynucleotide encoding the above protein into a plant or part thereof to provide a genetically altered plant or part thereof, wherein said chimeric protein comprising a first domain, a second domain, and wherein said first domain comprises a cannabinoid synthase N-terminal extracellular targeting sequence, and a second domain comprises a glycosyltransferase sequence. These domains may be separated by a third domain or linker. This linker may be any nucleotide sequence that may separate a first domain from a second domain such that the first domain and the second domain can each fold into its appropriate three-dimensional shape and retain its activity.

One preferred embodiment of the invention may include a genetically altered plant or cell expressing a cytosolic-targeted cannabinoid synthase protein having a cannabinoid synthase N-terminal extracellular targeting sequence (SEQ IDs. 40-41) inactivated or removed. In one embodiment, a cytosolic targeted THCA synthase (ctTHCAs) may be identified as SEQ ID NO. 46, while in another embodiment cytosolic targeted CBDA synthase (cytCBDAs) is identified as SEQ ID NO. 22-23). Such cytosolic-targeted cannabinoid synthase protein may be operably linked to a promoter. Another embodiment provides a method for constructing a genetically altered plant or part thereof having glycosylation of cannabinoids in the plant's cytosol compared to a non-genetically altered plant or part thereof, the method comprising the steps of: introducing a polynucleotide encoding the above protein into a plant or part thereof to provide a genetically altered plant or part thereof, wherein said a cannabinoid synthase N-terminal extracellular targeting sequence has been disrupted or removed.

Yet another embodiment of the invention may include an in vivo method of cannabinoid glycosylation in a cannabis cell culture. In one preferred embodiment, to facilitate glycosylation of cannabinoids in cannabis cell culture, which would lack an extracellular trichrome structure, a cannabinoid synthase gene may be genetically modified to remove or disrupt, for example through a directed mutation, the extra-cellular N-terminal targeting domain which may then be used to transform a Cannabis plant cell in a cell culture. In this embodiment, without this targeting domain the cannabinoid synthase, for example THCA or CBDA synthases, may remain within the plant cell, as opposed to being actively transported out of the cell, where it may be expressed with one or more glycosyltransferases, such as UDP glycosyltransferase in the cytoplasm.

Another embodiment of the inventive technology may include systems and methods for enhanced production and/or accumulation of cannabinoid compounds in an in vivo system. In one preferred embodiment, the invention may include the generation of a genetically modified or transgenic Cannabis plant that may produce and/or accumulate one or more cannabinoids at higher than wild-type levels. In one embodiment, a transgenic Cannabis plant may be generated to express one or more Cannabis sativa transcription factors that may enhance the cannabinoid metabolic pathway(s). In one preferred embodiment, a polynucleotide may be generated that encodes for one or more Cannabis sativa myb transcription factors genes, and/or one or more exogenous ortholog genes that enhance the metabolite flux through the cannabinoid biosynthetic pathway.

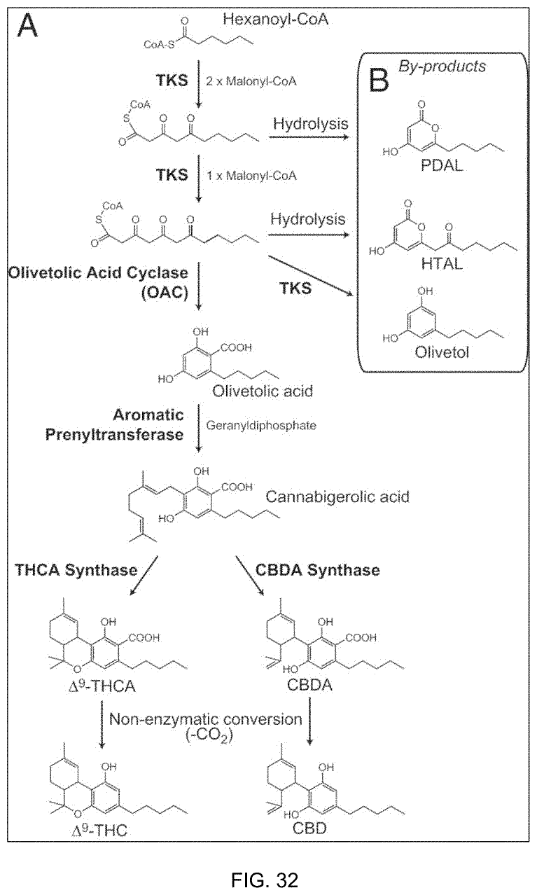

In this preferred embodiment, a polynucleotide may be generated that encodes for one or more Cannabis sativa myb transcription factors genes, such as CAN833 and/or CAN738 that. As shown in FIG. 32, these transcriptions factors may drive the production of olivetolic acid, which is a precursor of CBGA, which in turn is a precursor in the biosynthetic pathway of THCs, CBDs and CBC. In an alternative embodiment, a polynucleotide may be generated that encodes for one or more Cannabis sativa myb transcription factors genes orthologs, specifically cannabis Myb12 (SEQ IDs. 11-12), Myb8 (SEQ ID NO. 43), AtMyb12 (SEQ ID NO.44), and/or MYB112 (SEQ ID NO. 45) that may also drive the production of olivetolic acid, which is a precursor of CBGA, which in turn is a precursor in the biosynthetic pathway of THCs, CBDs and CBC.

In one preferred embodiment, the invention may include methods of generating a polynucleotide that expresses one or more of the SEQ IDs related to enhanced cannabinoid production identified herein. In certain preferred embodiments, the proteins of the invention may be expressed using any of a number of systems, such as in whole plants, as well as plant cell and/or yeast suspension cultures. Typically, the polynucleotide that encodes the protein or component thereof is placed under the control of a promoter that is functional in the desired host cell. An extremely wide variety of promoters may be available and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes” or “constructs.” Accordingly, the nucleic acids that encode the joined polypeptides are incorporated for high level expression in a desired host cell.

Additional embodiments of the invention may include selecting a genetically altered plant or part thereof that expresses the cannabinoid production transcription factor protein, wherein the expressed protein has increased cannabinoid biosynthesis capabilities. In certain embodiments, a polynucleotide encoding the cannabinoid production transcription factor protein is introduced via transforming said plant with an expression vector comprising said polynucleotide operably linked to a promoter. The cannabinoid production transcription factor protein may comprise a SEQ ID selected from the group consisting of SEQ ID NO: 11-2 or 43-45, or a homologue thereof.

As noted above, one embodiment of the invention may include systems and methods for general and/or localized detoxification of cannabinoid biosynthesis in an in vivo system. In one preferred embodiment, the invention may include the generation of a genetically modified or transgenic Cannabis or other plant that may be configured to be capable of detoxifying hydrogen peroxide by-products resulting from cannabinoid biosynthesis at higher than wild-type levels. In addition, this detoxification may be configured to be localized to the cytosol and/or trichome structure of the Cannabis plant where cannabinoids are actively being synthesized in a whole plant system. In this preferred embodiment of the invention, a transgenic plant, such as a cannabis or tobacco plant or cell, that express one or more genes that may up-regulate hydrogen peroxide detoxification. In an alternative embodiment, the invention may include the generation of a genetically modified plant cell and/or yeast cell suspension cultures that may be configured to be capable of expressing an exogenous catalase, or over expressing an endogenous catalase or both. In this example, the catalase expressed in the plant and/or yeast cell culture may act to detoxify hydrogen peroxide by-products resulting from cannabinoid biosynthesis at higher than wild-type levels. In some embodiment, the catalase expressed in a plant, and/or plant cell or yeast cell culture may be heterologous or exogenous, while in other embodiments, it may be an endogenous catalase that may be operably linked to a promoter to allow constitutive, inducible, and/or overexpression.

In one preferred embodiment, a polynucleotide may be generated that encodes for one or more endogenous and/or exogenous transcription catalase genes, and/or orthologs that catalyze the reduction of hydrogen peroxide:

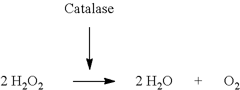

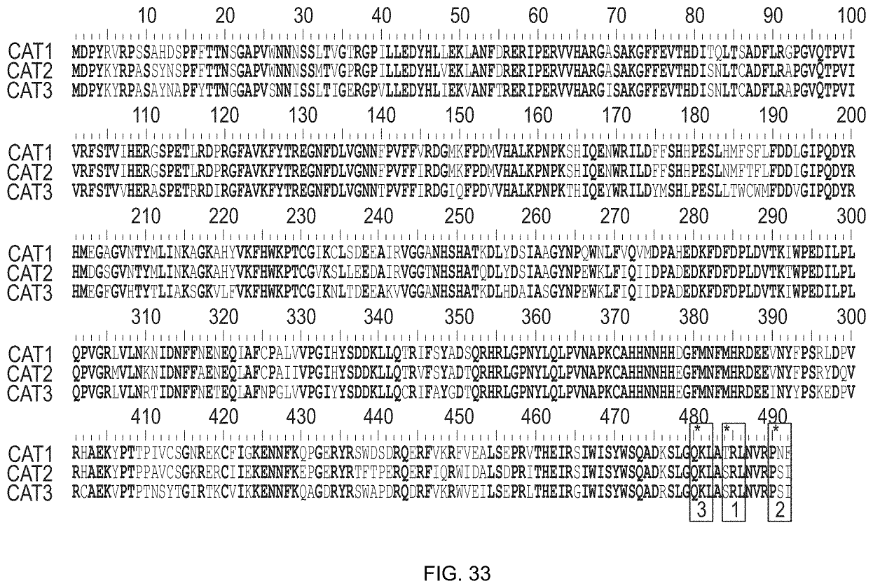

As such, in one embodiment, the invention comprises the generation of a polynucleotide encoding an exogenous catalase protein that may be expressed within a transformed plant and/or cell culture. In a preferred embodiment, a catalase enzyme configured reduce hydrogen peroxide (H₂O₂) generated during cannabinoid synthesis may be used to transform a cannabis or other plant, such as a tobacco plant. While a number of generic catalase enzymes may be included in this first domain, as merely one exemplary model, a first domain may include an exogenous catalase derived from Arabidopsis (SEQ ID NO. 13-14; see also FIG. 33), or Escherichia coli (SEQ ID NO. 15-16), or any appropriate catalase ortholog, protein fragment, or catalases with a homology between about 70%- and approximately 100% as herein defined.

Another embodiment of the current invention may include localization of the catalase enzyme to a trichome structure. As generally outlined above, in this embodiment a trichome targeting sequence from a cannabinoid synthase may be coupled with one or more catalase enzymes in a fusion or chimera—the terms being generally interchangeable in this application. This artificial trichome-target catalase gene may be used to transform a plant having trichome structures, such as Cannabis or tobacco. In a preferred embodiment, a trichome-targeted catalase from Arabidopsis thaliana with a THCA synthase trichome targeting domain is identified as SEQ ID NO. 47, while a trichome-targeted catalase Arabidopsis thaliana with a CBDA synthase trichome targeting domain is identified as SEQ ID NO. 48. In another embodiment, a trichome-targeted catalase from Escherichia coli with a THCA synthase trichome targeting domain is identified as SEQ ID NO. 49, while a trichome-targeted catalase Escherichia coli with a CBDA synthase trichome targeting domain is identified as SEQ ID NO. 50.

Another embodiment of the invention comprises generating a polynucleotide of a nucleic acid sequence encoding the chimeric/fusion catalase protein. Another embodiment includes an expression vector comprising this polynucleotide operably linked to a promoter. A genetically altered plant or parts thereof and its progeny comprising this polynucleotide operably linked to a promoter, wherein said plant or parts thereof and its progeny produce said fusion protein is yet another embodiment. For example, seeds and pollen contain this polynucleotide sequence or a homologue thereof, a genetically altered plant cell comprising this polynucleotide operably linked to a promoter such that said plant cell produces said chimeric protein. Another embodiment comprises a tissue culture comprising a plurality of the genetically altered plant cells.

In a preferred embodiment, a polynucleotide encoding a trichome-targeted fusion protein may be operably linked to a promoter that may be appropriate for protein expression in a Cannabis, tobacco or other plant. Exemplary promotors may include, but not be limited to: a non-constitutive promotor; an inducible promotor, a tissue-preferred promotor; a tissue-specific promotor, a plant-specific promotor, or a constitutive promotor. In a preferred embodiment, one or more select genes may be operably linked to a leaf-specific gene promotor, such as Cab 1. Additional promoters and operable configurations for expression, as well as co-expression of one or more of the selected genes are generally known in the art.

Another embodiment of the invention may provide for a method for constructing a genetically altered plant or part thereof having increased resistance to hydrogen peroxide cytotoxicity generated during cannabinoid synthesis compared to a non-genetically altered plant or part thereof, the method comprising the steps of: introducing a polynucleotide encoding a fusion protein into a plant or part thereof to provide a genetically altered plant or part thereof, wherein said fusion protein comprising a catalase and a trichome-targeting sequence from a cannabinoid synthase.

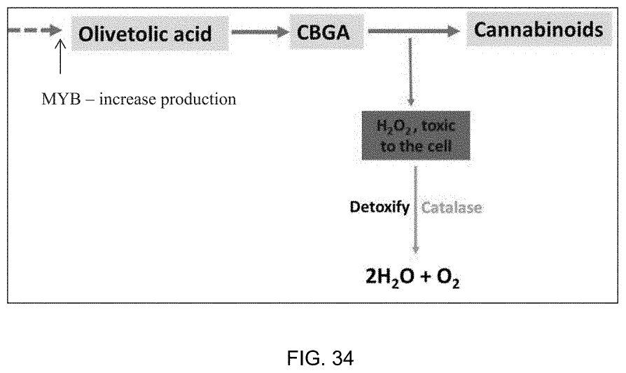

In one embodiment, the invention may encompass a system to increase overall cannabinoid production and accumulation in trichomes while preventing potential cytotoxicity effects. As generally shown in FIG. 34, the system may include, in a preferred embodiment, creating a transgenic Cannabis, tobacco or other plant or suspension culture plant that overexpresses at least one Myb transcription factor to increase overall cannabinoid biosynthesis. In further preferred embodiments, this transgenic plant may co-express a catalase enzyme to reduce oxidative damage resulting from hydrogen peroxide production associated with cannabinoid synthesis reducing cell toxicity. In certain preferred embodiments, this catalase may be fused with an N-terminal synthase trichome targeting domain, for example from THCA and/or CBDA synthase, helping localize the catalase to the trichome in the case of whole plant systems, and reduce potentially toxic levels of hydrogen peroxide produced by THCA, CBCA and/or CBDA synthase activity.

Another embodiment of the invention may comprise a combination polynucleotide of a nucleic acid sequence encoding a combination of: 1) a cannabinoid production transcription factor protein, such as a myb gene; and/or a catalase protein, or any homologue thereof, which may further include a trichome targeting or localization signal. A genetically altered plant or parts thereof and its progeny comprising this combination polynucleotide operably linked to a promoter, wherein said plant or parts thereof and its progeny produce said protein is yet another embodiment. For example, seeds and pollen contain this polynucleotide sequence or a homologue thereof, a genetically altered plant cell comprising this polynucleotide operably linked to a promoter such that said plant cell produces said proteins. Another embodiment comprises a tissue culture comprising a plurality of the genetically altered plant cells.

Another embodiment of the invention may provide for a method for constructing a genetically altered plant or part thereof having: 1) increased cannabinoid production compared to a non-genetically altered plant or part thereof; and/or 2) increased resistance to hydrogen peroxide cytotoxicity generated during cannabinoid synthesis compared to a non-genetically altered plant or part thereof, the method comprising the steps of: introducing a combination polynucleotide into a plant or part thereof to provide a genetically altered plant or part thereof.

Additional embodiments of the invention may include selecting a genetically altered plant or part thereof that expresses one or more of the proteins, wherein the expressed protein(s) may have: 1) increased cannabinoid production capabilities, for example through overexpression of an endogenous myb gene; and 2) catalase with/or without a trichome localization capability, or any combination thereof. In certain embodiments, a combination polynucleotide encoding the proteins is introduced via transforming said plant with an expression vector comprising said combination polynucleotide operably linked to a promoter. The cannabinoid production transcription factor protein may comprise a SEQ ID selected from the sequences identified herein, or homologues thereof. Naturally, such combinations and expression combination strategies, such identified in Tables 7-8, 10 below and elsewhere, are exemplary, as multiple combinations of the elements as herein described is included in the invention.

In one preferred embodiment, the inventive technology may include systems, methods and compositions high levels of in vivo cannabinoid hydroxylation, acetylation and/or glycosylation and/or a combination of all three. In a preferred embodiment, the in vivo cannabinoid hydroxylation, acetylation and/or glycosylation and/or a combination of all three may occur in a cannabinoid-producing plant or cell culture system. While in alternative embodiments may include a non-cannabinoid producing plant or cell culture system such as a tobacco plant, like N. benthamiana, or a yeast cell culture.

In one embodiment, the invention may include a cannabinoid production, accumulation and modification system. In one preferred embodiment, a plant, such as cannabis or tobacco, as well as a yeast cell, may be genetically modified to express one or more heterologous cytochrome P450 genes. In this preferred embodiment, a heterologous cytochrome P450 (CYP3A4) SEQ ID NO. 1 may be expressed in a cannabinoid-producing plant or cell culture system. While in alternative embodiments, a heterologous human cytochrome P450 (CYP3A4) may be expressed non-cannabinoid producing plant or cell culture system such as a tobacco plant, like N. benthamiana or a yeast cell, such a P. pastoris. In this embodiment, the overexpression of a heterologous human cytochrome P450 protein, identified as SEQ ID NO. 2, may functionalize endogenously-created cannabinoids so that they can be more efficiently glycosylated and/or acetylated in vivo, rendering them water-soluble.

In an alternative embodiment, the invention may include a cannabinoid production, accumulation and modification system. In one preferred embodiment, a plant, such as cannabis or tobacco, may be genetically modified to express one or more heterologous cytochrome P450 oxidoreductase genes. In this preferred embodiment, a heterologous cytochrome P450 oxidoreductase (oxred) identified as SEQ ID NO. 3, and SEQ ID NO. 72, identified as an ortholog, may be expressed in a cannabinoid-producing plant or cell culture system. While in alternative embodiments a heterologous human heterologous cytochrome P450 oxidoreductase (oxred) may be expressed non-cannabinoid producing plant or cell culture system such as a tobacco plant, like BY2 tobacco cells, or yeast cells. In this embodiment, the overexpression of a heterologous cytochrome P450 oxidoreductase (oxred) protein, identified as SEQ ID NO. 4, may functionalize endogenously-created cannabinoids so that they can be more efficiently glycosylated and/or acetylated in vivo, rendering them water-soluble.

In one preferred embodiment, a tobacco cell suspension culture may be generated using BY2 cells. Such BY2 cell may express a heterologous cytochrome P450 oxidoreductase (oxred) identified as SEQ ID NO. 3, and/or a heterologous glycosyltransferases, such as GT76G1 (SEQ ID NO. 61). Further, in this embodiment, a BY2 tobacco cell culture may also be genetically modified to express one or more multi-drug ABC transporters, such as ABCG2 (SEQ ID NO. 67). In this embodiment, one or more cannabinoids may be introduced to the genetically modified yeast cells, preferably in in a suspension culture, and may be functionalize and/or directly glycosylated prior to their active transport out of the cell into the surrounding media through the action of an ABC transporter, such as ABCG2. In still further example, a yeast cell may be genetically modified to express an alpha-factor secretion signal to further facilitate secretion of the modified cannabinoids, or cannabinoid precursors out of the yeast cell and into a surrounding media. In this system, one or multiple cannabinoids and/or cannabinoid precursors may be introduced to the yeast cell culture to be modified, for example through an cannabinoid oil or other extract.

It should be noted that in one embodiment, one or more glycosyltransferases may have an affinity for either of the hydroxy groups located at positions 2,4 on the pentylbenzoate/pentlybenzoic ring of a cannabinoid, compound, such a CBDA (2,4-dihydroxy-3-[(6R)-3-methyl-6-(prop-1-en-2-yl)cyclohex-2-en-1-yl]-6-pentylbenzoate) and/or CBGA ((E)-3-(3,7-Dimethyl-2,6-octadienyl)-2,4-dihydroxy-6-pentylbenzoic acid).

On one embodiment, one or more glycosidase inhibitors may be introduced to a plant and/or yeast cell culture as well as a whole plant where the production of glycosylated cannabinoids may be occurring. In one preferred embodiment, one or more of the following glycosidase inhibitors may be utilized: D,L-1,2-Anhydro-myo-inositol (Conduritol B Epoxide (CBE)); 6-Epicastanospermine (Castanospermine); 6-bromocyclohex-4-ene-1,2,3-triol (Bromoconduritol); (+)-1-Deoxynojirimycin (Deoxynojirimycin); 1,5-Dideoxy-1,5-imino-D-sorbitol hydrochloride (1-Deoxynojirimycin Hydrochloride); 1R,2S,3 S,4R)-rel-5-Cyclohexene-1,2,3,4-tetrol (Conduritol B); (3R,4R,5R)-5-(Hydroxymethyl)-3,4-piperidinediol (2S,3S)-2,3-Dihydroxybutanedioate (Isofagomine D-Tartrate); O-(D-Glucopyranosylidene)amino N-Phenylcarbamate; and (3S,4S,5R,6R)-3,4,5-Trihydroxy-6-(hydroxymethyl)-2-piperidinone (D-Manno-γ-lactam). Such glycosidase inhibitors are exemplary only and should not be seen as limiting on the invention in any way.

In an alternative embodiment, a heterologous cytochrome P450 gene may be expressed in a genetically modified yeast strain. For example, heterologous cytochrome P450 (CYP3A4) (SEQ ID NO. 69), and/or CYP oxidoreductase (SEQ ID NO. 71), may be introduced and expressed in to a yeast cell. In this embodiment, such genes may further be codon optimized for expression in yeast. Such a heterologous human cytochrome P450 proteins may functionalize cannabinoids introduced to the yeast cell culture so that they can be more efficiently glycosylated and/or acetylated in vivo, rendering them water-soluble. In this embodiment, such yeast cells may further express one or more heterologous glycosyltransferases, which may further be codon optimized for expression in yeast cells. In one preferred embodiment, the invention may one or more codon optimized heterologous glycosyltransferases from tobacco, including but not limited to: NtGT1 (SEQ ID NO. 51); NtGT2 (SEQ ID NO. 53); NtGT3 (SEQ ID NO. 55); NtGT4 (SEQ ID NO. 57); and NtGT5 (SEQ ID NO. 59).

In one embodiment, the invention may include a cannabinoid production, accumulation and modification system in a non-cannabinoid producing plant. In one preferred embodiment, a plant, such as tobacco, may be genetically modified to express one or more heterologous cytochrome P450 oxidoreductase genes. In this preferred embodiment, a heterologous cytochrome P450 oxidoreductase (oxred) identified as SEQ ID NO. 3 may be expressed in a cannabinoid-producing plant or cell culture system. While in alternative embodiments a heterologous cytochrome P450 oxidoreductase (oxred) may be expressed non-cannabinoid producing plant or cell culture system such as a tobacco plant, like N. benthamiana. In this embodiment, the overexpression of a heterologous cytochrome P450 oxidoreductase (oxred) protein, identified as SEQ ID NO. 4, may help to functionalize cannabinoids introduced to the genetically modified plant or plant cell culture system so that they can be more efficiently glycosylated and/or acetylated, in vivo, rendering them water-soluble.

In a preferred embodiment cytochrome 450 and P450 oxidoreductase are co-expressed. In another embodiment, cytochrome P450 and P450 oxidoreductase may also be expressed as a fusion protein. It should be noted that any nucleic and or amino acid expressed in this system may be expressed single or as a fusion protein,

In another embodiment, the invention may include the expression of one or more exogenous or heterologous, the terms being generally interchangeable, cannabinoid synthase gene in a non-cannabinoid producing plant or plant-cell culture system. In one preferred embodiment, such a gene may include one or more of a CBG, THCA, CBDA or CBCA synthase genes. For example in one embodiment, a Cannabidiolic acid (CBDA) synthase, identified as SEQ ID NO. 5 (gene) or SEQ ID NO. 6 (protein) from Cannabis sativa may use expressed in a non-cannabis-producing plant, such as or plant cell suspension culture of N. benthamiana. In another preferred embodiment, a Tetrahydrocannabinolic acid (THCA) synthase, identified as SEQ ID NO. 42 (gene) from Cannabis sativa may use expressed in a non-cannabis-producing plant, such as a plant cell suspension culture of N. benthamiana.

In another preferred embodiment, such cannabinoid synthase genes expressed in a cannabinoid and/or non-cannabinoid plant or plant-cell suspension culture may be target or localized to certain parts of a cell. For example, in one preferred embodiment, cannabinoid production may be localized to the cytosol allowing cannabinoids to accumulate in the cytoplasm. In one exemplary embodiment, an artificially modified cannabinoids synthase protein may be generated. In this example embodiment, a CBDA synthase may have the trichome targeting sequence remove forming a cytosolic CBDA synthase (cytCBDAs) identified as SEQ ID NO. 22, (gene) or 23 (protein). Alternative embodiments would include generation of other artificial cytosol target synthase genes, such as cytosolic THCA synthase (cytTHCAs) identified as SEQ ID NO. 46 (gene).

These preferred embodiments may be particularly suited for cannabinoid cell-suspension culture cannabinoid expression systems, as such culture systems lack the trichomes present in whole plants. As such, in one preferred embodiment, a cannabinoid producing plant may be transformed to one or more of the artificial cytosolic targeted cannabinoid synthase genes lacking a trichome-targeting signal. In an alternative embodiment, such artificial cytosolic targeted cannabinoid synthase genes may be expressed in a cannabinoid producing plant suspension culture where the corresponding endogenous wild-type synthase gene has been inhibited and/or knocked out.

In one embodiment, the invention may include a cannabinoid production, accumulation and modification system that may generate water-soluble cannabinoids. In one preferred embodiment, a plant, such as cannabis or tobacco, may be genetically modified to express one or more heterologous glycosyltransferase genes, such as UDP glycosyltransferase. In this preferred embodiment, UDP glycosyltransferase (76G1) (SEQ ID NO. 7) (gene)/SEQ ID NO. 8 (protein) from Stevia rebaudiana may be expressed in cannabinoid producing plant or cell suspension culture. In a preferred embodiment, the cannabinoid producing plant or cell suspension culture may be Cannabis. In another embodiment, one or more glycosyltransferase from Nicotiana tabacum and/or a homologous glycosyltransferase from Nicotiana benthamiana, may be expressed in a cannabinoid-producing plant, such as cannabis, or may be over-expressed in an endogenous plant and/or plant cell culture system. In a preferred embodiment, a glycosyltransferase gene and/or protein may be selected from the exemplary plant, such as Nicotiana tabacum Such glycosyltransferase gene and/or protein may include, but not limited to: Glycosyltransferase (NtGT5a) Nicotiana tabacum (SEQ ID NO. 26) (Amino Acid); Glycosyltransferase (NtGT5a) Nicotiana tabacum (SEQ ID NO. 27) (DNA); Glycosyltransferase (NtGT5b) Nicotiana tabacum (SEQ ID NO. 28) (Amino Acid); Glycosyltransferase (NtGT5b) Nicotiana tabacum (SEQ ID NO. 29) (DNA); UDP-glycosyltransferase 73C3 (NtGT4) Nicotiana tabacum (SEQ ID NO. 30) (Amino Acid); UDP-glycosyltransferase 73C3 (NtGT4) Nicotiana tabacum (SEQ ID NO. 31) (DNA); Glycosyltransferase (NtGT1b) Nicotiana tabacum (SEQ ID NO. 32) (Amino Acid); Glycosyltransferase (NtGT1b) Nicotiana tabacum (SEQ ID NO. 33) (DNA); Glycosyltransferase (NtGT1a) Nicotiana tabacum (SEQ ID NO. 34) (Amino Acid); Glycosyltransferase (NtGT1a) Nicotiana tabacum (SEQ ID NO. 35) (DNA); Glycosyltransferase (NtGT3) Nicotiana tabacum (SEQ ID NO. 36) (Amino Acid); Glycosyltransferase (NtGT3) Nicotiana tabacum (SEQ ID NO. 37) (DNA); Glycosyltransferase (NtGT2) Nicotiana tabacum (SEQ ID NO. 38) (Amino Acid); and/or Glycosyltransferase (NtGT2) Nicotiana tabacum (SEQ ID NO. 39) (DNA). The sequences from Nicotiana tabacum are exemplary only as other tobacco and non-tobacco glycosyltransferase may be used.

As noted above, such glycosyltransferases may glycosylate the cannabinoids and/or functionalized cannabinoids in a plant or plant cell suspension culture as generally described here. Naturally, other glycosyltransferase genes from alternative sources may be included in the current invention.

As noted above, in one embodiment, one or more glycosyltransferases may be targeted or localized to a portion of the plant cell. For example, in this preferred embodiment, cannabinoid glycosylation may be localized to the trichome allowing cannabinoids to accumulate at higher-then wild-type levels in that structure. In one exemplary embodiment, an artificially modified glycosyltransferase may be generated. In this example embodiment, a UDP glycosyltransferase (76G1) may be fused with a trichome-targeting sequence at its N-terminal tail. This trichome targeting sequence may be recognized by the cell and cause it to be transported to the trichome. This artificial gene construct is identified as SEQ ID NO. 19 (gene), or SEQ ID NO. 20 (protein). In one embodiment, a trichome targeting sequence or domain may be derived from any number of synthases. For example, in one embodiment a THCA Synthase Trichome domain (SEQ ID NO. 40) may be coupled with a glycosyltransferase as generally described above. Moreover, in another example, a CBDA Synthase Trichome targeting domain (SEQ ID NO. 41) may be coupled with a glycosyltransferase as generally described above.

In one embodiment, the inventive technology may include the in vivo generation of one or more cannabinoid glucuronides. As also noted above, UDP-glucuronosyltransferases catalyze the transfer of the glucuronosyl group from uridine 5′-diphospho-glucuronic acid (UDP-glucuronic acid) to substrate molecules that contain oxygen, nitrogen, sulfur or carboxyl functional groups. Glucuronidation of a compound, such as a cannabinoid may modulate the bioavailability, activity, and clearance rate of a compound. As such, in one embodiment, the invention may include a cannabinoid production, accumulation and modification system that may generate water-soluble cannabinoid glucuronides. In one preferred embodiment, a plant, such as cannabis or tobacco, or another eukaryotic cell, such as yeast, may be genetically modified to express one or more endogenous and/or heterologous UDP-glucuronosyltransferases. Such a UDP-glucuronosyltransferases may be expressed in cannabinoid producing plant, non-cannabinoid producing plant, cell suspension culture, or yeast culture. Non-limiting examples of UDP-glucuronosyltransferases may include UGT1A1, UGT1A3, UGT1A4, UGT1A6, UGT1A7, UGT1A8, UGT1A9, UGT1A1O, UGT2B4, UGT2B7, UGT2BI5, and UGT2BI7—there nucleotide and amino acid sequences being generally know to those of ordinary skill in the art. These UDP-glucuronosyltransferases may be a recombinant UDP-glucuronosyltransferases. In additional embodiments, a UDP-glucuronosyltransferase may be codon optimized for expression in, for example yeast. Methods of making, transforming plant cells, and expressing recombinant UDP-glucuronosyltransferases are known in the art. In a preferred embodiment, the cannabinoid producing plant or cell suspension culture may be cannabis. In another embodiment, one or more UDP-glucuronosyltransferases and/or a homolog/ortholog of a UDP-glucuronosyltransferase, may be expressed in a cannabinoid-producing plant, such as cannabis, or may be over-expressed in an endogenous plant and/or plant cell culture system or in yeast. In a preferred embodiment, a UDP-glucuronosyltransferase may be targeted or localized to a portion of the plant cell. For example, in this preferred embodiment, cannabinoid glucuronidation may be localized to the trichome allowing cannabinoids to accumulate at higher-then wild-type levels in that structure.

In one exemplary embodiment, an artificially modified UDP-glucuronosyltransferase may be generated. In this embodiment, a UDP-glucuronosyltransferase may be fused with a trichome-targeting sequence at its N-terminal tail. This trichome targeting sequence may be recognized by the cell and cause it to be transported to the trichome. In one embodiment, a trichome targeting sequence or domain may be derived from any number of synthases. For example, in one embodiment a THCA Synthase trichome domain (SEQ ID NO. 40) may be coupled with a UDP-glucuronosyltransferase as generally described above. Moreover, in another example, a CBDA Synthase trichome targeting domain (SEQ ID NO. 41) may be coupled with a UDP-glucuronosyltransferase as generally described above. In another embodiment, a UDP-glucuronosyltransferase may further be targeted to the cytosol as generally described herein.

In another embodiment, invention may include an embodiment where transiently modified cannabinoids may be passively and/or actively excreted from a cell or into a cell wall. In one exemplary model, an exogenous ATP-binding cassette transporter (ABC transporters or ABCt) or other similar molecular structure may recognize the glycosyl or glucuronic acid or acetyl functional group (conjugate) on the transiently modified cannabinoid and actively transport it across the cell wall/membrane and into the surrounding media.

In one embodiment, a plant may be transformed to express a heterologous ABC transporter. In this embodiment, an ABCt may facilitate cannabinoid transport outside the cells in suspension cultures, such as a cannabis or tobacco cell suspension culture. In this preferred embodiment, a human multi-drug transported (ABCG2) may be expressed in a plant cell suspension culture of the same respectively. ABCG2 is a plasma membrane directed protein and may further be identified as SEQ ID NO. 9 (gene), or 10 (protein).

Generally, a trichome structure, such as in Cannabis or tobacco, will have very little to no substrate for a glycosyltransferase enzyme to use to effectuate glycosylation. To resolve this problem, in one embodiment, the invention may include systems, methods and compositions to increase substrates for glycosyltransferase, namely select sugars in a trichome. In one preferred embodiment, the invention may include the targeted or localization of sugar transport to the trichome. In this preferred embodiment, an exogenous or endogenous UDP-glucose/UDP-galactose transporter (UTR1) may be expressed in a trichome producing plant, such as cannabis or tobacco and the like. In this embodiment, the UDP-glucose/UDP-galactose transporter (UTR1) may be modified to include a plasma-membrane targeting sequence and/or domain. With this targeting domain, the UDP-glucose/UDP-galactose transporter (UTR1) may allow the artificial fusion protein to be anchored to the plasma membrane. In this configuration, sugar substrates from the cytosol may pass through the plasma membrane bound UDP-glucose/UDP-galactose transporter (PM-UTR1) into the trichome. In this embodiment, substrates for glycosyltransferase may be localized to the trichome and allowed to accumulate further allowing enhanced glycosylation of cannabinoids in the trichome. In one example, SEQ ID NO. 21 is identified as the polynucleotide gene sequence for a heterologous UDP-glucose/galactose transporter (UTR1) from Arabidopsis thaliana having a plasma-membrane targeting sequence replacing a tonoplast targeting sequence. The plasma membrane targeting sequence of this exemplary fusion protein may include the following sequence (see SEQ ID NO 21) TGCTCCATAATGAACTTAATGTGTGGGTCTACCTGCGCCGCT, or a sequence having 70-99% homology with the sequence.

It should be noted that a number of combinations and permutations of the genes/proteins described herein may be co-expressed and thereby accomplish one or more of the goals of the current invention. Such combinations are exemplary of preferred embodiments only, and not limiting in any way.

In one embodiment, a gene, such as a cannabinoid synthase, or a gene fragment corresponding with, for example a signal domain may be inhibited, downregulated, disrupted, or may even be knocked-out. One of ordinary skill in the art will recognize the many processes that can accomplish this without undue experimentation. In other embodiment, a knock-out may mean overexpression of a modified endo- or exogenous gene compared to the wild-type version.

For example, in one embodiment high levels of cannabinoid glycosylation may be generated by co-expressing CYP3A4 and CYP oxidoreductase (cytochrome P450 with P450 oxidoreductase) and at least one endogenous glycosyltransferases in N. benthamiana. In another embodiment, one or more of the endogenous or exogenous gene may be expressed in a plant or plant cell culture with the co-expression of myb and/or a catalase. In this configuration, there exists an additive effect of over-expressing a Myb transcription factor and a catalase, one or more of which may be targeted or localized, in the synthesis of water-soluble cannabinoids (glycosylated and hydroxylated) in Cannabis sativa.

In certain embodiments, endocannabinoids may be functionalized and/or acetylated and/or glycosylated as generally described herein.

All sequences described herein include sequences having between 70-99% homology with the sequence identified.

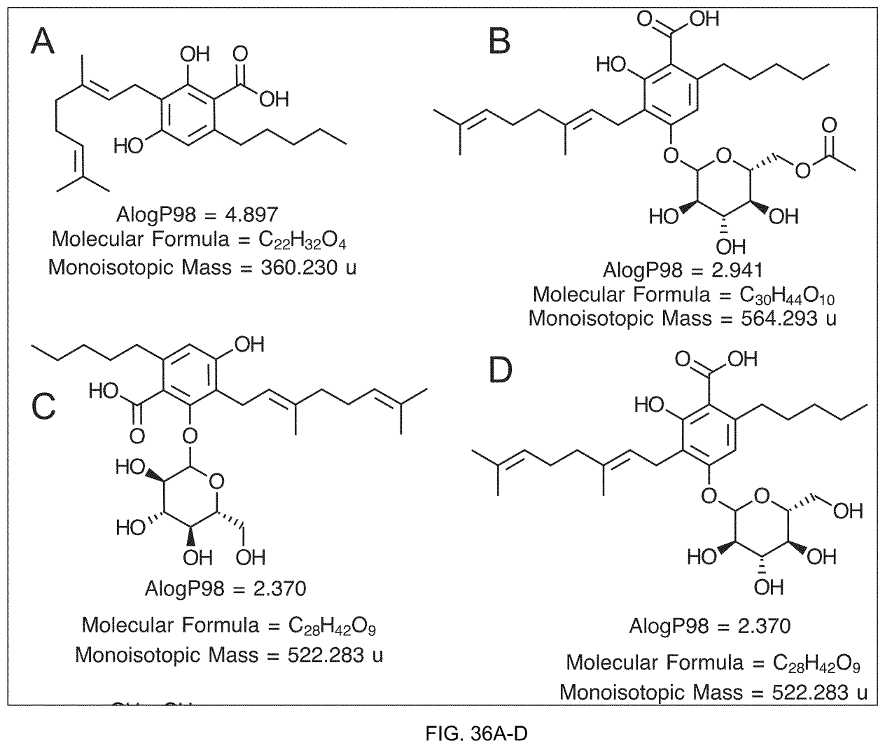

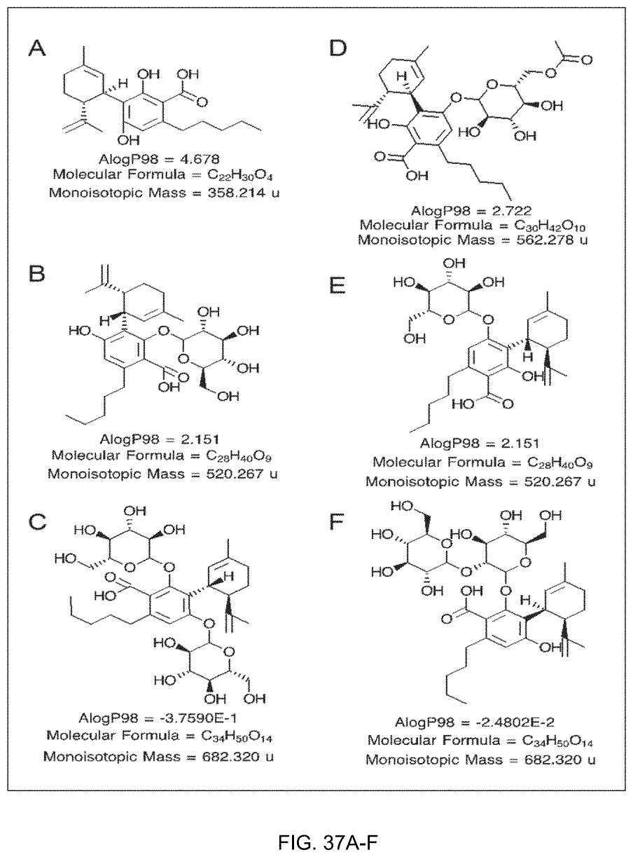

The inventive technology may further include novel cannabinoid compounds as well as their in vivo generation. As demonstrated in FIGS. 36 and 37 respectively, the invention includes modified cannabinoid compounds identified as: 36B, 36C, 36D, 37A, 37B, 37C, 37D, 37E and 37F and/or a physiologically acceptable salt thereof. In one preferred embodiment, the invention may include a pharmaceutical composition as active ingredient an effective amount or dose of one or more compounds identified as 36A, 36B, 36C, 36D, 37B, 37C, 37D, 37E and 37F and/or a physiologically acceptable salt thereof, wherein the active ingredient is provided together with pharmaceutically tolerable adjuvants and/or excipients in the pharmaceutical composition. Such pharmaceutical composition may optionally be in combination with one or more further active ingredients. In one embodiment, one of the aforementioned compositions may act as a prodrug. The term “prodrug” is taken to mean compounds according to the invention which have been modified by means of, for example, sugars and which are cleaved in the organism to form the effective compounds according to the invention. The terms “effective amount” or “effective dose” or “dose” are interchangeably used herein and denote an amount of the pharmaceutical compound having a prophylactically or therapeutically relevant effect on a disease or pathological conditions, i.e. which causes in a tissue, system, animal or human a biological or medical response which is sought or desired, for example, by a researcher or physician. Pharmaceutical formulations can be administered in the form of dosage units which comprise a predetermined amount of active ingredient per dosage unit. The concentration of the prophylactically or therapeutically active ingredient in the formulation may vary from about 0.1 to 100 wt %. Preferably, the compound of formula (I) or the pharmaceutically acceptable salts thereof are administered in doses of approximately 0.5 to 1000 mg, more preferably between 1 and 700 mg, most preferably 5 and 100 mg per dose unit. Generally, such a dose range is appropriate for total daily incorporation. In other terms, the daily dose is preferably between approximately 0.02 and 100 mg/kg of body weight. The specific dose for each patient depends, however, on a wide variety of factors as already described in the present specification (e.g. depending on the condition treated, the method of administration and the age, weight and condition of the patient). Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

In the meaning of the present invention, the compound is further defined to include pharmaceutically usable derivatives, solvates, prodrugs, tautomers, enantiomers, racemates and stereoisomers thereof, including mixtures thereof in all ratios.

In one embodiment, the current invention may include systems, methods and compositions for the efficient production of cannabidiolic acid (CBDA) in yeast coupled with a system of hydrogen peroxide detoxification. In this embodiment, the inventive technology may include the generation of a genetically modified yeast cell.

In one embodiment, the inventive system may include: 1) transforming a yeast cell with a first nucleotide sequence comprising the nucleotide sequence expressing an acyl-activating enzyme and expressing a mutant prenyltransferase; and 2) transforming the yeast cell with a second nucleotide sequence comprising the nucleotide sequence expressing olivetolic synthase, expressing olivetolic acid cyclase and expressing aromatic prenyltransferase; 3) and transforming a yeast cell with a third nucleotide sequence expressing a catalase gene.

In another embodiment, the inventive system may include the step of: 1) transforming a yeast cell with a first nucleotide sequence expressing an acyl-activating enzyme and expressing a mutant prenyltransferase; 2) transforming a yeast cell with a second nucleotide sequence expressing olivetolic synthase and expressing olivetolic acid cyclase; and transforming a yeast cell with a third nucleotide sequence expressing aromatic prenyltransferase and expressing cannabidiolic acid synthase; and 3) transforming a yeast cell with a third nucleotide sequence expressing a catalase gene.

Additional embodiments of the invention may further include: 1) transforming a yeast strain with a first nucleotide sequence expressing an acyl-activating enzyme; 2) transforming the yeast strain with a second nucleotide sequence expressing a mutant prenyltransferase; 3) transforming the yeast strain with a third nucleotide sequence expressing olivetolic synthase; 4) transforming the yeast strain with a fourth nucleotide sequence expressing olivetolic acid cyclase; 5) transforming the yeast strain with a fifth nucleotide sequence expressing aromatic prenyltransferase; 6) transforming the yeast strain with a sixth nucleotide expressing cannabidiolic acid synthase; and 7) transforming the yeast strain with a sixth nucleotide expressing a catalase.

Additional embodiments of the invention may further include: 1) transforming a yeast cell with a first nucleotide sequence expressing an acyl-activating enzyme and expressing a mutant prenyltransferase; 2) transforming the yeast cell with a second nucleotide sequence expressing olivetolic synthase and expressing olivetolic acid cyclase; 3) and transforming the yeast cell with a third nucleotide sequence expressing aromatic prenyltransferase and expressing cannabidiolic acid synthase; and 7) transforming the yeast cell with a fourth nucleotide expressing a catalase.

Additional embodiments of the invention may further include: 1) transforming a yeast cell with a first nucleotide sequence expressing an acyl-activating enzyme and expressing a mutant prenyltransferase; 2) transforming the yeast cell with a second nucleotide sequence expressing olivetolic synthase and expressing olivetolic acid cyclase; 3) transforming the yeast cell with a third nucleotide sequence expressing aromatic prenyltransferase and expressing cannabidiolic acid synthase, and 7) transforming the yeast cell with a fourth nucleotide expressing a catalase.

Sequence listings for the above identified sequences can be found in specification index NOs 1 and 2 filed in application Ser. No. 15/815,651, both of which are incorporated herein by reference. In particular, the following sequences are specifically incorporated by reference: iSEQ. ID. NO. 1; iSEQ. ID. NO. 2; iSEQ. ID. NO. 4; iSEQ. ID. NO. 5; iSEQ. ID. NO. 6; iSEQ. ID. NO. 7; iSEQ. ID. NO. 8; iSEQ. ID. NO. 9; iSEQ. ID. NO. 10; iSEQ. ID. NO. 11; iSEQ. ID. NO. 12; iSEQ. ID. NO. 13; iSEQ. ID. NO. 14; iSEQ. ID. NO. 15; iSEQ. ID. NO. 16; iSEQ. ID. NO. 23; iSEQ. ID. NO. 24; iSEQ. ID. NO. 22; iSEQ. ID. NO. 25; iSEQ. ID. NO. 26; iSEQ. ID. NO. 27; and iSEQ. ID. NO. 28. (The above sequences are marked with an “i” to denote their incorporation by reference.

In one embodiment, the invention may include systems, methods and compositions for the expression of exogenous, or heterologous genes in a yeast cell that may allow the biomodification and/or secretion of cannabinoids generated in a yeast cell. Specifically, the invention may allow the generation of cannabinoids and/or cannabinoid precursors in a genetically modified yeast cell, which may further be functionalized and/or modified into a water-soluble form. This embodiment may include transforming a yeast cell to express one or more of the following: heterologous cytochrome P450, and/or a heterologous P450 oxidoreductase, and/or a glycosyltransferase and/or heterologous ABC transporter. Similar to the above example, the genes may further be codon optimized for expression in a yeast cell that is configured to produce one or more cannabinoids or cannabinoid precursors, such as those genetically modified yeast cells described in U.S. Pat. No. 9,822,384, and U.S. patent application Ser. No. 15/815,651. In this embodiment, the exogenous catalase may be capable of generating water-soluble cannabinoid in one or more of the yeast cells identified in U.S. Pat. No. 9,822,384, and U.S. patent application Ser. No. 15/815,651, both of which are hereby incorporated in their entirety.

In one embodiment, the current invention may include systems, methods and compositions for the efficient production of cannabidiolic acid (CBDA) in yeast coupled with a system of biotransformation of the cannabinoids into a water-soluble form. In this embodiment, the inventive technology may include the generation of a genetically modified yeast cell.

In one embodiment, the inventive system may include: 1) transforming a yeast cell with a first nucleotide sequence comprising the nucleotide sequence expressing an acyl-activating enzyme and expressing a mutant prenyltransferase; and 2) transforming the yeast cell with a second nucleotide sequence comprising the nucleotide sequence expressing olivetolic synthase, expressing olivetolic acid cyclase and expressing aromatic prenyltransferase; 3) and transforming a yeast cell to express one or more of the following: heterologous cytochrome P450, and/or a heterologous P450 oxidoreductase, and/or a glycosyltransferase and/or heterologous ABC transporter, and/or a catalase. In this embodiment, the heterologous cytochrome P450, and/or a heterologous P450 oxidoreductase, and/or a glycosyltransferase and/or heterologous ABC transporter, and/or a catalase, the sequences identified herein may further be codon optimized for expression in yeast. Such codon optimization being generally within the knowledge and ability of one of ordinary skill in the art.

In another embodiment, the inventive system may include the step of: 1) transforming a yeast cell with a first nucleotide sequence expressing an acyl-activating enzyme and expressing a mutant prenyltransferase; 2) transforming a yeast cell with a second nucleotide sequence expressing olivetolic synthase and expressing olivetolic acid cyclase; and transforming a yeast cell with a third nucleotide sequence expressing aromatic prenyltransferase and expressing cannabidiolic acid synthase; 3) and transforming a yeast cell to express one or more of the following: heterologous cytochrome P450, and/or a heterologous P450 oxidoreductase, and/or a glycosyltransferase and/or heterologous ABC transporter, and/or a catalase.

Additional embodiments of the invention may further include: 1) transforming a yeast strain with a first nucleotide sequence expressing an acyl-activating enzyme; 2) transforming the yeast strain with a second nucleotide sequence expressing a mutant prenyltransferase; 3) transforming the yeast strain with a third nucleotide sequence expressing olivetolic synthase; 4) transforming the yeast strain with a fourth nucleotide sequence expressing olivetolic acid cyclase; 5) transforming the yeast strain with a fifth nucleotide sequence expressing aromatic prenyltransferase; 6) transforming the yeast strain with a sixth nucleotide expressing cannabidiolic acid synthase; and 7) and transforming a yeast cell to express one or more of the following: heterologous cytochrome P450, and/or a heterologous P450 oxidoreductase, and/or a glycosyltransferase and/or heterologous ABC transporter, and/or a catalase.

Additional embodiments of the invention may further include: 1) transforming a yeast cell with a first nucleotide sequence expressing an acyl-activating enzyme and expressing a mutant prenyltransferase; 2) transforming the yeast cell with a second nucleotide sequence expressing olivetolic synthase and expressing olivetolic acid cyclase; 3) and transforming the yeast cell with a third nucleotide sequence expressing aromatic prenyltransferase and expressing cannabidiolic acid synthase; and 7) and transforming a yeast cell to express one or more of the following: heterologous cytochrome P450, and/or a heterologous P450 oxidoreductase, and/or a glycosyltransferase and/or heterologous ABC transporter, and/or a catalase.

Additional embodiments of the invention may further include: 1) transforming a yeast cell with a first nucleotide sequence expressing an acyl-activating enzyme and expressing a mutant prenyltransferase; 2) transforming the yeast cell with a second nucleotide sequence expressing olivetolic synthase and expressing olivetolic acid cyclase; 3) transforming the yeast cell with a third nucleotide sequence expressing aromatic prenyltransferase and expressing cannabidiolic acid synthase, and 7) and transforming a yeast cell to express one or more of the following: heterologous cytochrome P450, and/or a heterologous P450 oxidoreductase, and/or a glycosyltransferase and/or heterologous ABC transporter, and/or a catalase.

Sequence listings for the above identified sequences can be found in specification index NOs 1 and 2 filed in application Ser. No. 15/815,651, both of which are incorporated herein by reference. In particular, the following sequences are specifically incorporated by reference: iSEQ. ID. NO. 1; iSEQ. ID. NO. 2; iSEQ. ID. NO. 4; iSEQ. ID. NO. 5; iSEQ. ID. NO. 6; iSEQ. ID. NO. 7; iSEQ. ID. NO. 8; iSEQ. ID. NO. 9; iSEQ. ID. NO. 10; iSEQ. ID. NO. 11; iSEQ. ID. NO. 12; iSEQ. ID. NO. 13; iSEQ. ID. NO. 14; iSEQ. ID. NO. 15; iSEQ. ID. NO. 16; iSEQ. ID. NO. 23; iSEQ. ID. NO. 24; iSEQ. ID. NO. 22; iSEQ. ID. NO. 25; iSEQ. ID. NO. 26; iSEQ. ID. NO. 27; and iSEQ. ID. NO. 28. (The above sequences are marked with an “i” to denote their incorporation by reference.

The invention may further include systems, method and compositions for the generation of water-soluble cannabinoids in a cell culture system expressing an endogenous glycosyltransferase. In this embodiment, one or more cannabinoids, such as in the form of a cannabinoid extract, may be introduced to a tobacco cell culture expressing one or more endogenous glycosyltransferase that may generate water-soluble cannabinoids. In some embodiment, a tobacco cell culture may be further genetically modified to express an endogenous glycosyltransferase which may be operably linked to a promoter. In this embodiment, such a promotor may be an inducible, constitutive or other promotor. In this preferred embodiment, such an endogenous glycosyltransferase may cause the overexpression of the protein generating a more robust cannabinoid biotransformation system.

As noted above, present invention allows the scaled production of water-soluble cannabinoids. Because of this enhanced solubility, the invention allows for the addition of such water-soluble cannabinoid to a variety of compositions without requiring oils and or emulsions that are generally required to maintain the non-modified cannabinoids in suspension. As a result, the present invention may all for the production of a variety of compositions for both the food and beverage industry, as well as pharmaceutical applications that do not required oils and emulsion suspensions and the like.

In one embodiment the invention may include aqueous compositions containing one or more water-soluble cannabinoids that may be introduced to a food or beverage. In a preferred embodiment, the invention may include an aqueous solution containing one or more dissolved water-soluble cannabinoids. In this embodiment, such water-soluble cannabinoid may include a glycosylated cannabinoid, and/or an acetylated cannabinoid, and/or a mixture of both. Here, the glycosylated cannabinoid, and/or said acetylated cannabinoid were generated in vivo as generally described herein, or in vitro. In additional embodiment, the water-soluble cannabinoid may be an isolated non-psychoactive, such as CBD and the like. Moreover, in this embodiment, the aqueous may contain one or more of the following: saline, purified water, propylene glycol, deionized water, and/or an alcohol such as ethanol as well as a pH buffer that may allow the aqueous solution to be maintained at a pH below 7.4. Additional embodiments may include the addition an acid of base, such as formic acid, or ammonium hydroxide.

In another embodiment, the invention may include a consumable food additive having at least one water-soluble cannabinoid, such as a glycosylated and/or an acetylated cannabinoid, and/or a mixture of both, where such water-soluble cannabinoids may be generated in vivo and/or in vitro. This consumable food additive may further include one or more a food additive polysaccharides, such as dextrin and/or maltodextrin, as well as an emulsifier. Example emulisifiers may include, but not be limited to: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.

The consumable food additive of the invention may be a homogenous composition and may further comprising a flavoring agent. Exemplary flavoring agents may include: sucrose (sugar), glucose, fructose, sorbitol, mannitol, corn syrup, high fructose corn syrup, saccharin, aspartame, sucralose, acesulfame potassium (acesulfame-K), neotame. The consumable food additive of the invention may also contain one or more coloring agents. Exemplary coloring agents may include: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate.

The consumable food additive of the invention may also contain one or more surfactants, such as glycerol monostearate and polysorbate 80. The consumable food additive of the invention may also contain one or more preservatives. Exemplary preservatives may include ascorbic acid, citric acid, sodium benzoate, calcium propionate, sodium erythorbate, sodium nitrite, calcium sorbate, potassium sorbate, BHA, BHT, EDTA, tocopherols. The consumable food additive of the invention may also contain one or more nutrient supplements, such as: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.

In one embodiment, the invention may include a consumable fluid containing at least one dissolved water-soluble cannabinoid. In one preferred embodiment, this consumable fluid may be added to a drink or beverage to infused it with the dissolved water-soluble cannabinoid generated in an in vivo system as generally herein described, or through an in vitro process, for example as identified by Zipp et al. which is incorporated herein by reference. As noted above, such water-soluble cannabinoid may include a water-soluble glycosylated cannabinoid and/or a water-soluble acetylated cannabinoid, and/or a mixture of both. The consumable fluid may include a food additive polysaccharide such as maltodextrin and/or dextrin, which may further be in an aqueous form and/or solution. For example, in one embodiment, and aqueous maltodextrin solution may include a quantity of sorbic acid and an acidifying agent to provide a food grade aqueous solution of maltodextrin having a pH of 2-4 and a sorbic acid content of 0.02-0.1% by weight.

In certain embodiments, the consumable fluid may include water, as well as an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water. The consumable fluid may further include at least one additional ingredients, including but not limited to: xanthan gum, cellulose gum, whey protein hydrolysate, ascorbic acid, citric acid, malic acid, sodium benzoate, sodium citrate, sugar, phosphoric acid, and water.

In one embodiment, the invention may include a consumable gel having at least one water-soluble cannabinoid and gelatin in an aqueous solution. In a preferred embodiment, the consumable gel may include a water-soluble glycosylated cannabinoid and/or a water-soluble acetylated cannabinoid, or a mixture of both, generated in an in vivo system, such as a whole plant or cell suspension culture system as generally herein described.

Additional embodiments may include a liquid composition having at least one water-soluble cannabinoid solubilized in a first quantity of water; and at least one of: xanthan gum, cellulose gum, whey protein hydrolysate, ascorbic acid, citric acid, malic acid, sodium benzoate, sodium citrate, sugar, phosphoric acid, and/or a sugar alcohol. In this embodiment, a water-soluble cannabinoid may include a glycosylated water-soluble cannabinoid, an acetylated water-soluble cannabinoid, or a mixture of both. In one preferred embodiment, the composition may further include a quantity of ethanol. Here, the amount of water-soluble cannabinoid may include: less than 10 mass % water; more than 95 mass % water; about 0.1 mg to about 1000 mg of the water-soluble cannabinoid; about 0.1 mg to about 500 mg of the water-soluble cannabinoid; about 0.1 mg to about 200 mg of the water-soluble cannabinoid; about 0.1 mg to about 100 mg of the water-soluble cannabinoid; about 0.1 mg to about 100 mg of the water-soluble cannabinoid; about 0.1 mg to about 10 mg of the water-soluble cannabinoid; about 0.5 mg to about 5 mg of the water-soluble cannabinoid; about 1 mg/kg to 5 mg/kg (body weight) in a human of the water-soluble cannabinoid.

In alternative embodiment, the composition may include at least one water-soluble cannabinoid in the range of 50 mg/L to 300 mg/L; at least one water-soluble cannabinoid in the range of 50 mg/L to 100 mg/L; at least one water-soluble cannabinoid in the range of 50 mg/L to 500 mg/L; at least one water-soluble cannabinoid over 500 mg/L; at least one water-soluble cannabinoid under 50 mg/L. Additional embodiments may include one or more of the following additional components: a flavoring agent; a coloring agent; a coloring agent; and/or caffeine.

In one embodiment, the invention may include a liquid composition having at least one water-soluble cannabinoid solubilized in said first quantity of water and a first quantity of ethanol in a liquid state. In a preferred embodiment, a first quantity of ethanol in a liquid state may be between 1% to 20% weight by volume of the liquid composition. In this embodiment, a water-soluble cannabinoid may include a glycosylated water-soluble cannabinoid, an acetylated water-soluble cannabinoid, or a mixture of both. Such water-soluble cannabinoids may be generated in an in vivo and/or in vitro system as herein identified. In a preferred embodiment, the ethanol, or ethyl alcohol component may be up to about ninety-nine point nine-five percent (99.95%) by weight and the water-soluble cannabinoid about zero point zero five percent (0.05%) by weight. In another embodiment, Examples of the preferred embodiment may include liquid ethyl alcohol compositions having one or more water-soluble cannabinoids wherein said ethyl alcohol has a proof greater than 100, and/or less than 100. Additional examples of a liquid composition containing ethyl alcohol and at least one water-soluble cannabinoid may include, beer, wine and/or distilled spirit.

Additional embodiments of the invention may include a chewing gum composition having a first quantity of at least one water-soluble cannabinoid. In a preferred embodiment, a chewing gum composition may further include a gum base comprising a buffering agent selected from the group consisting of acetates, glycinates, phosphates, carbonates, glycerophosphates, citrates, borates, and mixtures thereof. Additional components may include at least one sweetening agent; and at least one flavoring agent. As noted above, in a preferred embodiment, a water-soluble cannabinoid may include at least one water-soluble acetylated cannabinoid, and/or at least one water-soluble glycosylated cannabinoid, or a mixture of the two. In this embodiment, such water soluble glycosylated cannabinoid, and/or said acetylated cannabinoid may have been glycosylated and/or acetylated in vivo respectively.

In one embodiment, the chewing gum composition described above may include:
0.01 to 1% by weight of at least one water-soluble cannabinoid;
25 to 85% by weight of a gum base;
10 to 35% by weight of at least one sweetening agent; and
1 to 10% by weight of a flavoring agent.

Here, such flavoring agents may include: menthol flavor, eucalyptus, mint flavor and/or L-menthol. Sweetening agents may include one or more of the following: xylitol, sorbitol, isomalt, aspartame, sucralose, acesulfame potassium, and saccharin. Additional preferred embodiment may include a chewing gum having a pharmaceutically acceptable excipient selected from the group consisting of: fillers, disintegrants, binders, lubricants, and antioxidants. The chewing gum composition may further be non-disintegrating and also include one or more coloring and/or flavoring agents.

The invention may further include a composition for a water-soluble cannabinoid infused solution comprising essentially of: water and/or purified water, at least one water-soluble cannabinoid, and at least one flavoring agent. A water-soluble cannabinoid infused solution of the invention may further include a sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components of the water-soluble cannabinoid infused solution may include, but not be limited to: sodium chloride, sodium chloride solution, glycerin, a coloring agent, and a demulcent. As to this last potential component, in certain embodiment, a demulcent may include: pectin, glycerin, honey, methylcellulose, and/or propylene glycol. As noted above, in a preferred embodiment, a water-soluble cannabinoid may include at least one water-soluble acetylated cannabinoid, and/or at least one water-soluble glycosylated cannabinoid, or a mixture of the two. In this embodiment, such water soluble glycosylated cannabinoid, and/or said acetylated cannabinoid may have been glycosylated and/or acetylated in vivo respectively.

The invention may further include a composition for a water-soluble cannabinoid infused anesthetic solution having water, or purified water, at least one water-soluble cannabinoid, and at least one oral anesthetic. In a preferred embodiment, an anesthetic may include benzocaine, and/or phenol in a quantity of between 0.1% to 15% volume by weight.

Additional embodiments may include a water-soluble cannabinoid infused anesthetic solution having a sweetener which may be selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components of the water-soluble cannabinoid infused solution may include, but not be limited to: sodium chloride, sodium chloride solution, glycerin, a coloring agent a demulcent. In a preferred embodiment, a demulcent may selected from the group consisting of: pectin, glycerin, honey, methylcellulose, and propylene glycol. As noted above, in a preferred embodiment, a water-soluble cannabinoid may include at least one water-soluble acetylated cannabinoid, and/or at least one water-soluble glycosylated cannabinoid, or a mixture of the two. In this embodiment, such water soluble glycosylated cannabinoid, and/or said acetylated cannabinoid may have been glycosylated and/or acetylated in vivo respectively.

The invention may further include a composition for a hard lozenge for rapid delivery of water-soluble cannabinoids through the oral mucosa. In this embodiment, such a hard lozenge composition may include: a crystalized sugar base, and at least one water-soluble cannabinoid, wherein the hard lozenge has a moisture content between 0.1 to 2%. In this embodiment, the water-soluble cannabinoid may be added to the sugar based when it is in a liquefied form and prior to the evaporation of the majority of water content. Such a hard lozenge may further be referred to as a candy.

In a preferred embodiment, a crystalized sugar base may be formed from one or more of the following: sucrose, invert sugar, corn syrup, and isomalt or a combination of the same. Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine, and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The hard lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a water-soluble cannabinoid may include at least one water-soluble acetylated cannabinoid, and/or at least one water-soluble glycosylated cannabinoid, or a mixture of the two. In this embodiment, such water soluble glycosylated cannabinoid, and/or said acetylated cannabinoid may have been glycosylated and/or acetylated in vivo respectively.

The invention may include a chewable lozenge for rapid delivery of water-soluble cannabinoids through the oral mucosa. In a preferred embodiment, the compositions may include: a glycerinated gelatin base, at least one sweetener; and at least one water-soluble cannabinoid dissolved in a first quantity of water. In this embodiment, a sweetener may include sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.

Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine, and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The chewable lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a water-soluble cannabinoid may include at least one water-soluble acetylated cannabinoid, and/or at least one water-soluble glycosylated cannabinoid, or a mixture of the two. In this embodiment, such water soluble glycosylated cannabinoid, and/or said acetylated cannabinoid may have been glycosylated and/or acetylated in vivo respectively.

The invention may include a soft lozenge for rapid delivery of water-soluble cannabinoids through the oral mucosa. In a preferred embodiment, the compositions may include: polyethylene glycol base, at least one sweetener; and at least one water-soluble cannabinoid dissolved in a first quantity of water. In this embodiment, a sweetener may include sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine, and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The soft lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a water-soluble cannabinoid may include at least one water-soluble acetylated cannabinoid, and/or at least one water-soluble glycosylated cannabinoid, or a mixture of the two. In this embodiment, such water soluble glycosylated cannabinoid, and/or said acetylated cannabinoid may have been glycosylated and/or acetylated in vivo respectively.

In another embodiment, the invention may include a tablet or capsule consisting essentially of a water-soluble glycosylated cannabinoid and a pharmaceutically acceptable excipient. Example may include solid, semi-solid and aqueous excipients such as: maltodextrin, whey protein isolate, xanthan gum, guar gum, diglycerides, monoglycerides, carboxymethyl cellulose, glycerin, gelatin, polyethylene glycol and water-based excipients.

In a preferred embodiment, a water-soluble cannabinoid may include at least one water-soluble acetylated cannabinoid, and/or at least one water-soluble glycosylated cannabinoid, or a mixture of the two. In this embodiment, such water soluble glycosylated cannabinoid, and/or said acetylated cannabinoid may have been glycosylated and/or acetylated in vivo respectively. Examples of such in vivo systems being generally described herein, including in plant, as well as cell culture systems including cannabis cell culture, tobacco cell culture and yeast cell culture systems. In one embodiment, a tablet or capsule may include an amount of water-soluble cannabinoid of 5 milligrams or less. Alternative embodiments may include an amount of water-soluble cannabinoid between 5 milligrams and 200 milligrams. Still other embodiments may include a tablet or capsule having amount of water-soluble cannabinoid that is more than 200 milligrams.

The invention may further include a method of manufacturing and packaging a cannabinoid dosage, consisting of the following steps: 1) preparing a fill solution with a desired concentration of a water-soluble cannabinoid in a liquid carrier wherein said cannabinoid solubilized in said liquid carrier; 2) encapsulating said fill solution in capsules; 3) packaging said capsules in a closed packaging system; and 4) removing atmospheric air from the capsules. In one embodiment, the step of removing of atmospheric air consists of purging the packaging system with an inert gas, such as, for example, nitrogen gas, such that said packaging system provides a room temperature stable product. In one preferred embodiment, the packaging system may include a plaster package, which may be constructed of material that minimizes exposure to moisture and air.

In one embodiment a preferred liquid carrier may include a water-based carrier, such as for example an aqueous sodium chloride solution. In a preferred embodiment, a water-soluble cannabinoid may include at least one water-soluble acetylated cannabinoid, and/or at least one water-soluble glycosylated cannabinoid, or a mixture of the two. In this embodiment, such water soluble glycosylated cannabinoid, and/or said acetylated cannabinoid may have been glycosylated and/or acetylated in vivo respectively. Examples of such in vivo systems being generally described herein, including in plant, as well as cell culture systems including cannabis cell culture, tobacco cell culture and yeast cell culture systems. In one embodiment, a desired cannabinoid concentration may be about 1-10% w/w, while in other embodiments it may be about 1.5-6.5% w/w. Alternative embodiments may include an amount of water-soluble cannabinoid between 5 milligrams and 200 milligrams. Still other embodiments may include a tablet or capsule having amount of water-soluble cannabinoid that is more than 200 milligrams.

The invention may include an oral pharmaceutical solution, such as a sub-lingual spray, consisting essentially of a water-soluble cannabinoid, 30-33% w/w water, about 50% w/w alcohol, 0.01% w/w butylated hydroxylanisole (BHA) or 0.1% w/w ethylenediaminetetraacetic acid (EDTA) and 5-21% w/w co-solvent, having a combined total of 100%, wherein said co-solvent is selected from the group consisting of propylene glycol, polyethylene glycol and combinations thereof, and wherein said water-soluble cannabinoid is a glycosylated cannabinoid, an acetylated cannabinoid or a mixture of the two. In an alternative embodiment, such a oral pharmaceutical solution may consist essentially of 0.1 to 5% w/w of said water-soluble cannabinoid, about 50% w/w alcohol, 5.5% w/w propylene glycol, 12% w/w polyethylene glycol and 30-33% w/w water. In a preferred composition, the alcohol component may be ethanol.

The invention may include an oral pharmaceutical solution, such as a sublingual spray, consisting essentially of about 0.1% to 1% w/w water-soluble cannabinoid, about 50% w/w alcohol, 5.5% w/w propylene glycol, 12% w/w polyethylene glycol, 30-33% w/w water, 0.01% w/w butylated hydroxyanisole, having a combined total of 100%, and wherein said water-soluble cannabinoid is a glycosylated cannabinoid, an acetylated cannabinoid or a mixture of the two wherein that were generated in vivo. In an alternative embodiment, such a oral pharmaceutical solution may consist essentially of 0.54% w/w water-soluble cannabinoid, 31.9% w/w water, 12% w/w polyethylene glycol 400, 5.5% w/w propylene glycol, 0.01% w/w butylated hydroxyanisole, 0.05% w/w sucralose, and 50% w/w alcohol, wherein the a the alcohol components may be ethanol.

The invention may include a solution for nasal and/or sublingual administration of a cannabinoid including: 1) an excipient of propylene glycol, ethanol anhydrous, or a mixture of both; and 2) a water-soluble cannabinoid which may include glycosylated cannabinoid an acetylated cannabinoid or a mixture of the two generated in vivo and/or in vitro. In a preferred embodiment, the composition may further include a topical decongestant, which may include phenylephrine hydrochloride, Oxymetazoline hydrochloride, and Xylometazoline in certain preferred embodiments. The composition may further include an antihistamine, and/or a steroid. Preferably, the steroid component is a corticosteroid selected from the group consisting of: neclomethasone dipropionate, budesonide, ciclesonide, flunisolide, fluticasone furoate, fluticasone propionate, mometasone, triamcinolone acetonide. In alternative embodiment, the solution for nasal and/or sublingual administration of a cannabinoid may further comprise at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.

The invention may further include an aqueous solution for nasal and/or sublingual administration of a cannabinoid comprising: a water and/or saline solution; and a water-soluble cannabinoid which may include a glycosylated cannabinoid, an acetylated cannabinoid or a mixture of the two generated in vivo and/or in vitro. In a preferred embodiment, the composition may further include a topical decongestant, which may include phenylephrine hydrochloride, Oxymetazoline hydrochloride, and Xylometazoline in certain preferred embodiments. The composition may further include an antihistamine, and/or a steroid. Preferably, the steroid component is a corticosteroid selected from the group consisting of: neclomethasone dipropionate, budesonide, ciclesonide, flunisolide, fluticasone furoate, fluticasone propionate, mometasone, triamcinolone acetonide. In alternative embodiment, the aqueous solution may further comprise at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.

The invention may include a topical formulation for the transdermal delivery of water-soluble cannabinoid. In a preferred embodiment, a topical formulation for the transdermal delivery of water-soluble cannabinoid may include a water-soluble glycosylated cannabinoid, and/or water-soluble acetylated cannabinoid, or a mixture of both, and a pharmaceutically acceptable excipient. Here, a glycosylated cannabinoid and/or acetylated cannabinoid may be generated in vivo and/or in vitro. Preferably a pharmaceutically acceptable excipient may include one or more: gels, ointments, cataplasms, poultices, pastes, creams, lotions, plasters and jellies or even polyethylene glycol. Additional embodiments may further include one or more of the following components: a quantity of capsaicin; a quantity of benzocaine; a quantity of lidocaine; a quantity of camphor; a quantity of benzoin resin; a quantity of methylsalicilate; a quantity of triethanolamine salicylate; a quantity of hydrocortisone; a quantity of salicylic acid.

The invention may include a gel for transdermal administration of a water soluble-cannabinoid which may be generated in vitro and/or in vivo. In this embodiment, the mixture preferably contains from 15% to about 90% ethanol, about 10% to about 60% buffered aqueous solution or water, about 0.1 to about 25% propylene glycol, from about 0.1 to about 20% of a gelling agent, from about 0.1 to about 20% of a base, from about 0.1 to about 20% of an absorption enhancer and from about 1% to about 25% polyethylene glycol and a water-soluble cannabinoid such as a glycosylated cannabinoid, and/or acetylated cannabinoid, and/or a mixture of the two.

In another embodiment, the invention may further include a transdermal composition having a pharmaceutically effective amount of a water-soluble cannabinoid for delivery of the cannabinoid to the bloodstream of a user. This transdermal composition may include a pharmaceutically acceptable excipient and at least one water-soluble cannabinoid, such as a glycosylated cannabinoid, an acetylated cannabinoid, and a mixture of both, wherein the cannabinoid is capable of diffusing from the composition into the bloodstream of the user. In a preferred embodiment, a pharmaceutically acceptable excipient to create a transdermal dosage form selected from the group consisting of: gels, ointments, cataplasms, poultices, pastes, creams, lotions, plasters and jellies. The transdermal composition may further include one or more surfactants. In one preferred embodiment, the surfactant may include a surfactant-lecithin organogel, which may further be present in an amount of between about between about 95% and about 98% w/w. In an alternative embodiment, a surfactant-lecithin organogel comprises lecithin and PPG-2 myristyl ether propionate and/or high molecular weight polyacrylic acid polymers. The transdermal composition may further include a quantity of isopropyl myristate.

The invention may further include transdermal composition having one or more permeation enhancers to facilitate transfer of the water-soluble cannabinoid across a dermal layer. In a preferred embodiment, a permeation enhancer may include one or more of the following: propylene glycol monolaurate, diethylene glycol monoethyl ether, an oleoyl macrogolglyceride, a caprylocaproyl macrogolglyceride, and an oleyl alcohol,

The invention may also include a liquid cannabinoid liniment composition consisting of water, isopropyl alcohol solution and a water-soluble cannabinoid, such as glycosylated cannabinoid, and/or said acetylated cannabinoid which may further have been generated in vivo. This liquid cannabinoid liniment composition may further include approximately 97.5% to about 99.5% by weight of 70% isopropyl alcohol solution and from about 0.5% to about 2.5% by weight of a water-soluble cannabinoid mixture.

Based on the improved solubility and other physical properties, as well as cost advantage and scalability of the invention's in vivo water-soluble production platform, the invention may include one or more commercial infusions. For example, commercially available products, such a lip balm, soap, shampoos, lotions, creams and cosmetics may be infused with one or more water-soluble cannabinoids.

As generally described herein, the invention may include one or more plants, such as a tobacco plant and/or cell culture that may be genetically modified to produce, for example water-soluble glycosylated cannabinoids in vivo. As such, in one preferred embodiment, the invention may include a tobacco plant and or cell that contain at least one water-soluble cannabinoid. In a preferred embodiment, a tobacco plant containing a quantity of water-soluble cannabinoids may be used to generate a water-soluble cannabinoid infused tobacco product such as a cigarette, pipe tobacco, chewing tobacco, cigar, and smokeless tobacco. In one embodiment, the tobacco plant may be treated with one or more glycosidase inhibitors. In a preferred embodiment, since the cannabinoid being introduced to the tobacco plant may be controlled, the inventive tobacco plant may generate one or more selected water-cannabinoids. For example, in one embodiment, the genetically modified tobacco plant may be introduced to a single cannabinoid, such as a non-psychoactive CBD compound, while in other embodiment, the genetically modified tobacco plant may be introduced to a cannabinoid extract containing a full and/or partial entourage of cannabinoid compounds.

The invention may further include a novel composition that may be used to supplement a cigarette, or other tobacco-based product. In this embodiment, the composition may include at least one water-soluble cannabinoid dissolved in an aqueous solution. This aqueous solution may be wherein said composition may be introduced to a tobacco product, such as a cigarette and/or a tobacco leaf such that the aqueous solution may evaporate generating a cigarette and/or a tobacco leaf that contains the aforementioned water-soluble cannabinoid(s), which may further have been generated in vivo as generally described herein.

On one embodiment the invention may include one or more method of treating a medical condition in a mammal. In this embodiment, the novel method may include of administering a therapeutically effective amount of a water-soluble cannabinoid, such as an in vivo generated glycosylated cannabinoid, and/or an acetylated cannabinoid, and/or a mixture of both or a pharmaceutically acceptable salt thereof, wherein the medical condition is selected from the group consisting of: obesity, post-traumatic stress syndrome, anorexia, nausea, emesis, pain, wasting syndrome, HIV-wasting, chemotherapy induced nausea and vomiting, alcohol use disorders, anti-tumor, amyotrophic lateral sclerosis, glioblastoma multiforme, glioma, increased intraocular pressure, glaucoma, cannabis use disorders, Tourette's syndrome, dystonia, multiple sclerosis, inflammatory bowel disorders, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, and juvenile rheumatoid arthritis. In a preferred embodiment, the pharmaceutical composition may be administered by a route selected from the group consisting of: transdermal, topical, oral, buccal, sublingual, intra-venous, intra-muscular, vaginal, rectal, ocular, nasal and follicular. The amount of water-soluble cannabinoids may be a therapeutically effective amount, which may be determined by the patient's age, weight, medical condition cannabinoid-delivered, route of delivery and the like. In one embodiment, a therapeutically effective amount may be 50 mg or less of a water-soluble cannabinoid. In another embodiment, a therapeutically effective amount may be 50 mg or more of a water-soluble cannabinoid.

It should be noted that for any of the above composition, unless otherwise stated, an effective amount of water-soluble cannabinoids may include amounts between: 0.01 mg to 0.1 mg; 0.01 mg to 0.5 mg; 0.01 mg to 1 mg; 0.01 mg to 5 mg; 0.01 mg to 10 mg; 0.01 mg to 25 mg; 0.01 mg to 50 mg; 0.01 mg to 75 mg; 0.01 mg to 100 mg; 0.01 mg to 125 mg; 0.01 mg to 150 mg; 0.01 mg to 175 mg; 0.01 mg to 200 mg; 0.01 mg to 225 mg; 0.01 mg to 250 mg; 0.01 mg to 275 mg; 0.01 mg to 300 mg; 0.01 mg to 225 mg; 0.01 mg to 350 mg; 0.01 mg to 375 mg; 0.01 mg to 400 mg; 0.01 mg to 425 mg; 0.01 mg to 450 mg; 0.01 mg to 475 mg; 0.01 mg to 500 mg; 0.01 mg to 525 mg; 0.01 mg to 550 mg; 0.01 mg to 575 mg; 0.01 mg to 600 mg; 0.01 mg to 625 mg; 0.01 mg to 650 mg; 0.01 mg to 675 mg; 0.01 mg to 700 mg; 0.01 mg to 725 mg; 0.01 mg to 750 mg; 0.01 mg to 775 mg; 0.01 mg to 800 mg; 0.01 mg to 825 mg; 0.01 mg to 950 mg; 0.01 mg to 875 mg; 0.01 mg to 900 mg; 0.01 mg to 925 mg; 0.01 mg to 950 mg; 0.01 mg to 975 mg; 0.01 mg to 1000 mg; 0.01 mg to 2000 mg; 0.01 mg to 3000 mg; 0.01 mg to 4000 mg; 01 mg to 5000 mg; 0.01 mg to 0.1 mg/kg; 0.01 mg to 0.5 mg/kg; 01 mg to 1 mg/kg; 0.01 mg to 5 mg/kg; 0.01 mg to 10 mg/kg; 0.01 mg to 25 mg/kg; 0.01 mg to 50 mg/kg; 0.01 mg to 75 mg/kg; and 0.01 mg to 100 mg/kg.

The modified cannabinoids compounds of the present invention are useful for a variety of therapeutic applications. For example, the compounds are useful for treating or alleviating symptoms of diseases and disorders involving CB1 and CB2 receptors, including appetite loss, nausea and vomiting, pain, multiple sclerosis and epilepsy. For example, they may be used to treat pain (i.e. as analgesics) in a variety of applications including but not limited to pain management. In additional embodiments, such modified cannabinoids compounds may be used as an appetite suppressant. Additional embodiment may include administering the modified cannabinoids compounds.

By “treating” the present inventors mean that the compound is administered in order to alleviate symptoms of the disease or disorder being treated. Those of skill in the art will recognize that the symptoms of the disease or disorder that is treated may be completely eliminated, or may simply be lessened. Further, the compounds may be administered in combination with other drugs or treatment modalities, such as with chemotherapy or other cancer-fighting drugs.

Implementation may generally involve identifying patients suffering from the indicated disorders and administering the compounds of the present invention in an acceptable form by an appropriate route. The exact dosage to be administered may vary depending on the age, gender, weight and overall health status of the individual patient, as well as the precise etiology of the disease. However, in general, for administration in mammals (e.g. humans), dosages in the range of from about 0.01 to about 300 mg of compound per kg of body weight per 24 hr., and more preferably about 0.01 to about 100 mg of compound per kg of body weight per 24 hr., are effective.

Administration may be oral or parenteral, including intravenously, intramuscularly, subcutaneously, intradermal injection, intraperitoneal injection, etc., or by other routes (e.g. transdermal, sublingual, oral, rectal and buccal delivery, inhalation of an aerosol, etc.). In a preferred embodiment of the invention, the water-soluble cannabinoid analogs are provided orally or intravenously.

In particular, the phenolic esters of the invention are preferentially administered systemically in order to afford an opportunity for metabolic activation via in vivo cleavage of the ester. In addition, the water soluble compounds with azole moieties at the pentyl side chain do not require in vivo activation and may be suitable for direct administration (e.g. site specific injection).

The compounds may be administered in the pure form or in a pharmaceutically acceptable formulation including suitable elixirs, binders, and the like (generally referred to a “carriers”) or as pharmaceutically acceptable salts (e.g. alkali metal salts such as sodium, potassium, calcium or lithium salts, ammonium, etc.) or other complexes. It should be understood that the pharmaceutically acceptable formulations include liquid and solid materials conventionally utilized to prepare both injectable dosage forms and solid dosage forms such as tablets and capsules and aerosolized dosage forms. In addition, the compounds may be formulated with aqueous or oil based vehicles. Water may be used as the carrier for the preparation of compositions (e.g. injectable compositions), which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, stabilizers, elixirs, and binders or encapsulants (lactose, liposomes, etc). Solid diluents and excipients include lactose, starch, conventional disintergrating agents, coatings and the like. Preservatives such as methyl paraben or benzalkium chloride may also be used. Depending on the formulation, it is expected that the active composition will consist of about 1% to about 99% of the composition and the vehicular “carrier” will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

The administration of the compounds of the present invention may be intermittent, bolus dose, or at a gradual or continuous, constant or controlled rate to a patient. In addition, the time of day and the number of times per day that the pharmaceutical formulation is administered may vary are and best determined by a skilled practitioner such as a physician. Further, the effective dose can vary depending upon factors such as the mode of delivery, gender, age, and other conditions of the patient, as well as the extent or progression of the disease. The compounds may be provided alone, in a mixture containing two or more of the compounds, or in combination with other medications or treatment modalities. The compounds may also be added to blood ex vivo and then be provided to the patient.

Genes encoding by a combination polynucleotide and/or a homologue thereof, may be introduced into a plant, and/or plant cell using several types of transformation approaches developed for the generation of transgenic plants. Standard transformation techniques, such as Ti-plasmid Agrobacterium-mediated transformation, particle bombardment, microinjection, and electroporation may be utilized to construct stably transformed transgenic plants.

As used herein, a “cannabinoid” is a chemical compound (such as cannabinol, THC or cannabidiol) that is found in the plant species Cannabis among others like Echinacea; Acmella Oleracea; Helichrysum Umbraculigerum; Radula Marginata (Liverwort) and Theobroma Cacao, and metabolites and synthetic analogues thereof that may or may not have psychoactive properties. Cannabinoids therefore include (without limitation) compounds (such as THC) that have high affinity for the cannabinoid receptor (for example Ki<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6,12-dihydro-6-hydroxy-cannabidiol (described in U.S. Pat. No. 5,227,537, incorporated by reference); (3 S,4R)-7-hydroxy-A6-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3.1.1]hept-2-en, and other 4-phenylpinene derivatives disclosed in U.S. Pat. No. 5,434,295, which is incorporated by reference; and cannabidiol (−)(CBD) analogs such as (−)CBD-monomethylether, (−)CBD dimethyl ether; (−)CBD diacetate; (−)3′-acetyl-CBD monoacetate; and ±AF 11, all of which are disclosed in Consroe et al., J. Clin. Phannacol. 21:428S-436S, 1981, which is also incorporated by reference. Many other cannabinoids are similarly disclosed in Agurell et al., Pharmacol. Rev. 38:31-43, 1986, which is also incorporated by reference.

As claimed herein, the term “cannabinoid” may also include different modified forms of a cannabinoid such as a hydroxylated cannabinoid or cannabinoid carboxylic acid. For example, if a glycosyltransferase were to be capable of glycosylating a cannabinoid, it would include the term cannabinoid as defined elsewhere, as well as the aforementioned modified forms. It may further include multiple glycosylation moieties.

Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3,4,5,6-tetrahydro-7-hydroxy-alpha-alpha-2-trimethyl-9-n-propyl-2,6-methano-2H-1-benzoxocin-5-methanol-cannabiripsol, trihydroxy-delta-9-tetrahydrocannabinol, and cannabinol. Examples of cannabinoids within the context of this disclosure include tetrahydrocannabinol and cannabidiol.

The term “endocannabinoid” refer to compounds including arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z),11(Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA). Particularly preferred endocannabinoids are AEA, 2-AG, 1-AG, and DHEA.

Hydroxylation is a chemical process that introduces a hydroxyl group (—OH) into an organic compound. Acetylation is a chemical reaction that adds an acetyl chemical group. Glycosylation is the coupling of a glycosyl donor, to a glycosyl acceptor forming a glycoside.

The term “prodrug” refers to a precursor of a biologically active pharmaceutical agent (drug). Prodrugs must undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety to form the biologically active pharmaceutical agent.

The term “glycosidase inhibitor” and as used in the present invention is used to mean a compound, which can inhibit glycosidase enzymes which catalyst the hydrolysis of glycosidic bonds. Techniques for determining whether a compound acts as a glycosidase inhibitor will be well known to the skilled person, but may include, for example use of substrates such as p-nitrophenyl-glycosides, where the presence of an inhibitor will reduce the release of the colored p-nitrophenol when an appropriate glycosidase is present.

As used herein, the term “homologous” with regard to a contiguous nucleic acid sequence, refers to contiguous nucleotide sequences that hybridize under appropriate conditions to the reference nucleic acid sequence. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters which initiate transcription only in certain tissues are referred to as “tissue-specific.”

A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most cell or tissue types.

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that responds to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone are general examples (Schena et al. (1991) Proc. Natl. Acad. Sci. USA 88:0421).

As used herein, the term “transformation” or “genetically modified” refers to the transfer of one or more nucleic acid molecule(s) into a cell. A plant is “transformed” or “genetically modified” by a nucleic acid molecule transduced into the plant when the nucleic acid molecule becomes stably replicated by the plant. As used herein, the term “transformation” or “genetically modified” encompasses all techniques by which a nucleic acid molecule can be introduced into, such as a plant.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria.

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. Table 1a, infra, contains information about which nucleic acid codons encode which amino acids.

TABLE 4
Amino acid Nucleic acid codons

| Amino Acid | Nucleic Acid Codons |
|---|---|
| Ala/A | GCT, GCC, GCA, GCG |
| Arg/R | CGT, CGC, CGA, CGG, AGA, |
| | AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

The term “plant” or “plant system” includes whole plants, plant organs, progeny of whole plants or plant organs, embryos, somatic embryos, embryo-like structures, protocorms, protocorm-like bodies (PLBs), and culture and/or suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The invention may also include Cannabaceae and other Cannabis strains, such as C. sativa generally.

The term “expression,” as used herein, or “expression of a coding sequence” (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acetylated amino acid), and cRNA (complementary RNA). The term “deoxyribonucleic acid” (DNA) is inclusive of cDNA, genomic DNA, and DNA-RNA hybrids. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.

As used herein with respect to DNA, the term “coding sequence,” “structural nucleotide sequence,” or “structural nucleic acid molecule” refers to a nucleotide sequence that is ultimately translated into a polypeptide, via transcription and mRNA, when placed under the control of appropriate regulatory sequences. With respect to RNA, the term “coding sequence” refers to a nucleotide sequence that is translated into a peptide, polypeptide, or protein. The boundaries of a coding sequence are determined by a translation start codon at the 5′-terminus and a translation stop codon at the 3′-terminus. Coding sequences include, but are not limited to: genomic DNA; cDNA; EST; and recombinant nucleotide sequences.

The term “sequence identity” or “identity,” as used herein in the context of two nucleic acid or polypeptide sequences, refers to the residues in the two sequences that are the same when aligned for maximum correspondence over a specified comparison window.

The term “recombinant” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed—over-expressed, under expressed or not expressed at all.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise.

As used herein, “heterologous” or “exogenous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention. By “host cell” is meant a cell which contains an introduced nucleic acid construct and supports the replication and/or expression of the construct. Host cells may be prokaryotic cells such as E. coli, or eukaryotic cells such as fungi, yeast, insect, amphibian, nematode, or mammalian cells. Alternatively, the host cells are monocotyledonous or dicotyledonous plant cells. An example of a monocotyledonous host cell is a maize host cell.

### EXAMPLES

### Example 1: Functionalization of Cannabinoids by Cytochrome P450s

The present inventors have demonstrated that cannabinoids can be functionalized in an in vivo plant system. Specifically, the present inventors utilized cytochrome P450 monooxygenases (CYP) to modify or functionalize the chemical structure of cannabinoids. As shown below, CYPs do this by inserting an oxygen atom into hydrophobic molecules to make them more reactive and hydrophilic. A representative reaction may include the generalized reaction in FIG. 13.

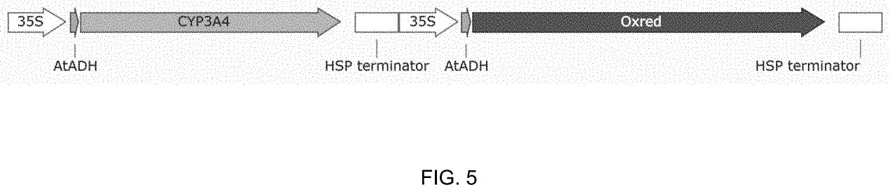

The P450 enzyme system involves several cytochrome P450 species and nonspecific cytochrome P450 oxidoreductases. As shown in FIG. 5, the present inventors used a human cytochrome P450 (CYP3A4) in a double construct with an exemplary human cytochrome P450 oxidoreductase, both expressed under the control of the constitutive CaMV 35S promoter with 5′ untranslated regions to enhance translation. Protein and DNA sequences for the functionalization of cannabinoids (CYP3A4 and P450 oxidoreductase) are identified as SEQ ID NO's. 1-4. Expression was confirmed using RT-PCR utilizing the forward and reverse primers identified in Table 3 below. As noted above, the present inventors demonstrated that overexpressing of P450s generated functionalized cannabinoids which could then be glycosylated, rendering them water-soluble.

### Example 2: P450 Overexpression Enhances In Vivo Hydroxylation and Glycosylation of Cannabinoids in Plant Systems

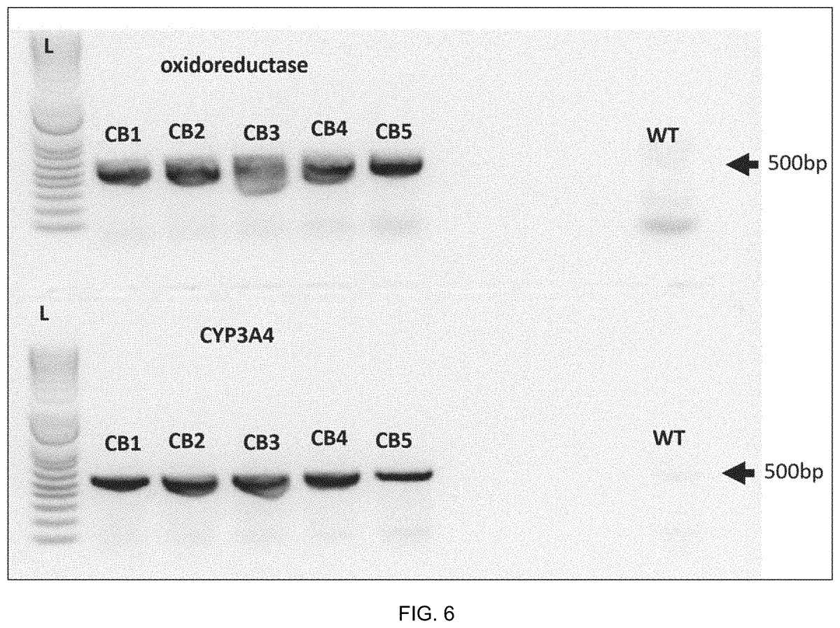

The present inventors have demonstrated that overexpression enhanced in vivo hydroxylation and glycosylation of CBDA in an exemplary plant system. Specifically, as generally shown in FIG. 6, the present inventors demonstrate that infiltration of tobacco leaves with Agrobacterium carrying CYP3A4 and P450 oxidoreductase was accomplished as described in herein. Confirmation of expression was done using RT-PCR 2-3 days after infiltration (FIG. 6).

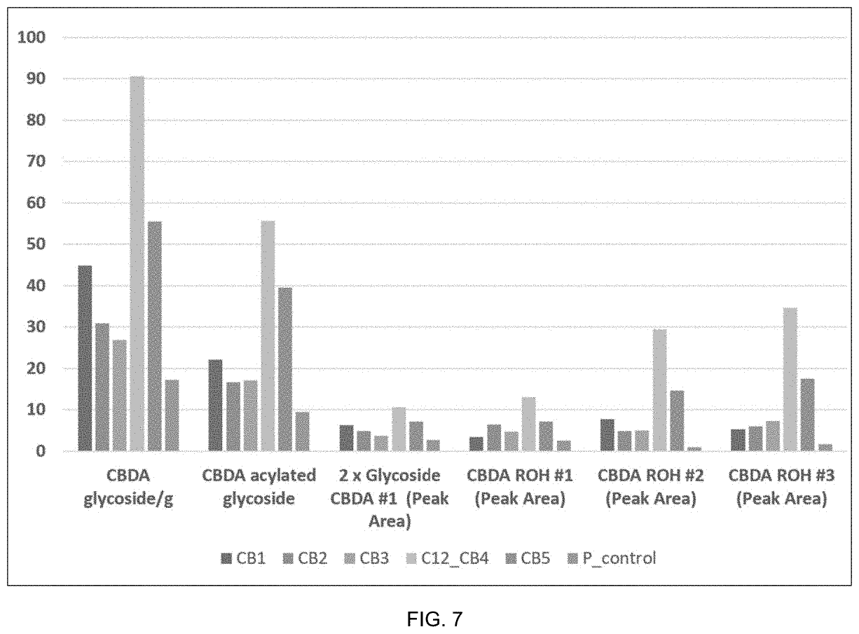

As generally shown in FIG. 7, the present inventors demonstrate that overexpression of the CYP3A4+P450 oxidoreductase construct and subsequent feeding of at least one cannabinoid, in this case CBDA, upon confirmation of expression resulted in in vivo glycosylation of CBDA in tobacco leaves (FIG. 7). On average, glycosylation increased 3-fold in transgenic N. benthamiana plants compared to the control while hydroxylation increased up to 13-fold. As such, in certain embodiment, tobacco glycosyltransferases may be utilized as key targets in the current inventive technology for glycosylation of cannabinoids.

### Example 3: Identification of Modified Water-Soluble Cannabinoids by Mass Spectrometry

The present inventors demonstrated the biosynthesis of modified functionalized as well as water-soluble cannabinoids in both in vitro as well as in vivo plant system. Specifically, the present inventors identified the cannabinoid biotransformations associated with the gene constructs in both in vitro assays and transient leaf expression. Through the use of accurate mass spectrometry measurements, the present inventors were able to identify and confirm the biosynthesis of modified water-soluble cannabinoids.

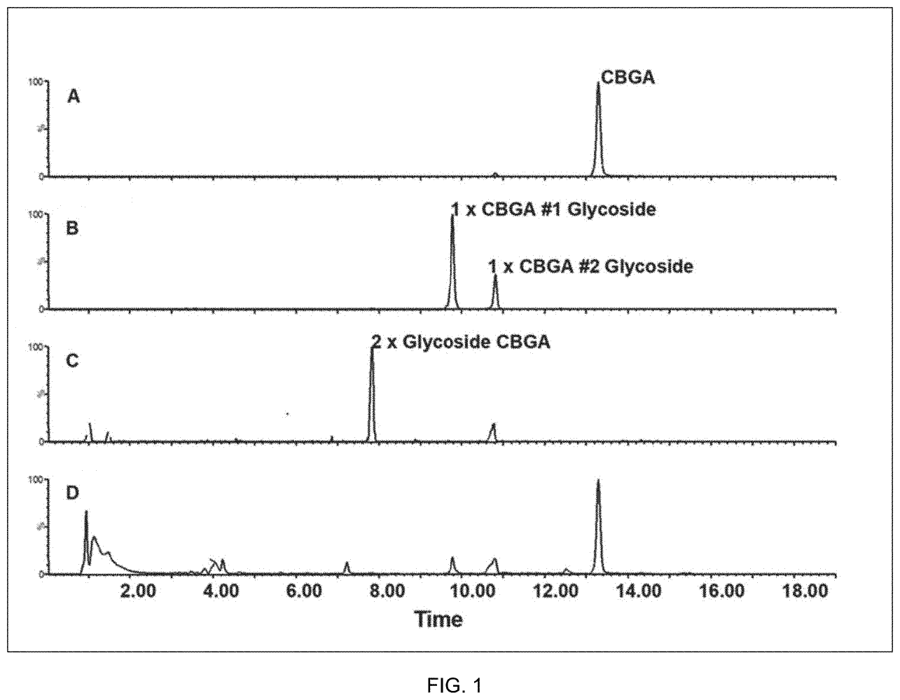

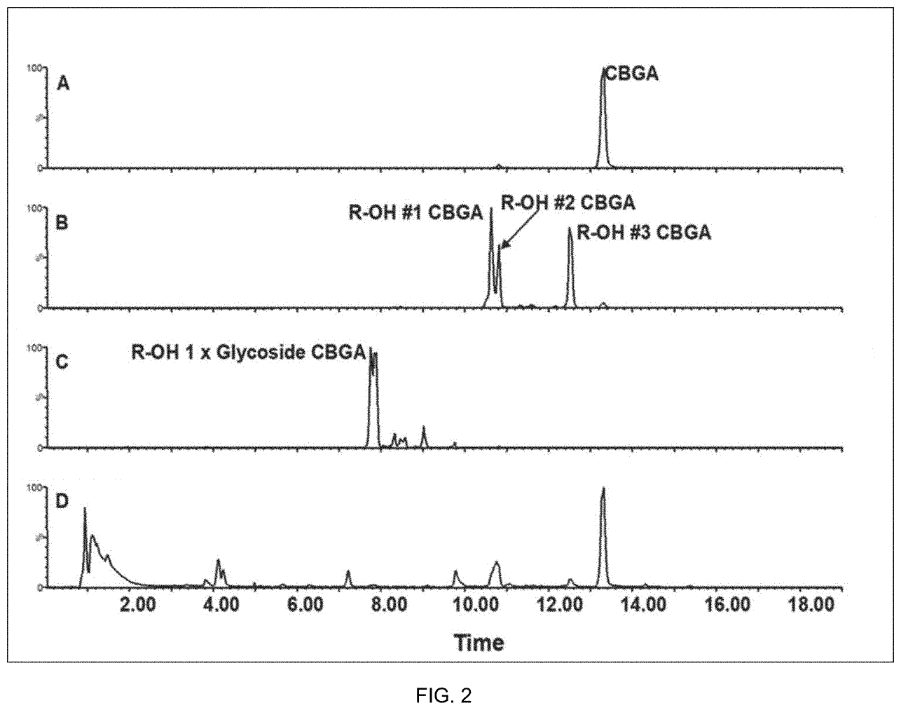

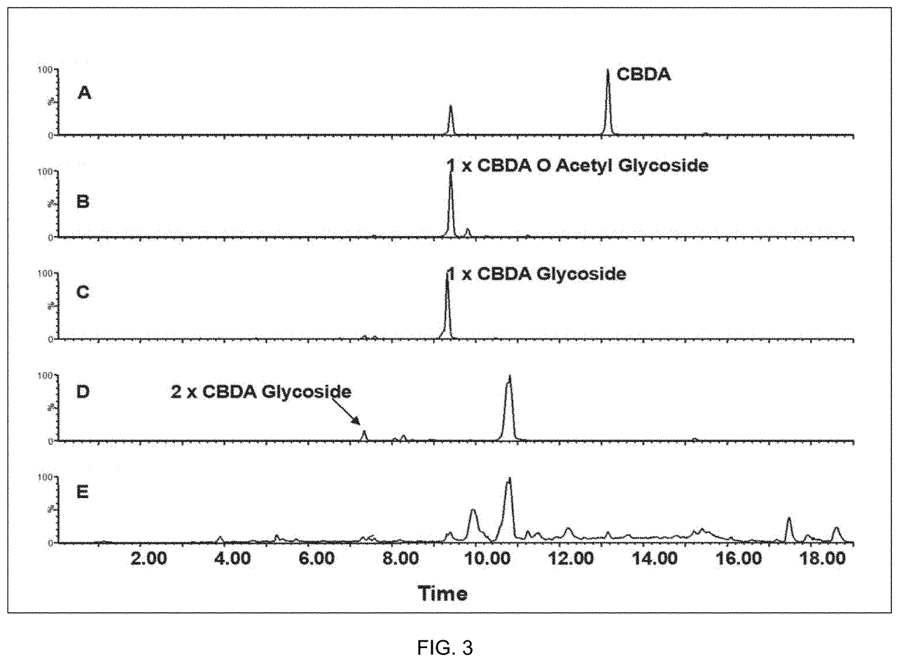

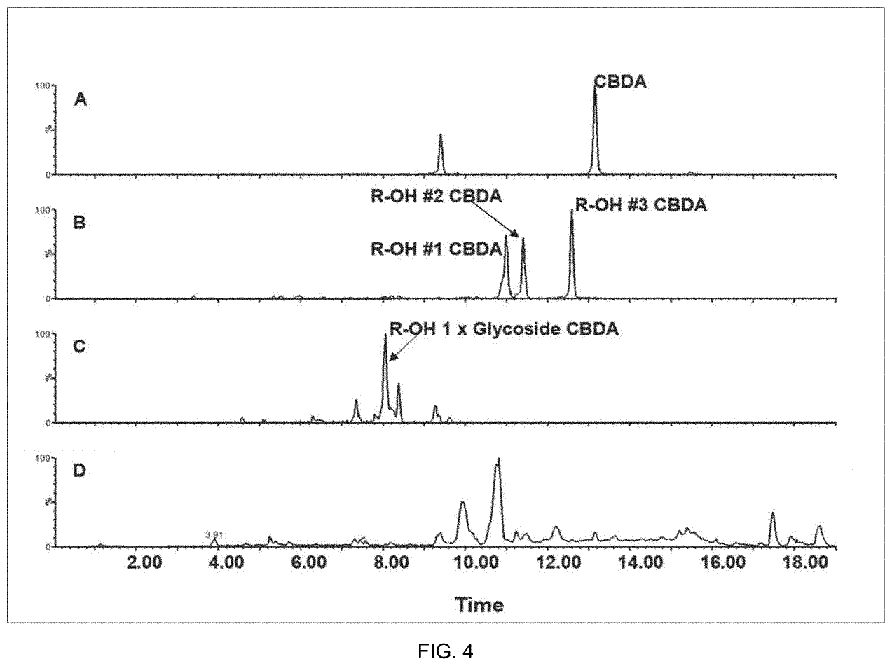

Specifically, as generally shown in FIGS. 1-4, the present inventors were able to identify the glycosylated water-soluble cannabinoids in the chromatographic analysis and were able to produce extracted ion chromatograms for peak integration. For example, FIG. 1 panel B, illustrates the identification of multiple constitutional cannabinoid isomers of a single glycoside moiety, while in FIG. 2 panel B, an example of multiple constitutional isomers of the cytochrome P450 oxidation are illustrated. Peak areas for each identified molecule were used for relative quantification between treatments. Based on these results we confirmed biosynthesis of modified cannabinoid molecules containing up to two glycosides moieties, O acetyl glycoside, as well as hydroxylation (R—OH) biotransformations. Summaries of those identifications are presented in FIGS. 36 and 37 for CBGA and CBDA respectively.

Tables 1 and 2 are provided below further demonstrating the production of the select modified cannabinoid molecules. Generally referring to Tables 1-2 below, the present inventors demonstrated that based on the reduced retention time in the water: acetonitrile HPLC gradient, the glycosylated and hydroxylated cannabinoids, which eluted earlier than their non-modified forms, are demonstrated to be more water soluble than their non-modified forms.

### Example 4: Generation of Heterologous Cytosolic Synthesis and Glycosylation Gene Constructs for Expressions in Tobacco Leaves and Cell Suspensions

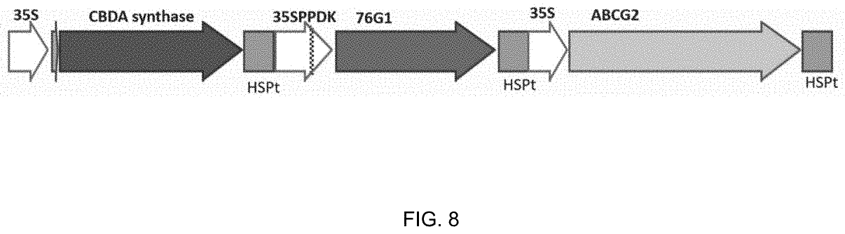

As shown in FIG. 8, the present inventors generated a triple gene construct for expression of cannabidiolic acid (CBDA) synthase in which the trichome targeting sequence had been removed, and the glycosyltransferase 76G1 from Stevia rebaudiana. In this construct the multi-drug ABC transporter ABCG2 was also included.

In one embodiment of the present inventive technology, the gene construct may be used to transform a plant cell that may further be configured to be cultured in a suspension culture. In one preferred embodiment, a cannabis cell may be transformed with the construct generally outline in FIG. 8. In this preferred embodiment, cannabinoids produced by the cannabis cells in the cell culture may be functionalize through the overexpression of the CYP3A4+P450 oxidoreductase as described above, and further glycosylated by the expression and action of the heterologous UDP glycosyltransferase (76G1) from Stevia rebaudiana referend above.

Moreover, as generally outline herein, the cannabinoids may be modified so as to be functionalized and/or glycosylated, or generally water-soluble, and may then be secreted into the cell wall area, in the case of a whole plant, or the surrounding media in suspension cultures, with the aid of the ABC transporter. In one embodiment, this construct may be used for synthesis and modification of cannabinoids in cell suspension cultures, utilizing tobacco bright yellow cells or cannabis cells.

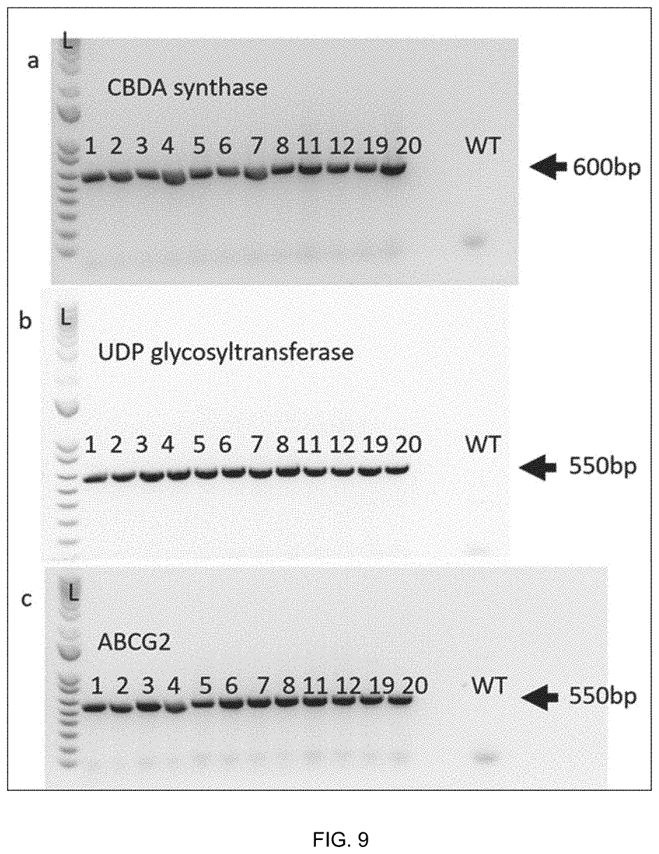

As generally shown in FIG. 9, in vivo expression of CBDA synthase, UDP glycosyltransferase 76G1 and ABCG2 was confirmed. Reverse and forward primers used in the RT-PCR reactions are provided below in Table 4 below.

The gene and protein sequence identifications for CBDA synthase are provided as SEQ ID NO's 5 and 6 respectively. It should be noted that a variety of cannabinoid synthase genes/proteins may be used with the current inventive technology, CBDA synthase being exemplary only. Indeed, it is specifically contemplated that the synthase enzyme associated with any of the cannabinoids identified herein may be incorporated into the current invention without undue experimentation. In one embodiment, one or more of such exogenous or endogenous synthase enzyme may further have the trichome targeting sequence excised, again, a step that can be readily accomplished without undue experimentation. Example may THCA synthase, CBG synthase, THCA synthase, CBDA synthase or CBCA synthase, which may in this embodiment have their trichome targeting sequence had been removed.

The gene and protein sequence identifications for glycosyltransferase 76G1 from Stevia rebaudiana are provided as SEQ ID NO's. 7, and 8 respectively. The gene and protein sequence identifications for the multi-drug ABC transporter ABCG2 are provided as SEQ ID NO's 9 and 10 respectively.

### Example 5: In Vivo Cytosolic Synthesis and Glycosylation of Cannabinoids in N. benthamiana Leaves and Cell Suspensions

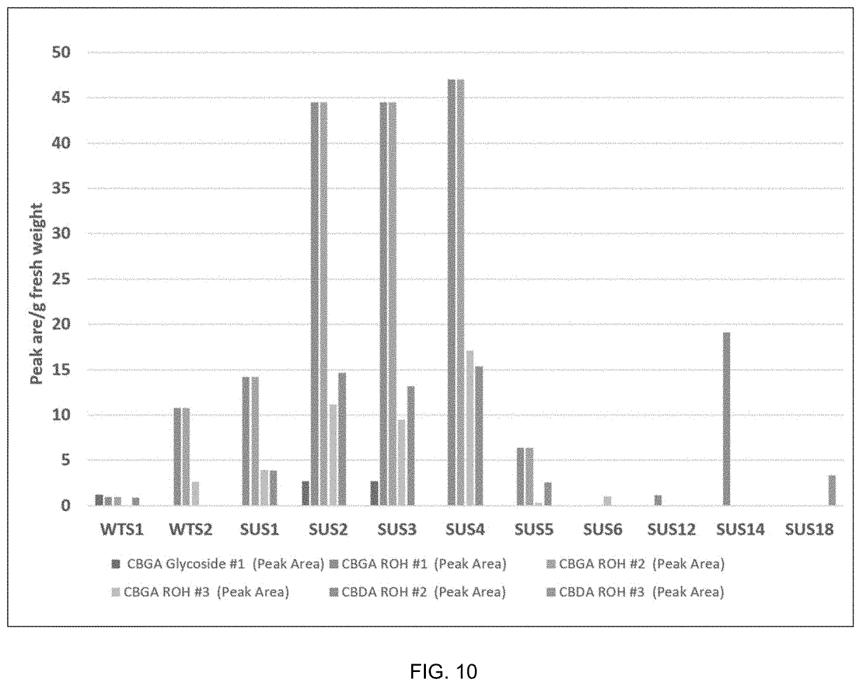

As shown in FIG. 10, the present inventors demonstrate that in plants, in this embodiment N. benthamiana, expressing the above referenced cytosolic construct, glycosylation of CBGA occurred as well as formation of modified or hydroxylated CBDA. The glycosylation of CBGA evidences in vivo glycosylation of cannabinoids by overexpressing a glycosyltransferase in N. benthamiana plants. The presence of glycosylated cannabinoids in wild type plants suggests the presence of a strong glycosyltransferase in tobacco. As such, in one embodiment, over expression of a heterologous or homologous tobacco glycosyltransferase may expressed or overexpressed resulting in the enhanced in vivo biosynthesis of water-soluble cannabinoids in whole plants, as well as in suspension cultures. For example, in one embodiment, a heterologous tobacco glycosyltransferase may be expressed in a cannabis plant or cell culture resulting in the in vivo biosynthesis of water-soluble cannabinoids in the Cannabis plant and/or a Cannabis suspension cultures.

### Example 6: Water Soluble Cannabinoid Production Systems Utilizing MTB Transcription Factor and/or Catalase

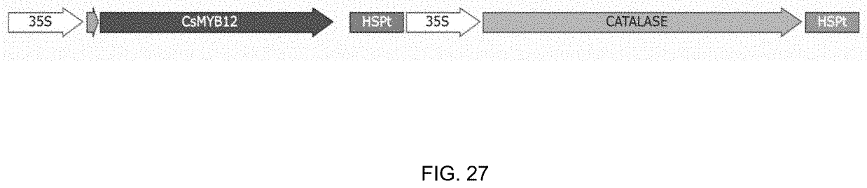

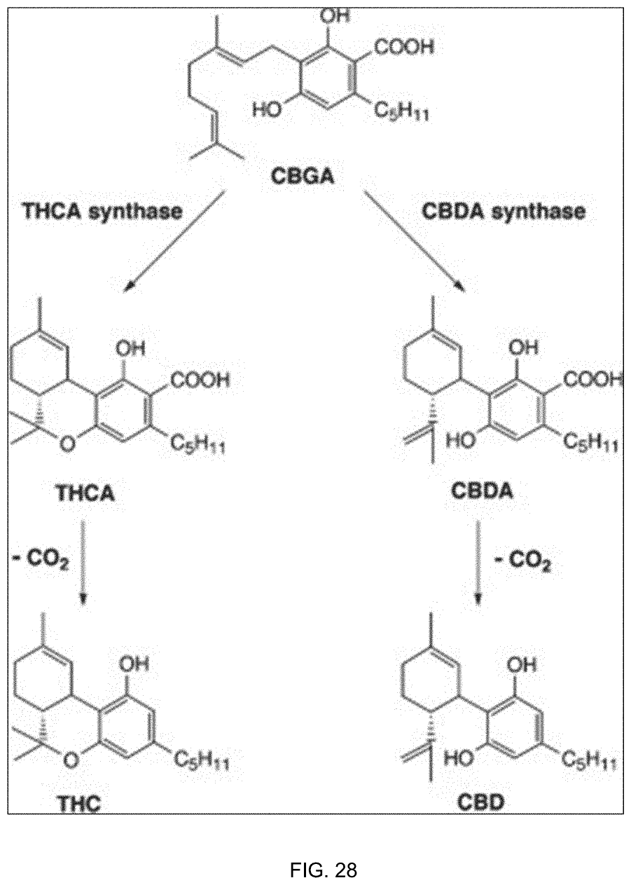

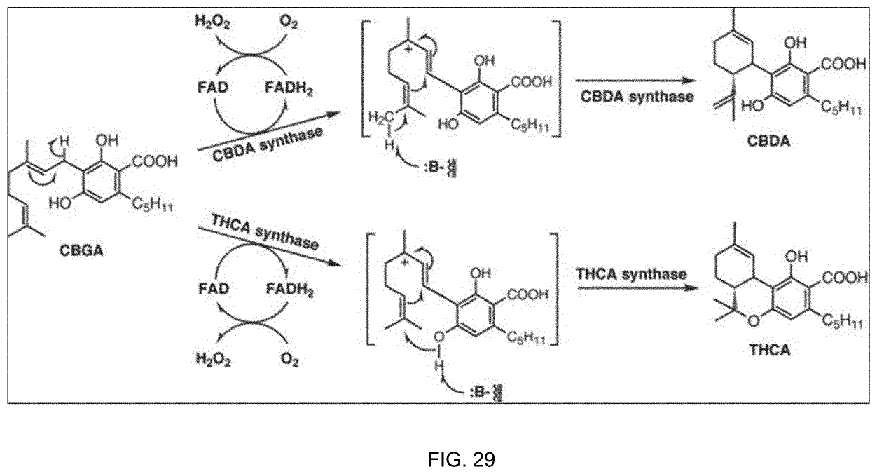

The present inventors have developed a plurality of systems for the biosynthesis and modification of cannabinoids based on cellular location using novel methods of protein targeting. As shown in Table 10, the present inventors designed such novel systems and methods to enhance production and modification (glycosylation, acetylation and functionalization) of cannabinoids as well as to mitigate toxicity resulting from cannabinoid accumulation. Certain embodiments, included the expression of a MYB transcription factor and a catalase (FIG. 27) to degrade hydrogen peroxide resulting from CBDA synthase activity. In one preferred embodiment, the present inventors used Arabidopsis thaliana or an E. coli catalase gene and a predicted Cannabis MYB transcription factor involved in elevating genes involved in cannabinoid biosynthesis. DNA and protein sequences for Cannabis predicted MYB transcription factor (SEQ ID NOs. 11-12, DNA and amino acid sequences respectively), Arabidopsis thaliana catalase SEQ ID NOs. 13-14, DNA and amino acid sequences respectively) and/or E. coli catalase (SEQ ID NO. 15-16, DNA and amino acid sequences).

### Example 7: Enhanced In Vivo Cytosolic Synthesis and Glycosylation of Cannabinoids in Tobacco Leaves and Cell Suspensions

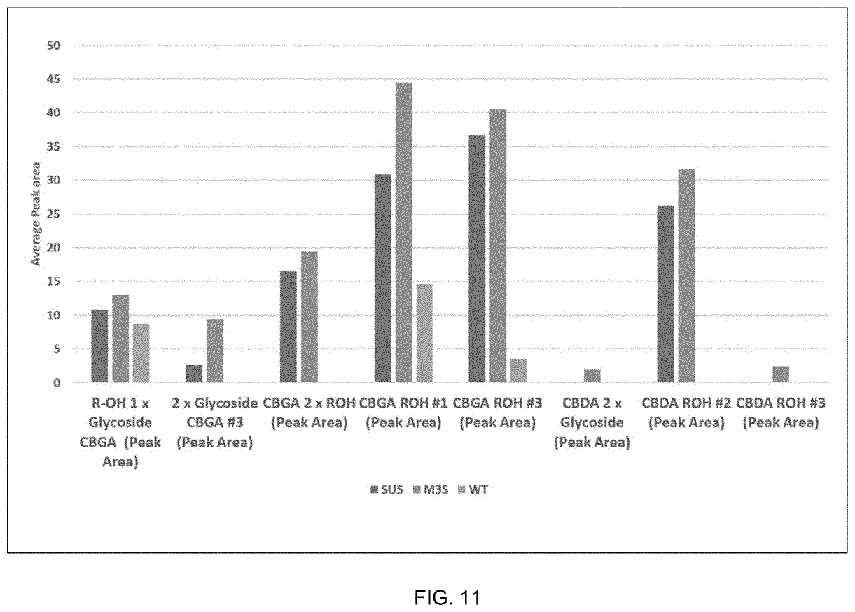

The present inventors have demonstrated the enhanced in vivo modification of cannabinoids in transgenic plants co-infected with constructs for glycosylation, P450-mediated functionalization (hydroxylation) and detoxification of hydrogen peroxide by catalase. As further shown in FIG. 11, functionalization and glycosylation, mainly of the substrate CBGA was observed in transgenic tobacco plants overexpressing CBDA synthase, UDP glycosyltransferase and ABC transporter but increased when overexpression of this construct was coupled with cytochrome P450, MYB transcription factor and catalase. As previously noted, overexpression of a cytochrome P450 enhanced glycosylation of cannabinoids. As such, the present inventor demonstrated the formation and glycosylation of CBDA in vivo in transiently transformed tobacco leaves fed with the precursor CBGA.

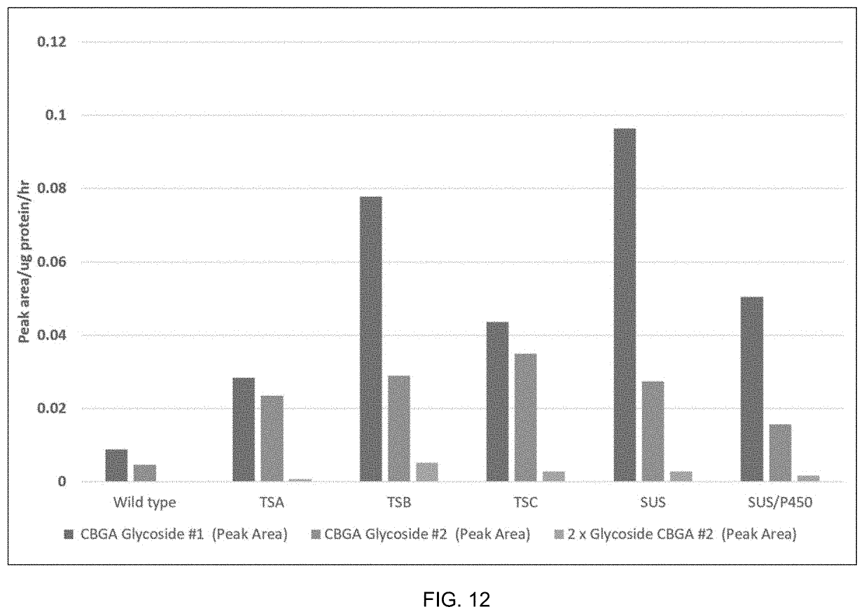

The present inventors also compared the activities of endogenous and transgenic glycosyltransferase activities in tobacco. Specifically, as shown in FIG. 12, the present inventor performed in vitro assays of UDP glycosyltransferase and CBDA synthase. Short assays of 3 hours at 30° C. did not reveal any difference in glycosylation of CBGA between the wild type and transgenic N. benthamiana plants, suggesting endogenous glycosylation. In extended assays (14 hours), there was a significant difference in the detection of glycosylated CBGA in transgenic plants compared to the wild type demonstrating increased glycosylation activity in transgenic plants.

In certain embodiment, glycosyltransferases from tobacco, or other plants may be used as herein described. In one embodiment, one or more heterologous or homologous glycosyltransferases may be expressed or over expressed in a plant, such as tobacco or Cannabis. Gene and protein sequences for exemplary glycosyltransferases are identified below in Table 9.

### Example 8: Generation of Trichome-Targeted Cannabinoid Synthesis and Glycosylation Constructs of Cannabidiolic Acid (CBDA)

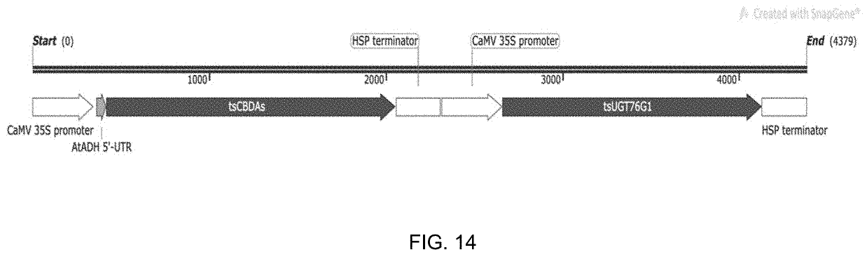

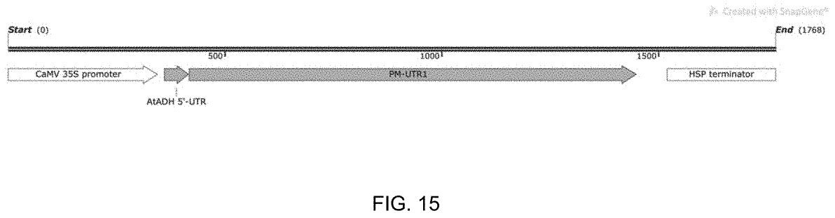

As shown in FIGS. 14-15, the present inventors demonstrated a system of trichome-targeted synthesis and synthesis and glycosylation of cannabinoid compounds, such as CBDA.

By targeting CBDA synthase, a UDP-glucose/UDP-galactose transporter (PM-UTR1) targeted to the plasma, and a Stevia UDP-glycosyltransferase 76G1 (tsUGT) to the trichomes, these genes may produce and accumulate, in this case CBDA and its glycosylated derivatives (primary, secondary glycoside), as well as novel CBDA derivatives, in the trichomes.

SEQ ID NO. 17 is identified as the polynucleotide gene sequence for a CBDA synthase having a trichome targeting sequence. SEQ ID NO. 18 is identified as the corresponding protein sequence for a CBDA synthase having a trichome targeting domain.

SEQ ID NO. 19 is identified as the polynucleotide gene sequence for a trichome-targeted UDP-glycosyltransferase (76G1) coding sequence, in this instance being optimized for Arabidopsis thaliana expression, although other codon optimized versions fall within the scope of this invention. SEQ ID NO. 20 is identified as the corresponding protein sequence for a UDP-glycosyltransferase (76G1) having a trichome targeting domain. SEQ ID NO. 21 is identified as the polynucleotide gene sequence for a UDP-glucose/galactose transporter (UTR1) having a plasma-membrane targeting sequence.

### Example 9: Trichome-Targeted Synthesis and Glycosylation of Cannabidiolic Acid (CBDA)

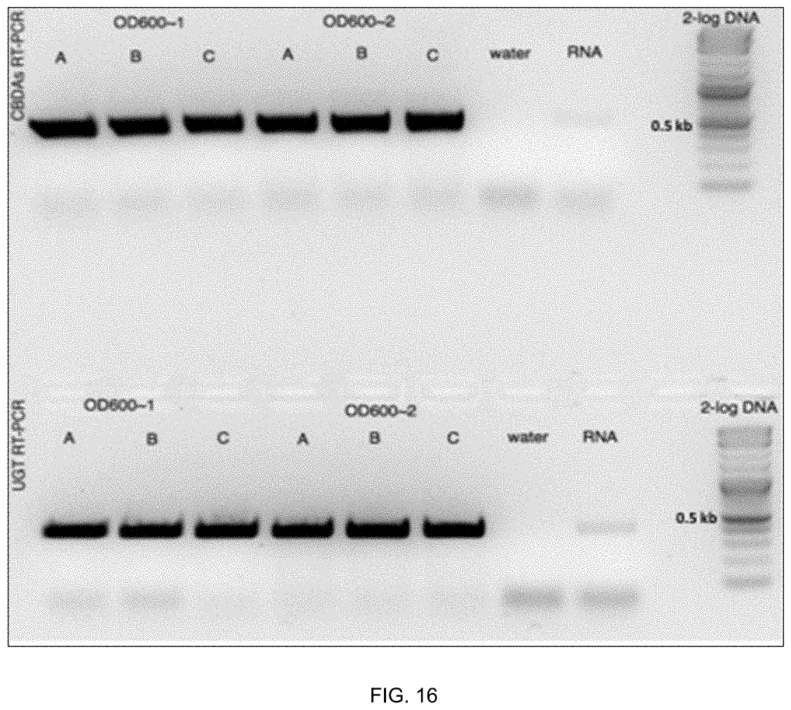

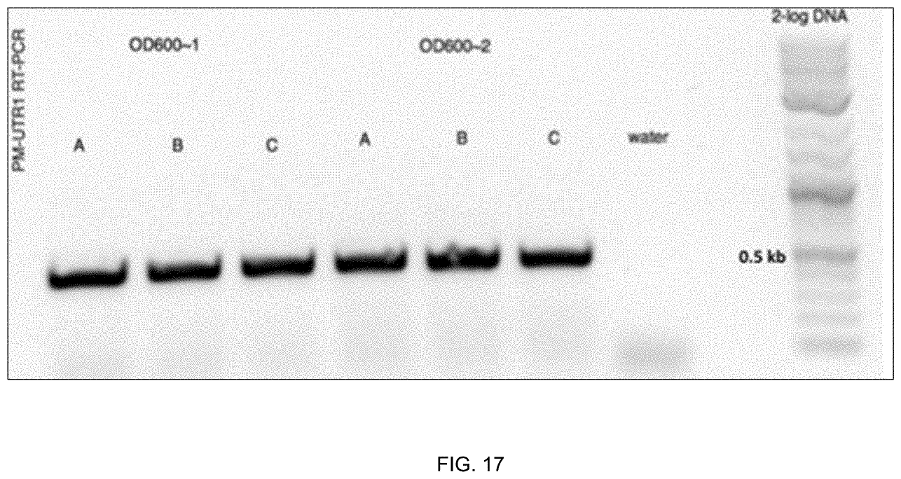

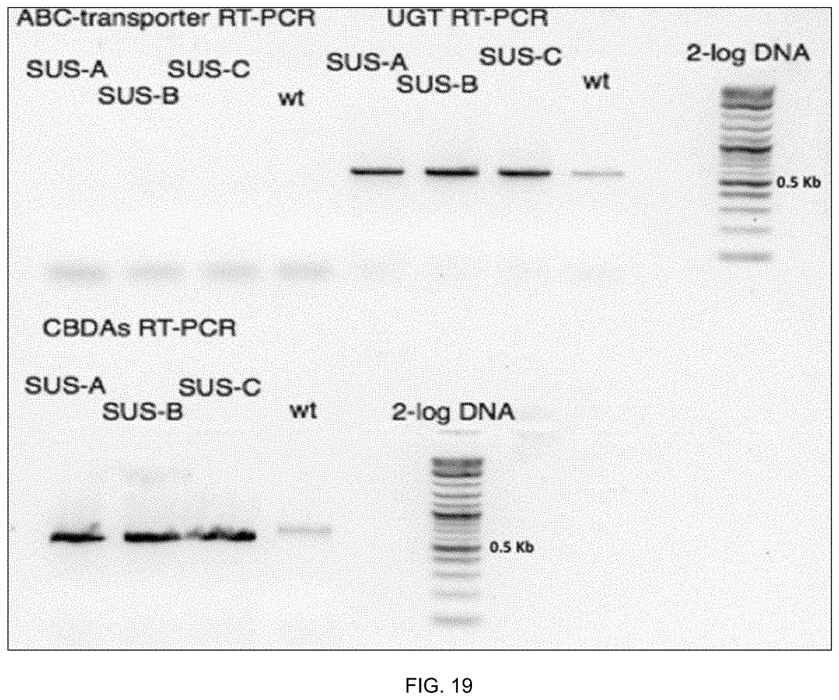

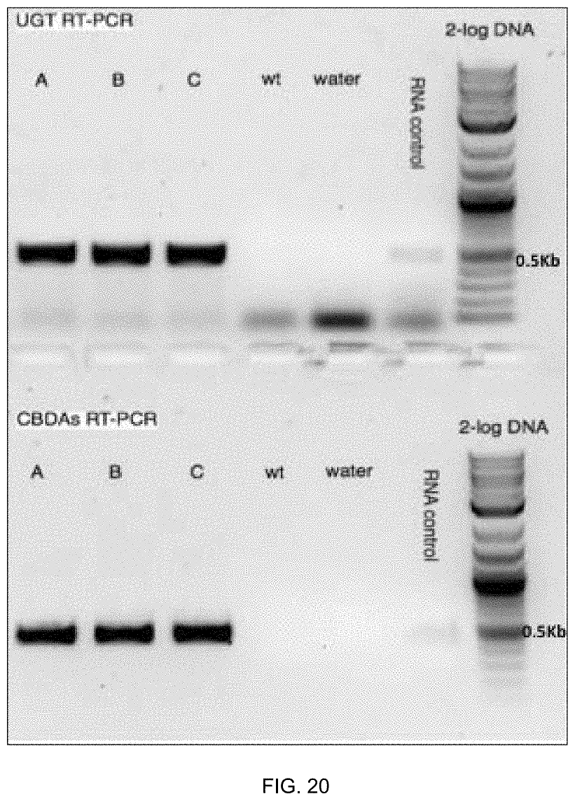

As shown in FIGS. 16-17, gene expression of CBDA synthase, tsUGT and PM-UTR1 in N. benthamiana infiltrated leaves was confirmed 2 DPI (Days Post Infiltration of Agrobacterium Ti-plasmid constructs) via RT-PCR (FIGS. 19 and 20). As expected, CBGA substrate was detected in all infiltrated leaves and wild type control (no Agrobacterium infiltration). CBGA primary and secondary glycosides were also detected in all infiltrated leaves and wild-type control, further demonstrating an endogenous glycosyltransferase activity acting upon CBGA. Moreover, CBGA acetylated primary glycoside was detected in all samples, including WT control, providing evidence of endogenous acetylation. CBDA was detected at marginal levels in samples infiltrated with both trichome and cell suspension constructs, but not in wild type plants.

### Example 10: Cytosolic-Targeted Synthesis and Glycosylation of Cannabidiolic Acid (CBDA)

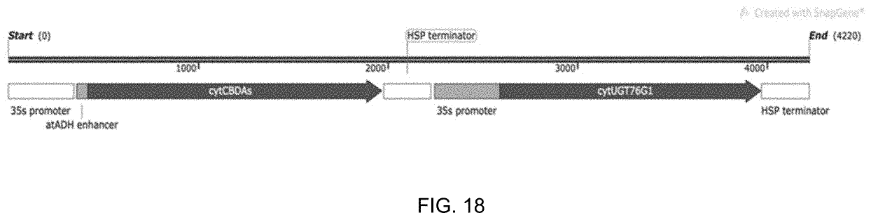

The present inventors have demonstrated a system of cytosolic-targeted cannabinoid synthesis and glycosylation. By targeting or localizing, CBDA synthase (CBDAs) and UDP-glycosyltransferase 76G1 (UGT) to the cytosol, the present inventors demonstrated that plants expressing these heterologous genes produce and accumulate, in this embodiment, CBDA and its glycosylated derivatives (primary, secondary glycoside), as well as other CBDA derivatives, in the cytosol. As shown in FIG. 18, a gene expression vector for the cytosolic cannabinoid production system was generated. This construct included a cauliflower mosaic 35S promoter; AtADH 5′-UTR, enhancer element; cytCBDAs, cannabidiolic acid synthase with the trichome target sequence removed; HSP terminator; cytUGT76G1, UDP glycosyltransferase from Stevia rebaudiana.

SEQ ID NO. 22 is identified as the polynucleotide gene sequence for a, cannabidiolic acid synthase with the trichome target sequence removed (cytCBDAs). SEQ ID NO. 23 is identified as the corresponding protein sequence of cytCBDAs.

SEQ ID NO. 24 is identified as the polynucleotide gene sequence for a, Cytosolic-targeted UDP-glycosyltransferase (UGT76G1) coding sequence (optimized for Arabidopsis thaliana expression) (cytUGT76G1 or cytUTG). SEQ ID NO. 25 is identified as the corresponding protein sequence of cytUGT76G1 or cytUTG.

As an exemplary plant model, N. benthamiana plants were grown from seed and after 4 weeks of vegetative growth, leaves were co-infiltrated with Agrobacterium tumefaciens GV3101 carrying the following constructs: Cytosolic CBDAs+Cytosolic UGT in pRI201-AN or cell suspension construct, Myb/catalase in pRI201-AN, and p19 silencing suppressor in pDGB3alpha2. Agrobacterium density was normalized to 2 at absorbance of 600 nm using a spectrophotometer and cultures co-infiltrated in same ratio (1:1:1). After 2 and 4 days post-Agrobacterium infiltration (DPI), 1 mL CBGA (2.7 mM) dissolved in 0.1% Tween 20 (Sigma-Aldrich) or 0.1% Triton X-100 (Sigma-Aldrich) was infiltrated to each leaf. In a second embodiment using the cytosolic construct, 4 mM UDP-glucose was added to the CBGA media before feeding. Three biological replicates were used. RT-PCR primers are outlined in Table 5 below.

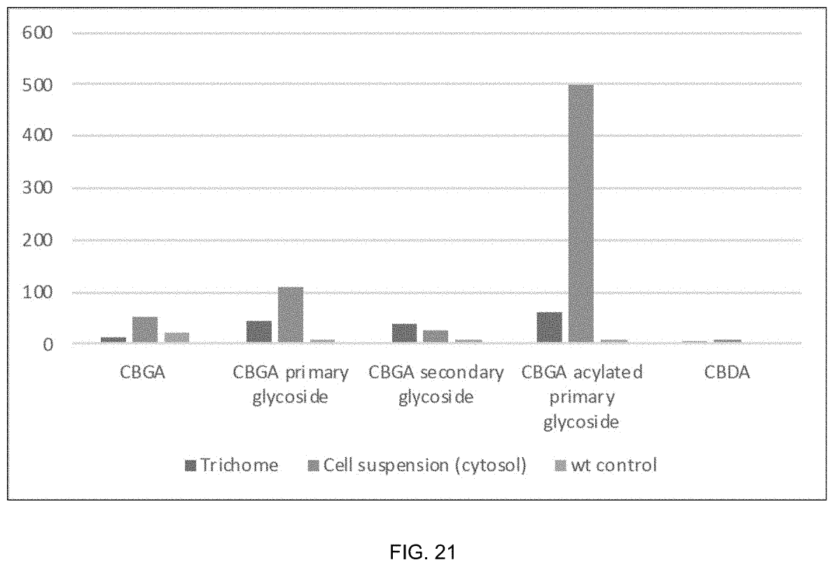

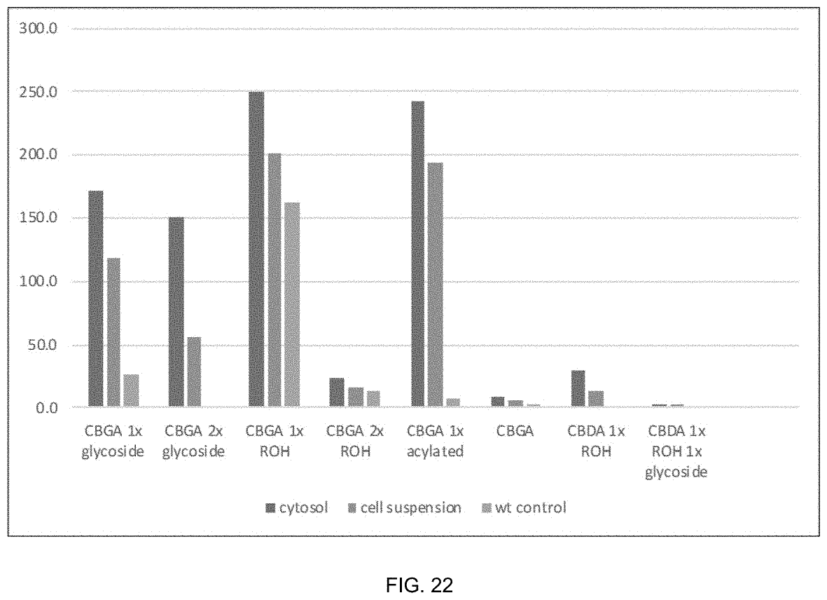

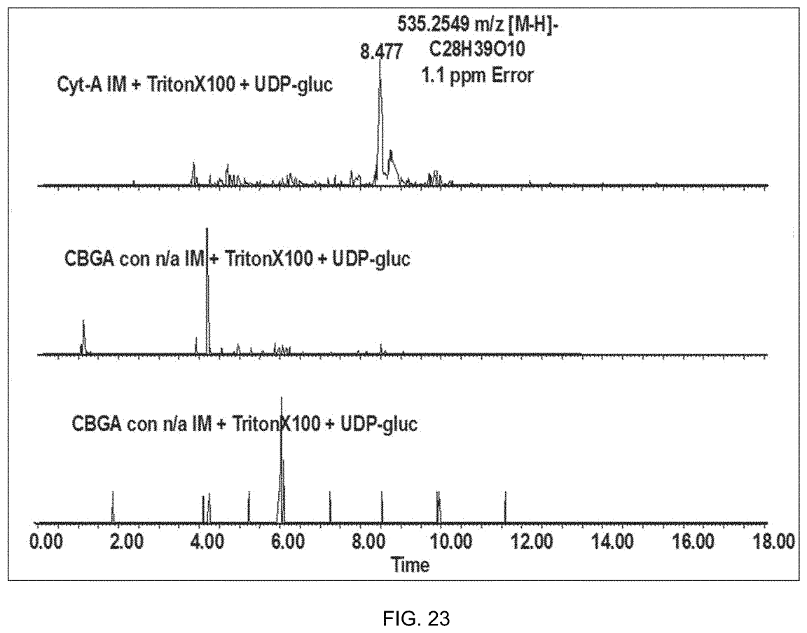

As shown in FIGS. 19-20, gene expression of cytCBDAs and cytUGT was confirmed via RT-PCR after 1 and 2 DPI. No expression of ABC transporter (ABCt) was observed after 1 DPI in leaves infiltrated cells suspension construct. This does not impact this experiment as the role of ABCt was to facilitate cannabinoid transport outside the cells in suspension cultures. As shown in FIG. 21, CBGA and its glycosylated and acetylated derivatives were detected in concentrations higher than in the trichome construct infiltrated leaves, except for secondary glycosides. Moreover, CBDA was detected in higher concentrations (up to 34 ppm) in leaves infiltrated with the cell suspension construct, compared to the trichome construct experiments (up to 2.6 ppm). As shown in FIG. 22, when UDP-glucose 4 mM (substrate for UGT) was provided together with CBGA (substrate for CBDAs), the present inventors detected low levels of glycosylated and hydroxylated CBDA in leaves infiltrated with both the cytosolic and cell suspension construct, but not in the WT control. This result demonstrates the novel in plant synthesis, glycosylation and hydroxylation of CBDA in the surrogate plant N. benthamiana, as demonstrated by the Extracted Ion Chromatograms shown in FIG. 23.

### Example 11: Hydroxylation and Glycosylation of Cannabinoids in Cannabis Sativa

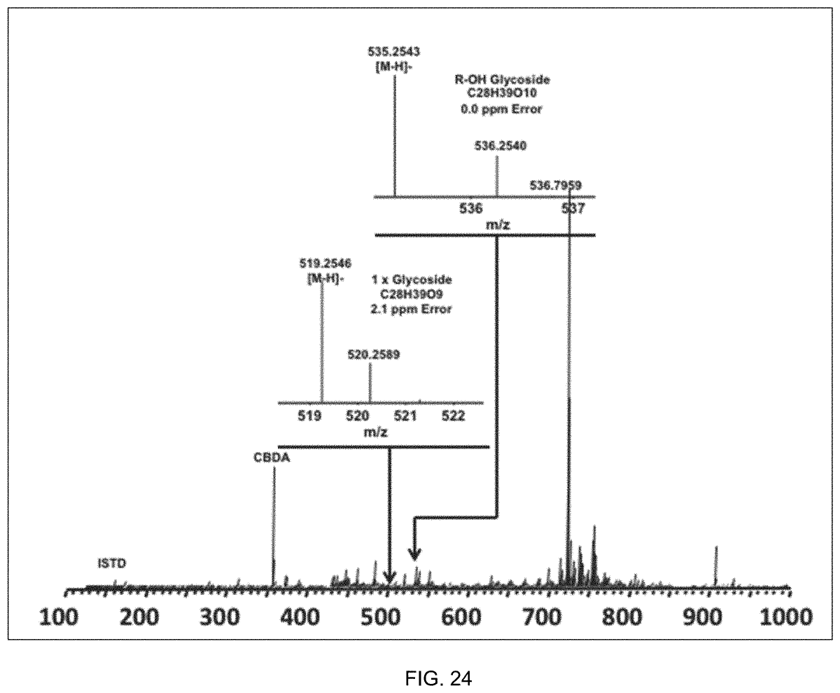

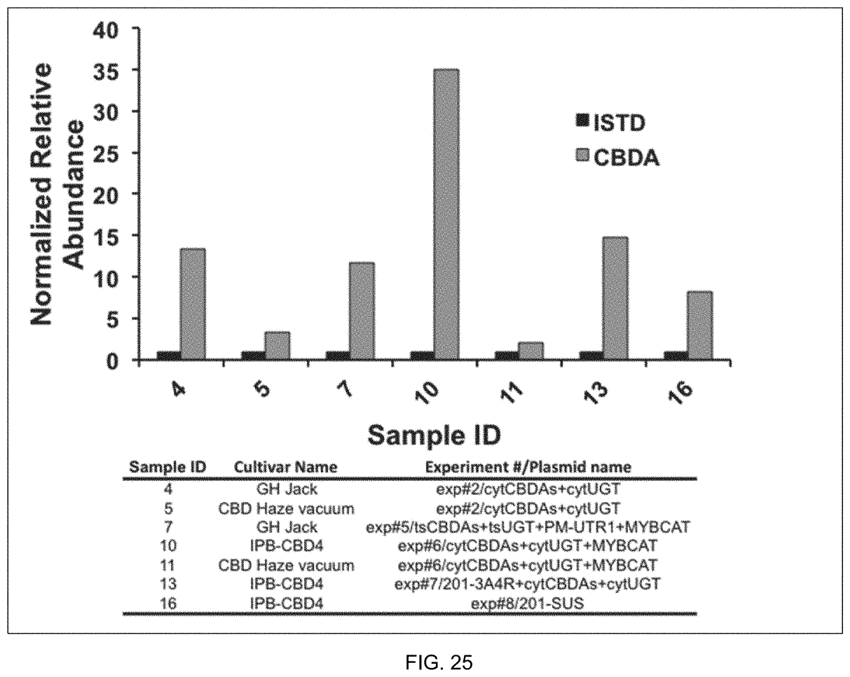

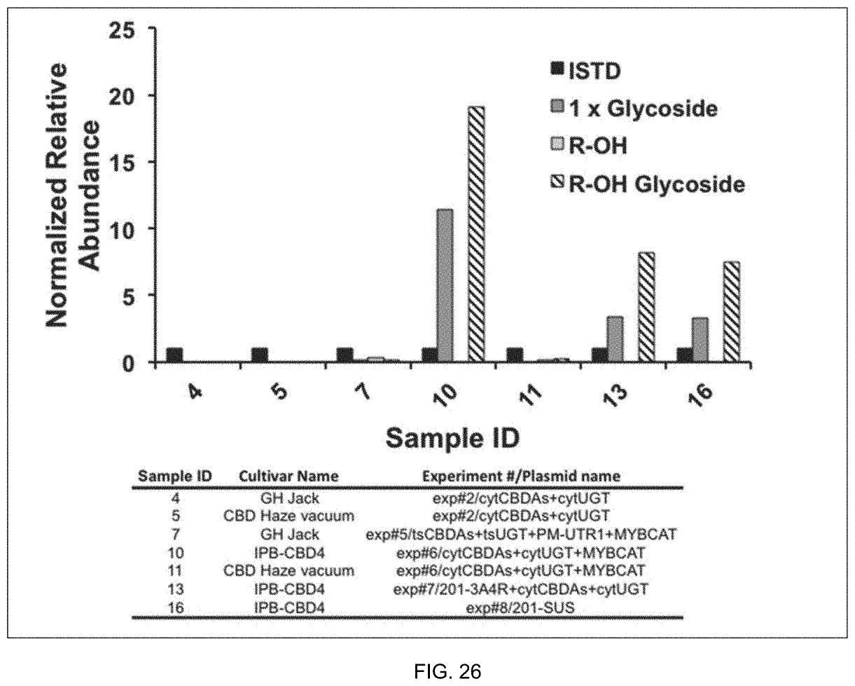

The present inventors demonstrate the glycosylation and hydroxylation of cannabinoids in Cannabis sativa. To further confirm our findings using N. benthamiana as a plant model, we performed Agrobacterium infiltration of the same plasmid constructs described in the section above in various strains of Cannabis sativa (see FIG. 24 Sample IDs). As shown in FIGS. 24-26, expression of the select genetic constructs in C. sativa, as in N. benthamiana, demonstrate synthesis and accumulation of hydroxylated and/or glycosylated cannabinoids, in this case CBDA. A comparison of the results using different Agrobacterium genetic constructs is presented in Table 8 below.

As the present inventors have demonstrated, in one embodiment, where the cytosolic construct was con-transformed with the Myb/catalase (MYBCAT) expression vector, yielded the highest detection of CBDA and CBDA glycoside, demonstrating the role of these genes in mitigating toxicity effects due to hydrogen peroxide accumulation (catalase) and overall increase in cannabinoid synthesis (Myb transcription factor).

### Example 12: Intracellular Expression of Glycosyltransferases in Yeast Cells

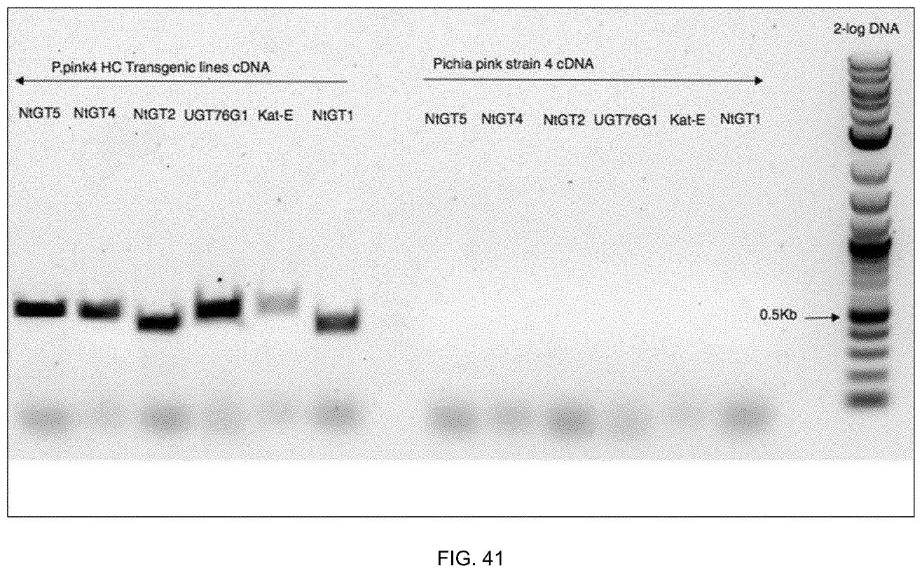

Four glycosyltransferases from Nicotiana tabacum (NtGT1, NtGT2, NtGT4, NtGT5), one from Stevia rebaudiana (UGT76G1), and Escherichia coli catalase E (Kat-E) encoding sequences were codon-optimized for expression in Pichiapastoris, synthesized by Genewiz, and cloned into pPink-HC or pPINK-aHC vector as described in the PichiaPink expression system manual (Invitrogen). The assembled constructs were verified by restriction enzyme digestion and DNA sequencing. Each of the constructs was used to transform the wild-type strain (strain 4). Transgene expression in transgenic yeast and expression was verified by RT-PCR (FIG. 41). The list of primers used in PCR verification of transgene expression is shown in Table 13. Codon optimized DNA and corresponding amino acid sequence identities are as follows: NtGT1 (SEQ ID NO. 51, and SEQ ID NO. 52 respectively); NtGT2 (SEQ ID NO. 53, and SEQ ID NO. 54 respectively); NtGT3 (SEQ ID NO. 55, and SEQ ID NO. 56 respectively); NtGT4 (SEQ ID NO. 57, and SEQ ID NO. 58 respectively); NtGT5 (SEQ ID NO. 59, and SEQ ID NO. 60 respectively); UGT76G1 (SEQ ID NO. 61, and SEQ ID NO. 62 respectively); Kat-E (SEQ ID NO. 65, and SEQ ID NO. 66 respectively). Additional codon optimized exogenous glycosyltransferases that may be used with the current invention may include, but not be limited to: UGT73A10 (SEQ ID NO. 63, and SEQ ID NO. 64 respectively);

### Example 13: Introducing CBDA to Yeast Cells in Acetonitrile

The present inventors demonstrated that after transformation of vectors into the wild type yeast strain, white colonies were selected and transferred into 250 mL flasks containing 50 mL YPG media (yeast extract, peptone, glycerol). After overnight growth (the cultures reached an OD₆₀₀˜1), 100% methanol was added at 5% v/v to induce gene expression overnight. The following morning, cultures were aliquoted into 3×10 mL cultures, centrifuged and resuspended in fresh YPG media with 2.5% v/v methanol, and 50 uL of CBDA in acetonitrile (1 mg/mL, Cayman Chem) was added to a final concentration of 14 uM. After 72 hs, the samples were centrifuged down and the cell pellet and supernatant were separately frozen in liquid nitrogen and stored at −80° C. for further LC/MS analysis of cannabinoids.

### Example 14: Glycosylation of CBDA by NtGT4

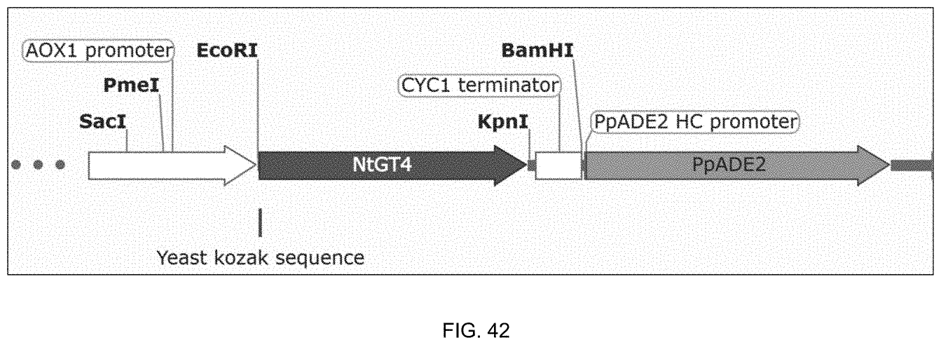

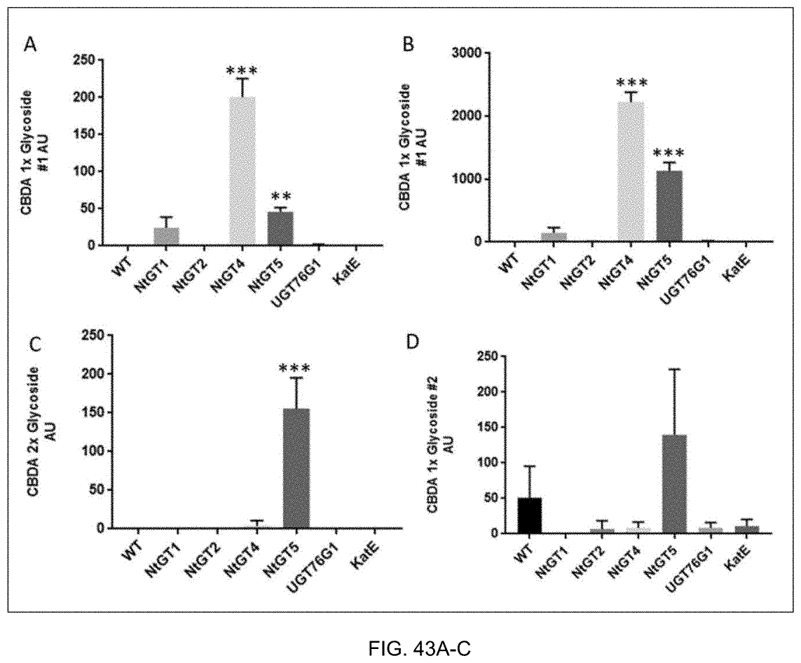

The present inventors demonstrate that intracellular expression of NtGT4 (construct outlined in FIG. 42), the UGT 73-like glycosyltransferase from Nicotiana tabacum, led to the highest level of glycosylation of CBDA (FIGS. 43 A and B). The CBDA glycoside was detected in the pellet (FIG. 43B) as well as in the supernatant (FIG. 9A) suggesting that the yeast is secreting the product into the media, presumably by an endogenous ABC transporter. Overall, glycosylation by NtGT4 was significantly higher than by any other glycosyltransferase tested. NtGT1, NtGT2 and the Stevia UGT76G1 had only trace levels of CBDA glycosides that were not significantly different in yield from the untransformed wild-type strain.

### Example 15: Glycosylation of CBDA by NtGT5

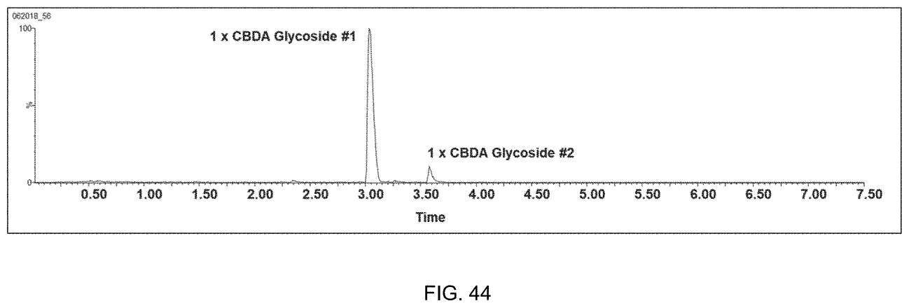

The present inventors demonstrate that intracellular expression of NtGT5, the 7-deoxyloganetin glycosyltransferase-like from Nicotiana tabacum, led to glycosylation of CBDA (FIGS. 43 and 44). The present inventors further demonstrate that NtGT5 is not only capable of catalyzing the same R—OH position as NtGT4 but preferentially glycosylates a different and less water-soluble position than NtGT4. (Generally panels B &E of FIG. 37)

### Example 16: Introducing CBD Oil Extract to Yeast Cells

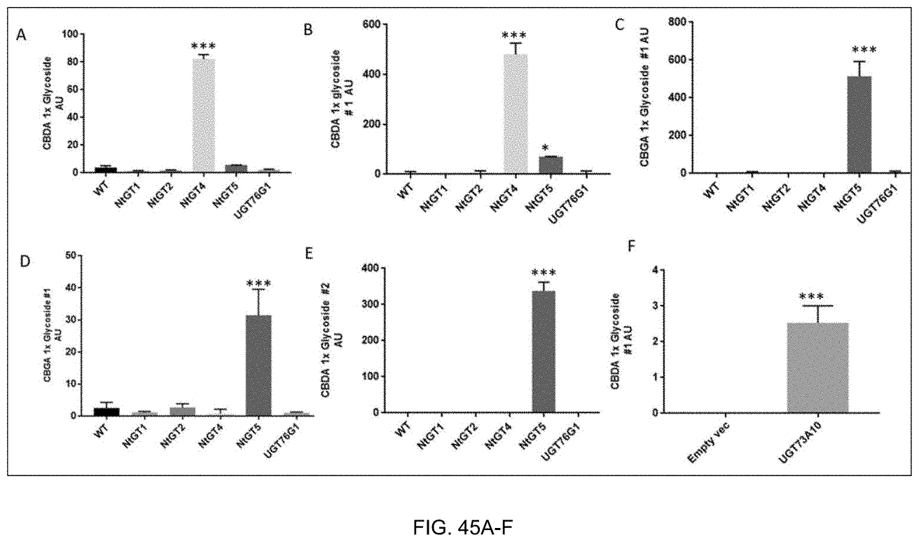

50 mL cultures of transgenic yeast were induced with methanol after 24 hours of growth in YPG and fed with 227 uM cannabidiol (CBD) in the form of a commercial diluted CBD oil (Minnerva Canna). After 72 hs, the samples were centrifuged down and pellet and supernatant were separately frozen in liquid nitrogen and stored at −80° C. for LC/MS analysis of cannabinoids. As in the CBDA feeding experiments, the present inventors demonstrate that NtGT4 and NtGT5 yielded the highest levels of glycosylation in different positions on CBD oil feeding experiments (FIG. 44). CBDA glycosides were detected in both supernatant (FIGS. 45A, D and F) and pellet (FIGS. 45 B, C and E). Oil extract feeding allowed the present inventors to investigate glycosylation of other cannabinoids. NtGT5 glycosylated the cannabinoid precursor CBGA (FIG. 45).

### Example 17: Extracellular Glycosylation of Cannabinoids

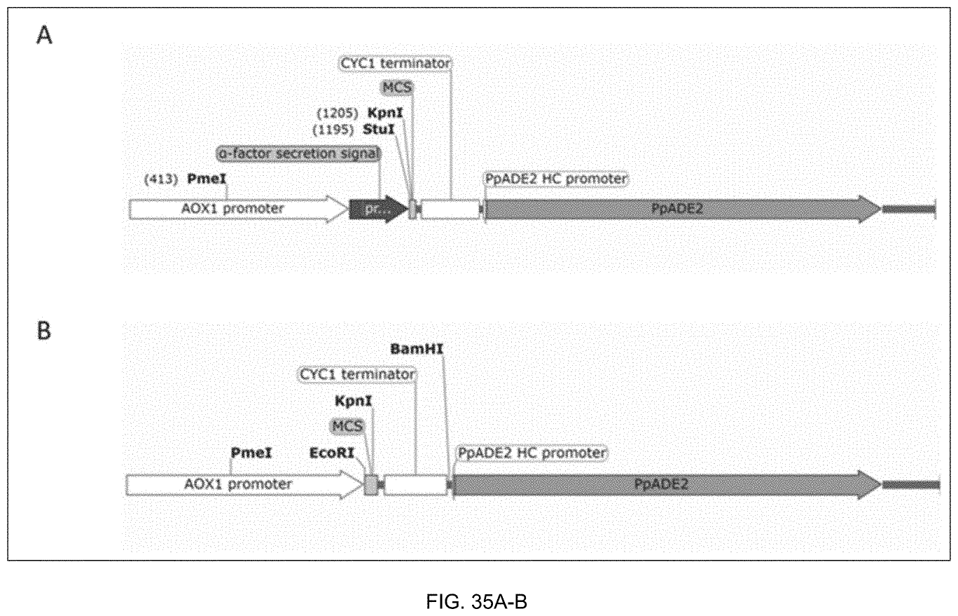

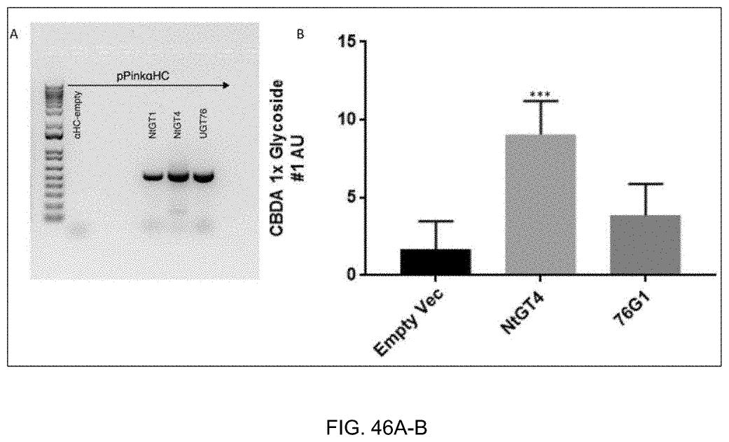

As described above, in one exemplary embodiment, an expression vector was used by the present inventors to secrete proteins into the media for an extracellular glycosylation of cannabinoids. Transgenic yeast lines expressing glycosyltransferases with the α-factor secretion signal (FIG. 35A) were fed CBD oil extract as previously described and analyzed for glycosylated cannabinoids. There was no glycosylation in the pellets as expected since the enzymes were secreted into the media. There was only minimal glycosylation in the supernatant (FIG. 46) in comparison with the intracellular system.

### Example 18: Time Course Analysis of Intracellular Cannabinoid Glycosylation

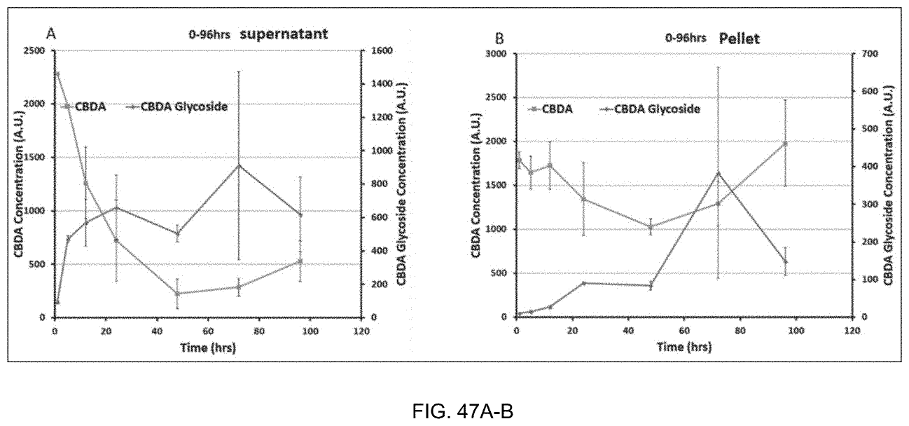

To determine the optimum time for cannabinoid glycosylation in yeast, the present inventors set up a time course experiment. Transgenic yeast expressing NtGT4 intracellularly were fed with CBDA (27 M) and incubated for up to 96 hours. Samples were collected at different time points during the incubation and analyzed for the formation of CBDA glycosides (See FIG. 47). In both pellet and supernatant, a reciprocal relationship was observed by the present inventors between CBDA loss and CBDA glycoside production. For the supernatant (media), CBDA depletion was most likely due to uptake by the yeast. In the pellet, CBDA depletion can be explained by its glycosylation into CBDA glycosides. The optimal time for CBDA glycosylation was 48 hours, after which CBDA levels increased and CBDA glycosides dropped, suggesting that there is possibly an inducible and competing glycosidase activity present in the yeast that is turning over the CBDA glycoside. To prevent this glycosidase, the present inventors may introduce glycosidase inhibitors to preserve CBDA glycosides or suppress the expression of the endogenous glycosidases. In additional embodiment, the present inventors may overexpress an ABC transporter to speed up secretion of CBDA glycosides into the media. One example may include the expression of a multi-drug resistant transporter ABCG2 (SEQ ID No. 67 and SEQ ID No. 67) in tobacco. Through transcriptomics may also be employed by to identify possible candidates in yeast overexpressing glycosyltransferases.

### Example 17: Glycosylation of Cannabinoids in Tobacco Bright Yellow Cells

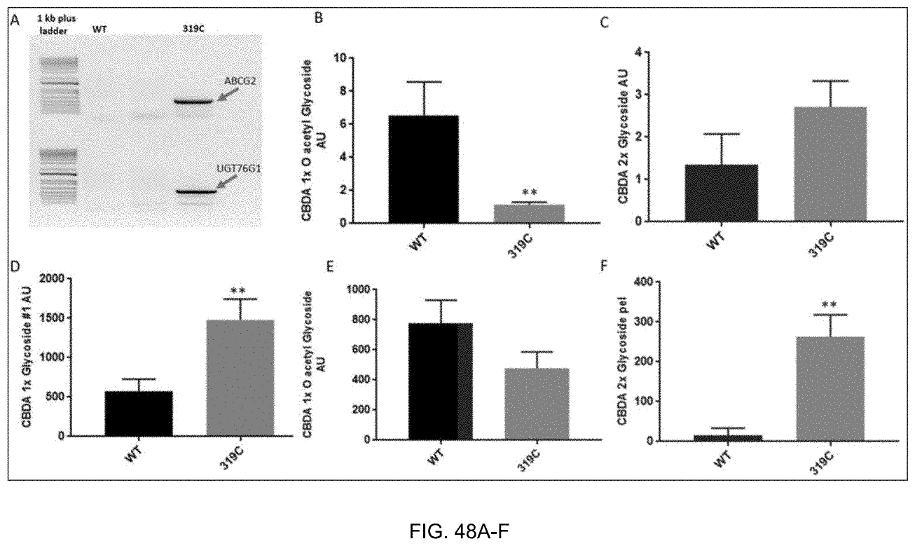

Similar to yeast suspension cultures, plant cell cultures are a viable platform for the production of recombinant proteins because they can be cultivated under sterile conditions and can be scaled up in fermenters for industrial level production. One of the most widely used cell lines in plant biology is the tobacco Bright Yellow 2 (BY2) cell line developed in 1968 at the Hatano Tobacco Experimental Station, Japan Tobacco Company. BY2 cells have a doubling time of 16-24 hours, multiplying up to a 100-fold in 7 days t al., 2016), can be easily transformed by Agrobacterium mediated transformation and require basic plant growth media for maintenance. As described above, the prevent inventors demonstrated endogenous glycosylation in tobacco leading to the possibility of using tobacco suspension cultures as a postharvest glycosylation platform. In this embodiment, the present inventors introduced wild-type and transgenic BY2 cells expressing the Stevia glycosyltransferase UGT76G1 (SEQ ID NO. 61 and SEQ ID NO. 62) and the multidrug resistance transporter ABCG2 (319C) (SEQ ID NO. 67 and SEQ ID NO. 67) with 5 M CBDA in acetonitrile and grew the cultures for 3 days. Confirmation of transgene expression in BY2 cells was done by RT-PCR with primers amplifying a ≈0.5 kb region of the transgene (FIG. 48).

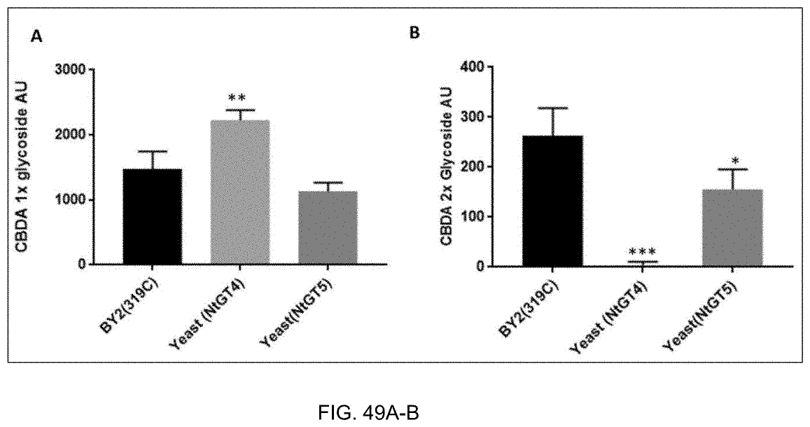

For the CBDA 1× O acetyl glycoside, glycosylation was observed in the wild type more than in transgenic lines. For all other forms of glycosylated CBDA, the transgenic line 319C had increased glycosylation compared to the wild type. The present inventors ran a comparison between glycosylation in yeast and tobacco yields, normalizing with pellet mass (FIG. 49).

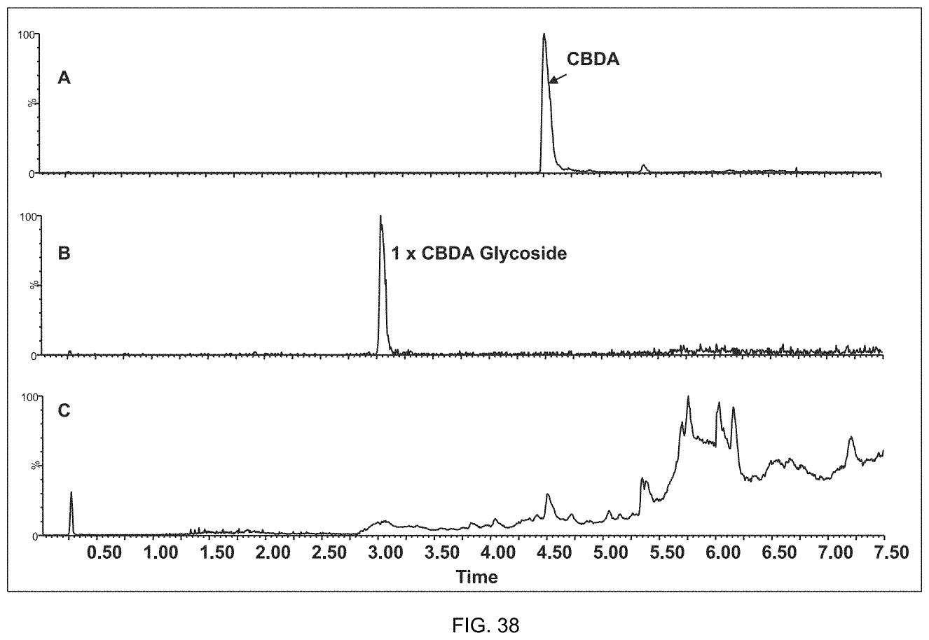

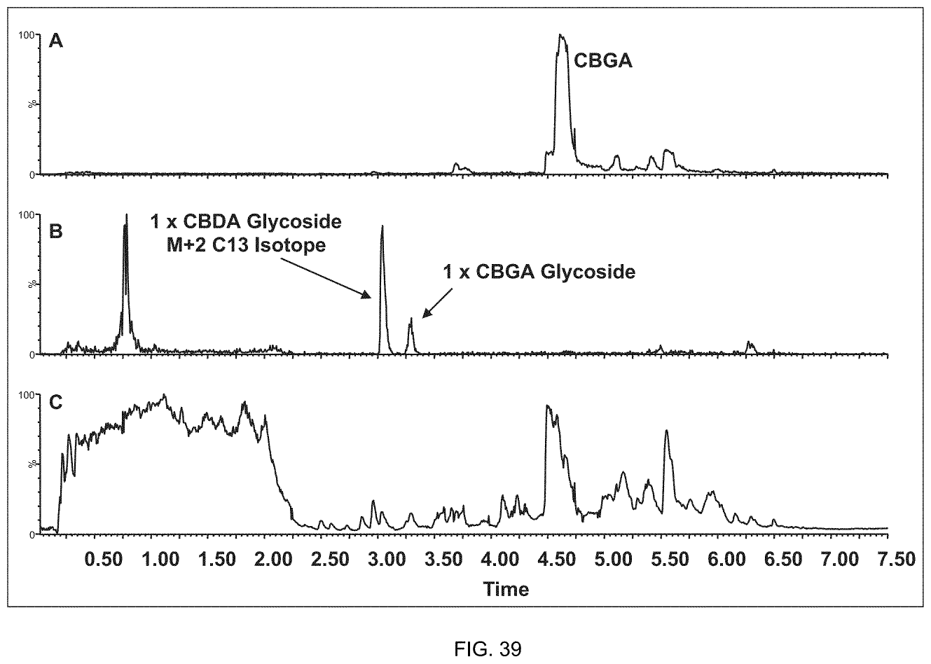

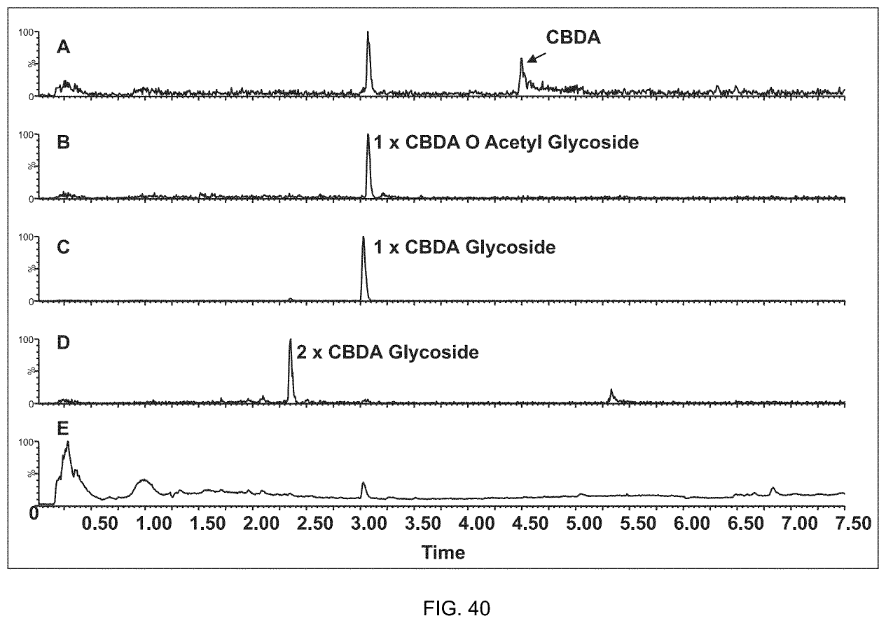

As demonstrated in the figures, in general, a more diverse range of glycosylated products were obtained in tobacco compared to yeast (see chromatograms in FIGS. 38, 39 and 40). The common compounds produced were the CBDA 1× glycoside and the CBDA 2× glycoside. For the 1× glycoside, glycosylation in yeast lines overexpressing NtGT4 was significantly higher than in the BY2 cells overexpressing the Stevia UGT76G1. However, for the 2× glycoside (predicted to be more water-soluble than 1× glycoside), BY2 cells demonstrated higher glycosylation rate than the yeast (FIG. 49B). However, in BY2 cell cultures, low amounts of CBDA glycosides (<6 arbitrary units, normalized to fresh weight) compared to yeast cells (50-200 normalized arbitrary units) were detected in the supernatant, suggesting lower secretion in tobacco suspension cultures. In certain embodiments, co-expressing the tobacco NtGT4 and NtGT5 with an ABC transporter, such as ABCG2, under constitutive promoters in BY2 cells may increase glycosylation in tobacco and make BY2 cell cultures providing an alternative platform for production of water-soluble cannabinoids.

Additional embodiments of the current invention may include the transformation of tobacco, yeast or plant cells, such as Cannabis, with one or more exogenous P450 genes. In one preferred embodiment, this may include Cytochrome P450 (CYP3A4) from Mus musculus (SEQ ID NO. 69 and 70) as well as P450 oxidoreductase gene (CYP oxidoreductase) from Mus musculus (SEQ ID NO. 71 and 72). In some embodiment, the aforementioned gene may be codon optimized for expression in yeast cells.

### Example 18: Acetylated Cannabinoid Glycosides Exhibit Enhanced Stability in Solution

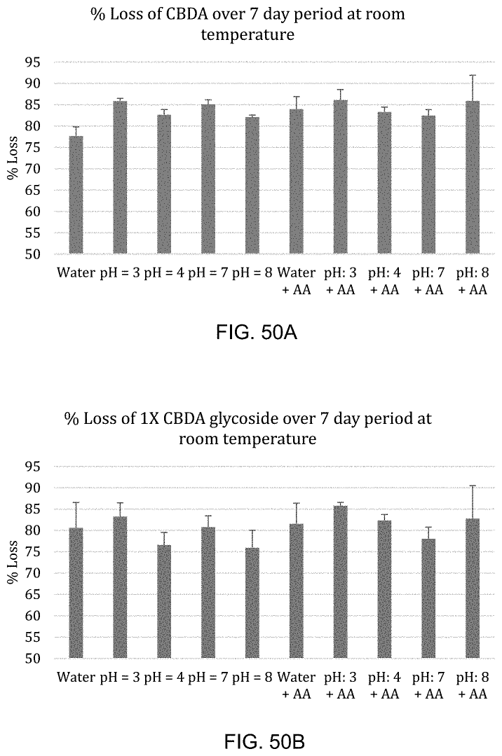

As generally described in FIGS. 50A-B and 51A-B, and Tables 14-19, the present inventors demonstrate that acetylated cannabinoid glycosides exhibit enhanced stability of the sugar moiety in solution compared to non-glycosylated cannabinoids, or cannabinoid glycosides. Due to this enhanced stability of the sugar moiety, acetylated cannabinoid glycosides exhibit both improved water solubility and/or chemical stability in solution, as well as enhanced resistance to chemical degradation into alternative cannabinoid forms over time.

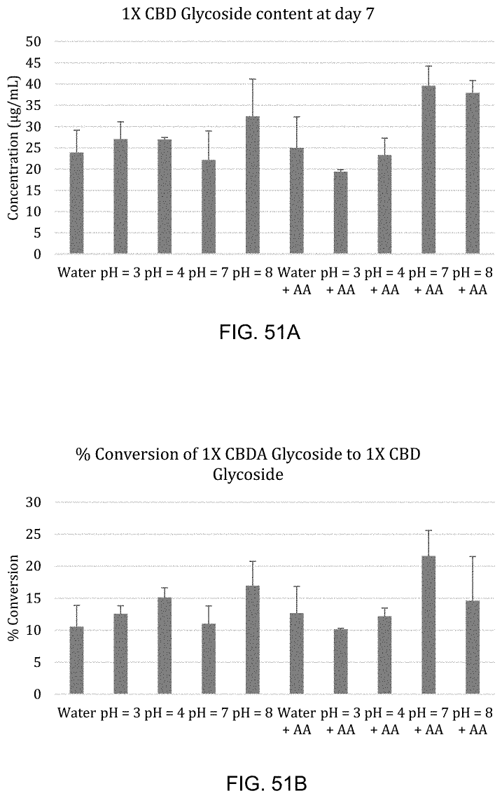

In FIGS. 50A-B and 51A-B, the present inventors demonstrated the loss of CBDA and 1×CBDA glycoside and the quantity of 1×CBD glycoside formed, presumably from decarboxylation of 1×CBDA glycoside over time. As also demonstrated in FIGS. 50 and 51, and Tables 14-19, the present inventors demonstrate that CBD O-Acetyl Glycoside was not found to decrease in any sample after day 7. Rather, levels increased slightly in all treatments. The absence of any residual UGTs were cross-check and confirmed as the samples were purified by SPE, washed and eluted in 30% and 65%-100% ethanol, respectively.

### Materials and Methods

### Materials and Methods Example 1: Use of a Tobacco as an Exemplary Plant System for the In Vivo Functionalization and Glycosylation of Cannabinoids

The present inventors demonstrated the in vivo functionalization and glycosylation of cannabinoids in a model plant system. Specifically, the present inventors used N. benthamiana (tobacco) as a model system to demonstrate in vivo functionalization and glycosylation of cannabinoids. In this embodiment, transient transformation through Agrobacterium infiltration was performed in N. benthamiana. The present inventors demonstrated expression of heterologous genes that were expressed in transformed N. benthamiana using a number of heterologous gene expression vectors (described below). In this exemplary embodiment, upon confirmation of expression of the heterologous genes that would functionalize and glycosylate cannabinoid molecules, the present inventors introduced to the plants select cannabinoid compounds. In this embodiment, the present inventors introduced to the transgenic N. benthamiana plants cannabigerolic acid (CBGA) and/or cannabidiolic acid (CBDA). The present inventors also demonstrated the in vivo functionalization and glycosylation of cannabinoids in a cell suspension culture. Specifically, the inventors used exemplary tobacco bright yellow (BY2) cells as a cell suspension system for studies of cannabinoid production, functionalization and/or glycosylation.

### Materials and Methods Example 2: Transient Transformation of the Exemplary Plant Model Nicotiana benthamiana

The present inventors used Agrobacterium tumefaciens Ti-plasmid-mediated transformation with the plant expression vector pRI201-AN (Takara Bio USA), a binary vector for high-level expression of a foreign gene in dicotyledonous plants carrying the constitutive 35S promoter and an Arabidopsis thaliana Alcohol dehydrogenase (AtAdh) as a translational enhancer (Matsui et al. 2012). N. benthamiana was transiently transformed according to the method described by Sparkes et al. 2006. Overnight cultures of Agrobacterium strain GV3101 were transferred to a 250 mL flask with 50 mL LB medium supplemented with 50 mg/L of Kanamycin, 50 mg/L of Gentamycin and 10 mg/L of Rifampicin and grown for 4-8 hours until the optical density at 600 nm (OD600) reached approximately between 0.75 and 1. The cells were pelleted in a centrifuge at room temperature and resuspended in 45 mL of infiltration medium containing 5 g/L D-glucose, 10 mM MES, 10 mM MgCl2 and 100 μM acetosyringone. 1 ml of the solution was used to infiltrate the leaves using a 1 mL syringe. Expression of the transgene(s) was confirmed 2-4 days after infiltration by RT-PCR. For RT-PCR analysis, 100 mg of leaf tissue were frozen in liquid nitrogen and ground in a TissueLyser (QIAGEN Inc, USA). RNA was extracted following the EZNA plant RNA extraction kit (Omega Bio-tek Inc, USA). Up to a microgram of total RNA was used to synthesize cDNA using the superscript III cDNA synthesis kit (Thermo Fisher Scientific, USA). The cDNA was used to check for the expression of transgene(s) by RT-PCR.

### Materials and Methods Example 3: Introduction of Select Cannabinoid Substrate(s) to the Transgenic N. benthamiana Strain

Select enzyme substrates were introduced to the transgenic or genetically modified N. benthamiana strain two days after Agrobacterium infiltration and upon confirmation of transgene expression by RT-PCR. In this example, approximately 277 μM cannabigerolic acid (CBGA) and/or cannabidiolic acid (CBDA) was dissolved in 1 mL of buffer containing 10 mM MES, 10 mM MgCl₂ and 0.1% Triton X100 or 0.1% Tween20 and applied to the transformed leaves either by infiltration or by dabbing with a cotton applicator. Plants were harvested after 1-4 days, weighed for fresh weight and frozen at −80° C. before conducting LC-MS analysis for the presence of modified cannabinoids.

### Materials and Methods Example 4: In Vitro Assays for CBDA Synthase and Glycosyltransferase Activity

CBDA synthase is generally active in the pH range 4-6 (Taura et al. 1996) while glycosyltransferases are typically active in the pH range 5.0 to 7.0 (Rini and Esko, 2017). Based on this difference in optimal pH for enzyme activity, the present inventors generated a single extraction buffer for a combined assay of CBDA synthase and UDP glycosyltransferase at pH 6 and 30° C. in in vitro assays (Priest et al., 2006). The present inventors ground the transformed leaf tissue in liquid nitrogen. A grinding buffer was added consisting of 50 mM MES, pH 6, 1 mM EDTA, 5 mM β-mercaptoethanol and 0.1% Triton X-100 was added at 5:1 ratio of buffer to fresh weight of plant using a mortar and pestle. The extract was filtered on ice through 2 layers of cheesecloth to remove debris and centrifuged at 21000 g for 5 minutes at 4° C. The supernatant was used in subsequent assays. Protein concentration of the supernatant was quantified by the Bradford assay, using bovine serum albumin as the standard. To start the reaction, 100-200 μg of crude total protein was used. The assay was carried out with and without UDP-glucose to check if glycosylation of cannabinoid substrate was preventing downstream reactions or transport of CBGA. Wild type plants were used as controls to separate endogenous from overexpressed UDP glycosyltransferase activity. The reaction was started by adding 100 μg of protein, and 8 mM uridine diphosphate glucose (UDPG) as the sugar-nucleotide donor to a reaction mixture consisting of approximately 277 μM CBGA, 0.1% (w/v) Triton X-100, 3 mM MgCl₂ and 50 mM MES (pH 6.0). The reaction was incubated at 30° C. for 3 h or overnight for 14 hours. The reaction was terminated by freezing in liquid nitrogen and the samples were stored at −80° C. before LC-MS analysis.

### Materials and Methods Example 5: Trichome-Targeted Synthesis and Glycosylation

As an exemplary plant model, N. benthamiana plants were grown from seed and, after 4 weeks of vegetative growth, the leaves were co-infiltrated with Agrobacterium tumefaciens GV3101 carrying the following constructs: Trichome CBDAs+trichome UGT in pRI201-AN (trichome construct), PM-UTR1 in pRI201-AN, and p19 silencing suppressor in pDGB3alpha2. In a second experiment, leaves were also infiltrated with the Agrobacterium expressing a Ti-plasmid with the Myb/catalase genes. Agrobacterium density was normalized to 1 or 2 at absorbance of 600 nm using a spectrophotometer and cultures co-infiltrated in same ratio (1:1:1). After 1 and 4 days post-Agrobacterium infiltration (DPI), 1 mL CBGA (277 μM) dissolved in 0.1% Tween20 (Sigma-Aldrich) or 3% DMSO (Sigma-Aldrich) was infiltrated to each leaf. Three biological replicates were used. The experiment was repeated twice. After preliminary results, Agrobacterium densities of 2 at OD₆₀₀ were selected for all following infiltration experiments. Moreover, 0.1% Tween20 was chosen over DMSO 3% due to better solubilizing CBGA substrate.

In this embodiment, leaf samples were collected at 2 DPI and immediately frozen in liquid nitrogen. RNA extraction was done using RNA plant mini-kit as described by manufacturer (Qiagen). cDNA was synthesized using RNA to cDNA Ecodry Premix as described by manufacturer (Takara). Template cDNA was normalized to 50 ng of corresponding total RNA per reaction. Annealing temperature in Celsius: 60. Extension time: 15s. 35 cycles. Q5 DNA polymerase kit used as described by manufacturer (New England Biolabs). RT-PCR primers are outlined in Table 5 below.

### Materials and Methods Example 6: Transient Transformation of Cannabis sativa

The present inventors performed Agrobacterium tumefaciens-mediated transient transformation of Cannabis sativa. The experimental groups consisted of young leaves of high CBD variety (˜10% in dried flowers) and trichome leaves of high THC variety (˜20% dried flowers).

To transform leaves of high CBD varieties, the present inventors germinated 100 seeds three times; this was done to ensure that a sufficient number of plants would be available for all 9 independent transformation events. To transform trichome leaves, the present inventors used small trichome-containing leaves of several varieties known to be high THC varieties. Experimental set up consisted of 2 different Agrobacterium tumefaciens strains. For transient transformation of Agrobacterium strain EHA 105, the present inventors grew cells in 10 ml of LB medium supplemented with 100 mg/L of Rifampicin and 50 mg/L of Kanamycin and for Agrobacterium strain GV3101::6000 cells were grown with 50 mg/L of Kanamycin, 25 mg/L of Gentamycin and 50 mg/L of Rifampicin. A single Agrobacterium colony was used for inoculation and grown overnight. Then, 1 ml of this culture was inoculated into 500 ml of aforementioned LB medium supplemented with 20 μM acetosyringone. Agrobacteria were grown to OD₆₀₀ of approximately between 1 and 1.5. The cells were pelleted in a centrifuge at room temperature and resuspended in infiltration medium containing 10 mM MES, 10 mM MgCl₂ and 200 μM acetosyringone to an OD₆₀₀ of 0.5.

Bacterial culture was then used for three different types of Cannabis Sativa transformations. In all cases, transformation was done in the form of co-transformation, mixing all relevant strains (plasmids) in equal proportion of cell numbers. First, for the present inventors infiltrated young (two weeks old) fully expended Cannabis sativa plants using 1 ml syringe. Prior to transformation, plants were kept under plastic cover, to ensure maximum softness of the leaves. Infiltration was performed from abaxial side, ensuring that the entire surface of the leaf is infiltrated at 12/h/12 h day/night at 22° C.

Second, the present inventors vacuum infiltrated detached young (two weeks old) fully expended Cannabis sativa leaves. Prior to transformation, plants were kept under plastic cover, to ensure maximum softness of the leaves. Leaves were then placed on half-strength Murashige and Skoog (1962) (½ MS) agar supplemented with 61.8 mM ammonium nitrate and incubated for 5 days at 12/h/12 h day/night at 22° C.

Third, trichome leaves were detached, placed into 50 ml Falcon tubes and vacuum infiltrated with aforementioned bacterial solution 2× for 10 min each. Leaves were then placed on ½ MS agar supplemented with 61.8 mM ammonium nitrate and incubated for 5 days.

All experiments were done in triplicates, with the fourth replicate done for collection of DNA/RNA and staining X-gluc for measuring the activity of beta-glucuronidase (GUS) after co-infiltration with Agrobacterium-containing GUS gene. In all cases, leaves were harvested after 5 days of transformation, frozen in liquid nitrogen and stored at −80° C.

### Materials and Methods Example 7: Extraction of Water-Soluble Cannabinoids from N. benthamiana

Fresh transformed plant material was harvested from greenhouse experiments in 15 or 50 mL polypropylene centrifuge tubes and flash frozen in liquid N₂. The frozen plant material was enzymatically quenched by submersing the plant material in boiling methanol for 2 min. The methanol-quenched material was homogenised using a P-10-35 homogenizer (Kinematica, Bohemia N.Y.). The homogenate was extracted by brief agitation in a final volume of 10 mL or 30 mL 70% methanol (v/v) respective to tube size. The resulting extracts were clarified by centrifugation at 2,500 rpm at 4° C. for 15 minutes in a Beckman J-6B floor centrifuge (Beckman Coulter, Indianapolis Ind.). The supernatant was transferred into a polypropylene tube and evaporated under a stream of N₂ at 45° C. until dried. The extracts were reconstituted in methanol containing 20 μg/mL of the internal standard 7-Hydroxyoumarin (Sigma-Aldrich, H24003). The reconstituted extracts were placed into 1.5 mL microfuge tubes and clarified in a microcentrifuge at 10,000 g for 15 min. 500 L of the supernatant was transferred to a 2 mL auto sampler vial and kept at 4° C. until analysis. In vitro assays sample preparation: samples were syringed filtered through 0.45 μm PVDF membrane into a 2 mL auto sampler vial.

### Materials and Methods Example 8: Extraction of Water-Soluble Cannabinoids from Cannabis sativa

Fresh plant material was harvested from plants grown in chamber in 1.5 mL polypropylene centrifuge tubes and flash frozen in liquid N₂. The frozen plant material was homogenized using pestle and mortar and enzymatically quenched by submersing the plant material in boiling 100% ethanol for 2 min. Homogenized solution was diluted to 70% ethanol. The resulting extracts were clarified by centrifugation at 2,500 rpm at 4° C. for 15 minutes in Eppendorf centrifuge (Centrifuge 5415 R). The supernatant was transferred into a polypropylene tube and concentrated three times using vacuum centrifuge (Speedvac SC110, Savant). 2 μl of 20 g/mL of the internal standard Umbelliferone (Sigma-Aldrich, H24003) was added to 98 μl of concentrated extract and taken for analysis.

### Materials and Methods Example 9: Liquid Chromatography Mass Spectrometry Used to Confirm Functionalization and Glycosylation of Cannabinoids

The present inventor used liquid chromatography mass spectrometry to confirm functionalization and glycosylation of cannabinoids in the exemplary plant systems described herein. Specifically, mass spectrometry was performed on a quadrupole time-of-flight (QTOF) mass spectrometer (QTOF Micro, Waters, Manchester, UK) equipped with a Lockspray™ electrospray ion source coupled to a Waters Acquity UPLC system (Waters, Manchester, UK). Mass spectra were collected in the negative electrospray ionization mode (ESI−). The nebulization gas was set to 400 L/h at a temperature of 350° C., the cone gas was set to 15 L/H and the source temperature was set to 110° C. A capillary voltage and cone voltage were set to 2500 and 35 V, respectively. The MCP detector voltage was set to 2500 V. The Q-TOF micro MS acquisition rate was set to 1.0 s with a 0.1 s interscan delay. The scan range was from 100 to 1500 m/z. Data was collected in continuum mode. A lockmass solution of 50 ppm raffinose (503.1612 m/z) in 50:50 water:methanol was delivered at 20 μL/min through an auxiliary pump and acquired every 10 s during the MS acquisition. Separations were performed on a Waters HSS T3 C18 column (2.1×100 mm, particle size 1.8 μm) using a Waters ACQUITY UPLC System, equipped with an ACQUITY Binary Solvent Manager, ACQUITY Column Manager and ACQUITY Sample Manager (10 μL sample loop, partial loop injection mode, 5 μL injection volume, 4° C.). Eluents A and B were water and acetonitrile, respectively, both containing 0.1% formic acid. Elution was performed isocratically for 0.5 min at 10% eluent B and then linear gradient 100% eluent B in 14.5 min, and isocratically for 3 min at 100% eluent B. The column was re-equilibrated for 6 min. The flow rate was set to 250 μL/min and the column temperature was maintained at 30° C.

### Materials and Methods Example 10: Data Processing

Identification of individual cannabinoid analogs was performed by the present inventors, by their corresponding accurate mass shifts by Metabolynx (Waters Corp., Milford, USA). The method parameters for data processing were set as follows: retention time range 0.1-18 min, mass range 100-1500 Da, retention time tolerance 0.2 min, mass tolerance 0.05 Da, peak intensity threshold 14. Accurate mass measure of the continuum data was performed using the raffinose lock mass. Raw chromatographic data were additionally processed for extracted ion chromatogram sand peak area integration using Masslynx 4.1 (Waters Corp., Milford, USA).

The select cannabinoids, CBGA and CBDA were identified and quantitated using certified reference materials (Cerilliant, Round Rock, Tex.). All chemical structures and physiochemical and constitutional properties were generated using ChemDoodle version 8.1.0 (IChemLabs™, Chesterfield, Va.).

### Materials and Methods Example 11: Yeast Cell Gene Expression System

The present inventors generated an exemplary yeast-cell expression system based on the methylotrophic yeast Pichia pastoris (Komagataella phaffli) was used in this work. The Pichiapink™ system includes protease-deficient host strains and allows both intracellular as well as secreted protein production. In addition, the use of the inducible promoter alcohol oxidase (AOX1) uncouples growth from production of desired proteins, so that cells are not stressed by the accumulation of recombinant protein during growth phase yeast strain 4 (herein referred to as wild-type, WT), a double knockout for proteases prb1, pep4 (to avoid degradation of desired protein), was the background strain in the present inventor's yeast transformations. For secretion of proteins into the media, genes of interest were cloned in frame into the vector pPINK-aHC which contains the Saccharomyces cerevisiae α-mating factor pre-sequence for secreted expression of recombinant proteins. For intracellular production of proteins, the vector pPINK-HC was used. Both vectors contained the ADE2 marker for selection on minimal media lacking adenine (FIG. 35). Transformation and selection of transformants was conducted according to the manufacturer's instructions (Invitrogen). Such example is non-limiting, as a variety of expression vectors may be used with the current invention.

### Materials and Methods Example 12: Analysis of Yeast System Transgene Expression

Expression analysis for introduced transgenes was carried out by RT-PCR. For yeast, 2 mL of a 2-day old culture induced by methanol was centrifuged in a microfuge tube. The pellet was ground in a TissueLyser (QIAGEN Inc, USA). RNA was extracted following the EZNA plant RNA extraction kit (Omega Bio-tek Inc, USA). Up to a microgram of total RNA was used to synthesize cDNA using the superscript III cDNA synthesis kit (Thermo Fisher Scientific, USA). The cDNA was used to check for the expression of transgenes by RT-PCR.

### Materials and Methods Example 13: Transformation of Tobacco BY2 Cells for Cell Suspension Expression System

The present inventors used Agrobacterium Ti-plasmid mediated transformation with the plant expression vector pRI201-AN (Takara Bio USA), a binary vector for high-level expression of a foreign gene in dicotyledonous plants carrying the constitutive 35S promoter and an Arabidopsis Alcohol dehydrogenase (AtAdh) as a translational enhancer. 5 mL of LB containing 50 mg/L kanamycin was inoculated with a single colony of Agrobacterium tumefaciens strain GV3101 carrying a binary vector for the expression of the glycosyltransferase 76G1 from Stevia rebaudiana (SEQ ID NO. 61 and SEQ ID NO. 62) and the multi-drug ABC transporter ABCG2 (SEQ ID NO. 67 and SEQ ID NO. 68). The Agrobacterium culture was grown overnight at 180 rpm and 28° C. to an OD600 of 0.6 to 0.8. For transformation, 10 ml of 3-day old BY2 cell cultures was incubated with 500 ul of the Agrobacterium culture and 10 μl of 100 mM acetosyringone for 48 hours in the dark at room temperature in sterile 50 mL falcon tubes. After 48 hours, the cells were washed twice in Murashige and Skoog medium supplemented with 500 mg/L carbenicillin before plating on selective media (Murashige and Skoog supplemented with 500 mg/L carbenicillin and 50 mg/L kanamycin). Calli were picked at 4 weeks and re-plated for further screening for transgene expression.

### Materials and Methods Example 14: Statistical Analysis of Yeast and Tobacco Expressions Systems

All experimental treatments were carried out in triplicates. Data were analyzed using GraphPad Prism software package (http://www.graphpad.com/prism/Prism.htm). Student's t-test and one-way analysis of variance (ANOVA) with Dunnett's Multiple Comparison test for comparing multiple lines with the control were used. All analyses for significant differences were performed at P≤0.05.

### Materials and Methods Example 15: Yeast and/or Tobacco Cell Suspension Sample Preparation for the Analysis of Water-Soluble Cannabinoids

Cell suspension cultures were harvested by centrifugation in 15 or 50 mL polypropylene centrifuge tubes. The supernatants were transferred to a new centrifuge tube and both the cell pellet and supernatant was flash frozen in liquid N2. Cell pellets were freeze dried and ˜100 mg of material was extracted by bead milling with 250 uL volume of 0.1 mm zirconia beads in 1 mL of 70% methanol:water (v/v) containing 20 μg/mL of the internal standard 7-hydroxyoumarin (Sigma-Aldrich, H24003). The resulting extracts were clarified by centrifugation at 13,000 rcf for 10 minutes. The clarified supernatant was transferred into a 2 mL autosampler. Supernatants were concentrated by freeze drying 2-fold and spiked at 20 μg/mL of the internal standard 7-hydroxyoumarin final concentration. A 1 mL aliquot was transferred to a 2 mL autosampler.

### Materials and Methods Example 16: Liquid Chromatography Mass Spectrometry for Yeast and Tobacco Suspension Culture Systems

Mass spectrometry was performed on a quadrupole time-of-flight (QTOF) mass spectrometer (QTOF Ultima, Waters, Manchester, UK) equipped with a Lockspray™ electrospray ion source coupled to a Waters Acquity UPLC system (Waters, Manchester, UK). Mass spectra were collected in the negative electrospray ionization mode (ESI−). The nebulization gas was set to 650 L/h at a temperature of 500° C., the cone gas was set to 15 L/H and the source temperature was set to 110° C. A capillary voltage and cone voltage were set to 2500 and 35 V, respectively. The MCP detector voltage was set to 2200 V. The Q-TOF Ultima MS acquisition rate was set to 0.25 s with a 0.1 s interscan delay. The scan range was from 100 to 1500 m/z. Data was collected in continuum mode. A lockmass solution of 50 ppm raffinose (503.1612 m/z) in 50:50 water:methanol was delivered at 20 μL/min through an auxiliary pump and acquired every 10 s during the MS acquisition. Separations were performed on a Waters BEH C18 column (2.1×50 mm, particle size 1.8 μm) using a Waters ACQUITY UPLC System, equipped with an ACQUITY Binary Solvent Manager, and ACQUITY Sample Manager (20 μL sample loop, partial loop injection mode, 5 μL (Cell extracts) or 10 μL (Supernatant) injection volume, 4° C.). Eluents A and B were water and acetonitrile, respectively, both containing 0.1% formic acid. Elution was performed isocratically for 0.1 min at 8% eluent B and then linear gradient 100% eluent B in 6.0 min, and isocratically for 1 min at 100% eluent B. The column was re-equilibrated for 1.5 min. The flow rate was set to 500 μL/min and the column temperature was maintained at 40° C.

### Materials and Methods Example 17: Data Processing for Individual Cannabinoid Analogs in Yeast and Tobacco Suspension Culture Systems

Identification of individual cannabinoid analogs was performed, by their corresponding accurate mass shifts by Metabolynx (Waters Corp., Milford, USA). The method parameters for data processing were set as follows: retention time range 0.1-7.5 min, mass range 100-1500 Da, retention time tolerance 0.2 min, mass tolerance 0.05 Da, peak intensity threshold 14. Accurate mass measure of the continuum data was performed using the raffinose lock mass. Raw chromatographic data were additionally processed for extracted ion chromatogram sand peak area integration using Masslynx 4.1 (Waters Corp., Milford, USA). CBGA and CBDA were identified and quantitated using certified reference materials (Cerilliant, Round Rock, Tex.). All chemical structures and physiochemical and constitutional properties were generated using ChemDoodle version 8.1.0 (IChemLabs™, Chesterfield, Va.).

### Materials and Methods Example 18: Spectral Analysis of Water Soluble Cannabinoids Identification of Modified Cannabinoids by Mass Spectrometry

The present inventors identified the cannabinoid bio-transformations associated with the gene constructs expressed in tobacco cell suspension and yeast cultures. Based on the predicted glycosylation reactions and empirical information from the chromatographic assays, we predicted the most likely glycosylation events that would occur to the parent molecules CBGA and CBDA along with their physiochemical and constitutional properties (FIGS. 36 and 37, respectively). With this information and through the use of accurate mass measurements, we were able to identify the molecules in the chromatographic analysis and produce extracted ion chromatograms for peak integration as illustrated in FIGS. 38-40. Peak areas for each identified molecule were used for relative quantification between treatments. Based on these results we identified cannabinoid molecules containing up to two glycosides moieties and an O-acetyl glycoside. Summaries of those identifications are presented in Tables 11 and 12 for exemplary cannabinoids CBGA and CBDA respectively.

### Materials and Methods Example 19: Cannabinoid Stability in Solution

The present inventors generated a stock solution of the CBD/CBDA+glycosides mixture in water which was further diluted 10× in all treatment solutions. A 10 mL starting volume was used for all treatments, and all treatments were prepared in triplicate. The initial molar concentration of each substrate was as follows: CBD: 1.85 μM; CBDA: 5.83 μM; 1×CBDA Gly: 0.40 μM; and 1×CBD O-Acetyl Gly: 0.22 μM. Each sample was incubated at room temperature. Samples were stored in 15 mL polypropylene centrifuge tubes. All samples were shielded from light—tubes were wrapped in aluminum foil. Each sample was prepared in triplicate in the following buffers: water only; pH=3; pH=4; pH=7; pH=8; water only+10 mM ascorbic acid; pH=3+10 mM ascorbic acid; pH=4+10 mM ascorbic acid; pH=7+10 mM ascorbic acid; and pH=8+10 mM ascorbic acid.

Those skilled in the art will appreciate, or be able to ascertain using no more than routine experimentation, further features and advantages of the invention based on the above-described embodiments. Accordingly, the invention is not to be limited by what has been particularly shown and described. All publications and references are herein expressly incorporated by reference in their entirety.

### TABLES

TABLE 1
CBGA Biotransformed Products

| | | | | | | Molecular |
|---|---|---|---|---|---|---|
| | RRT to | Expected | | Error | Error | Formula [M − |
| Product | Parent | m/z | Found m/z | (mDa) | (ppm) | H]− |
| R-OH 1 x Glycoside | 0.58 | 537.2700 | 537.2703 | −0.30 | 0.6 | C28H41O10 |
| 2 x Glycoside | 0.59 | 683.3279 | 683.3258 | 2.10 | −3.1 | C34H51O14 |
| 1 x O acetyl Glycoside | 0.73 | 563.2856 | 563.2844 | 1.20 | −2.1 | C30H43O10 |
| 1 x Glycoside #1 | 0.74 | 521.2751 | 521.2734 | 1.70 | −3.3 | C28H41O9 |
| R-OH #1 | 0.80 | 375.2171 | 375.2224 | −5.30 | 14.1 | C22H31O5 |
| 1 x Glycoside #2 | 0.81 | 521.2751 | 521.2727 | 2.40 | −4.6 | C28H41O9 |
| R-OH #2 | 0.81 | 375.2171 | 375.2237 | −6.60 | 17.6 | C22H31O5 |
| R-OH #3 | 0.94 | 375.2171 | 375.2192 | −2.10 | 5.6 | C22H31O5 |
| CBGA | 1.00 | 359.2222 | 359.2245 | −2.30 | 6.4 | C22H31O4 |

RRT Relative Retention Time to Parent Molecule
R-OH Functionalized by addition of O atom

TABLE 2
CBDA Biotransformed Products

| | | | | | | Molecular |
|---|---|---|---|---|---|---|
| | RRT to | Expected | Found | Error | Error | Formula |
| Product | Parent | m/z | m/z | (mDa) | (ppm) | [M − H]− |
| 2 x Glycoside | 0.56 | 681.3122 | 681.3097 | 2.50 | −3.7 | C34H49O14 |
| R-OH 1 x Glycoside | 0.61 | 535.2543 | 535.2599 | −5.60 | 10.5 | C28H39O10 |
| 1 x Glycoside | 0.71 | 519.2601 | 519.2594 | 0.70 | 1.3 | C28H39O9 |
| 1 x O acetyl Glycoside | 0.71 | 561.2700 | 561.2700 | 0.00 | 0 | C30H41O10 |
| R-OH #1 | 0.84 | 373.2015 | 373.2074 | −5.90 | 15.8 | C22H29O5 |
| R-OH #2 | 0.87 | 373.2015 | 373.2034 | −1.90 | 5.1 | C22H29O5 |
| R-OH #3 | 0.96 | 373.2015 | 373.2040 | −2.50 | −8 | C22H29O5 |
| CBDA | 1.00 | 357.2066 | 357.2122 | −5.60 | 15.7 | C22H29O4 |

RRT Relative Retention Time to Parent Molecule
R-OH Functionalized by addition of O atom’

TABLE 3
Forward and reverse primers for
RT-PCR of CYP3A4 and P450 oxidoreductase.
SEQ. ID NO. 76 represents the forward
primer of CYP3A4; SEQ. ID NO. 77 represents
the reverse primer of CYP3A4; SEQ. ID NO. 78
represents the forward primer of P450
oxidoreductase; and SEQ. ID NO. 79
represents the reverse primer of P450
oxidoreductase.

| | | P450 |
|---|---|---|
| Sequence | CYP3A4 | oxidoreductase |
| Primers | Forward | Forward |
| for | TGCCTAATAAAG | GGAAGAGCTTTG |
| RT-PCR | CTCCTCCTACT | GTTCCTATGT |
| | Reverse | Reverse |
| | GCTCCTGAAACA | GCTCCCAATTCA |
| | GTTCCATCTC | GCAACAATATC |

TABLE 4
Forward and reverse primers for CBDA
synthase, UGT76G1 and ABCG2. SEQ. ID NO. 80
represents the forward primer of CBDA
synthase; SEQ. ID NO. 81 represents the reverse
primer of CBDA synthase; SEQ. ID NO. 82
represents the forward primer of UGT76G1;
SEQ. ID NO. 83 represents the reverse primer
of UGT76G1; SEQ. ID NO. 84 represents the
forward primer of ABCG2; and SEQ. ID NO. 85
represents the reverse primer of ABCG2.

| | CBDA | | |
|---|---|---|---|
| Sequence | synthase | UGT76G1 | ABCG2 |
| Primers | Forward | Forward | Forward |
| for | primer: | primer: | primer: |
| RT-PCR | ACATCACA | GATTGGAA | CCTTCAGG |
| | ATCACACA | GAACAAGC | ATTGTCAG |
| | AAACTAAC | TTCAGGAT | GAGATG |
| | AAAAG | TTCC | |
| | Reverse | Reverse | Reverse |
| | primer: | primer: | primer: |
| | GGCCATAG | CCATCCTG | GCAGGTCC |
| | TTTCTCAT | AATGAGTC | ATGAAACA |
| | CAATGG | CAAAAAGC | TCAATC |
| | | TC | |

TABLE 5
Trichome-targeted CBDA synthase (CBDAs),
Trichome-targeted UGT and PM-targeted UTR1.
SEQ. ID NO. 86 represents the forward primer
of Trichome-targeted CBDAs; SEQ. ID NO. 87
represents the reverse primer of Trichome-
targeted CBDAs; SEQ. ID NO. 88 represents the
forward primer of Trichome-targeted UGT;
SEQ. ID NO. 89 represents the reverse primer
of Trichome-targeted UGT; SEQ. ID NO. 90
represents the forward primer of Plasma
membrane-targeted UTRI; and SEQ. ID NO. 91
represents the reverse primer of Plasma
membrane-targeted UTRI.

| | | | Plasma |
|---|---|---|---|
| | Trichome- | Trichome- | membrane- |
| | targeted | targeted | targeted |
| Sequence | CBDAs | UGT | UTR1 |
| Primers | Forward | Forward | Forward |
| for | primer: | primer: | primer: |
| RT-PCR | AAAGATCA | AGTGCTCA | TTGTTCCT |
| | AAAGCAAG | ACATTCTC | TAAACCTC |
| | TTCTTCAC | CTTTTGGT | GCCTTTGA |
| | TGT | T | C |
| | Reverse | Reverse | Reverse |
| | primer: | primer: | primer: |
| | CCATGCAG | TCTGAAGC | TCATTATG |
| | TTTGGCTA | CAACATCA | GAGCACTC |
| | TGAACATC | ACAATTCC | CACTCTCT |
| | T | A | G |

TABLE 6
Cytosolic-targeted CBDA synthase (cytCBDAs),
Cytosolic-targeted UGT (cytUGT). SEQ. ID NO. 92
represents the forward primer of Cytosolic-
targeted CBDA synthase; SEQ. ID NO. 93
represents the reverse primer of Cytosolic-
targeted CBDA synthase; SEQ. ID NO. 94
represents the forward primer of Cytosolic-
targeted UGT; and SEQ. ID NO. 95 represents the
reverse primer of Cytosolic-targeted UGT.

| | Cytosolic-targeted | Cytosolic- |
|---|---|---|
| Sequence | CBDA synthase | targeted UGT |
| Primers | Forward primer: | Forward primer: |
| for | AAAGATCAAAAGCAA | AGAACTGGAAGAATC |
| RT-PCR | GTTCTTCACTGT | CGAACTGGAA |
| | Reverse primer: | Reverse primer: |
| | ATAAACTTCTCCAAG | AAATCATCGGGACAC |
| | GGTAGCTCCG | CTTCACAAAC |

TABLE 7
Summary of results from glycosylation and functionalization
experiments in N. benthamiana leaves.

| | | | | CBGA | | | |
|---|---|---|---|---|---|---|---|
| | | | CBGA | glycoside + | | CBDA | CBDA |
| | | CBGA | glycoside | acetylated | CBDA | glycoside | Hydroxyl |
| Agrobacterium | Substrate | (relative | (relative | (relative | (relative | (relative | (relative |
| Constructs | fed | amount) | amount) | amount) | amount) | amount) | amount) |
| Trichome CBDA | CBGA | + | + | + | + | ND | ND |
| synthase + trichome | | | | | | | |
| glycosyltransferase + | | | | | | | |
| PM-UTR1) + | | | | | | | |
| Myb/catalase* + P19 | | | | | | | |
| silencing supressor* | | | | | | | |
| Cytosolic CBDA | CBGA | + | +++ | +++ | +++ | ND | ND |
| synthase, | | | | | | | |
| glycosyltransferase and | | | | | | | |
| plasma membrane ABC | | | | | | | |
| transporter) + | | | | | | | |
| Myb/catalase + P19 | | | | | | | |
| silencing suppressor | | | | | | | |
| 201-SUS (cytosolic | CBGA | + | +++ | ++++ | + | + | + |
| CBDA synthase, | | | | | | | |
| glycosyltransferase and | | | | | | | |
| plasma membrane ABC | | | | | | | |
| transporter) | | | | | | | |
| CYP3A4 + oxidoreductase | CBDA | ND | + | ND | +++ | +++++ | +++++ |
| (cytochrome P450 with | | | | | | | |
| P450 oxidoreductase) | | | | | | | |
| Cytosolic CBDA | CBGA | ++++ | +++++ | +++++ | ND | ++ | ++ |
| synthase + cytosolic | | | | | | | |
| glycosyltransferase + | | | | | | | |
| Myb/catalase* + P19 | | | | | | | |
| silencing suppressor* | | | | | | | |
| P450/ | CBGA | + | ++++ | + | ND | ++ | ++ |
| MYBcatalase/cytosolic | | | | | | | |
| CBDA synthase, | | | | | | | |
| glycosyltransferase and | | | | | | | |
| plasma membrane ABC | | | | | | | |
| transporter | | | | | | | |
| No agrobacterium | CBGA | + | + | + | ND | ND | ND |
| (negative control) | | | | | | | |

*Co-infiltration with and without construct was tested in different replicates

TABLE 8
Summary of results from glycosylation and functionalization
experiments in Cannabis sativa leaves.

| | | CBDA | CBDA |
|---|---|---|---|
| | CBDA | glycoside | Hydroxyl |
| | (relative | (relative | (relative |
| Agrobacterium Constructs | amount) | amount) | amount) |
| Trichome CBDA synthase + | ++ | trace | trace |
| trichome glycosyltransferase + | | | |
| plasma membrane-targeted | | | |
| sugar transporter) + | | | |
| Myb/catalase | | | |
| cytosolic CBDA synthase, | ++ | ++++ | +++++ |
| cytosolic glycosyltransferase + | | | |
| Myb/catalase | | | |
| 201-SUS (cytosolic CBDA | ++ | ++ | ++ |
| synthase, glycosyltransferase and | | | |
| plasma membrane ABC | | | |
| transporter) | | | |

TABLE 9
Exemplary Glycosyltransferase sequence identification

| SEQ ID NO. | Name | Organism | Type |
|---|---|---|---|
| SEQ ID NO. 26 | NtGT5a | Nicotiana tabacum | Amino Acid |
| SEQ ID NO. 27 | NtGT5a | Nicotiana tabacum | DNA |
| SEQ ID NO. 28 | NtGT5b | Nicotiana tabacum | Amino Acid |
| SEQ ID NO. 29 | NtGT5b | Nicotiana tabacum | DNA |
| SEQ ID NO. 30 | NtGT4 | Nicotiana tabacum | Amino Acid |
| SEQ ID NO. 31 | NtGT4 | Nicotiana tabacum | DNA |
| SEQ ID NO. 32 | NtGT1b | Nicotiana tabacum | Amino Acid |
| SEQ ID NO. 33 | NtGT1b | Nicotiana tabacum | DNA |
| SEQ ID NO. 34 | NtGT1a | Nicotiana tabacum | Amino Acid |
| SEQ ID NO. 35 | NtGT1a | Nicotiana tabacum | DNA |
| SEQ ID NO. 36 | NtGT3 | Nicotiana tabacum | Amino Acid |
| SEQ ID NO. 37 | NtGT3 | Nicotiana tabacum | DNA |
| SEQ ID NO. 38 | NtGT2 | Nicotiana tabacum | Amino Acid |
| SEQ ID NO. 39 | NtGT2 | Nicotiana tabacum | DNA |

TABLE 10
Cannabinoid production cellular compartmentalization models. Different shaded
columns and rows correspond to different exemplary expression constructs used.

| | | | | | | Catalase |
|---|---|---|---|---|---|---|
| | | | | | | to |
| | | | | | | degrade |
| Cannabinoid | | | | | Myb | H₂O₂ |
| production/ | | UDP | Cannabinoid | | transcription | from |
| accumulation | CBDA | glycosyl | ABC | UDP glucose | factor for | CBDA |
| system | Synthase | transferase | transporter | transporter | cannabinoids | Synthase |
| Cytoplasmic | Minus | Required but | No gene | No gene | Express | Express |
| accumulation | trichome | no targeting | required | required | | |
| | target | change | | | | |
| | sequence | | | | | |
| Trichome | No change | Add | No gene | Target to | Express | Express |
| (low pH) | | trichome | required | plasma | | |
| synthesis | | target | | membrane | | |
| | | sequence | | | | |
| Cell | Minus | Required but | Target to | No gene | Express | Express |
| suspension | trichome | no targeting | plasma | required | | |
| cultures | target | change | membrane | | | |
| | sequence | | (PM) | | | |

TABLE 11
CBGA Biotransformed Products

| | | | | | | Molecular |
|---|---|---|---|---|---|---|
| | RRT to | Expected | Found | Error | Error | Formula |
| Product | Parent | m/z | m/z | (mDa) | (ppm) | [M − H]− |
| 1 x | 0.72 | 521.2751 | 521.2700 | −5.1 | −9.8 | C28H41O9 |
| Glycoside | | | | | | |
| CBGA | 1.00 | 359.2222 | 359.2190 | −3.2 | −8.9 | C22H31O4 |

RRT Relative Retention Time to Parent Molecule

TABLE 12
CBDA Biotransformed Products

| | | | | | | Molecular |
|---|---|---|---|---|---|---|
| | RRT | Expected | Found | Error | Error | Formula |
| Product | to Parent | m/z | m/z | (mDa) | (ppm) | [M − H]− |
| 2 x Glycoside | 0.52 | 681.3122 | 681.3076 | −4.76 | −6.8 | C34H49O14 |
| 1 x Glycoside #1 | 0.67 | 519.2594 | 519.2583 | −1.1 | −2.1 | C28H39O9 |
| 1 x O acetyl Glycoside | 0.68 | 561.2700 | 561.2653 | −4.7 | −8.4 | C30H41O10 |
| 1 x Glycoside #2 | 0.80 | 519.2594 | 519.2681 | 8.8 | 16.7 | C28H39O9 |
| CBDA | 1.00 | 357.2066 | 357.2091 | 2.5 | 7.0 | C22H29O4 |

RRT Relative Retention Time to Parent Molecule

Based on the reduced retention time in the HPLC gradient. The glycosylated cannabinoids, which eluted earlier than their non-modified forms, are demonstrated to be more water-soluble than their non-modified forms.

TABLE 13
RT-PCR primers for confirmation of gene
expression in transgenic intracellular Pichia
and tobacco cultures. SEQ. ID NO. 96 represents
the forward primer of NtGT1; SEQ. ID NO. 97
represents the reverse primer of NtGT1; SEQ. ID
NO. 98 represents the forward primer of NtGT2;
SEQ. ID NO. 99 represents the reverse primer of
NtGT2; SEQ. ID NO. 100 represents the forward
primer of NtGT3; SEQ. ID NO. 101 represents the
reverse primer of NtGT3; SEQ. ID NO. 102
represents the forward primer of NtGT4; SEQ. ID
NO. 103 represents the reverse primer of NtGT4;
SEQ. ID NO. 104 represents the forward primer
of NtGT5; SEQ. ID NO. 105 represents the
reverse primer of NtGT5; SEQ. ID NO. 106
represents the forward primer for Kat-E; SEQ.
ID NO. 107 represents the reverse primer for
Kat-E; SEQ. ID NO. 108 represents the forward
primer of UGT76G1; SEQ. ID NO. 109 represents
the reverse primer of UGT76G1; SEQ. ID NO. 110
represents the forward primer of UGT76G1 (for
tobacco BY2 cells); SEQ. ID NO. 111 represents
the reverse primer of UGT76G1 (for tobacco BY2
cells); SEQ. ID NO. 112 represents the forward
primer of ABCG2 (for tobacco BY2 cells); and
SEQ. ID NO. 113 represents the reverse primer
of ABCG2 (for tobacco BY2 cells).

| Target | | |
|---|---|---|
| gene | Forward primer | Reverse primer |
| NtGT1 | ATGAAAACAACAGA | TGAAGTTGTAGGCC |
| | ACTTGTCTTCA | TAGCATGG |
| NtGT2 | ATGGTTCAACCACA | TTGAATACACCAGT |
| | CGTCTTACTGG | TGGGGTCG |
| NtGT3 | ATGAAAGAGACTAA | CATCACGCAGATTT |
| | AAAAATTGAGT | TGAATATGG |
| NtGT4 | ATGGCTACTCAGGT | GGCCTTAGTTAGCT |
| | GCATAAATTGC | CGACACGG |
| NtGT5 | ATGGGCTCTATCGG | CGGGGATGAAGTCC |
| | TGCAGAACTAA | AAGGTTGT |
| Kat-E | ATGTCTCAACATAA | CGTAGCAAATCCCC |
| | CGAGAAAAACC | TGATGTCT |
| UGT76G1 | ATGGAGAACAAAAC | CCTTTAGCATGGGA |
| | CGAGACAACCG | AAACCGGA |
| UGT76G1 | GATTGGAAGAACAA | CCATCCTGAATGAG |
| (for | GCTTCAGGATTTCC | TCCAAAAAGCTC |
| tobacco | | |
| BY2 | | |
| cells) | | |
| ABCG2 | CCTTCAGGATTGTC | GCAGGTCCATGAAA |
| (for | AGGAGATG | CATCAATC |
| tobacco | | |
| BY2 | | |
| cells) | | |

TABLE 14
Day 0 time course incubation of unmodified, glycosylated, and
acetylated cannabinoid glycoside compounds in solution.

| Day 0 (ug/mL) | CBD | CBDA | 1X CBDA Gly | 1X CBD O-Acetyl Gly |
|---|---|---|---|---|
| Water | 399.81 ± 111.06 | 1689.45 ± 345.00 | 230.85 ± 36.38 | 137.46 ± 35.49 |
| pH = 3 | 568.71 ± 115.90 | 2278.25 ± 279.51 | 217.58 ± 45.40 | 136.49 ± 43.90 |
| pH = 4 | 505.97 ± 5.10 | 1941.33 ± 141.84 | 179.48 ± 20.25 | 101.60 ± 31.49 |
| pH = 7 | 528.04 ± 91.17 | 2098.05 ± 71.62 | 199.19 ± 24.03 | 107.55 ± 20.03 |
| pH = 8 | 513.35 ± 39.23 | 1907.82 ± 81.03 | 190.06 ± 16.22 | 108.41 ± 12.42 |
| Water + AA | 561.85 ± 86.83 | 1925.54 ± 266.50 | 198.81 ± 27.15 | 102.24 ± 12.18 |
| pH = 3 + AA | 565.75 ± 40.96 | 2099.04 ± 78.79 | 190.77 ± 3.98 | 110.44 ± 13.31 |
| pH = 4 + AA | 524.40 ± 34.95 | 1928.33 ± 22.80 | 190.28 ± 14.45 | 118.27 ± 1.16 |
| pH = 7 + AA | 500.03 ± 21.45 | 1956.80 ± 114.66 | 185.33 ± 20.89 | 108.19 ± 19.12 |
| pH = 8 + AA | 1136.36 ± 996.36 | 3072.96 ± 1630.46 | 315.93 ± 180.23 | 137.53 ± 6.71 |

TABLE 15
Day 1 time course incubation of unmodified, glycosylated, and acetylated
cannabinoid glycoside compounds in solution.

| Day 1 (ug/mL) | CBD | CBDA | 1X CBDA Gly | 1X CBD Gly | 1X CBD O-Acetyl Gly |
|---|---|---|---|---|---|
| Water | 629.00 ± 383.79 | 2132.80 ± 1412.98 | 205.52 ± 198.82 | 1.20 ± 21.68 | 136.86 ± 139.68 |
| pH = 3 | 713.93 ± 41.47 | 2550.09 ± 85.93 | 299.02 ± 9.63 | 8.83 ± 1.80 | 216.46 ± 9.61 |
| pH = 4 | 380.77 ± 233.10 | 1504.66 ± 600.27 | 177.59 ± 62.72 | below LOQ | 150.94 ± 27.25 |
| pH = 7 | 880.54 ± 5.87 | 2577.70 ± 0.44 | 345.49 ± 23.25 | 15.45 ± 0.07 | 250.75 ± 6.23 |
| pH = 8 | 747.61 ± 101.87 | 2572.31 ± 140.62 | 326.74 ± 21.82 | 12.86 ± 4.15 | 221.10 ± 16.30 |
| Water + AA | 428.63 ± 198.77 | 1765.03 ± 561.85 | 246.74 ± 47.28 | 7.99 ± 4.17 | 204.63 ± 24.09 |
| pH = 3 + AA | 650.49 ± 12.10 | 2330.65 ± 45.55 | 283.54 ± 46.51 | 9.78 ± 5.27 | 223.32 ± 48.67 |
| pH = 4 + AA | 406.17 ± 210.69 | 1614.31 ± 504.49 | 230.30 ± 51.72 | 5.82 ± 3.11 | 198.56 ± 24.37 |
| pH = 7 + AA | 505.88 ± 276.58 | 1944.55 ± 741.75 | 249.00 ± 83.03 | 5.73 ± 4.70 | 185.02 ± 35.46 |
| pH = 8 + AA | 291.15 ± 78.65 | 1331.89 ± 302.72 | 195.97 ± 48.06 | 1.04 ± 4.15 | 180.83 ± 33.60 |

TABLE 16
Day 4 time course incubation of unmodified, glycosylated, and
acetylated cannabinoid glycoside compounds in solution.

| Day 4 (ug/mL) | CBD | CBDA | 1X CBDA Gly | 1X CBD Gly | 1X CBD O-Acetyl Gly |
|---|---|---|---|---|---|
| Water | 432.54 ± 251.39 | 348.83 ± 126.90 | 50.55 ± 10.85 | 15.11 ± 10.77 | 160.23 ± 23.75 |
| pH = 3 | 191.94 ± 55.68 | 215.62 ± 28.65 | 35.62 ± 8.29 | 18.94 ± 4.88 | 174.88 ± 14.16 |
| pH = 4 | 278.33 ± 85.33 | 264.51 ± 58.26 | 65.29 ± 9.09 | 29.03 ± 8.17 | 225.84 ± 39.59 |
| pH = 7 | 539.55 ± 260.69 | 373.85 ± 102.13 | 51.73 ± 7.48 | 19.64 ± 3.44 | 190.05 ± 24.38 |
| pH = 8 | 560.90 ± 69.27 | 461.23 ± 80.64 | 61.23 ± 21.53 | 26.99 ± 15.29 | 174.09 ± 59.87 |
| Water + AA | 182.66 ± 25.03 | 197.39 ± 11.23 | 33.74 ± 8.21 | 17.55 ± 5.99 | 153.06 ± 28.19 |
| pH = 3 + AA | 232.39 ± 113.88 | 217.83 ± 61.23 | 31.98 ± 6.06 | 17.99 ± 4.60 | 157.19 ± 1.58 |
| pH = 4 + AA | 305.20 ± 97.35 | 267.13 ± 59.52 | 40.84 ± 3.72 | 17.02 ± 5.68 | 144.44 ± 18.85 |
| pH = 7 + AA | 398.16 ± 391.86 | 286.12 ± 150.45 | 41.31 ± 15.16 | 19.96 ± 9.83 | 176.48 ± 33.58 |
| pH = 8 + AA | 173.43 ± 17.53 | 182.43 ± 4.69 | 35.59 ± 2.84 | 15.86 ± 2.63 | 152.03 ± 5.79 |

TABLE 17
Day 5 time course incubation of unmodified, glycosylated, and
acetylated cannabinoid glycoside compounds in solution.

| Day 5 (ug/mL) | CBD | CBDA | 1X CBDA Gly | 1X CBD Gly | 1X CBD O-Acetyl Gly |
|---|---|---|---|---|---|
| Water | 466.09 ± 130.53 | 232.98 ± 82.28 | 27.85 ± 10.52 | 14.60 ± 1.31 | 131.77 ± 33.91 |
| pH = 3 | 330.79 ± 138.28 | 151.00 ± 27.30 | 14.46 ± 6.76 | 18.00 ± 0.59 | 83.72 ± 35.56 |
| pH = 4 | 510.50 ± 76.06 | 166.21 ± 10.23 | 33.83 ± 4.89 | 18.26 ± 0.52 | 164.84 ± 35.61 |
| pH = 7 | 593.92 ± 6.30 | 214.85 ± 24.56 | 36.02 ± 8.21 | 17.50 ± 1.63 | 186.93 ± 5.65 |
| pH = 8 | 498.21 ± 92.88 | 324.72 ± 32.71 | 37.06 ± 3.83 | 19.73 ± 0.55 | 162.75 ± 31.35 |
| Water + AA | 448.64 ± 33.51 | 134.25 ± 11.78 | 18.19 ± 2.31 | 19.70 ± 0.80 | 146.60 ± 10.68 |
| pH = 3 + AA | 320.48 ± 92.87 | 119.25 ± 35.92 | 13.83 ± 7.04 | 19.29 ± 3.26 | 97.81 ± 29.67 |
| pH = 4 + AA | 366.65 ± 92.35 | 141.46 ± 34.95 | 17.99 ± 5.54 | 19.34 ± 1.90 | 113.89 ± 31.15 |
| pH = 7 + AA | 353.25 ± 135.11 | 160.93 ± 99.51 | 18.25 ± 14.93 | 19.05 ± 3.91 | 110.66 ± 48.12 |
| pH = 8 + AA | 287.24 ± 39.56 | 110.83 ± 6.09 | 12.07 ± 2.03 | 20.14 ± 1.73 | 92.06 ± 15.47 |

TABLE 18
Day 6 time course incubation of unmodified, glycosylated, and
acetylated cannabinoid glycoside compounds in solution.

| Day 6 (ug/mL) | CBD | CBDA | 1X CBDA Gly | 1X CBD Gly | 1X CBD O-Acetyl Gly |
|---|---|---|---|---|---|
| Water | 179.13 ± 17.88 | 177.27 ± 31.31 | 24.40 ± 9.39 | 8.56 ± 7.04 | 148.36 ± 33.57 |
| pH = 3 | 607.98 ± 416.07 | 299.37 ± 129.48 | 29.72 ± 11.20 | 21.59 ± 7.97 | 175.06 ± 31.37 |
| pH = 4 | 897.52 ± 65.92 | 437.55 ± 32.34 | 47.69 ± 7.79 | 32.87 ± 8.08 | 231.03 ± 36.34 |
| pH = 7 | 1115.01 ± 169.64 | 516.99 ± 40.18 | 67.01 ± 7.94 | 46.80 ± 10.12 | 267.45 ± 30.83 |
| pH = 8 | 911.61 ± 84.17 | 492.64 ± 27.37 | 62.45 ± 1.31 | 44.10 ± 3.45 | 218.64 ± 22.05 |
| Water + AA | 957.17 ± 315.70 | 446.90 ± 93.03 | 46.98 ± 17.51 | 34.98 ± 9.28 | 200.20 ± 37.42 |
| pH = 3 + AA | 801.14 ± 230.88 | 407.88 ± 70.00 | 37.19 ± 11.66 | 27.67 ± 9.51 | 190.69 ± 36.71 |
| pH = 4 + AA | 777.26 ± 56.02 | 408.97 ± 19.32 | 41.59 ± 4.42 | 31.67 ± 4.29 | 215.72 ± 16.69 |
| pH = 7 + AA | 828.02 ± 269.38 | 448.43 ± 75.60 | 47.26 ± 13.89 | 30.43 ± 4.82 | 205.61 ± 26.64 |
| pH = 8 + AA | 847.26 ± 88.56 | 439.50 ± 16.67 | 46.68 ± 2.47 | 32.97 ± 1.31 | 217.66 ± 11.23 |

TABLE 19
Day 7 time course incubation of unmodified, glycosylated, and
acetylated cannabinoid glycoside compounds in solution.

| Day 7 (ug/mL) | CBD | CBDA | 1X CBDA Gly | 1X CBD Gly | 1X CBD O-Acetyl Gly |
|---|---|---|---|---|---|
| Water | 680.13 ± 229.24 | 373.94 ± 53.04 | 43.73 ± 8.52 | 23.89 ± 5.27 | 185.62 ± 22.87 |
| pH = 3 | 563.80 ± 65.50 | 320.95 ± 26.33 | 35.76 ± 6.26 | 27.06 ± 4.05 | 195.77 ± 15.07 |
| pH = 4 | 641.86 ± 33.49 | 335.80 ± 1.93 | 41.68 ± 3.18 | 26.96 ± 0.46 | 201.23 ± 6.99 |
| pH = 7 | 544.28 ± 57.97 | 312.75 ± 23.38 | 38.14 ± 5.56 | 22.14 ± 6.83 | 187.10 ± 28.07 |
| pH = 8 | 580.54 ± 94.41 | 341.39 ± 18.75 | 45.78 ± 9.19 | 32.42 ± 8.76 | 193.61 ± 21.84 |
| Water + AA | 592.44 ± 87.97 | 303.62 ± 20.47 | 36.23 ± 7.46 | 24.99 ± 7.29 | 167.42 ± 22.79 |
| pH = 3 + AA | 464.35 ± 105.43 | 291.96 ± 59.24 | 27.10 ± 1.76 | 19.38 ± 0.46 | 147.21 ± 8.07 |
| pH = 4 + AA | 557.34 ± 60.39 | 321.77 ± 18.46 | 33.65 ± 4.13 | 23.30 ± 3.95 | 166.23 ± 17.10 |
| pH = 7 + AA | 549.93 ± 39.69 | 342.03 ± 11.62 | 40.31 ± 2.01 | 39.57 ± 4.63 | 157.89 ± 17.21 |
| pH = 8 + AA | 630.99 ± 2.44 | 367.78 ± 11.21 | 45.19 ± 2.45 | 37.93 ± 2.89 | 181.76 ± 29.53 |

### PRESERVED CLAUSES

Each of the below clauses is specifically incorporated into the specification of the current application. Each of the below clauses may be amended and presented as a formal claim and further represents an independent invention. It should be noted that for each instance that a preserved clause indicates a glycosylated cannabinoid, and/or an acetylated cannabinoid, such clause should also expressly include and/or a cannabinoid glucuronide or other water-soluble cannabinoid.

1. A composition comprising:
an aqueous solution;
water-soluble cannabinoid dissolved in said aqueous solution wherein said water-soluble cannabinoid comprises a glycosylated cannabinoid, and/or an acetylated cannabinoid, and/or a mixture of both;
wherein said composition may be introduced to a food or beverage.
   2. The composition of clause 1, wherein said glycosylated cannabinoid, and/or said acetylated cannabinoid were generated in vivo.
   3. The composition of clause 1, wherein said glycosylated cannabinoid, and/or said acetylated cannabinoid were generated in vitro.
   4. The composition of clause 1, wherein said water-soluble cannabinoid is non-psychoactive.
   5. The composition of clause 1, wherein said aqueous solution comprises an aqueous solution selected from the group consisting of: saline, purified water, ethanol.
   6. The composition of clause 1, wherein said aqueous solution comprises propylene glycol, deionized water, an alcohol.
   7. The composition of clause 1, wherein said alcohol comprises ethanol.
   8. The composition of clause 7, further comprising a buffer.
   9. The composition of clause 8, wherein said buffer maintains said aqueous solution at a pH below 7.4.
   10. The composition of clause 7, further comprising formic acid, or ammonium hydroxide.
   11. A consumable food additive comprising at least one water-soluble glycosylated cannabinoid.
   12. A consumable food additive as described in clause 11 and further comprising a food additive polysaccharide.
   13. A consumable food additive as described in clause 12 wherein said food additive polysaccharide comprises dextrin and/or maltodextrin.
   14. A consumable food additive as described in clause 11 and further comprising a emulsifier.
   15. A consumable food additive as described in clause 14 wherein said emulsifier is selected from the group consisting of: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.
   16. A consumable food additive as described in clause 11, wherein said water-soluble glycosylated cannabinoid is a non-psychoactive cannabinoid.
   17. A consumable food additive as described in clause 11, wherein said water-soluble glycosylated cannabinoid is generated in vivo.
   18. A consumable food additive as described in clause 11, wherein said water-soluble glycosylated cannabinoid is generated in vitro.
   19. A consumable food additive as described in clause 13, wherein said consumable food additive is a homogenous composition.
   20. A consumable food additive as described in clause 11, and further comprising a flavoring agent.
   21. A consumable food additive as described in clause 20 wherein said flavoring agent comprises a flavoring agent selected from the group consisting of: Sucrose (sugar), glucose, fructose, sorbitol, mannitol, corn syrup, high fructose corn syrup, saccharin, aspartame, sucralose, acesulfame potassium (acesulfame-K), neotame.
   22. A consumable food additive as described in clause 11, and further comprising a coloring agent.
   23. A consumable food additive as described in clause 22 wherein said coloring agent comprises a coloring agent selected from the group consisting of: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate
   24. A consumable food additive as described in clause 11, and further comprising a surfactant.
   25. A consumable food additive as described in clause 24 wherein said surfactant comprises a surfactant selected from the group consisting of glycerol monostearate and polysorbate 80.
   26. A consumable food additive as described in clause 11, and further comprising a preservative.
   27. A consumable food additive as described in clause 26, wherein said preservative comprises a preservative selected from the group consisting of: ascorbic acid, citric acid, sodium benzoate, calcium propionate, sodium erythorbate, sodium nitrite, calcium sorbate, potassium sorbate, BHA, BHT, EDTA, tocopherols.
   28. A consumable food additive as described in clause 11 and further comprising a nutrient supplement.
   29. A consumable food additive as described in clause 28, wherein said nutrient supplement comprises a nutrient supplement selected from the group consisting of: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.
   30. A consumable food additive as described in clause 11 and further comprising at least one water-soluble acetylated cannabinoid.
   31. A consumable food additive comprising at least one water-soluble acetylated cannabinoid.
   32. A consumable food additive as described in clause 31 and further comprising a food additive polysaccharide.
   33. A consumable food additive as described in clause 32 wherein said food additive polysaccharide comprises dextrin and/or maltodextrin.
   34. A consumable food additive as described in clause 32 and further comprising a emulsifier.
   35. A consumable food additive as described in clause 34 wherein said emulsifier is selected from the group consisting of: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.
   36. A consumable food additive as described in clause 31, wherein said water-soluble acetylated cannabinoid is a non-psychoactive cannabinoid.
   37. A consumable food additive as described in clause 31, wherein said water-soluble acetylated cannabinoid is generated in vivo.
   38. A consumable food additive as described in clause 31, wherein said water-soluble acetylated cannabinoid is generated in vitro.
   39. A consumable food additive as described in clause 31, wherein said consumable food additive is a homogenous composition.
   40. A consumable food additive as described in clause 31, and further comprising a flavoring agent.
   41. A consumable food additive as described in clause 40 wherein said flavoring agent comprises a flavoring agent selected from the group consisting of: Sucrose (sugar), glucose, fructose, sorbitol, mannitol, corn syrup, high fructose corn syrup, saccharin, aspartame, sucralose, acesulfame potassium (acesulfame-K), neotame.
   42. A consumable food additive as described in clause 31, and further comprising a coloring agent.
   43. A consumable food additive as described in clause 42 wherein said coloring agent comprises a coloring agent selected from the group consisting of: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate
   44. A consumable food additive as described in clause 31, and further comprising a surfactant.
   45. A consumable food additive as described in clause 44 wherein said surfactant comprises a surfactant selected from the group consisting of glycerol monostearate and polysorbate 80.
   46. A consumable food additive as described in clause 31, and further comprising a preservative.
   47. A consumable food additive as described in clause 46, wherein said preservative comprises a preservative selected from the group consisting of: ascorbic acid, citric acid, sodium benzoate, calcium propionate, sodium erythorbate, sodium nitrite, calcium sorbate, potassium sorbate, BHA, BHT, EDTA, tocopherols
   48. A consumable food additive as described in clause 31 and further comprising a nutrient supplement.
   49. A consumable food additive as described in clause 48, wherein said nutrient supplement comprises a nutrient supplement selected from the group consisting of: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.
   50. A consumable food additive as described in clause 31 and further comprising at least one water-soluble glycosylated cannabinoid.
   51. A consumable food additive comprising a mixture of at least one water-soluble glycosylated cannabinoid and at least one water-soluble acetylated cannabinoid.
   52. A consumable food additive as described in clause 51 and further comprising a food additive polysaccharide.
   53. A consumable food additive as described in clause 52 wherein said food additive polysaccharide comprises dextrin and/or maltodextrin.
   54. A consumable food additive as described in clause 51 and further comprising a emulsifier.
   55. A consumable food additive as described in clause 54 wherein said emulsifier is selected from the group consisting of: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.
   56. A consumable food additive as described in clause 51, wherein said water-soluble acetylated cannabinoid and said water-soluble glycosylated cannabinoid are non-psychoactive cannabinoids.
   57. A consumable food additive as described in clause 51, wherein said water-soluble acetylated cannabinoid and said water-soluble glycosylated cannabinoid are generated in vivo.
   58. A consumable food additive as described in clause 51, wherein said water-soluble acetylated cannabinoid and said water-soluble glycosylated cannabinoid are generated in vitro.
   59. A consumable food additive as described in clause 51, wherein said consumable food additive is a homogenous composition.
   60. A consumable food additive as described in clause 51, and further comprising a flavoring agent.
   61. A consumable food additive as described in clause 60 wherein said flavoring agent comprises a flavoring agent selected from the group consisting of: Sucrose (sugar), glucose, fructose, sorbitol, mannitol, corn syrup, high fructose corn syrup, saccharin, aspartame, sucralose, acesulfame potassium (acesulfame-K), neotame.
   62. A consumable food additive as described in clause 51, and further comprising a coloring agent.
   63. A consumable food additive as described in clause 62 wherein said coloring agent comprises a coloring agent selected from the group consisting of: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate
   64. A consumable food additive as described in clause 51, and further comprising a surfactant.
   65. A consumable food additive as described in clause 64 wherein said surfactant comprises a surfactant selected from the group consisting of glycerol monostearate and polysorbate 80.
   66. A consumable food additive as described in clause 51, and further comprising a preservative.
   67. A consumable food additive as described in clause 66, wherein said preservative comprises a preservative selected from the group consisting of: ascorbic acid, citric acid, sodium benzoate, calcium propionate, sodium erythorbate, sodium nitrite, calcium sorbate, potassium sorbate, BHA, BHT, EDTA, tocopherols
   68 A consumable food additive as described in clause 51 and further comprising a nutrient supplement.
   69. A consumable food additive as described in clause 68, wherein said nutrient supplement comprises a nutrient supplement selected from the group consisting of: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.
   70. A consumable fluid comprising at least one water-soluble glycosylated cannabinoid.
   71. A consumable fluid as described in clause 70, further comprising a food additive polysaccharide.
   72. A consumable fluid as described in clause 70, wherein said food additive polysaccharide comprises maltodextrin and/or dextrin.
   73. A consumable fluid as described in clause 73, wherein said maltodextrin is an aqueous maltodextrin solution.
   74. A consumable fluid as described in clause 73, wherein said aqueous maltodextrin solution further comprises sorbic acid and an acidifying agent to provide a food grade aqueous solution of maltodextrin having a pH of 2-4 and a sorbic acid content of 0.02-0.1% by weight.
   75. A consumable fluid as described in clause 70, wherein said consumable fluid is water.
   76. A consumable fluid as described in clause 75, wherein said consumable fluid is selected from the group consisting of: an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water.
   77. A consumable fluid as described in clause 70, wherein said water-soluble glycosylated cannabinoid is a non-psychoactive cannabinoid.
   78. A consumable fluid as described in clause 70, wherein said water-soluble glycosylated cannabinoid is generated in vivo.
   79 A consumable fluid as described in clause 70, wherein said water-soluble glycosylated cannabinoid is generated in vitro.
   80. A consumable fluid as described in clause 70 further comprising at least one of: xanthan gum, cellulose gum, whey protein hydrolysate, ascorbic acid, citric acid, malic acid, sodium benzoate, sodium citrate, sugar, phosphoric acid, and water.
   81. A consumable fluid comprising at least one water-soluble acetylated cannabinoid.
   82. A consumable fluid as described in clause 81 further comprising a food additive polysaccharide.
   83. A consumable fluid as described in clause 81 wherein said food additive polysaccharide comprises maltodextrin and/or dextrin.
   84. A consumable fluid as described in clause 83, wherein said maltodextrin is an aqueous maltodextrin solution.
   85. A consumable fluid as described in clause 84, wherein said aqueous maltodextrin solution further comprises sorbic acid and an acidifying agent to provide a food grade aqueous solution of maltodextrin having a pH of 2-4 and a sorbic acid content of 0.02-0.1% by weight.
   86. A consumable fluid as described in clause 81, wherein said consumable fluid is water.
   87. A consumable fluid as described in clause 81, wherein said consumable fluid is selected from the group consisting of: an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water.
   88. A consumable fluid as described in clause 81, wherein said water-soluble acetylated cannabinoid is a non-psychoactive cannabinoid.
   89. A consumable fluid as described in clause 81, wherein said water-soluble acetylated cannabinoid is generated in vivo.
   90. A consumable fluid as described in clause 81, wherein said water-soluble acetylated cannabinoid is generated in vitro.
   91. A consumable fluid as described in clause 81 further comprising at least one of: xanthan gum, cellulose gum, whey protein hydrolysate, ascorbic acid, citric acid, malic acid, sodium benzoate, sodium citrate, sugar, phosphoric acid, and water.
   92. A consumable gel comprising at least one water-soluble glycosylated cannabinoid and gelatin in an aqueous solution.
   93. A consumable gel as described in clause 92 wherein said water-soluble glycosylated cannabinoid is generated in vivo.
   94. A consumable gel as described in clause 92 wherein said water-soluble glycosylated cannabinoid is generated in vitro.
   95. A consumable gel comprising at least one water-soluble acetylated cannabinoid and gelatin in an aqueous solution.
   96. A consumable gel as described in clause 95 wherein said water-soluble acetylated cannabinoid is generated in vivo.
   97. A consumable gel as described in clause 95 wherein said water-soluble acetylated cannabinoid is generated in vitro.
   98. A consumable gel comprising at least one water-soluble acetylated cannabinoid, at least one water-soluble glycosylated cannabinoid and gelatin in an aqueous solution.
   99. A consumable gel as described in clause 98 wherein said water-soluble acetylated cannabinoid and said water-soluble acetylated cannabinoid are generated in vivo.
   100. A consumable gel as described in clause 99 wherein said water-soluble acetylated cannabinoid and said water-soluble acetylated cannabinoid are generated in vitro.
   101. A method of making a consumable fluid additive comprising the steps:
solubilizing a water-soluble glycosylated cannabinoid with a food additive polysaccharide to provide an aqueous solution containing said water-soluble glycosylated cannabinoid and said food additive polysaccharide; and
adding said water-soluble glycosylated cannabinoid and food additive polysaccharide aqueous solution to a consumable fluid.
   102. The method of clause 101, wherein said food additive polysaccharide is selected from the group consisting of: maltodextrin and/or dextrin.
   103. The method of clause 102, wherein said food additive polysaccharide is maltodextrin.
   104. The method of clause 103, wherein said maltodextrin is an aqueous maltodextrin solution.
   105. The method of clause 104, wherein said aqueous maltodextrin solution further comprises sorbic acid and an acidifying agent to provide a food grade aqueous solution of maltodextrin having a pH of 2-4 and a sorbic acid content of 0.02-0.1% by weight.
   106. The method of clause 104, wherein said consumable fluid is water.
   107. The method of clause 106, wherein said consumable fluid is selected from the group consisting of: an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water.
   108. The method of clause 101, wherein said water-soluble glycosylated cannabinoid is a non-psychoactive cannabinoid.
   109. The method of clause 101, wherein said water-soluble glycosylated cannabinoid is generated in vivo.
   110. The method of clause 101, wherein said water-soluble glycosylated cannabinoid is generated in vitro.
   110. The method of clause 101, and further comprising the step of adding a flavor to said consumable fluid.
   111. The method of clause 101, further comprising the step of adding at least one of: xanthan gum, cellulose gum, whey protein hydrolysate, ascorbic acid, citric acid, malic acid, sodium benzoate, sodium citrate, sugar, phosphoric acid, and water.
   112. A composition comprising:
a first quantity of water;
a water-soluble cannabinoid solubilized in said first quantity of water; and
at least one of: xanthan gum, cellulose gum, whey protein hydrolysate, ascorbic acid, citric acid, malic acid, sodium benzoate, sodium citrate, sugar, phosphoric acid, and/or a sugar alcohol.
   113. The composition of clause 112, wherein said water-soluble cannabinoid comprises a glycosylated water-soluble cannabinoid, an acetylated water-soluble cannabinoid or a mixture of both.
   114. The composition of clause 113, wherein said water-soluble cannabinoid is non-psychoactive.
   115. The composition of clause 112, and further comprising ethanol.
   116. The composition of clause 112, comprising less than 10 mass % water.
   117. The composition of clause 112, comprising more than 95 mass % water.
   118. The composition of clause 113, comprising about 0.1 mg to about 1000 mg of the water-soluble cannabinoid.
   119. The composition of clause 113, comprising about 0.1 mg to about 500 mg of the water-soluble cannabinoid.
   120. The composition of clause 113, comprising about 0.1 mg to about 200 mg of the water-soluble cannabinoid.
   121. The composition of clause 113, comprising about 0.1 mg to about 100 mg of the water-soluble cannabinoid.
   122. The composition of clause 113, comprising about 0.1 mg to about 100 mg of the water-soluble cannabinoid.
   123. The composition of clause 113, comprising about 0.1 mg to about 10 mg of the water-soluble cannabinoid.
   124. The composition of clause 113, comprising about 0.5 mg to about 5 mg of the water-soluble cannabinoid.
   125. The composition of clause 113, comprising about 1 mg/kg to 5 mg/kg (body weight) in a human of the water-soluble cannabinoid.
   126. The composition of clause 113, comprising water-soluble cannabinoid in the range of 50 mg/L to 300 mg/L.
   127. The composition of clause 113, comprising water-soluble cannabinoid in the range of 50 mg/L to 100 mg/L.
   128. The composition of clause 113, comprising water-soluble cannabinoid in the range of 50 mg/L to 500 mg/L.
   129. The composition of clause 113, comprising water-soluble cannabinoid over 500 mg/L.
   130. The composition of clause 113, comprising water-soluble cannabinoid under 50 mg/L.
   131. The composition of clause 112, wherein the composition is homogeneous.
   132. The composition of clause 112, comprising a flavoring agent.
   133. The composition of clause 112, comprising a coloring agent.
   134. The composition of clause 112, comprising caffeine.
   135. The composition of clause 112, comprising a coloring agent.
   136. A composition comprising:
a first quantity of water;
a water-soluble cannabinoid solubilized in said first quantity of water; and
a first quantity of ethanol in a liquid state.
   137. A composition according to clause 136 wherein said water-soluble cannabinoid is a glycosylated cannabinoid.
   138. A composition according to clause 136 wherein said water-soluble cannabinoid is an acetylated cannabinoid.
   139. A composition according to clause 136 wherein said water-soluble cannabinoid is a mixture of glycosylated cannabinoids and acetylated cannabinoid.
   140. A composition according to clause 137 wherein said glycosylated cannabinoid is glycosylated in vivo.
   141. A composition according to clause 137 wherein said glycosylated cannabinoid is glycosylated in vitro.
   142. A composition according to clause 138 wherein said acetylated cannabinoid is acetylated in vivo.
   143. A composition according to clause 138 wherein said acetylated cannabinoid is acetylated in vitro.
   144. A composition according to clause 139 wherein said acetylated cannabinoid is acetylated in vivo and glycosylated cannabinoid is glycosylated in vivo.
   145. A composition according to clause 139 wherein said acetylated cannabinoid is acetylated in vitro and glycosylated cannabinoid is glycosylated in vitro.
   146. A composition according to clause 136 wherein said ethanol can be up to about ninety-nine point nine-five percent (99.95%) by weight and said water-soluble cannabinoid about zero point zero five percent (0.05%) by weight.
   147. A composition according to clause 136, wherein said water-soluble cannabinoid is non-psychoactive.
   148. A composition according to clause 136, wherein said ethanol is an ethyl alcohol.
   149. A cannabinoid enriched alcohol composition according to clause 148, wherein said ethyl alcohol has a proof greater than 100.
   150. A composition according to clause 148, wherein said ethyl alcohol has a proof less than 100.
   151. A composition according to clause 148, wherein said ethyl alcohol is a spirit.
   152. A composition according to clause 148, wherein said ethyl alcohol is beer, and/or wine.
   153. A cannabinoid enriched alcohol composition for human consumption, said composition comprising by weight about:
a first quantity of water;
a water-soluble cannabinoid solubilized in said first quantity of water; and
a first quantity of ethanol in a liquid state wherein said first quantity of ethanol is between 1% to 20% weight by volume.
   154. A cannabinoid enriched alcohol composition according to clause 153 wherein said water-soluble cannabinoid is a glycosylated cannabinoid.
   155. A cannabinoid enriched alcohol composition according to clause 153 wherein said water-soluble cannabinoid is an acetylated cannabinoid.
   156. A cannabinoid enriched alcohol composition according to clause 153 wherein said water-soluble cannabinoid is a mixture of glycosylated cannabinoids and acetylated cannabinoid.
   157. A cannabinoid enriched alcohol composition according to clause 154 wherein said glycosylated cannabinoid is glycosylated in vivo.
   158. A cannabinoid enriched alcohol composition according to clause 154 wherein said glycosylated cannabinoid is glycosylated in vitro.
   159. A cannabinoid enriched alcohol composition according to clause 155 wherein said acetylated cannabinoid is acetylated in vivo.
   160. A cannabinoid enriched alcohol composition according to clause 155 wherein said acetylated cannabinoid is acetylated in vitro.
   161. A cannabinoid enriched alcohol composition according to clause 156 wherein said acetylated cannabinoid is acetylated in vivo and glycosylated cannabinoid is glycosylated in vivo.
   162. A cannabinoid enriched alcohol composition according to clause 156 wherein said acetylated cannabinoid is acetylated in vitro and glycosylated cannabinoid is glycosylated in vitro.
   163. A cannabinoid enriched alcohol composition according to clause 153, wherein said water-soluble cannabinoid is non-psychoactive.
   164. A cannabinoid enriched alcohol composition according to clause 153, wherein said ethanol is an ethyl alcohol.
   165. A cannabinoid enriched alcohol composition according to clause 164, wherein said ethyl alcohol has a proof greater than 100.
   166. A cannabinoid enriched alcohol composition according to clause 164, wherein said ethyl alcohol is beer.
   167. A cannabinoid enriched alcohol composition according to clause 164, wherein said ethyl alcohol is wine.
   168. A cannabinoid enriched alcohol composition according to clause 164, wherein said ethyl alcohol is a distilled spirit.
   169. A chewing gum composition comprising:
a first quantity of at least one water-soluble cannabinoid;
a gum base comprising a buffering agent selected from the group consisting of acetates, glycinates, phosphates, carbonates, glycerophosphates, citrates, borates, and mixtures thereof;
at least one sweetening agent; and
at least one flavoring agent.
   170. The chewing gum composition of clause 169, wherein said water-soluble cannabinoid comprises at least one water-soluble glycosylated cannabinoid.
   171. The chewing gum composition of clause 169, wherein said water-soluble cannabinoid comprises at least one water-soluble acetylated cannabinoid.
   172. The chewing gum composition of clause 169, wherein said water-soluble cannabinoid comprises at least one water-soluble acetylated cannabinoid, and at least one water-soluble glycosylated cannabinoid.
   173. The chewing gum composition of clause 172, wherein said water soluble glycosylated cannabinoid, and/or said acetylated cannabinoid were glycosylated and acetylated in vivo respectively
   174. The chewing gum composition of clause 169, comprising
0.01 to 1% by weight of said water-soluble cannabinoid;
25 to 85% by weight of said gum base;
10 to 35% by weight of said at least one sweetening agent; and
1 to 10% by weight of said flavoring agent.
   175. The chewing gum composition of clause 174, wherein said flavoring agents comprise a flavoring agent selected from the group consisting of menthol flavor, eucalyptus, mint flavor and/or L-menthol.
   176. The chewing gum composition of clause 174, wherein said sweetening agent comprises a sweetening agent selected from the group consisting of xylitol, sorbitol, isomalt, aspartame, sucralose, acesulfame potassium, and saccharin.
   177. The chewing gum composition according to clause 169, wherein the chewing gum composition comprises an antioxidant.
   178. The chewing gum composition according to clause 169, wherein the chewing gum composition comprises a pharmaceutically acceptable excipient selected from the group consisting of fillers, disintegrants, binders, lubricants, and antioxidants.
   179. The chewing gum composition according to clause 169, wherein the chewing gum composition is non-disintegrating.
   180. The chewing gum composition according to clause 169, wherein the chewing gum comprises natural flavors.
   181. The chewing gum composition according to clause 169, and further comprising a coloring agent.
   182. The chewing gum composition according to clause 169, and further comprising a flavoring agent.
   183. The chewing gum composition according to clause 169, wherein said water-soluble cannabinoid is non-psychoactive.
   184. A composition for a water-soluble cannabinoid infused solution comprising:
purified water;
at least one water-soluble cannabinoid;
at least one flavoring agent.
   185. The composition of clause 1, and further comprising a sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.
   186. The composition of clause 184, and further comprising sodium chloride.
   187. The composition of clause 184, and further comprising glycerin.
   188. The composition of clause 184, and further comprising a coloring agent.
   189. The composition of clause 184, and further comprising a first quantity of a demulcent.
   190. The composition of clause 184, wherein said demulcent is selected from the group consisting of: pectin, glycerin, honey, methylcellulose, and propylene glycol.
   191. The composition of clause 184, wherein said water-soluble cannabinoid is selected from the group consisting of: a water soluble glycosylated cannabinoid, a water soluble acetylated cannabinoid, or a mixture of both.
   192. The composition of clause 191, wherein said water soluble glycosylated cannabinoid, and/or said acetylated cannabinoid were glycosylated and acetylated in vivo respectively.
   193. The composition of clause 184, wherein said water-soluble cannabinoid is non-psychoactive.
   194. A composition for a water-soluble cannabinoid infused anesthetic solution comprising:
purified water;
at least one water-soluble cannabinoid;
at least one oral anesthetic.
   195. The composition of clause 194, and further comprising a sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.
   196. The composition of clause 194, and further comprising sodium chloride.
   197. The composition of clause 194, and further comprising glycerin.
   198. The composition of clause 194, and further comprising a coloring agent.
   199. The composition of clause 194, wherein said anesthetic is selected from the group consisting of: benzocaine, and phenol.
   200. The composition of clause 199, wherein said first quantity of anesthetic is between 0.1% to 15% volume by weight.
   201. The composition of clause 194, and further comprising a first quantity of a demulcent.
   202. The composition of clause 201, wherein said demulcent is selected from the group consisting of: pectin, glycerin, honey, methylcellulose, and propylene glycol.
   203. The composition of clause 194, wherein said water-soluble cannabinoid is selected from the group consisting of: a water soluble glycosylated cannabinoid, a water soluble acetylated cannabinoid, or a mixture of both.
   204. The composition of clause 203, wherein said water soluble glycosylated cannabinoid, and/or said acetylated cannabinoid were glycosylated and acetylated in vivo respectively.
   205. The composition of clause 203, wherein said water soluble glycosylated cannabinoid, and/or said acetylated cannabinoid were glycosylated and acetylated in vitro respectively.
   206. The composition of clause 194, wherein said water-soluble cannabinoid is non-psychoactive.
   207. A composition for a hard lozenge for rapid delivery of water-soluble cannabinoids through the oral mucosa, the lozenge comprising:
a crystalized sugar base;
at least one water-soluble cannabinoid;
wherein said hard lozenge has a moisture content between 0.1 to 2%.
   208. The composition of clause 207, wherein said crystalized sugar base comprises a crystalized sugar base selected from the group consisting of: sucrose, invert sugar, corn syrup, and isomalt or a combination of the same.
   209. The composition of clause 207, and further comprising at least one acidulant.
   210. The composition of clause 209, wherein said acidulant is selected from the group consisting of: citric acid, tartaric acid, fumaric acid, and malic acid.
   211. The composition of clause 209, and further comprising at least one pH adjustor.
   212. The composition of clause 211, wherein said pH adjustor is selected from the group consisting of: calcium carbonate, sodium bicarbonate, and magnesium trisilicate.
   213. The composition of clause 207, and further comprising at least one anesthetic.
   214. The composition of clause 213, wherein said anesthetic is selected from the group consisting of: benzocaine, and phenol.
   215. The composition of clause 213, wherein said first quantity of anesthetic is between 1 mg to 15 mg.
   216. The composition of clause 1207, and further comprising a first quantity of menthol.
   217. The composition of clause 216, wherein said first quantity of menthol is between 1 mg to 20 mg.
   218. The composition of clause 207, and further comprising a first quantity of a demulcent.
   219. The composition of clause 218, wherein said demulcent is selected from the group consisting of: pectin, glycerin, honey, methylcellulose, propylene glycol, and glycerine.
   220. The composition of clause 218, wherein said first quantity of demulcent is between 1 mg to 10 mg.
   221. The composition of clause 207, wherein said water-soluble cannabinoid is selected from the group consisting of: a water soluble glycosylated cannabinoid, an acetylated cannabinoid, or a mixture of both.
   222. The composition of clause 221, wherein said water soluble glycosylated cannabinoid, and/or said acetylated cannabinoid were glycosylated and acetylated in vivo respectively
   223. The composition of clause 221, wherein said water soluble glycosylated cannabinoid, and/or said acetylated cannabinoid were glycosylated and acetylated in vitro respectively
   224. The composition of clause 221, wherein the water-soluble cannabinoid is below 50 mg.
   225. The composition of clause 221, wherein the water-soluble cannabinoid is above 50 mg.
   226. The composition of clause 221, wherein said water-soluble cannabinoid is non-psychoactive.
   227. A chewable lozenge for rapid delivery of water-soluble cannabinoids through the oral mucosa, the lozenge comprising:
a glycerinated gelatin base;
at least one sweetener; and
at least one water-soluble cannabinoid dissolved in a first quantity of water.
   228. The composition of clause 227, wherein said sweetener comprises a sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.
   229. The composition of clause 227, and further comprising at least one acidulant.
   230. The composition of clause 229, wherein said acidulant is selected from the group consisting of: citric acid, tartaric acid, fumaric acid, and malic acid.
   231. The composition of clause 229, and further comprising at least one pH adjustor.
   232. The composition of clause 231, wherein said pH adjustor is selected from the group consisting of: calcium carbonate, sodium bicarbonate, and magnesium trisilicate.
   233. The composition of clause 227, and further comprising at least one anesthetic.
   234. The composition of clause 233, wherein said anesthetic is selected from the group consisting of: benzocaine, and phenol.
   235. The composition of clause 233, wherein said first quantity of anesthetic is between 1 mg to 15 mg.
   236. The composition of clause 227, and further comprising a first quantity of menthol.
   237. The composition of clause 236, wherein said first quantity of menthol is between 1 mg to 20 mg.
   238. The composition of clause 227, and further comprising a first quantity of a demulcent.
   239. The composition of clause 238, wherein said demulcent is selected from the group consisting of: pectin, glycerin, honey, methylcellulose, propylene glycol, and glycerine.
   240. The composition of clause 238, wherein said first quantity of demulcent is between 1 mg to 10 mg.
   241. The composition of clause 227, wherein said water-soluble cannabinoid is selected from the group consisting of: a water soluble glycosylated cannabinoid, an acetylated cannabinoid, or a mixture of both.
   242. The composition of clause 241, wherein said water soluble glycosylated cannabinoid, and/or said acetylated cannabinoid were glycosylated and acetylated in vivo respectively
   243. The composition of clause 241, wherein the water-soluble cannabinoid is below 50 mg.
   244. The composition of clause 241, wherein the water-soluble cannabinoid is above 50 mg.
   245. The composition of clause 227, wherein said water-soluble cannabinoid is non-psychoactive.
   246. A soft lozenge for rapid delivery of cannabinoids through the oral mucosa, the lozenge comprising:
a polyethylene glycol base;
at least one sweetener; and
at least one water-soluble cannabinoid.
   247. The composition of clause 246, wherein said sweetener comprises a crystalized sugar base selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.
   248. The composition of clause 246, and further comprising at least one acidulant.
   249. The composition of clause 248, wherein said acidulant is selected from the group consisting of: citric acid, tartaric acid, fumaric acid, and malic acid.
   250. The composition of clause 248, and further comprising at least one pH adjustor.
   251. The composition of clause 250, wherein said pH adjustor is selected from the group consisting of: calcium carbonate, sodium bicarbonate, and magnesium trisilicate.
   252. The composition of clause 247, and further comprising at least one anesthetic.
   253. The composition of clause 252, wherein said anesthetic is selected from the group consisting of: benzocaine, and phenol.
   254. The composition of clause 252, wherein said first quantity of anesthetic is between 1 mg to 15 mg.
   255. The composition of clause 246, and further comprising a first quantity of menthol.
   256. The composition of clause 255, wherein said first quantity of menthol is between 1 mg to 20 mg.
   257. The composition of clause 246, and further comprising a first quantity of a demulcent.
   258. The composition of clause 257, wherein said demulcent is selected from the group consisting of: pectin, glycerin, honey, methylcellulose, propylene glycol, and glycerine.
   259. The composition of clause 2258, wherein said first quantity of demulcent is between 1 mg to 10 mg.
   260. The composition of clause 246, wherein said water-soluble cannabinoid is selected from the group consisting of: a water soluble glycosylated cannabinoid, an acetylated cannabinoid, or a mixture of both.
   261. The composition of clause 260, wherein said water soluble glycosylated cannabinoid, and/or said acetylated cannabinoid were glycosylated and acetylated in vivo respectively. 262 The composition of clause 260, wherein the water-soluble cannabinoid is below 50 mg.
   263. The composition of clause 260, wherein the water-soluble cannabinoid is above 50 mg.
   264. The composition of clause 246, wherein said water-soluble cannabinoid is non-psychoactive.
   265. A tablet or capsule consisting essentially of a water-soluble glycosylated cannabinoid and maltodextrin.
   266. The tablet or capsule of clause 265, wherein said water-soluble glycosylated cannabinoid comprises a water-soluble glycosylated cannabinoid generated in vivo.
   267. The tablet or capsule of clause 265, wherein said water-soluble glycosylated cannabinoid comprises a water-soluble glycosylated cannabinoid generated in vitro.
   268. The tablet or capsule of clause 265, wherein said water-soluble glycosylated cannabinoid comprises a non-psychoactive water-soluble glycosylated cannabinoid.
   269. The tablet or capsule of clause 265, wherein the amount of water-soluble glycosylated cannabinoid is 5 milligrams or less.
   270. The tablet or capsule of clause 265, wherein the amount of water-soluble glycosylated cannabinoid 5 milligrams and 200 milligrams.
   271. The tablet or capsule of clause 265, wherein the wherein the amount of water-soluble glycosylated cannabinoid is more than 200 milligrams.
   272. A tablet or capsule consisting essentially of a water-soluble glycosylated cannabinoid and whey protein isolate.
   273. The tablet or capsule of clause 272, wherein said water-soluble glycosylated cannabinoid comprises a water-soluble glycosylated cannabinoid generated in vivo.
   274. The tablet or capsule of clause 272, wherein said water-soluble glycosylated cannabinoid comprises a water-soluble glycosylated cannabinoid generated in vitro.
   274. The tablet or capsule of clause 272, wherein said water-soluble glycosylated cannabinoid comprises a non-psychoactive water-soluble glycosylated cannabinoid.
   275. The tablet or capsule of clause 272, wherein the amount of water-soluble glycosylated cannabinoid is 5 milligrams or less.
   276. The tablet or capsule of clause 272, wherein the amount of water-soluble glycosylated cannabinoid 5 milligrams and 200 milligrams.
   277. The tablet or capsule of clause 272, wherein the wherein the amount of water-soluble glycosylated cannabinoid is more than 200 milligrams.
   278. A tablet or capsule consisting essentially of a water-soluble glycosylated cannabinoid and xanthan gum.
   279. The tablet or capsule of clause 278, wherein said water-soluble glycosylated cannabinoid comprises a water-soluble glycosylated cannabinoid generated in vivo.
   280. The tablet or capsule of clause 278, wherein said water-soluble glycosylated cannabinoid comprises a water-soluble glycosylated cannabinoid generated in vitro.
   281. The tablet or capsule of clause 278, wherein said water-soluble glycosylated cannabinoid comprises a non-psychoactive water-soluble glycosylated cannabinoid.
   282. The tablet or capsule of clause 278, wherein the amount of water-soluble glycosylated cannabinoid is 5 milligrams or less.
   283. The tablet or capsule of clause 278, wherein the amount of water-soluble glycosylated cannabinoid 5 milligrams and 200 milligrams.
   284. The tablet or capsule of clause 278, wherein the wherein the amount of water-soluble glycosylated cannabinoid is more than 200 milligrams.
   285. A tablet or capsule consisting essentially of a water-soluble glycosylated cannabinoid and guar gum.
   286. The tablet or capsule of clause 285, wherein said water-soluble glycosylated cannabinoid comprises a water-soluble glycosylated cannabinoid generated in vivo.
   287. The tablet or capsule of clause 285, wherein said water-soluble glycosylated cannabinoid comprises a water-soluble glycosylated cannabinoid generated in vitro.
   288. The tablet or capsule of clause 285, wherein said water-soluble glycosylated cannabinoid comprises a non-psychoactive water-soluble glycosylated cannabinoid.
   289. The tablet or capsule of clause 285, wherein the amount of water-soluble glycosylated cannabinoid is 5 milligrams or less.
   290. The tablet or capsule of clause 285, wherein the amount of water-soluble glycosylated cannabinoid 5 milligrams and 200 milligrams.
   291. The tablet or capsule of clause 285, wherein the wherein the amount of water-soluble glycosylated cannabinoid is more than 200 milligrams.
   292. A tablet or capsule consisting essentially of water-soluble glycosylated cannabinoid and diglycerides.
   293. The tablet or capsule of clause 292 wherein the diglycerides are in a mix with monoglycerides.
   294. The tablet or capsule of clause 292, wherein said water-soluble glycosylated cannabinoid comprises a water-soluble glycosylated cannabinoid generated in vivo.
   295. The tablet or capsule of clause 292, wherein said water-soluble glycosylated cannabinoid comprises a water-soluble glycosylated cannabinoid generated in vitro.
   296. The tablet or capsule of clause 292, wherein said water-soluble glycosylated cannabinoid comprises a non-psychoactive water-soluble glycosylated cannabinoid.
   297. The tablet or capsule of clause 292, wherein the amount of water-soluble glycosylated cannabinoid is 5 milligrams or less.
   298. The tablet or capsule of clause 292, wherein the amount of water-soluble glycosylated cannabinoid 5 milligrams and 200 milligrams.
   299. The tablet or capsule of clause 292, wherein the wherein the amount of water-soluble glycosylated cannabinoid is more than 200 milligrams.
   300. A tablet or capsule consisting essentially of a water-soluble glycosylated cannabinoid and guar gum.
   301. The tablet or capsule of clause 300, wherein said water-soluble glycosylated cannabinoid comprises a water-soluble glycosylated cannabinoid generated in vivo.
   302. The tablet or capsule of clause 300, wherein said water-soluble glycosylated cannabinoid comprises a water-soluble glycosylated cannabinoid generated in vitro.
   303. The tablet or capsule of clause 300, wherein said water-soluble glycosylated cannabinoid comprises a non-psychoactive water-soluble glycosylated cannabinoid.
   304. The tablet or capsule of clause 300, wherein the amount of water-soluble glycosylated cannabinoid is 5 milligrams or less.
   305. The tablet or capsule of clause 300, wherein the amount of water-soluble glycosylated cannabinoid 5 milligrams and 200 milligrams.
   306. The tablet or capsule of clause 300, wherein the wherein the amount of water-soluble glycosylated cannabinoid is more than 200 milligrams.
   307. A tablet or capsule consisting essentially of water-soluble glycosylated cannabinoid and carboxymethyl cellulose.
   308. The tablet or capsule of clause 307, wherein said water-soluble glycosylated cannabinoid comprises a water-soluble glycosylated cannabinoid generated in vivo.
   309. The tablet or capsule of clause 307, wherein said water-soluble glycosylated cannabinoid comprises a water-soluble glycosylated cannabinoid generated in vitro.
   310. The tablet or capsule of clause 307, wherein said water-soluble glycosylated cannabinoid comprises a non-psychoactive water-soluble glycosylated cannabinoid.
   311. The tablet or capsule of clause 307, wherein the amount of water-soluble glycosylated cannabinoid is 5 milligrams or less.
   312. The tablet or capsule of clause 307, wherein the amount of water-soluble glycosylated cannabinoid 5 milligrams and 200 milligrams.
   313. The tablet or capsule of clause 307, wherein the wherein the amount of water-soluble glycosylated cannabinoid is more than 200 milligrams.
   314. A tablet or capsule consisting essentially a water-soluble glycosylated cannabinoid and glycerin.
   315. The tablet or capsule of clause 314, wherein said water-soluble glycosylated cannabinoid comprises a water-soluble glycosylated cannabinoid generated in vivo.
   316. The tablet or capsule of clause 314, wherein said water-soluble glycosylated cannabinoid comprises a water-soluble glycosylated cannabinoid generated in vitro.
   317. The tablet or capsule of clause 314, wherein said water-soluble glycosylated cannabinoid comprises a non-psychoactive water-soluble glycosylated cannabinoid.
   318. The tablet or capsule of clause 314, wherein the amount of water-soluble glycosylated cannabinoid is 5 milligrams or less.
   319. The tablet or capsule of clause 314, wherein the amount of water-soluble glycosylated cannabinoid 5 milligrams and 200 milligrams.
   320. The tablet or capsule of clause 314, wherein the wherein the amount of water-soluble glycosylated cannabinoid is more than 200 milligrams.
   321. A tablet or capsule consisting essentially of a water-soluble glycosylated cannabinoid and gelatin.
   322. The tablet or capsule of clause 321, wherein said water-soluble glycosylated cannabinoid comprises a water-soluble glycosylated cannabinoid generated in vivo.
   323. The tablet or capsule of clause 321, wherein said water-soluble glycosylated cannabinoid comprises a water-soluble glycosylated cannabinoid generated in vitro.
   324. The tablet or capsule of clause 321, wherein said water-soluble glycosylated cannabinoid comprises a non-psychoactive water-soluble glycosylated cannabinoid.
   325. The tablet or capsule of clause 321, wherein the amount of water-soluble glycosylated cannabinoid is 5 milligrams or less.
   326. The tablet or capsule of clause 321, wherein the amount of water-soluble glycosylated cannabinoid 5 milligrams and 200 milligrams.
   327. The tablet or capsule of clause 321, wherein the wherein the amount of water-soluble glycosylated cannabinoid is more than 200 milligrams.
   328. A tablet or capsule consisting essentially of water-soluble glycosylated cannabinoid and polyethylene glycol.
   329. The tablet or capsule of clause 328, wherein said water-soluble glycosylated cannabinoid comprises a water-soluble glycosylated cannabinoid generated in vivo.
   330. The tablet or capsule of clause 328, wherein said water-soluble glycosylated cannabinoid comprises a water-soluble glycosylated cannabinoid generated in vitro.
   331. The tablet or capsule of clause 328, wherein said water-soluble glycosylated cannabinoid comprises a non-psychoactive water-soluble glycosylated cannabinoid.
   332. The tablet or capsule of clause 328, wherein the amount of water-soluble glycosylated cannabinoid is 5 milligrams or less.
   333. The tablet or capsule of clause 328, wherein the amount of water-soluble glycosylated cannabinoid 5 milligrams and 200 milligrams.
   334. The tablet or capsule of clause 328, wherein the wherein the amount of water-soluble glycosylated cannabinoid is more than 200 milligrams.
   335. A tablet or capsule consisting essentially of a water-soluble acetylated cannabinoid and maltodextrin.
   336. The tablet or capsule of clause 335, wherein said water-soluble acetylated cannabinoid comprises a water-soluble acetylated cannabinoid generated in vivo.
   337. The tablet or capsule of clause 335, wherein said water-soluble acetylated cannabinoid comprises a water-soluble acetylated cannabinoid generated in vitro.
   338. The tablet or capsule of clause 335, wherein said water-soluble acetylated cannabinoid comprises a non-psychoactive water-soluble acetylated cannabinoid.
   339. The tablet or capsule of clause 335, wherein the amount of water-soluble acetylated cannabinoid is 5 milligrams or less.
   340. The tablet or capsule of clause 335, wherein the amount of water-soluble acetylated cannabinoid 5 milligrams and 200 milligrams.
   340. The tablet or capsule of clause 335, wherein the wherein the amount of water-soluble acetylated cannabinoid is more than 200 milligrams.
   341. A tablet or capsule consisting essentially of a water-soluble acetylated cannabinoid and whey protein isolate.
   342. The tablet or capsule of clause 341, wherein said water-soluble acetylated cannabinoid comprises a water-soluble acetylated cannabinoid generated in vivo.
   343. The tablet or capsule of clause 341, wherein said water-soluble acetylated cannabinoid comprises a water-soluble acetylated cannabinoid generated in vitro.
   344. The tablet or capsule of clause 341, wherein said water-soluble acetylated cannabinoid comprises a non-psychoactive water-soluble acetylated cannabinoid.
   345. The tablet or capsule of clause 341, wherein the amount of water-soluble acetylated cannabinoid is 5 milligrams or less.
   346. The tablet or capsule of clause 341, wherein the amount of water-soluble acetylated cannabinoid 5 milligrams and 200 milligrams.
   347. The tablet or capsule of clause 341, wherein the wherein the amount of water-soluble acetylated cannabinoid is more than 200 milligrams.
   348. A tablet or capsule consisting essentially of a water-soluble acetylated cannabinoid and xanthan gum.
   349. The tablet or capsule of clause 348, wherein said water-soluble acetylated cannabinoid comprises a water-soluble acetylated cannabinoid generated in vivo.
   350. The tablet or capsule of clause 348, wherein said water-soluble acetylated cannabinoid comprises a water-soluble acetylated cannabinoid generated in vitro.
   351. The tablet or capsule of clause 348, wherein said water-soluble acetylated cannabinoid comprises a non-psychoactive water-soluble acetylated cannabinoid.
   352. The tablet or capsule of clause 348, wherein the amount of water-soluble acetylated cannabinoid is 5 milligrams or less.
   353. The tablet or capsule of clause 348, wherein the amount of water-soluble acetylated cannabinoid 5 milligrams and 200 milligrams.
   354. The tablet or capsule of clause 348, wherein the wherein the amount of water-soluble acetylated cannabinoid is more than 200 milligrams.
   355. A tablet or capsule consisting essentially of a water-soluble acetylated cannabinoid and guar gum.
   356. The tablet or capsule of clause 355, wherein said water-soluble acetylated cannabinoid comprises a water-soluble acetylated cannabinoid generated in vivo.
   357. The tablet or capsule of clause 355, wherein said water-soluble acetylated cannabinoid comprises a water-soluble acetylated cannabinoid generated in vitro.
   358. The tablet or capsule of clause 355, wherein said water-soluble acetylated cannabinoid comprises a non-psychoactive water-soluble acetylated cannabinoid.
   359. The tablet or capsule of clause 355, wherein the amount of water-soluble acetylated cannabinoid is 5 milligrams or less.
   360. The tablet or capsule of clause 355, wherein the amount of water-soluble acetylated cannabinoid 5 milligrams and 200 milligrams.
   361. The tablet or capsule of clause 355, wherein the wherein the amount of water-soluble acetylated cannabinoid is more than 200 milligrams.
   362. A tablet or capsule consisting essentially of water-soluble acetylated cannabinoid and diglycerides.
   363. The tablet or capsule of clause 362 wherein the diglycerides are in a mix with monoglycerides.
   364. The tablet or capsule of clause 362, wherein said water-soluble acetylated cannabinoid comprises a water-soluble acetylated cannabinoid generated in vivo.
   365. The tablet or capsule of clause 362, wherein said water-soluble acetylated cannabinoid comprises a water-soluble acetylated cannabinoid generated in vitro.
   366. The tablet or capsule of clause 362, wherein said water-soluble acetylated cannabinoid comprises a non-psychoactive water-soluble acetylated cannabinoid.
   367. The tablet or capsule of clause 362, wherein the amount of water-soluble acetylated cannabinoid is 5 milligrams or less.
   368. The tablet or capsule of clause 362, wherein the amount of water-soluble acetylated cannabinoid 5 milligrams and 200 milligrams.
   369. The tablet or capsule of clause 362, wherein the wherein the amount of water-soluble acetylated cannabinoid is more than 200 milligrams.
   370. A tablet or capsule consisting essentially of a water-soluble acetylated cannabinoid and guar gum.
   371. The tablet or capsule of clause 370, wherein said water-soluble acetylated cannabinoid comprises a water-soluble acetylated cannabinoid generated in vivo.
   372. The tablet or capsule of clause 370, wherein said water-soluble acetylated cannabinoid comprises a water-soluble acetylated cannabinoid generated in vitro.
   373. The tablet or capsule of clause 370, wherein said water-soluble acetylated cannabinoid comprises a non-psychoactive water-soluble acetylated cannabinoid.
   374. The tablet or capsule of clause 370, wherein the amount of water-soluble acetylated cannabinoid is 5 milligrams or less.
   375. The tablet or capsule of clause 370, wherein the amount of water-soluble acetylated cannabinoid 5 milligrams and 200 milligrams.
   376. The tablet or capsule of clause 370, wherein the wherein the amount of water-soluble acetylated cannabinoid is more than 200 milligrams.
   377. A tablet or capsule consisting essentially of a water-soluble acetylated cannabinoid and carboxymethyl cellulose.
   378. The tablet or capsule of clause 377, wherein said water-soluble acetylated cannabinoid comprises a water-soluble acetylated cannabinoid generated in vivo.
   379. The tablet or capsule of clause 377, wherein said water-soluble acetylated cannabinoid comprises a water-soluble acetylated cannabinoid generated in vitro.
   380. The tablet or capsule of clause 377, wherein said water-soluble acetylated cannabinoid comprises a non-psychoactive water-soluble acetylated cannabinoid.
   390. The tablet or capsule of clause 377, wherein the amount of water-soluble acetylated cannabinoid is 5 milligrams or less.
   391. The tablet or capsule of clause 377, wherein the amount of water-soluble acetylated cannabinoid 5 milligrams and 200 milligrams.
   392. The tablet or capsule of clause 377, wherein the wherein the amount of water-soluble acetylated cannabinoid is more than 200 milligrams.
   393. A tablet or capsule consisting essentially a water-soluble acetylated cannabinoid and glycerin.
   394. The tablet or capsule of clause 393, wherein said water-soluble acetylated cannabinoid comprises a water-soluble acetylated cannabinoid generated in vivo.
   395. The tablet or capsule of clause 393, wherein said water-soluble acetylated cannabinoid comprises a water-soluble acetylated cannabinoid generated in vitro.
   396. The tablet or capsule of clause 393, wherein said water-soluble acetylated cannabinoid comprises a non-psychoactive water-soluble acetylated cannabinoid.
   397. The tablet or capsule of clause 393, wherein the amount of water-soluble acetylated cannabinoid is 5 milligrams or less.
   398. The tablet or capsule of clause 393, wherein the amount of water-soluble acetylated cannabinoid 5 milligrams and 200 milligrams.
   399. The tablet or capsule of clause 393, wherein the wherein the amount of water-soluble acetylated cannabinoid is more than 200 milligrams.
   400. A tablet or capsule consisting essentially of a water-soluble acetylated cannabinoid and gelatin.
   401. The tablet or capsule of clause 400, wherein said water-soluble acetylated cannabinoid comprises a water-soluble acetylated cannabinoid generated in vivo.
   402. The tablet or capsule of clause 400, wherein said water-soluble acetylated cannabinoid comprises a water-soluble acetylated cannabinoid generated in vitro.
   403. The tablet or capsule of clause 400, wherein said water-soluble acetylated cannabinoid comprises a non-psychoactive water-soluble acetylated cannabinoid.
   404. The tablet or capsule of clause 400, wherein the amount of water-soluble acetylated cannabinoid is 5 milligrams or less.
   405. The tablet or capsule of clause 400, wherein the amount of water-soluble acetylated cannabinoid 5 milligrams and 200 milligrams.
   406. The tablet or capsule of clause 400, wherein the wherein the amount of water-soluble acetylated cannabinoid is more than 200 milligrams.
   407. A tablet or capsule consisting essentially of a water-soluble acetylated cannabinoid and polyethylene glycol.
   408. The tablet or capsule of clause 407, wherein said water-soluble acetylated cannabinoid comprises a water-soluble acetylated cannabinoid generated in vivo.
   409. The tablet or capsule of clause 407, wherein said water-soluble acetylated cannabinoid comprises a water-soluble acetylated cannabinoid generated in vitro.
   410. The tablet or capsule of clause 407, wherein said water-soluble acetylated cannabinoid comprises a non-psychoactive water-soluble acetylated cannabinoid.
   411. The tablet or capsule of clause 407, wherein the amount of water-soluble acetylated cannabinoid is 5 milligrams or less.
   412. The tablet or capsule of clause 407, wherein the amount of water-soluble acetylated cannabinoid is between 5 milligrams and 200 milligrams.
   413. The tablet or capsule of clause 407, wherein the wherein the amount of water-soluble acetylated cannabinoid is more than 200 milligrams.
   414. A tablet or capsule consisting essentially of a water-soluble acetylated cannabinoid and a water-soluble glycosylated cannabinoid and a water-soluble glycosylated cannabinoid and maltodextrin.
   415. The tablet or capsule of clause 414, wherein said water-soluble acetylated cannabinoid comprises a water-soluble acetylated cannabinoid and a water-soluble glycosylated cannabinoid and a water-soluble glycosylated cannabinoid generated in vivo.
   416. The tablet or capsule of clause 414, wherein said water-soluble acetylated cannabinoid comprises a water-soluble acetylated cannabinoid and a water-soluble glycosylated cannabinoid and a water-soluble glycosylated cannabinoid generated in vitro.
   417. The tablet or capsule of clause 414, wherein said water-soluble acetylated cannabinoid comprises a non-psychoactive water-soluble acetylated cannabinoid.
   418. The tablet or capsule of clause 414, wherein the amount of water-soluble acetylated cannabinoid is 5 milligrams or less.
   419. The tablet or capsule of clause 414, wherein the amount of water-soluble acetylated cannabinoid 5 milligrams and 200 milligrams.
   420. The tablet or capsule of clause 414, wherein the wherein the amount of water-soluble acetylated cannabinoid is more than 200 milligrams.
   421. A tablet or capsule consisting essentially of a water-soluble acetylated cannabinoid and a water-soluble glycosylated cannabinoid and a water-soluble glycosylated cannabinoid and whey protein isolate.
   422. The tablet or capsule of clause 421, wherein said water-soluble acetylated cannabinoid comprises a water-soluble acetylated cannabinoid and a water-soluble glycosylated cannabinoid and a water-soluble glycosylated cannabinoid generated in vivo.
   423. The tablet or capsule of clause 421, wherein said water-soluble acetylated cannabinoid comprises a water-soluble acetylated cannabinoid and a water-soluble glycosylated cannabinoid and a water-soluble glycosylated cannabinoid generated in vitro.
   424. The tablet or capsule of clause 421, wherein said water-soluble acetylated cannabinoid comprises a non-psychoactive water-soluble acetylated cannabinoid.
   425. The tablet or capsule of clause 421, wherein the amount of water-soluble acetylated cannabinoid is 5 milligrams or less.
   426. The tablet or capsule of clause 421, wherein the amount of water-soluble acetylated cannabinoid 5 milligrams and 200 milligrams.
   427. The tablet or capsule of clause 421, wherein the wherein the amount of water-soluble acetylated cannabinoid is more than 200 milligrams.
   428. A tablet or capsule consisting essentially of a water-soluble acetylated cannabinoid and a water-soluble glycosylated cannabinoid and a water-soluble glycosylated cannabinoid and xanthan gum.
   429. The tablet or capsule of clause 428, wherein said water-soluble acetylated cannabinoid comprises a water-soluble acetylated cannabinoid and a water-soluble glycosylated cannabinoid and a water-soluble glycosylated cannabinoid generated in vivo.
   430. The tablet or capsule of clause 428, wherein said water-soluble acetylated cannabinoid comprises a water-soluble acetylated cannabinoid and a water-soluble glycosylated cannabinoid and a water-soluble glycosylated cannabinoid generated in vitro.
   431. The tablet or capsule of clause 428, wherein said water-soluble acetylated cannabinoid comprises a non-psychoactive water-soluble acetylated cannabinoid.
   432. The tablet or capsule of clause 428, wherein the amount of water-soluble acetylated cannabinoid is 5 milligrams or less.
   433. The tablet or capsule of clause 428, wherein the amount of water-soluble acetylated cannabinoid 5 milligrams and 200 milligrams.
   432. The tablet or capsule of clause 428, wherein the wherein the amount of water-soluble acetylated cannabinoid is more than 200 milligrams.
   433. A tablet or capsule consisting essentially of a water-soluble acetylated cannabinoid and a water-soluble glycosylated cannabinoid and a water-soluble glycosylated cannabinoid and guar gum.
   434. The tablet or capsule of clause 433, wherein said water-soluble acetylated cannabinoid comprises a water-soluble acetylated cannabinoid and a water-soluble glycosylated cannabinoid and a water-soluble glycosylated cannabinoid generated in vivo.
   435. The tablet or capsule of clause 433, wherein said water-soluble acetylated cannabinoid comprises a water-soluble acetylated cannabinoid and a water-soluble glycosylated cannabinoid and a water-soluble glycosylated cannabinoid generated in vitro.
   436. The tablet or capsule of clause 433, wherein said water-soluble acetylated cannabinoid comprises a non-psychoactive water-soluble acetylated cannabinoid.
   437. The tablet or capsule of clause 433, wherein the amount of water-soluble acetylated cannabinoid is 5 milligrams or less.
   438. The tablet or capsule of clause 433, wherein the amount of water-soluble acetylated cannabinoid 5 milligrams and 200 milligrams.
   439. The tablet or capsule of clause 433, wherein the wherein the amount of water-soluble acetylated cannabinoid is more than 200 milligrams.
   440. A tablet or capsule consisting essentially of a water-soluble acetylated cannabinoid and a water-soluble glycosylated cannabinoid and a water-soluble glycosylated cannabinoid and diglycerides.
   441. The tablet or capsule of clause 440 wherein the diglycerides are in a mix with monoglycerides.
   442. The tablet or capsule of clause 440, wherein said water-soluble cannabinoid comprises a water-soluble acetylated cannabinoid and a water-soluble glycosylated cannabinoid and a water-soluble glycosylated cannabinoid generated in vivo.
   443. The tablet or capsule of clause 440, wherein said water-soluble cannabinoid comprises a water-soluble acetylated cannabinoid and a water-soluble glycosylated cannabinoid and a water-soluble glycosylated cannabinoid generated in vitro.
   444. The tablet or capsule of clause 440, wherein said water-soluble acetylated cannabinoid comprises a non-psychoactive water-soluble acetylated cannabinoid.
   445. The tablet or capsule of clause 440, wherein the amount of water-soluble acetylated cannabinoid is 5 milligrams or less.
   446. The tablet or capsule of clause 440, wherein the amount of water-soluble acetylated cannabinoid 5 milligrams and 200 milligrams.
   447. The tablet or capsule of clause 440, wherein the wherein the amount of a water-soluble acetylated cannabinoid and a water-soluble glycosylated cannabinoid is more than 200 milligrams.
   448. A tablet or capsule consisting essentially of a water-soluble acetylated cannabinoid and a water-soluble glycosylated cannabinoid and guar gum.
   449. The tablet or capsule of clause 448, wherein said a water-soluble acetylated cannabinoid and a water-soluble glycosylated cannabinoid comprises a water-soluble acetylated cannabinoid and a water-soluble glycosylated cannabinoid generated in vivo.
   450. The tablet or capsule of clause 448, wherein said a water-soluble acetylated cannabinoid and a water-soluble glycosylated cannabinoid comprises a water-soluble acetylated cannabinoid and a water-soluble glycosylated cannabinoid generated in vitro.
   451. The tablet or capsule of clause 448, wherein said a water-soluble acetylated cannabinoid and a water-soluble glycosylated cannabinoid comprises a non-psychoactive a water-soluble acetylated cannabinoid and a water-soluble glycosylated cannabinoid.
   452. The tablet or capsule of clause 448, wherein the amount of a water-soluble acetylated cannabinoid and a water-soluble glycosylated cannabinoid is 5 milligrams or less.
   453. The tablet or capsule of clause 448, wherein the amount of a water-soluble acetylated cannabinoid and a water-soluble glycosylated cannabinoid 5 milligrams and 200 milligrams.
   454. The tablet or capsule of clause 448, wherein the wherein the amount of a water-soluble acetylated cannabinoid and a water-soluble glycosylated cannabinoid is more than 200 milligrams.
   455. A tablet or capsule consisting essentially of comprises a water-soluble acetylated cannabinoid and a water-soluble glycosylated cannabinoid and a water-soluble glycosylated cannabinoid and carboxymethyl cellulose.
   456. The tablet or capsule of clause 455, wherein said a water-soluble acetylated cannabinoid and a water-soluble glycosylated cannabinoid comprises a water-soluble acetylated cannabinoid and a water-soluble glycosylated cannabinoid generated in vivo.
   457. The tablet or capsule of clause 455, wherein said a water-soluble acetylated cannabinoid and a water-soluble glycosylated cannabinoid comprises a water-soluble acetylated cannabinoid and a water-soluble glycosylated cannabinoid generated in vitro.
   458. The tablet or capsule of clause 455, wherein said a water-soluble acetylated cannabinoid and a water-soluble glycosylated cannabinoid comprises a non-psychoactive a water-soluble acetylated cannabinoid and a water-soluble glycosylated cannabinoid.
   459. The tablet or capsule of clause 455, wherein the amount of a water-soluble acetylated cannabinoid and a water-soluble glycosylated cannabinoid is 5 milligrams or less.
   460. The tablet or capsule of clause 455, wherein the amount of a water-soluble acetylated cannabinoid and a water-soluble glycosylated cannabinoid 5 milligrams and 200 milligrams.
   461. The tablet or capsule of clause 455, wherein the wherein the amount of a water-soluble acetylated cannabinoid and a water-soluble glycosylated cannabinoid is more than 200 milligrams.
   462. A tablet or capsule consisting essentially a water-soluble acetylated cannabinoid and a water-soluble glycosylated cannabinoid and glycerin.
   463. The tablet or capsule of clause 462, wherein said a water-soluble acetylated cannabinoid and a water-soluble glycosylated cannabinoid comprises a water-soluble acetylated cannabinoid and a water-soluble glycosylated cannabinoid generated in vivo.
   464. The tablet or capsule of clause 462, wherein said a water-soluble acetylated cannabinoid and a water-soluble glycosylated cannabinoid comprises a water-soluble acetylated cannabinoid and a water-soluble glycosylated cannabinoid generated in vitro.
   465. The tablet or capsule of clause 462, wherein said a water-soluble acetylated cannabinoid and a water-soluble glycosylated cannabinoid comprises a non-psychoactive a water-soluble acetylated cannabinoid and a water-soluble glycosylated cannabinoid.
   466. The tablet or capsule of clause 462, wherein the amount of a water-soluble acetylated cannabinoid and a water-soluble glycosylated cannabinoid is 5 milligrams or less.
   467. The tablet or capsule of clause 462, wherein the amount of a water-soluble acetylated cannabinoid and a water-soluble glycosylated cannabinoid 5 milligrams and 200 milligrams.
   468. The tablet or capsule of clause 462, wherein the wherein the amount of a water-soluble acetylated cannabinoid and a water-soluble glycosylated cannabinoid is more than 200 milligrams.
   462. A tablet or capsule consisting essentially of a water-soluble acetylated cannabinoid and a water-soluble glycosylated cannabinoid and a water-soluble glycosylated cannabinoid and gelatin.
   470. The tablet or capsule of clause 462, wherein said a water-soluble acetylated cannabinoid and a water-soluble glycosylated cannabinoid comprises a water-soluble acetylated cannabinoid and a water-soluble glycosylated cannabinoid generated in vivo.
   471. The tablet or capsule of clause 462, wherein said a water-soluble acetylated cannabinoid and a water-soluble glycosylated cannabinoid comprises a water-soluble acetylated cannabinoid and a water-soluble glycosylated cannabinoid generated in vitro.
   472. The tablet or capsule of clause 462, wherein said a water-soluble acetylated cannabinoid and a water-soluble glycosylated cannabinoid comprises a non-psychoactive a water-soluble acetylated cannabinoid and a water-soluble glycosylated cannabinoid.
   473. The tablet or capsule of clause 462, wherein the amount of a water-soluble acetylated cannabinoid and a water-soluble glycosylated cannabinoid is 5 milligrams or less.
   474. The tablet or capsule of clause 462, wherein the amount of a water-soluble acetylated cannabinoid and a water-soluble glycosylated cannabinoid 5 milligrams and 200 milligrams.
   475. The tablet or capsule of clause 462, wherein the wherein the amount of a water-soluble acetylated cannabinoid and a water-soluble glycosylated cannabinoid is more than 200 milligrams.
   476. A tablet or capsule consisting essentially of a water-soluble acetylated cannabinoid and a water-soluble glycosylated cannabinoid and a water-soluble glycosylated cannabinoid and polyethylene glycol.
   477. The tablet or capsule of clause 476, wherein said a water-soluble acetylated cannabinoid and a water-soluble glycosylated cannabinoid comprises a water-soluble acetylated cannabinoid and a water-soluble glycosylated cannabinoid generated in vivo.
   478. The tablet or capsule of clause 476, wherein said a water-soluble acetylated cannabinoid and a water-soluble glycosylated cannabinoid comprises a water-soluble acetylated cannabinoid and a water-soluble glycosylated cannabinoid generated in vitro.
   479. The tablet or capsule of clause 476, wherein said a water-soluble acetylated cannabinoid and a water-soluble glycosylated cannabinoid comprises a non-psychoactive a water-soluble acetylated cannabinoid and a water-soluble glycosylated cannabinoid.
   480. The tablet or capsule of clause 476, wherein the amount of a water-soluble acetylated cannabinoid and a water-soluble glycosylated cannabinoid is 5 milligrams or less.
   481. The tablet or capsule of clause 476, wherein the amount of a water-soluble acetylated cannabinoid and a water-soluble glycosylated cannabinoid is between 5 milligrams and 200 milligrams.
   482. The tablet or capsule of clause 476, wherein the wherein the amount of a water-soluble acetylated cannabinoid and a water-soluble glycosylated cannabinoid is more than 200 milligrams.
   483. A method of manufacturing and packaging a cannabinoid dosage, consisting of the following steps:
preparing a fill solution with a desired concentration of a water-soluble cannabinoid in a liquid carrier wherein said cannabinoid solubilized in said liquid carrier;
encapsulating said fill solution in capsules;
packaging said capsules in a closed packaging system; and
removing atmospheric air from the capsules,
   wherein the removing of atmospheric air consists solely of purging said packaging system with an inert gas, and wherein said packaging system provides a room temperature stable product.
   484. The method of clause 483, wherein the packaging system is a blister package.
   485. The method of clause 484 wherein the blister package is constructed of material that minimizes exposure to moisture and air.
   486. The method of clause 483, wherein the cannabinoid is a glycosylated cannabinoid, a acetylated cannabinoid or a mixture of the two.
   487. The method of clause 486, wherein said glycosylated cannabinoid and/or said acetylated cannabinoid are generated in vivo.
   488. The method of clause 486, wherein said glycosylated cannabinoid and/or said acetylated cannabinoid are generated in vitro.
   489. The method of clause 483, wherein the liquid carrier is water-based carrier.
   490. The method of clause 487, wherein the water-based carrier is an aqueous sodium chloride solution.
   491. The method of clause 483, wherein the capsules are soft gelatin capsules.
   492. The method of clause 483, wherein the inert gas is nitrogen.
   493. The method of clause 483, wherein the desired cannabinoid concentration is about 1-10% w/w.
   494. The method of clause 493 wherein the desired concentration is about 1.5-6.5% w/w.
   495. An oral pharmaceutical solution consisting essentially of a water-soluble cannabinoid, 30-33% w/w water, about 50% w/w alcohol, 0.01% w/w butylated hydroxylanisole (BHA) or 0.1% w/w ethylenediaminetetraacetic acid (EDTA) and 5-21% w/w co-solvent, having a combined total of 100%, wherein said co-solvent is selected from the group consisting of propylene glycol, polyethylene glycol and combinations thereof, and wherein said water-soluble cannabinoid is a glycosylated cannabinoid, an acetylated cannabinoid or a mixture of the two.
   496. The oral pharmaceutical solution of clause 495 consisting essentially of 0.1 to 5% w/w of said water-soluble cannabinoid, about 50% w/w alcohol, 5.5% w/w propylene glycol, 12% w/w polyethylene glycol and 30-33% w/w water.
   497. The oral pharmaceutical solution of clause 496, wherein said alcohol is ethanol.
   498. An oral pharmaceutical solution consisting essentially of about 0.1% to 1% w/w water-soluble cannabinoid, about 50% w/w alcohol, 5.5% w/w propylene glycol, 12% w/w polyethylene glycol, 30-33% w/w water, 0.01% w/w butylated hydroxyanisole, having a combined total of 100%, and wherein said water-soluble cannabinoid is a glycosylated cannabinoid, an acetylated cannabinoid or a mixture of the two wherein that were generated in vivo.
   499. The oral pharmaceutical solution of clause 498 in sublingual spray form.
   500. An oral pharmaceutical solution comprising 0.54% w/w water-soluble cannabinoid, 31.9% w/w water, 12% w/w polyethylene glycol 400, 5.5% w/w propylene glycol, 0.01% w/w butylated hydroxyanisole, 0.05% w/w sucralose, and 50% w/w alcohol.
   501. An solution for nasal and/or sublingual administration of a composition comprising:
an excipient of propylene glycol, ethanol anhydrous, or a mixture of both;
a water-soluble glycosylated cannabinoid;
   502. The solution of clause 501, wherein said glycosylated cannabinoid is generated in vivo.
   503. The solution of clause 501, wherein said glycosylated cannabinoid is generated in vitro.
   504. The solution of clause 501, wherein said glycosylated cannabinoid is non-psychoactive.
   505. The aqueous solution of clause 501, and further comprising a topical decongestant.
   506. The aqueous solution of clause 505, wherein said topical decongestant is selected from the group consisting of: phenylephrine hydrochloride, Oxymetazoline hydrochloride, and Xylometazoline.
   507. The aqueous solution of clause 501, and further comprising an antihistamine.
   508. The aqueous solution of clause 501, and further comprising a steroid.
   509. The aqueous solution of clause 509, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   510. The aqueous solution of clause 501, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   511. An solution for nasal and/or sublingual administration of a composition comprising:
an excipient of propylene glycol, ethanol anhydrous or a mixture of both; and
an water-soluble acetylated cannabinoid.
   512. The solution of clause 511, wherein said acetylated cannabinoid is generated in vivo.
   513. The solution of clause 511, wherein said acetylated cannabinoid is generated in vitro.
   514. The solution of clause 511, wherein said acetylated cannabinoid is non-psychoactive.
   515. The aqueous solution of clause 511, and further comprising a topical decongestant.
   516. The aqueous solution of clause 515, wherein said topical decongestant is selected from the group consisting of: phenylephrine hydrochloride, Oxymetazoline hydrochloride, and Xylometazoline.
   517. The aqueous solution of clause 511, and further comprising an antihistamine.
   518. The aqueous solution of clause 511, and further comprising a steroid.
   519. The aqueous solution of clause 518, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   520. The aqueous solution of clause 519, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   521. A solution for nasal and/or sublingual administration of a composition comprising:
an excipient of propylene glycol, ethanol anhydrous or a mixture of both; and
a water-soluble glycosylated cannabinoid and an water-soluble acetylated cannabinoid.
   522. The solution of clause 521, wherein said acetylated cannabinoid and said glycosylated cannabinoid is generated in vivo.
   523. The solution of clause 521, wherein said acetylated cannabinoid and said glycosylated cannabinoid is generated in vitro.
   524. The solution of clause 521, wherein said acetylated cannabinoid and said glycosylated cannabinoid are non-psychoactive.
   525. The aqueous solution of clause 521, and further comprising a topical decongestant.
   526. The aqueous solution of clause 525, wherein said topical decongestant is selected from the group consisting of: phenylephrine hydrochloride, Oxymetazoline hydrochloride, and Xylometazoline.
   527. The aqueous solution of clause 521, and further comprising an antihistamine.
   528. The aqueous solution of clause 521, and further comprising a steroid.
   529. The aqueous solution of clause 528, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   530. The aqueous solution of clause 529, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   531. An aqueous solution for nasal and/or sublingual administration of a compositions comprising:
a saline solution; and
a water-soluble glycosylated cannabinoid.
   532. The aqueous solution of clause 531, wherein said glycosylated cannabinoid is generated in vivo.
   533. The aqueous solution of clause 531, wherein said glycosylated cannabinoid is generated in vitro.
   534. The aqueous solution of clause 531, wherein said glycosylated cannabinoid is non-psychoactive.
   535. The aqueous solution of clause aqueous 531, and further comprising a topical decongestant.
   536. The aqueous solution of clause 535, wherein said topical decongestant is selected from the group consisting of: phenylephrine hydrochloride, Oxymetazoline hydrochloride, and Xylometazoline.
   537. The aqueous solution of clause 531, and further comprising an antihistamine.
   538. The aqueous solution of clause 531, and further comprising a steroid.
   539. The aqueous solution of clause 539, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   540. The aqueous solution of clause 531, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   541. An aqueous solution for nasal and/or sublingual administration of a composition comprising:
a saline solution; and
a water-soluble acetylated cannabinoid.
   542. The aqueous solution of clause 541, wherein said acetylated cannabinoid is generated in vivo.
   543. The aqueous solution of clause 541, wherein said acetylated cannabinoid is generated in vitro.
   544. The aqueous solution of clause 541, wherein said acetylated cannabinoid is non-psychoactive.
   545. The aqueous solution of clause 541, and further comprising a topical decongestant.
   546. The aqueous solution of clause 545, wherein said topical decongestant is selected from the group consisting of: phenylephrine hydrochloride, Oxymetazoline hydrochloride, and Xylometazoline.
   547. The aqueous solution of clause 546, and further comprising an antihistamine.
   548. The aqueous solution of clause 545, and further comprising a steroid.
   549. The aqueous solution of clause 548, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   550. The aqueous solution of clause 549, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   551. An aqueous solution for nasal and/or sublingual administration of a composition comprising:
a saline solution; and
a water-soluble acetylated cannabinoid and a water-soluble glycosylated cannabinoid.
   552. The aqueous solution of clause 551, wherein said acetylated cannabinoid and said glycosylated cannabinoid is generated in vivo.
   553. The aqueous solution of clause 551, wherein said acetylated cannabinoid and said glycosylated cannabinoid is generated in vitro.
   554. The aqueous solution of clause 551, wherein said acetylated cannabinoid and said glycosylated cannabinoid are non-psychoactive.
   555. The aqueous solution of clause 551, and further comprising a topical decongestant.
   556. The aqueous solution of clause 555, wherein said topical decongestant is selected from the group consisting of: phenylephrine hydrochloride, Oxymetazoline hydrochloride, and Xylometazoline.
   557. The aqueous solution of clause 551, and further comprising an antihistamine.
   558. The aqueous solution of clause 551, and further comprising a steroid.
   559. The aqueous solution of clause 557, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   560. The aqueous solution of clause 551, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   561. An aqueous solution for nasal and/or sublingual administration of a compositions comprising:
purified water; and
a water-soluble glycosylated cannabinoid.
   562. The aqueous solution of clause 561, wherein said glycosylated cannabinoid is generated in vivo.
   563. The aqueous solution of clause 561, wherein said glycosylated cannabinoid is generated in vitro.
   564. The solution of clause 561, wherein said glycosylated cannabinoid is non-psychoactive.
   565. The aqueous solution of clause 561, and further comprising a topical decongestant.
   566. The aqueous solution of clause 565, wherein said topical decongestant is selected from the group consisting of: phenylephrine hydrochloride, Oxymetazoline hydrochloride, and Xylometazoline.
   567. The aqueous solution of clause 561, and further comprising an antihistamine.
   568. The aqueous solution of clause 561, and further comprising a steroid.
   569. The aqueous solution of clause 568, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   570. The aqueous solution of clause 561, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   571. An aqueous solution for nasal and/or sublingual administration of a composition comprising:
purified water; and
a water-soluble acetylated cannabinoid.
   572. The aqueous solution of clause 571, wherein said acetylated cannabinoid is generated in vivo.
   573. The aqueous solution of clause 571, wherein said acetylated cannabinoid is generated in vitro.
   574. The solution of clause 571, wherein said acetylated cannabinoid is non-psychoactive.
   575. The aqueous solution of clause 571, and further comprising a topical decongestant.
   576. The aqueous solution of clause 575, wherein said topical decongestant is selected from the group consisting of: phenylephrine hydrochloride, Oxymetazoline hydrochloride, and Xylometazoline.
   577. The aqueous solution of clause 571, and further comprising an antihistamine.
   578. The aqueous solution of clause 571, and further comprising a steroid.
   579. The aqueous solution of clause 578, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   580. The aqueous solution of clause 579, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   581. An aqueous solution for nasal and/or sublingual administration of a composition comprising:
purified water; and
a water-soluble acetylated cannabinoid and a water-soluble glycosylated cannabinoid.
   582. The aqueous solution of clause 581, wherein said acetylated cannabinoid and said glycosylated cannabinoid is generated in vivo.
   583. The aqueous solution of clause 581, wherein said acetylated cannabinoid and said glycosylated cannabinoid is generated in vitro.
   584. The aqueous solution of clause 581, wherein said acetylated cannabinoid and said glycosylated cannabinoid are non-psychoactive.
   585. The aqueous solution of clause 581, and further comprising a topical decongestant.
   586. The aqueous solution of clause 585, wherein said topical decongestant is selected from the group consisting of: phenylephrine hydrochloride, Oxymetazoline hydrochloride, and Xylometazoline.
   587. The aqueous solution of clause 581, and further comprising an antihistamine.
   588. The aqueous solution of clause 581, and further comprising a steroid.
   589. The aqueous solution of clause 588, wherein said steroid is a corticosteroid selected from the group consisting of: neclomethasone dipropionate, budesonide, ciclesonide, flunisolide, fluticasone furoate, fluticasone propionate, mometasone, triamcinolone acetonide.
   590. The aqueous solution of clause 581, the solution further comprising at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, propylene glycol.
   591. A topical formulation consisting of a water-soluble glycosylated cannabinoid, and/or water-soluble acetylated cannabinoid, or a mixture of both, and a pharmaceutically acceptable excipient.
   592. The topical formulation according to clause 591, and further comprising a quantity of capsaicin.
   593. The topical formulation according to clause 591, and further comprising a quantity of benzocaine.
   594. The topical formulation according to clause 591, and further comprising a quantity of lidocaine.
   595. The topical formulation according to clause 591, and further comprising a quantity of camphor.
   596. The topical formulation according to clause 591, and further comprising a quantity of benzoin resin.
   597. The topical formulation according to clause 591, and further comprising a quantity of methylsalicilate.
   598. The topical formulation according to clause 591, and further comprising a quantity of triethanolamine salicylate.
   599. The topical formulation according to clause 591, and further comprising a quantity of hydrocortisone.
   600. The topical formulation according to clause 591, and further comprising a quantity of salicylic acid.
   601. The topical formulation according to clause 591, and further comprising a wherein the pharmaceutically acceptable excipient is selected from the group consisting of: gels, ointments, cataplasms, poultices, pastes, creams, lotions, plasters and jellies
   602. The topical formulation according to clause 591, and further comprising a polyethylene glycol.
   603. A gel for transdermal administration, the mixture preferably contains from 15% to about 90% ethanol, from about 10% to about 60% buffered aqueous solution or water, from about 0.1 to about 25% propylene glycol, from about 0.1 to about 20% of a gelling agent, from about 0.1 to about 20% of a base, from about 0.1 to about 20% of an absorption enhancer and from about 1% to about 25% polyethylene glycol and a water-soluble cannabinoid.
   604. The gel of clause 603, wherein said water-soluble cannabinoid comprises a water-soluble glycosylated cannabinoid, and/or water-soluble acetylated cannabinoid, or a mixture of both
   605. The gel of clause 604, wherein said glycosylated cannabinoid, and/or said acetylated cannabinoid were generated in vivo.
   606. The gel of clause 604, wherein said glycosylated cannabinoid, and/or said acetylated cannabinoid were generated in vitro.
   607. A formulation comprising the following volumetric amounts: (i) from about 15% to about 90% ethanol, (ii) a glycol selected from the group consisting of (a) propylene glycol from about 0.1% to about 25%, (b) polyethylene glycol from about 1 to about 30%, and (c) a combination of (a) and (b), (iii) from about 0.1 to about 20% of a gelling agent, (iv) from about 0.1 to about 20% of a base and (v) from about 0.1 to about 20% of an absorption enhancer, and a water-soluble cannabinoid, said formulation being suitable for transdermal administration.
   608. The formulation of clause 607, wherein said water-soluble cannabinoid comprises a water-soluble glycosylated cannabinoid, and/or water-soluble acetylated cannabinoid, or a mixture of both.
   609. The formulation of clause 608, wherein said glycosylated cannabinoid, and/or said acetylated cannabinoid were generated in vivo.
   610. The formulation of clause 608, wherein said glycosylated cannabinoid, and/or said acetylated cannabinoid were generated in vitro.
   611. A transdermal composition comprising a pharmaceutically effective amount of a water-soluble cannabinoid for delivery of the cannabinoid to the bloodstream of a user, said composition comprising:
a pharmaceutically acceptable excipient;
at least one water-soluble cannabinoid;
wherein the cannabinoid is capable of diffusing from the composition into the bloodstream of the user.
   612. The composition of clause 611, wherein the water-soluble cannabinoid is selected from the group consisting of: a glycosylated cannabinoid, an acetylated cannabinoid, and a mixture of both.
   613. The composition of clause 612, wherein said glycosylated cannabinoid, and/or said acetylated cannabinoid were generated in vivo.
   614. The composition of clause 611, wherein the transdermal composition further comprises one or more pharmaceutically acceptable excipients to create a transdermal dosage form selected from the group consisting of: gels, ointments, cataplasms, poultices, pastes, creams, lotions, plasters and jellies.
   615. The composition of clause 611, and further comprising a surfactant.
   616. The composition of clause 611, wherein the surfactant is a surfactant-lecithin organogel.
   617. The composition of clause 611, wherein the surfactant-lecithin organogel is present in an amount of between about between about 95% and about 98% w/w.
   618. The composition of clause 611, wherein the surfactant-lecithin organogel comprises lecithin and PPG-2 myristyl ether propionate.
   619. The composition of clause 611, wherein the surfactant-lecithin organogel comprises a surfactant comprising high molecular weight polyacrylic acid polymers.
   622. The composition of clause 611, wherein the composition further comprises isopropyl myristate.
   623. The composition of clause 611, wherein the water-soluble cannabinoid is non-psychoactive.
   624. The composition of clause 611, wherein the pharmaceutically acceptable excipients is selected from the group consisting of: gels, ointments, cataplasms, poultices, pastes, creams, lotions, plasters and jellies
   625. A transdermal composition comprising a pharmaceutically effective amount of a water-soluble cannabinoid for delivery of the cannabinoid to the bloodstream of a user, said composition comprising:
a permeation enhancer;
at least one water-soluble cannabinoid;
wherein the cannabinoid is capable of diffusing from the composition into the bloodstream of the user.
   626. The composition of clause 625, wherein the water-soluble cannabinoid is selected from the group consisting of: a glycosylated cannabinoid, an acetylated cannabinoid, and a mixture of both.
   627. The composition of clause 626, wherein said glycosylated cannabinoid, and/or said acetylated cannabinoid were generated in vivo.
   628. The composition of clause 625, wherein the permeation enhancer is selected from the group consisting of: propylene glycol monolaurate, diethylene glycol monoethyl ether, an oleoyl macrogolglyceride, a caprylocaproyl macrogolglyceride, and an oleyl alcohol.
   629. The composition of clause 625, herein the transdermal composition further comprises one or more pharmaceutically acceptable excipients to create a transdermal dosage form selected from the group consisting of: gels, ointments, cataplasms, poultices, pastes, creams, lotions, plasters and jellies.
   630. A liquid cannabinoid liniment composition consisting of water, isopropyl alcohol solution and a water-soluble cannabinoid.
   631. The composition of clause 630, wherein said water-soluble cannabinoid is selected from the group consisting of: a glycosylated cannabinoid, an acetylated cannabinoid, and a mixture of both.
   632. The composition of clause 632, wherein said glycosylated cannabinoid, and/or said acetylated cannabinoid were generated in vivo.
   633. The composition of clause 630, consisting of from about 97.5% to about 99.5% by weight of 70% isopropyl alcohol solution and from about 0.5% to about 2.5% by weight of a cannabinoid mixture
   634. A commercially available topical creme composition infused with a glycosylated cannabinoid, an acetylated cannabinoid, and a mixture of both.
   635. The composition of clause 634, wherein said glycosylated cannabinoid, and/or said acetylated cannabinoid were generated in vivo.
   636. A commercially available lip balm composition supplemented with a water-soluble cannabinoid wherein said comprises a glycosylated cannabinoid, and/or an acetylated cannabinoid, and/or a mixture of both.
   637. The composition of clause 636, wherein said glycosylated cannabinoid, and/or said acetylated cannabinoid were generated in vivo.
   638. A commercially available cosmetic composition supplemented with a water-soluble cannabinoid wherein said comprises a glycosylated cannabinoid, and/or an acetylated cannabinoid, and/or a mixture of both.
   639. The composition of clause 638, wherein said glycosylated cannabinoid, and/or said acetylated cannabinoid were generated in vivo.
   640. A tobacco plant containing at least one water-soluble cannabinoids.
   641. The tobacco plant in clause 640, wherein said water-soluble cannabinoid comprises a glycosylated cannabinoid, and/or an acetylated cannabinoid, and/or a mixture of both.
   642. The tobacco plant of clause 641, wherein said glycosylated cannabinoid, and/or said acetylated cannabinoid were generated in vivo.
   643. The tobacco plant of clause 641, wherein said glycosylated cannabinoid, and/or said acetylated cannabinoid were generated in vitro.
   644. The tobacco plant of clause 640, wherein said water-soluble cannabinoid is non-psychoactive.
   645. The tobacco plant of clause 640, wherein said tobacco plant is used to generate a water-soluble cannabinoid infused tobacco product.
   646. The tobacco plant of clause 645, wherein said cannabinoid infused tobacco product is a cigarette, pipe tobacco, chewing tobacco, cigar, smokeless tobacco.
   646. A composition comprising:
an aqueous solution;
water-soluble cannabinoid dissolved in said aqueous solution wherein said water-soluble cannabinoid comprises a glycosylated cannabinoid, and/or an acetylated cannabinoid, and/or a mixture of both;
wherein said composition may be introduced to a cigarette and/or a tobacco leaf such that said aqueous solution may evaporate generating a cigarette and/or a tobacco leaf that contains said water-soluble cannabinoid.
   647. The composition of clause 646, wherein said glycosylated cannabinoid, and/or said acetylated cannabinoid were generated in vivo.
   648. The composition of clause 646, wherein said glycosylated cannabinoid, and/or said acetylated cannabinoid were generated in vivo.
   649. The composition of clause 646, wherein said water-soluble cannabinoid is non-psychoactive.
   650. The composition of clause 646, wherein said aqueous solution comprises purified water.
   651. A method of treating a medical condition in a mammal comprising the step of administering a therapeutically effective amount of a glycosylated cannabinoid, and/or an acetylated cannabinoid, and/or a mixture of both or a pharmaceutically acceptable salt thereof, wherein the medical condition is selected from the group consisting of: obesity, post-traumatic stress syndrome, anorexia, nausea, emesis, pain, wasting syndrome, HIV-wasting, chemotherapy induced nausea and vomiting, alcohol use disorders, anti-tumor, amyotrophic lateral sclerosis, glioblastoma multiforme, glioma, increased intraocular pressure, glaucoma, cannabis use disorders, Tourette's syndrome, dystonia, multiple sclerosis, inflammatory bowel disorders, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, and juvenile rheumatoid arthritis.
   652. The method of clause 651 wherein the compound is administered by a route selected from the group consisting of: transdermal, topical, oral, buccal, sublingual, intra-venous, intra-muscular, vaginal, rectal, ocular, nasal and follicular.
   653. The method of clause 652, wherein said glycosylated cannabinoid, and/or an acetylated cannabinoid, and/or a mixture of both are glycosylated cannabinoid, and/or acetylated in vivo.

### REFERENCES

The following references are hereby incorporated in their entirety by reference:
[1] I von Ossowski, M R Mulvey, P A Leco, A Borys and P C Loewen, J. Bacteriol. 1991, 173(2):514.
[2] Behera, A., Behera, A., Mishra, S. C., Swain, S. K., & Author, C. (2003). Cannabinoid glycosides: In vitro production of a new class of cannabinoids with improved physicochemical properties. Proc. Intl. Soc. Mag. Reson. Med (Vol. 14).
[3] Holland, M. L., Lau, D. T. T., Allen, J. D., & Arnold, J. C. (2009). The multidrug transporter ABCG2 (BCRP) is inhibited by plant-derived cannabinoids. British Journal of Pharmacology, 152(5), 815-824. https://doi.org/10.1038/sj.bjp.0707467
[4] Ivanchenco. M., Vejlupkova. Z., Quatrano. R. S., Fowler. J. E. (2000) Maize ROP7 GTPase contains a unique, CaaX box-independent plasma membrane targeting signal. The Plant Journal, (24)1, 79-90.
[5] James M. Rini and Jeffrey D. Esko. Glycosyltransferases and Glycan-Processing Enzymes. In: Essentials of Glycobiology [Internet]. 3rd edition. https://www.ncbi.nlm.nih.gov/books/NBK310274/?report=reader
[6] Marks, M. D., Tian, L., Wenger, J. P., Omburo, S. N., Soto-Fuentes, W., He, J., . . . Dixon, R. A. (2009). Identification of candidate genes affecting A9-tetrahydrocannabinol biosynthesis in Cannabis sativa. Journal of Experimental Botany, 60(13), 3715-3726. https://doi.org/10.1093/jxb/erp210
[7] Nagaya, S., Kawamura, K., Shinmyo, A., & Kato, K. (2010). The HSP terminator of Arabidopsis thaliana increases gene expression in plant cells. Plant and Cell Physiology, 51(2), 328-332. https://doi.org/10.1093/pcp/pcp188
[8] Norambuena, L., Marchant, L., Berninsone, P., Hirschberg, C. B., Silva, H., & Orellana, A. (2002). Transport of UDP-galactose in plants. Identification and functional characterization of AtUTr1, an Arabidopsis thaliana UDP-galactose/UDP-glucose transporter. Journal of Biological Chemistry, 277(36), 32923-32929. https://doi.org/10.1074/jbc.M204081200
[9] Onofri, C., De Meijer, E. P. M., & Mandolino, G. (2015). Sequence heterogeneity of cannabidiolic- and tetrahydrocannabinolic acid-synthase in Cannabis sativa L. and its relationship with chemical phenotype. Phytochemistry, 116(1), 57-68. https://doi.org/10.1016/j.phytochem.2015.03.006
[9] Priest, D. M., Ambrose, S. J., Vaistij, F. E., Elias, L., Higgins, G. S., Ross, A. R. S., . . . Bowles, D. J. (2006). Use of the glucosyltransferase UGT71B6 to disturb abscisic acid homeostasis in Arabidopsis thaliana. Plant Journal, 46(3), 492-502. https://doi.org/10.1111/j.1365-313X.2006.02701.x
[10] Siritunga, D., and Sayre, R. T. (2003). Generation of cyanogen-free transgenic cassava. Planta 217, 367-373. doi: 10.1007/s00425-003-1005-8
[11] Sparkes, I. A., Runions, J., Kearns, A., & Hawes, C. (2006). Rapid, transient expression of fluorescent fusion proteins in tobacco plants and generation of stably transformed plants. Nature Protocols, 1(4), 2019-2025. https://doi.org/10.1038/nprot.2006.286
[13] Taura, F., Morimoto, S., & Shoyama, Y. (1996). Purification and characterization of cannabidiolic-acid synthase from Cannabis sativa L. Biochemical analysis of a novel enzyme that catalyzes the oxidocyclization of. Journal of Biological Chemistry, 271(29), 17411-17416. https://doi.org/10.1074/JBC.271.29.17411
[14] Taura, F., Sirikantaramas, S., Shoyama Y, Yoshikai K, Shoyama Y, Morimoto S. (2007) Cannabidiolic-acid synthase, the chemotype-determining enzyme in the fiber-type Cannabis sativa. Febbs letters, 581(16), 2929-34. DOI: 10.1016/i.febslet.2007.05.043
[15] Yoo, S. D., Cho, Y. H., & Sheen, J. (2007). Arabidopsis mesophyll protoplasts: A versatile cell system for transient gene expression analysis. Nature Protocols, 2(7), 1565-1572. https://doi.org/10.1038/nprot.2007.199
[16] Matsui, T., Matsuura, H., Sawada, K., Takita, E., Kinjo, S., Takenami, S., . . . Kato, K. (2012). High level expression of transgenes by use of 5′-untranslated region of the Arabidopsis thaliana arabinogalactan-protein 21 gene in dicotyledons. Plant Biotechnology, 29(3), 319-322. https://doi.org/10.5511/plantbiotechnology. 12.0322a
[17] Murashige, T., and Skoog, F. (1962). A revised medium for rapid growth and bioassays with tobacco tissue culture. Physiol. Plant. 15, 473-497. doi: 10.1111/j.1399-3054.1962.tb08052.x
[18] Zipp, et al., Cannabinoid glycosides: In vitro production of a new class of cannabinoids with improved physicochemical properties. bioRxiv preprint doi: http://dx.doi.org/10.1101/104349
[19] Mohamed, E. A., T. Iwaki, I. Munir, M. Tamoi, S. Shigeoka, and A. Wadano. 2003. Overexpression of bacterial catalase in tomato leaf chloroplasts enhances photo-oxidative stress tolerance. Plant Cell Environ. 26:2037-2046.
[20] Akhtar, M. T., 2013, Doctoral Thesis, Leiden University. Cannabinoids and zebrafish. 2013-05-22. http://hdl.handle.net/1887/20899
[21] Sayed Farag. Cannabinoids production in Cannabis sativa L.: An in vitro approach. Thesis ⋅ January 2014. DOI: 10.17877/DE290R-16298
[21] K, Watanabe, et al., Cytochrome P450 enzymes involved in the metabolism of tetrahydrocannabinols and cannabinol by human hepatic microsomes. Life Sciences. Volume 80, Issue 15, 20 Mar. 2007, Pages 1415-1419
[22] Flores-Sanchez I J. et al., Elicitation studies in cell suspension cultures of Cannabis sativa L. J Biotechnol. 2009 Aug. 20; 143(2): 157-68. doi: 10.1016/j.jbiotec.
[23] Stephen M. Stout & Nina M. Cimino (2013) Exogenous cannabinoids as substrates, inhibitors, and inducers of human drug metabolizing enzymes: a systematic review, Drug Metabolism Reviews, 46:1, 86-95, DOI: 10.3109/03602532.2013.849268
[24] Andre C M, Hausman J-F, Guerriero G. Cannabis sativa: The Plant of the Thousand and One Molecules. Frontiers in Plant Science. 2016; 7:19. doi:10.3389/fpls.2016.00019.
[25] Mahlberg P1. et al., Accumulation of Cannabinoids in Glandular Trichomes of Cannabis (Cannabaceae). Journal of Industrial Hemp 9(1):15-36 ⋅ June 2004 with 273 Reads DOI: 10.1300/J237v09n01_04.
[25] Katalin S., et al., Mini Rev Med Chem. 2017; 17(13):1223-1291. doi: 10.2174/1389557516666161004162133.
[26] Sirikantaramas S., et al., Tetrahydrocannabinolic Acid Synthase, the Enzyme Controlling Marijuana Psychoactivity, is Secreted into the Storage Cavity of the Glandular Trichomes. Plant and Cell Physiology, Volume 46, Issue 9, 1 Sep. 2005, Pages 1578-1582, https://doi.org/10.1093/pcp/pci166.
[26] Schilmiller A L, Last R L, Pichersky E (2008) Harnessing plant trichome biochemistry for the production of useful compounds. Plant Journal 54: 702-711.
[27] Matias-Hernandez, L. et al. AaMYB1 and its orthologue AtMYB61 affect terpene metabolism and trichome development in Artemisia annua and Arabidopsis thaliana. Plant J.
[28] Ahmad, M., Hirz, M., Pichler, H., & Schwab, H. (2014). Protein expression in Pichia pastoris: Recent achievements and perspectives for heterologous protein production. Applied Microbiology and Biotechnology, 98(12), 5301-5317.
[29] Cregg, J. M., Cereghino, J. L., Shi, J., & Higgins, D. R. (2000). Recombinant Protein Expression in Pichia pastoris. Molecular Biotechnology, 16(1), 23-52.
[30] Ellis, S. B., Brust, P. F., Koutz, P. J., Waters, A. N. N. F., Harpold, M. M., Gingeras, T. R., & Al, E. E. T. (1985). Isolation of Alcohol Oxidase and Two Other Methanol Regulatable Genes from the Yeast Pichia pastoris, 5(5), 1111-1121.
[31] Santos, R. B., Abranches, R., Fischer, R., Sack, M., & Holland, T. (2016). Putting the Spotlight Back on Plant Suspension Cultures. Frontiers in Plant Science, 7(March), 1-12.
[32] Nagata, T., Nemoto, Y., and Hasezawa, S. (1992). Tobacco BY-2 cell line as the “HeLa” cell in the cell biology of higher plants. Int. Rev. Cytol. 132, 1-30. doi: 10.1016/S0074-7696(08)62452-3

### SEQUENCE LISTINGS

As noted above, the instant application contains a full Sequence Listing which has been submitted electronically in ASCII format and is hereby incorporated by reference in its entirety. The following sequences are further provided herewith and are hereby incorporated into the specification in their entirety:

DNA
Cytochrome P450 (CYP3A4)
Human
SEQ ID NO. 1
ATGGCTTTGATTCCTGATTTGGCTATGGAAACTAGATTGTTGTTGGCTGTTTCATTGGTTTTGT
TGTATTTGTATGGAACTCATTCACATGGATTGTTTAAAAAATTGGGAATTCCTGGACCTACTCC
TTTGCCTTTTTTGGGAAATATTTTGTCATATCATAAAGGATTTTGCATGTTTGATATGGAATGC
CATAAAAAATATGGAAAAGTTTGGGGATTTTATGATGGACAACAACCTGTTTTGGCTATTACTG
ATCCTGATATGATTAAAACTGTTTTGGTTAAAGAATGCTATTCAGTTTTTACTAATAGAAGACC
TTTTGGACCTGTTGGATTTATGAAATCAGCTATTTCAATTGCTGAAGATGAAGAATGGAAAAGA
TTGAGATCATTGTTGTCACCTACTTTTACTTCAGGAAAATTGAAAGAAATGGTTCCTATTATTG
CTCAATATGGAGATGTTTTGGTTAGAAATTTGAGAAGAGAAGCTGAAACTGGAAAACCTGTTAC
TTTGAAAGATGTTTTTGGAGCTTATTCAATGGATGTTATTACTTCAACTTCATTTGGAGTTAAT
ATTGATTCATTGAATAATCCTCAAGATCCTTTTGTTGAAAATACTAAAAAATTGTTGAGATTTG
ATTTTTTGGATCCTTTTTTTTTGTCAATTACTGTTTTTCCTTTTTTGATTCCTATTTTGGAAGT
TTTGAATATTTGCGTTTTTCCTAGAGAAGTTACTAATTTTTTGAGAAAATCAGTTAAAAGAATG
AAAGAATCAAGATTGGAAGATACTCAAAAACATAGAGTTGATTTTTTGCAATTGATGATTGATT
CACAAAATTCAAAAGAAACTGAATCACATAAAGCTTTGTCAGATTTGGAATTGGTTGCTCAATC
AATTATTTTTATTTTTGCTGGATGCGAAACTACTTCATCAGTTTTGTCATTTATTATGTATGAA
TTGGCTACTCATCCTGATGTTCAACAAAAATTGCAAGAAGAAATTGATGCTGTTTTGCCTAATA
AAGCTCCTCCTACTTATGATACTGTTTTGCAAATGGAATATTTGGATATGGTTGTTAATGAAAC
TTTGAGATTGTTTCCTATTGCTATGAGATTGGAAAGAGTTTGCAAAAAAGATGTTGAAATTAAT
GGAATGTTTATTCCTAAAGGAGTTGTTGTTATGATTCCTTCATATGCTTTGCATAGAGATCCTA
AATATTGGACTGAACCTGAAAAATTTTTGCCTGAAAGATTTTCAAAAAAAAATAAAGATAATAT
TGATCCTTATATTTATACTCCTTTTGGATCAGGACCTAGAAATTGCATTGGAATGAGATTTGCT
TTGATGAATATGAAATTGGCTTTGATTAGAGTTTTGCAAAATTTTTCATTTAAACCTTGCAAAG
AAACTCAAATTCCTTTGAAATTGTCATTGGGAGGATTGTTGCAACCTGAAAAACCTGTTGTTTT
GAAAGTTGAATCAAGAGATGGAACTGTTTCAGGAGCT
Amino Acid
Cytochrome P450 (CYP3A4)
Human
SEQ ID NO. 2
MALIPDLAMETRLLLAVSLVLLYLYGTHSHGLFKKLGIPGPTPLPFLGNILSYHKGFCMFDMEC
HKKYGKVWGFYDGQQPVLAITDPDMIKTVLVKECYSVFTNRRPFGPVGFMKSAISIAEDEEWKR
LRSLLSPTFTSGKLKEMVPIIAQYGDVLVRNLRREAETGKPVTLKDVFGAYSMDVITSTSFGVN
IDSLNNPQDPFVENTKKLLRFDFLDPFFLSITVFPFLIPILEVLNICVFPREVTNFLRKSVKRM
KESRLEDTQKHRVDFLQLMIDSQNSKETESHKALSDLELVAQSIIFIFAGCETTSSVLSFIMYE
LATHPDVQQKLQEEIDAVLPNKAPPTYDTVLQMEYLDMVVNETLRLFPIAMRLERVCKKDVEIN
GMFIPKGVVVMIPSYALHRDPKYWTEPEKFLPERFSKKNKDNIDPYIYTPFGSGPRNCIGMRFA
LMNMKLALIRVLQNFSFKPCKETQIPLKLSLGGLLQPEKPVVLKVESRDGTVSGA
DNA
P450 oxidoreductase gene (oxred)
Human
SEQ ID NO. 3
ATGATTAATATGGGAGATTCACATGTTGATACTTCATCAACTGTTTCAGAAGCTGTTGCTGAAG
AAGTTTCATTGTTTTCAATGACTGATATGATTTTGTTTTCATTGATTGTTGGATTGTTGACTTA
TTGGTTTTTGTTTAGAAAAAAAAAAGAAGAAGTTCCTGAATTTACTAAAATTCAAACTTTGACT
TCATCAGTTAGAGAATCATCATTTGTTGAAAAAATGAAAAAAACTGGAAGAAATATTATTGTTT
TTTATGGATCACAAACTGGAACTGCTGAAGAATTTGCTAATAGATTGTCAAAAGATGCTCATAG
ATATGGAATGAGAGGAATGTCAGCTGATCCTGAAGAATATGATTTGGCTGATTTGTCATCATTG
CCTGAAATTGATAATGCTTTGGTTGTTTTTTGCATGGCTACTTATGGAGAAGGAGATCCTACTG
ATAATGCTCAAGATTTTTATGATTGGTTGCAAGAAACTGATGTTGATTTGTCAGGAGTTAAATT
TGCTGTTTTTGGATTGGGAAATAAAACTTATGAACATTTTAATGCTATGGGAAAATATGTTGAT
AAAAGATTGGAACAATTGGGAGCTCAAAGAATTTTTGAATTGGGATTGGGAGATGATGATGGAA
ATTTGGAAGAAGATTTTATTACTTGGAGAGAACAATTTTGGTTGGCTGTTTGCGAACATTTTGG
AGTTGAAGCTACTGGAGAAGAATCATCAATTAGACAATATGAATTGGTTGTTCATACTGATATT
GATGCTGCTAAAGTTTATATGGGAGAAATGGGAAGATTGAAATCATATGAAAATCAAAAACCTC
CTTTTGATGCTAAAAATCCTTTTTTGGCTGCTGTTACTACTAATAGAAAATTGAATCAAGGAAC
TGAAAGACATTTGATGCATTTGGAATTGGATATTTCAGATTCAAAAATTAGATATGAATCAGGA
GATCATGTTGCTGTTTATCCTGCTAATGATTCAGCTTTGGTTAATCAATTGGGAAAAATTTTGG
GAGCTGATTTGGATGTTGTTATGTCATTGAATAATTTGGATGAAGAATCAAATAAAAAACATCC
TTTTCCTTGCCCTACTTCATATAGAACTGCTTTGACTTATTATTTGGATATTACTAATCCTCCT
AGAACTAATGTTTTGTATGAATTGGCTCAATATGCTTCAGAACCTTCAGAACAAGAATTGTTGA
GAAAAATGGCTTCATCATCAGGAGAAGGAAAAGAATTGTATTTGTCATGGGTTGTTGAAGCTAG
AAGACATATTTTGGCTATTTTGCAAGATTGCCCTTCATTGAGACCTCCTATTGATCATTTGTGC
GAATTGTTGCCTAGATTGCAAGCTAGATATTATTCAATTGCTTCATCATCAAAAGTTCATCCTA
ATTCAGTTCATATTTGCGCTGTTGTTGTTGAATATGAAACTAAAGCTGGAAGAATTAATAAAGG
AGTTGCTACTAATTGGTTGAGAGCTAAAGAACCTGTTGGAGAAAATGGAGGAAGAGCTTTGGTT
CCTATGTTTGTTAGAAAATCACAATTTAGATTGCCTTTTAAAGCTACTACTCCTGTTATTATGG
TTGGACCTGGAACTGGAGTTGCTCCTTTTATTGGATTTATTCAAGAAAGAGCTTGGTTGAGACA
ACAAGGAAAAGAAGTTGGAGAAACTTTGTTGTATTATGGATGCAGAAGATCAGATGAAGATTAT
TTGTATAGAGAAGAATTGGCTCAATTTCATAGAGATGGAGCTTTGACTCAATTGAATGTTGCTT
TTTCAAGAGAACAATCACATAAAGTTTATGTTCAACATTTGTTGAAACAAGATAGAGAACATTT
GTGGAAATTGATTGAAGGAGGAGCTCATATTTATGTTTGCGGAGATGCTAGAAATATGGCTAGA
GATGTTCAAAATACTTTTTATGATATTGTTGCTGAATTGGGAGCTATGGAACATGCTCAAGCTG
TTGATTATATTAAAAAATTGATGACTAAAGGAAGATATTCATTGGATGTTTGGTCA
Amino Acid
P450 oxidoreductase
Human
SEQ ID NO. 4
MINMGDSHVDTSSTVSEAVAEEVSLFSMTDMILFSLIVGLLTYWFLFRKKKEEVPEFTKIQTLT
SSVRESSFVEKMKKTGRNIIVFYGSQTGTAEEFANRLSKDAHRYGMRGMSADPEEYDLADLSSL
PEIDNALVVFCMATYGEGDPTDNAQDFYDWLQETDVDLSGVKFAVFGLGNKTYEHFNAMGKYVD
KRLEQLGAQRIFELGLGDDDGNLEEDFITWREQFWLAVCEHFGVEATGEESSIRQYELVVHTDI
DAAKVYMGEMGRLKSYENQKPPFDAKNPFLAAVTTNRKLNQGTERHLMHLELDISDSKIRYESG
DHVAVYPANDSALVNQLGKILGADLDVVMSLNNLDEESNKKHPFPCPTSYRTALTYYLDITNPP
RTNVLYELAQYASEPSEQELLRKMASSSGEGKELYLSWVVEARRHILAILQDCPSLRPPIDHLC
ELLPRLQARYYSIASSSKVHPNSVHICAVVVEYETKAGRINKGVATNWLRAKEPVGENGGRALV
PMFVRKSQFRLPFKATTPVIMVGPGTGVAPFIGFIQERAWLRQQGKEVGETLLYYGCRRSDEDY
LYREELAQFHRDGALTQLNVAFSREQSHKVYVQHLLKQDREHLWKLIEGGAHIYVCGDARNMAR
DVQNTFYDIVAELGAMEHAQAVDYIKKLMTKGRYSLDVWS
DNA
cannabidiolic acid (CBDA) synthase
Cannabis sativa
SEQ ID NO. 5
ATGAATCCTCGAGAAAACTTCCTTAAATGCTTCTCGCAATATATTCCCAATAATGCAACAAATC
TAAAACTCGTATACACTCAAAACAACCCATTGTATATGTCTGTCCTAAATTCGACAATACACAA
TCTTAGATTCACCTCTGACACAACCCCAAAACCACTTGTTATCGTCACTCCTTCACATGTCTCT
CATATCCAAGGCACTATTCTATGCTCCAAGAAAGTTGGCTTGCAGATTCGAACTCGAAGTGGTG
GTCATGATTCTGAGGGCATGTCCTACATATCTCAAGTCCCATTTGTTATAGTAGACTTGAGAAA
CATGCGTTCAATCAAAATAGATGTTCATAGCCAAACTGCATGGGTTGAAGCCGGAGCTACCCTT
GGAGAAGTTTATTATTGGGTTAATGAGAAAAATGAGAATCTTAGTTTGGCGGCTGGGTATTGCC
CTACTGTTTGCGCAGGTGGACACTTTGGTGGAGGAGGCTATGGACCATTGATGAGAAACTATGG
CCTCGCGGCTGATAATATCATTGATGCACACTTAGTCAACGTTCATGGAAAAGTGCTAGATCGA
AAATCTATGGGGGAAGATCTCTTTTGGGCTTTACGTGGTGGTGGAGCAGAAAGCTTCGGAATCA
TTGTAGCATGGAAAATTAGACTGGTTGCTGTCCCAAAGTCTACTATGTTTAGTGTTAAAAAGAT
CATGGAGATACATGAGCTTGTCAAGTTAGTTAACAAATGGCAAAATATTGCTTACAAGTATGAC
AAAGATTTATTACTCATGACTCACTTCATAACTAGGAACATTACAGATAATCAAGGGAAGAATA
AGACAGCAATACACACTTACTTCTCTTCAGTTTTCCTTGGTGGAGTGGATAGTCTAGTCGACTT
GATGAACAAGAGTTTTCCTGAGTTGGGTATTAAAAAAACGGATTGCAGACAATTGAGCTGGATT
GATACTATCATCTTCTATAGTGGTGTTGTAAATTACGACACTGATAATTTTAACAAGGAAATTT
TGCTTGATAGATCCGCTGGGCAGAACGGTGCTTTCAAGATTAAGTTAGACTACGTTAAGAAACC
AATTCCAGAATCTGTATTTGTCCAAATTTTGGAAAAATTATATGAAGAAGATATAGGAGCTGGG
ATGTATGCGTTGTACCCTTACGGTGGTATAATGGATGAGATTTCAGAATCAGCAATTCCATTCC
CTCATCGAGCTGGAATCTTGTATGAGTTATGGTACATATGTAGTTGGGAGAAGCAAGAAGATAA
CGAAAAGCATCTAAACTGGATTAGAAATATTTATAACTTCATGACTCCTTATGTGTCCAAAAAT
TCAAGATTGGCATATCTCAATTATAGAGACCTTGATATAGGAATAAATGATCCCAAGAATCCAA
ATAATTACACACAAGCACGTATTTGGGGTGAGAAGTATTTTGGTAAAAATTTTGACAGGCTAGT
AAAAGTGAAAACCCTGGTTGATCCCAATAACTTTTTTAGAAACGAACAAAGCATCCCACCTCAA
CCACGGCATCGTCATTAA
Amino Acid
Cannabidiolic acid (CBDA) synthase
Cannabis sativa
SEQ ID NO. 6
MNPRENFLKCFSQYIPNNATNLKLVYTQNNPLYMSVLNSTIHNLRFTSDTTPKPLVIVTPSHVS
HIQGTILCSKKVGLQIRTRSGGHDSEGMSYISQVPFVIVDLRNMRSIKIDVHSQTAWVEAGATL
GEVYYWVNEKNENLSLAAGYCPTVCAGGHFGGGGYGPLMRNYGLAADNIIDAHLVNVHGKVLDR
KSMGEDLFWALRGGGAESFGIIVAWKIRLVAVPKSTMFSVKKIMEIHELVKLVNKWQNIAYKYD
KDLLLMTHFITRNITDNQGKNKTAIHTYFSSVFLGGVDSLVDLMNKSFPELGIKKTDCRQLSWI
DTIIFYSGVVNYDTDNFNKEILLDRSAGQNGAFKIKLDYVKKPIPESVFVQILEKLYEEDIGAG
MYALYPYGGIMDEISESAIPFPHRAGILYELWYICSWEKQEDNEKHLNWIRNIYNFMTPYVSKN
SRLAYLNYRDLDIGINDPKNPNNYTQARIWGEKYFGKNFDRLVKVKTLVDPNNFFRNEQSIPPQ
PRHRH
DNA
UDP glycosyltransferase 76G1
Stevia rebaudiana
SEQ ID NO. 7
ATGGAAAATAAAACTGAAACTACTGTTAGAAGAAGAAGAAGAATTATTTTGTTTCCTGTTCCTT
TTCAAGGACATATTAATCCTATTTTGCAATTGGCTAATGTTTTGTATTCAAAAGGATTTTCAAT
TACTATTTTTCATACTAATTTTAATAAACCTAAAACTTCAAATTATCCTCATTTTACTTTTAGA
TTTATTTTGGATAATGATCCTCAAGATGAAAGAATTTCAAATTTGCCTACTCATGGACCTTTGG
CTGGAATGAGAATTCCTATTATTAATGAACATGGAGCTGATGAATTGAGAAGAGAATTGGAATT
GTTGATGTTGGCTTCAGAAGAAGATGAAGAAGTTTCATGCTTGATTACTGATGCTTTGTGGTAT
TTTGCTCAATCAGTTGCTGATTCATTGAATTTGAGAAGATTGGTTTTGATGACTTCATCATTGT
TTAATTTTCATGCTCATGTTTCATTGCCTCAATTTGATGAATTGGGATATTTGGATCCTGATGA
TAAAACTAGATTGGAAGAACAAGCTTCAGGATTTCCTATGTTGAAAGTTAAAGATATTAAATCA
GCTTATTCAAATTGGCAAATTTTGAAAGAAATTTTGGGAAAAATGATTAAACAAACTAGAGCTT
CATCAGGAGTTATTTGGAATTCATTTAAAGAATTGGAAGAATCAGAATTGGAAACTGTTATTAG
AGAAATTCCTGCTCCTTCATTTTTGATTCCTTTGCCTAAACATTTGACTGCTTCATCATCATCA
TTGTTGGATCATGATAGAACTGTTTTTCAATGGTTGGATCAACAACCTCCTTCATCAGTTTTGT
ATGTTTCATTTGGATCAACTTCAGAAGTTGATGAAAAAGATTTTTTGGAAATTGCTAGAGGATT
GGTTGATTCAAAACAATCATTTTTGTGGGTTGTTAGACCTGGATTTGTTAAAGGATCAACTTGG
GTTGAACCTTTGCCTGATGGATTTTTGGGAGAAAGAGGAAGAATTGTTAAATGGGTTCCTCAAC
AAGAAGTTTTGGCTCATGGAGCTATTGGAGCTTTTTGGACTCATTCAGGATGGAATTCAACTTT
GGAATCAGTTTGCGAAGGAGTTCCTATGATTTTTTCAGATTTTGGATTGGATCAACCTTTGAAT
GCTAGATATATGTCAGATGTTTTGAAAGTTGGAGTTTATTTGGAAAATGGATGGGAAAGAGGAG
AAATTGCTAATGCTATTAGAAGAGTTATGGTTGATGAAGAAGGAGAATATATTAGACAAAATGC
TAGAGTTTTGAAACAAAAAGCTGATGTTTCATTGATGAAAGGAGGATCATCATATGAATCATTG
GATCATTGGTTTCATATATTTCATCATTG
Amino Acid
UPD glycosyltransferase 76G1
Stevia rebaudiana
SEQ ID NO. 8
MENKTETTVRRRRRIILFPVPFQGHINPILQLANVLYSKGFSITIFHTNFNKPKTSNYPHFTFR
FILDNDPQDERISNLPTHGPLAGMRIPIINEHGADELRRELELLMLASEEDEEVSCLITDALWY
FAQSVADSLNLRRLVLMTSSLFNFHAHVSLPQFDELGYLDPDDKTRLEEQASGFPMLKVKDIKS
AYSNWQILKEILGKMIKQTRASSGVIWNSFKELEESELETVIREIPAPSFLIPLPKHLTASSSS
LLDHDRTVFQWLDQQPPSSVLYVSFGSTSEVDEKDFLEIARGLVDSKQSFLWVVRPGFVKGSTW
VEPLPDGFLGERGRIVKWVPQQEVLAHGAIGAFWTHSGWNSTLESVCEGVPMIFSDFGLDQPLN
ARYMSDVLKVGVYLENGWERGEIANAIRRVMVDEEGEYIRQNARVLKQKADVSLMKGGSSYESL
ESLVSYISSL
DNA
ABC transporter ABCG2
Human
SEQ ID NO. 9
ATGTCATCATCAAATGTTGAAGTTTTTATTCCTGTTTCACAAGGAAATACTAATGGATTTCCTG
CTACTGCTTCAAATGATTTGAAAGCTTTTACTGAAGGAGCTGTTTTGTCATTTCATAATATTTG
CTATAGAGTTAAATTGAAATCAGGATTTTTGCCTTGCAGAAAACCTGTTGAAAAAGAAATTTTG
TCAAATATTAATGGAATTATGAAACCTGGATTGAATGCTATTTTGGGACCTACTGGAGGAGGAA
AATCATCATTGTTGGATGTTTTGGCTGCTAGAAAAGATCCTTCAGGATTGTCAGGAGATGTTTT
GATTAATGGAGCTCCTAGACCTGCTAATTTTAAATGCAATTCAGGATATGTTGTTCAAGATGAT
GTTGTTATGGGAACTTTGACTGTTAGAGAAAATTTGCAATTTTCAGCTGCTTTGAGATTGGCTA
CTACTATGACTAATCATGAAAAAAATGAAAGAATTAATAGAGTTATTCAAGAATTGGGATTGGA
TAAAGTTGCTGATTCAAAAGTTGGAACTCAATTTATTAGAGGAGTTTCAGGAGGAGAAAGAAAA
AGAACTTCAATTGGAATGGAATTGATTACTGATCCTTCAATTTTGTTTTTGGATGAACCTACTA
CTGGATTGGATTCATCAACTGCTAATGCTGTTTTGTTGTTGTTGAAAAGAATGTCAAAACAAGG
AAGAACTATTATTTTTTCAATTCATCAACCTAGATATTCAATTTTTAAATTGTTTGATTCATTG
ACTTTGTTGGCTTCAGGAAGATTGATGTTTCATGGACCTGCTCAAGAAGCTTTGGGATATTTTG
AATCAGCTGGATATCATTGCGAAGCTTATAATAATCCTGCTGATTTTTTTTTGGATATTATTAA
TGGAGATTCAACTGCTGTTGCTTTGAATAGAGAAGAAGATTTTAAAGCTACTGAAATTATTGAA
CCTTCAAAACAAGATAAACCTTTGATTGAAAAATTGGCTGAAATTTATGTTAATTCATCATTTT
ATAAAGAAACTAAAGCTGAATTGCATCAATTGTCAGGAGGAGAAAAAAAAAAAAAAATTACTGT
TTTTAAAGAAATTTCATATACTACTTCATTTTGCCATCAATTGAGATGGGTTTCAAAAAGATCA
TTTAAAAATTTGTTGGGAAATCCTCAAGCTTCAATTGCTCAAATTATTGTTACTGTTGTTTTGG
GATTGGTTATTGGAGCTATTTATTTTGGATTGAAAAATGATTCAACTGGAATTCAAAATAGAGC
TGGAGTTTTGTTTTTTTTGACTACTAATCAATGCTTTTCATCAGTTTCAGCTGTTGAATTGTTT
GTTGTTGAAAAAAAAATTGTTTATTCATGATATATTTCAGGATATTATAGAGTTTCATCATATT
TTTTGGGAAAATTGTTGTCAGATTTGTTGCCTATGAGAATGTTGCCTTCAATTATTTTTACTTG
CATTGTTTATTTTATGTTGGGATTGAAAGCTAAAGCTGATGCTTTTTTTGTTATGATGTTTACT
TTGATGATGGTTGCTTATTCAGCTTCATCAATGGCTTTGGCTATTGCTGCTGGACAATCAGTTG
TTTCAGTTGCTACTTTGTTGATGACTATTTGCTTTGTTTTTATGATGATTTTTTCAGGATTGTT
GGTTAATTTGACTACTATTGCTTCATGGTTGTCATGGTTGCAATATTTTTCAATTCCTAGATAT
GGATTTACTGCTTTGCAACATAATGAATTTTTGGGACAAAATTTTTGCCCTGGATTGAATGCTA
CTGGAAATAATCCTTGCAATTATGCTACTTGCACTGGAGAAGAATATTTGGTTAAACAAGGAAT
TGATTTGTCACCTTGGGGATTGTGGAAAAATCATGTTGCTTTGGCTTGCATGATTGTTATTTTT
TTGACTATTGCTTATTTGAAATTGTTGTTTTTGAAAAAATATTCA
Amino Acid
ABC transporter ABCG2
Human
SEQ ID NO. 10
MSSSNVEVFIPVSQGNTNGFPATASNDLKAFTEGAVLSFHNICYRVKLKSGFLPCRKPVEKEIL
SNINGIMKPGLNAILGPTGGGKSSLLDVLAARKDPSGLSGDVLINGAPRPANFKCNSGYVVQDD
VVMGTLTVRENLQFSAALRLATTMTNHEKNERINRVIQELGLDKVADSKVGTQFIRGVSGGERK
RTSIGMELITDPSILFLDEPTTGLDSSTANAVLLLLKRMSKQGRTIIFSIHQPRYSIFKLFDSL
TLLASGRLMFHGPAQEALGYFESAGYHCEAYNNPADFFLDIINGDSTAVALNREEDFKATEIIE
PSKQDKPLIEKLAEIYVNSSFYKETKAELHQLSGGEKKKKITVFKEISYTTSFCHQLRWVSKRS
FKNLLGNPQASIAQIIVTVVLGLVIGAIYFGLKNDSTGIQNRAGVLFFLTTNQCFSSVSAVELF
VVEKKLFIHEYISGYYRVSSYFLGKLLSDLLPMRMLPSIIFTCIVYFMLGLKAKADAFFVMMFT
LMMVAYSASSMALAIAAGQSVVSVATLLMTICFVFMMIFSGLLVNLTTIASWLSWLQYFSIPRY
GFTALQHNEFLGQNFCPGLNATGNNPCNYATCTGEEYLVKQGIDLSPWGLWKNHVALACMIVIF
LTIAYLKLLFLKKYS
DNA
MYB12-like
Cannabis
SEQ ID NO. 11
ATGAAGAAGAACAAATCAACTAGTAATAATAAGAACAACAACAGTAATAATATCATCAAAAACG
ACATCGTATCATCATCATCATCAACAACAACAACATCATCAACAACTACAGCAACATCATCATT
TCATAATGAGAAAGTTACTGTCAGTACTGATCATATTATTAATCTTGATGATAAGCAGAAACGA
CAATTATGTCGTTGTCGTTTAGAAAAAGAAGAAGAAGAAGAAGGAAGTGGTGGTTGTGGTGAGA
CAGTAGTAATGATGCTAGGGTCAGTATCTCCTGCTGCTGCTACTGCTGCTGCAGCTGGGGGCTC
ATCAAGTTGTGATGAAGACATGTTGGGTGGTCATGATCAACTGTTGTTGTTGTGTTGTTCTGAG
AAAAAAACGACAGAAATTTCATCAGTGGTGAACTTTAATAATAATAATAATAATAATAAGGAAA
ATGGTGACGAAGTTTCAGGACCGTACGATTATCATCATCATAAAGAAGAGGAAGAAGAAGAAGA
AGAAGATGAAGCATCTGCATCAGTAGCAGCTGTTGATGAAGGGATGTTGTTGTGCTTTGATGAC
ATAATAGATAGCCACTTGCTAAATCCAAATGAGGTTTTGACTTTAAGAGAAGATAGCCATAATG
AAGGTGGGGCAGCTGATCAGATTGACAAGACTACTTGTAATAATACTACTATTACTACTAATGA
TGATTATAACAATAACTTGATGATGTTGAGCTGCAATAATAACGGAGATTATGTTATTAGTGAT
GATCATGATGATCAGTACTGGATAGACGACGTCGTTGGAGTTGACTTTTGGAGTTGGGAGAGTT
CGACTACTACTGTTATTACCCAAGAACAAGAACAAGAACAAGATCAAGTTCAAGAACAGAAGAA
TATGTGGGATAATGAGAAAGAGAAACTGTTGTCTTTGCTATGGGATAATAGTGATAACAGCAGC
AGTTGGGAGTTACAAGATAAAAGCAATAATAATAATAATAATAATGTTCCTAACAAATGTCAAG
AGATTACCTCTGATAAAGAAAATGCTATGGTTGCATGGCTTCTCTCCTGA
Amino Acid
MYB12
Cannabis
SEQ ID NO. 12
MKKNKSTSNNKNNNSNNIIKNDIVSSSSSTTTTSSTTTATSSFHNEKVTVSTDHIINLDDKQKR
QLCRCRLEKEEEEEGSGGCGETVVMMLGSVSPAAATAAAAGGSSSCDEDMLGGHDQLLLLCCSE
KKTTEISSVVNFNNNNNNNKENGDEVSGPYDYHHHKEEEEEEEEDEASASVAAVDEGMLLCFDD
IIDSHLLNPNEVLTLREDSHNEGGAADQIDKTTCNNTTITTNDDYNNNLMMLSCNNNGDYVISD
DHDDQYWIDDVVGVDFWSWESSTTTVITQEQEQEQDQVQEQKNMWDNEKEKLLSLLWDNSDNSS
SWELQDKSNNNNNNNVPNKCQEITSDKENAMVAWLLS
DNA
Catalase
Arabidopsis thaliana
SEQ ID NO. 13
ATGGATCCTTATAAATATAGACCTGCTTCATCATATAATTCACCTTTTTTTACTACTAATTCAG
GAGCTCCTGTTTGGAATAATAATTCATCAATGACTGTTGGACCTAGAGGATTGATTTTGTTGGA
AGATTATCATTTGGTTGAAAAATTGGCTAATTTTGATAGAGAAAGAATTCCTGAAAGAGTTGTT
CATGCTAGAGGAGCTTCAGCTAAAGGATTTTTTGAAGTTACTCATGATATTTCAAATTTGACTT
GCGCTGATTTTTTGAGAGCTCCTGGAGTTCAAACTCCTGTTATTGTTAGATTTTCAACTGTTAT
TCATGCTAGAGGATCACCTGAAACTTTGAGAGATCCTAGAGGATTTGCTGTTAAATTTTATACT
AGAGAAGGAAATTTTGATTTGGTTGGAAATAATTTTCCTGTTTTTTTTATTAGAGATGGAATGA
AATTTCCTGATATTGTTCATGCTTTGAAACCTAATCCTAAATCACATATTCAAGAAAATTGGAG
AATTTTGGATTTTTTTTCACATCATCCTGAATCATTGAATATGTTTACTTTTTTGTTTGATGAT
ATTGGAATTCCTCAAGATTATAGACATATGGATGGATCAGGAGTTAATACTTATATGTTGATTA
ATAAAGCTGGAAAAGCTCATTATGTTAAATTTCATTGGAAACCTACTTGCGGAGTTAAATCATT
GTTGGAAGAAGATGCTATTAGATTGGGAGGAACTAATCATTCACATGCTACTCAAGATTTGTAT
GATTCAATTGCTGCTGGAAATTATCCTGAATGGAAATTGTTTATTCAAATTATTGATCCTGCTG
ATGAAGATAAATTTGATTTTGATCCTTTGGATGTTACTAAAACTTGGCCTGAAGATATTTTGCC
TTTGCAACCTGTTGGAAGAATGGTTTTGAATAAAAATATTGATAATTTTTTTGCTGAAAATGAA
CAATTGGCTTTTTGCCCTGCTATTATTGTTCCTGGAATTCATTATTCAGATGATAAATTGTTGC
AAACTAGAGTTTTTTCATATGCTGATACTCAAAGACATAGATTGGGACCTAATTATTTGCAATT
GCCTGTTAATGCTCCTAAATGCGCTCATCATAATAATCATCATGAAGGATTTATGAATTTTATG
CATAGAGATGAAGAAGTTAATTATTTTCCTTCAAGATATGATCAAGTTAGACATGCTGAAAAAT
ATCCTACTCCTCCTGCTGTTTGCTCAGGAAAAAGAGAAAGATGCATTATTGAAAAAGAAAATAA
TTTTAAAGAACCTGGAGAAAGATATAGAACTTTTACTCCTGAAAGACAAGAAAGATTTATTCAA
AGATGGATTGATGCTTTGTCAGATCCTAGAATTACTCATGAAATTAGATCAATTTGGATTTCAT
ATTGGTCACAAGCTGATAAATCATTGGGACAAAAATTGGCTTCAAGATTGAATGTTAGACCTTC
AATT
Amino Acid
Catalase
Arabidopsis thaliana
SEQ ID NO. 14
MDPYKYRPASSYNSPFFTTNSGAPVWNNNSSMTVGPRGLILLEDYHLVEKLANFDRERIPERVV
HARGASAKGFFEVTHDISNLTCADFLRAPGVQTPVIVRFSTVIHARGSPETLRDPRGFAVKFYT
REGNFDLVGNNFPVFFIRDGMKFPDIVHALKPNPKSHIQENWRILDFFSHHPESLNMFTFLFDD
IGIPQDYRHMDGSGVNTYMLINKAGKAHYVKFHWKPTCGVKSLLEEDAIRLGGTNHSHATQDLY
DSIAAGNYPEWKLFIQIIDPADEDKFDFDPLDVTKTWPEDILPLQPVGRMVLNKNIDNFFAENE
QLAFCPAIIVPGIHYSDDKLLQTRVFSYADTQRHRLGPNYLQLPVNAPKCAHHNNHHEGFMNFM
HRDEEVNYFPSRYDQVRHAEKYPTPPAVCSGKRERCIIEKENNFKEPGERYRTFTPERQERFIQ
RWIDALSDPRITHEIRSIWISYWSQADKSLGQKLASRLNVRPSI
DNA
Catalase HPII (KatE)
Escherichia coli
SEQ ID NO. 15
ATGTCGCAACATAACGAAAAGAACCCACATCAGCACCAGTCACCACTACACGATTCCAGCGAAG
CGAAACCGGGGATGGACTCACTGGCACCTGAGGACGGCTCTCATCGTCCAGCGGCTGAACCAAC
ACCGCCAGGTGCACAACCTACCGCCCCAGGGAGCCTGAAAGCCCCTGATACGCGTAACGAAAAA
CTTAATTCTCTGGAAGACGTACGCAAAGGCAGTGAAAATTATGCGCTGACCACTAATCAGGGCG
TGCGCATCGCCGACGATCAAAACTCACTGCGTGCCGGTAGCCGTGGTCCAACGCTGCTGGAAGA
TTTTATTCTGCGCGAGAAAATCACCCACTTTGACCATGAGCGCATTCCGGAACGTATTGTTCAT
GCACGCGGATCAGCCGCTCACGGTTATTTCCAGCCATATAAAAGCTTAAGCGATATTACCAAAG
CGGATTTCCTCTCAGATCCGAACAAAATCACCCCAGTATTTGTACGTTTCTCTACCGTTCAGGG
TGGTGCTGGCTCTGCTGATACCGTGCGTGATATCCGTGGCTTTGCCACCAAGTTCTATACCGAA
GAGGGTATTTTTGACCTCGTTGGCAATAACACGCCAATCTTCTTTATCCAGGATGCGCATAAAT
TCCCCGATTTTGTTCATGCGGTAAAACCAGAACCGCACTGGGCAATTCCACAAGGGCAAAGTGC
CCACGATACTTTCTGGGATTATGTTTCTCTGCAACCTGAAACTCTGCACAACGTGATGTGGGCG
ATGTCGGATCGCGGCATCCCCCGCAGTTACCGCACCATGGAAGGCTTCGGTATTCACACCTTCC
GCCTGATTAATGCCGAAGGGAAGGCAACGTTTGTACGTTTCCACTGGAAACCACTGGCAGGTAA
AGCCTCACTCGTTTGGGATGAAGCACAAAAACTCACCGGACGTGACCCGGACTTCCACCGCCGC
GAGTTGTGGGAAGCCATTGAAGCAGGCGATTTTCCGGAATACGAACTGGGCTTCCAGTTGATTC
CTGAAGAAGATGAATTCAAGTTCGACTTCGATCTTCTCGATCCAACCAAACTTATCCCGGAAGA
ACTGGTGCCCGTTCAGCGTGTCGGCAAAATGGTGCTCAATCGCAACCCGGATAACTTCTTTGCT
GAAAACGAACAGGCGGCTTTCCATCCTGGGCATATCGTGCCGGGACTGGACTTCACCAACGATC
CGCTGTTGCAGGGACGTTTGTTCTCCTATACCGATACACAAATCAGTCGTCTTGGTGGGCCGAA
TTTCCATGAGATTCCGATTAACCGTCCGACCTGCCCTTACCATAATTTCCAGCGTGACGGCATG
CATCGCATGGGGATCGACACTAACCCGGCGAATTACGAACCGAACTCGATTAACGATAACTGGC
CGCGCGAAACACCGCCGGGGCCGAAACGCGGCGGTTTTGAATCATACCAGGAGCGCGTGGAAGG
CAATAAAGTTCGCGAGCGCAGCCCATCGTTTGGCGAATATTATTCCCATCCGCGTCTGTTCTGG
CTAAGTCAGACGCCATTTGAGCAGCGCCATATTGTCGATGGTTTCAGTTTTGAGTTAAGCAAAG
TCGTTCGTCCGTATATTCGTGAGCGCGTTGTTGACCAGCTGGCGCATATTGATCTCACTCTGGC
CCAGGCGGTGGCGAAAAATCTCGGTATCGAACTGACTGACGACCAGCTGAATATCACCCCACCT
CCGGACGTCAACGGTCTGAAAAAGGATCCATCCTTAAGTTTGTACGCCATTCCTGACGGTGATG
TGAAAGGTCGCGTGGTAGCGATTTTACTTAATGATGAAGTGAGATCGGCAGACCTTCTGGCCAT
TCTCAAGGCGCTGAAGGCCAAAGGCGTTCATGCCAAACTGCTCTACTCCCGAATGGGTGAAGTG
ACTGCGGATGACGGTACGGTGTTGCCTATAGCCGCTACCTTTGCCGGTGCACCTTCGCTGACGG
TCGATGCGGTCATTGTCCCTTGCGGCAATATCGCGGATATCGCTGACAACGGCGATGCCAACTA
CTACCTGATGGAAGCCTACAAACACCTTAAACCGATTGCGCTGGCGGGTGACGCGCGCAAGTTT
AAAGCAACAATCAAGATCGCTGACCAGGGTGAAGAAGGGATTGTGGAAGCTGACAGCGCTGACG
GTAGTTTTATGGATGAACTGCTAACGCTGATGGCAGCACACCGCGTGTGGTCACGCATTCCTAA
GATTGACAAAATTCCTGCCTGA
Amino Acid
Catalase HPII (KatE)
Escherichia coli
SEQ ID NO. 16
MSQHNEKNPHQHQSPLHDSSEAKPGMDSLAPEDGSHRPAAEPTPPGAQPTAPGSLKAPDTRNEK
LNSLEDVRKGSENYALTTNQGVRIADDQNSLRAGSRGPTLLEDFILREKITHFDHERIPERIVH
ARGSAAHGYFQPYKSLSDITKADFLSDPNKITPVFVRFSTVQGGAGSADTVRDIRGFATKFYTE
EGIFDLVGNNTPIFFIQDAHKFPDFVHAVKPEPHWAIPQGQSAHDTFWDYVSLQPETLHNVMWA
MSDRGIPRSYRTMEGFGIHTFRLINAEGKATFVRFHWKPLAGKASLVWDEAQKLTGRDPDFHRR
ELWEAIEAGDFPEYELGFQLIPEEDEFKFDFDLLDPTKLIPEELVPVQRVGKMVLNRNPDNFFA
ENEQAAFHPGHIVPGLDFTNDPLLQGRLFSYTDTQISRLGGPNFHEIPINRPTCPYHNFQRDGM
HRMGIDTNPANYEPNSINDNWPRETPPGPKRGGFESYQERVEGNKVRERSPSFGEYYSHPRLFW
LSQTPFEQRHIVDGFSFELSKVVRPYIRERVVDQLAHIDLTLAQAVAKNLGIELTDDQLNITPP
PDVNGLKKDPSLSLYAIPDGDVKGRVVAILLNDEVRSADLLAILKALKAKGVHAKLLYSRMGEV
TADDGTVLPIAATFAGAPSLTVDAVIVPCGNIADIADNGDANYYLMEAYKHLKPIALAGDARKF
KATIKIADQGEEGIVEADSADGSFMDELLTLMAAHRVWSRIPKIDKIPA
DNA
Trichome-targeted CBDA synthase
Cannabis
SEQ ID NO. 17
ATGAAGTGCTCAACATTCTCCTTTTGGTTTGTTTGCAAGATAATATTTTTCTTTTTCTCATTCA
ATATCCAAACTTCCATTGCTAATCCTCGAGAAAACTTCCTTAAATGCTTCTCGCAATATATTCC
CAATAATGCAACAAATCTAAAACTCGTATACACTCAAAACAACCCATTGTATATGTCTGTCCTA
AATTCGACAATACACAATCTTAGATTCACCTCTGACACAACCCCAAAACCACTTGTTATCGTCA
CTCCTTCACATGTCTCTCATATCCAAGGCACTATTCTATGCTCCAAGAAAGTTGGCTTGCAGAT
TCGAACTCGAAGTGGTGGTCATGATTCTGAGGGCATGTCCTACATATCTCAAGTCCCATTTGTT
ATAGTAGACTTGAGAAACATGCGTTCAATCAAAATAGATGTTCATAGCCAAACTGCATGGGTTG
AAGCCGGAGCTACCCTTGGAGAAGTTTATTATTGGGTTAATGAGAAAAATGAGAATCTTAGTTT
GGCGGCTGGGTATTGCCCTACTGTTTGCGCAGGTGGACACTTTGGTGGAGGAGGCTATGGACCA
TTGATGAGAAACTATGGCCTCGCGGCTGATAATATCATTGATGCACACTTAGTCAACGTTCATG
GAAAAGTGCTAGATCGAAAATCTATGGGGGAAGATCTCTTTTGGGCTTTACGTGGTGGTGGAGC
AGAAAGCTTCGGAATCATTGTAGCATGGAAAATTAGACTGGTTGCTGTCCCAAAGTCTACTATG
TTTAGTGTTAAAAAGATCATGGAGATACATGAGCTTGTCAAGTTAGTTAACAAATGGCAAAATA
TTGCTTACAAGTATGACAAAGATTTATTACTCATGACTCACTTCATAACTAGGAACATTACAGA
TAATCAAGGGAAGAATAAGACAGCAATACACACTTACTTCTCTTCAGTTTTCCTTGGTGGAGTG
GATAGTCTAGTCGACTTGATGAACAAGAGTTTTCCTGAGTTGGGTATTAAAAAAACGGATTGCA
GACAATTGAGCTGGATTGATACTATCATCTTCTATAGTGGTGTTGTAAATTACGACACTGATAA
TTTTAACAAGGAAATTTTGCTTGATAGATCCGCTGGGCAGAACGGTGCTTTCAAGATTAAGTTA
GACTACGTTAAGAAACCAATTCCAGAATCTGTATTTGTCCAAATTTTGGAAAAATTATATGAAG
AAGATATAGGAGCTGGGATGTATGCGTTGTACCCTTACGGTGGTATAATGGATGAGATTTCAGA
ATCAGCAATTCCATTCCCTCATCGAGCTGGAATCTTGTATGAGTTATGGTACATATGTAGTTGG
GAGAAGCAAGAAGATAACGAAAAGCATCTAAACTGGATTAGAAATATTTATAACTTCATGACTC
CTTATGTGTCCAAAAATCCAAGATTGGCATATCTCAATTATAGAGACCTTGATATAGGAATAAA
TGATCCCAAGAATCCAAATAATTACACACAAGCACGTATTTGGGGTGAGAAGTATTTTGGTAAA
AATTTTGACAGGCTAGTAAAAGTGAAAACCCTGGTTGATCCCAATAACTTTTTTAGAAACGAAC
AAAGCATCCCACCTCTACCACGGCATCGTCATTAA
Amino Acid
Trichome-targeted CBDA synthase
Cannabis
SEQ ID NO. 18
MKCSTFSFWFVCKIIFFFFSFNIQTSIANPRENFLKCFSQYIPNNATNLKLVYTQNNPLYMSVL
NSTIHNLRFTSDTTPKPLVIVTPSHVSHIQGTILCSKKVGLQIRTRSGGHDSEGMSYISQVPFV
IVDLRNMRSIKIDVHSQTAWVEAGATLGEVYYWVNEKNENLSLAAGYCPTVCAGGHFGGGGYGP
LMRNYGLAADNIIDAHLVNVHGKVLDRKSMGEDLFWALRGGGAESFGIIVAWKIRLVAVPKSTM
FSVKKIMEIHELVKLVNKWQNIAYKYDKDLLLMTHFITRNITDNQGKNKTAIHTYFSSVFLGGV
DSLVDLMNKSFPELGIKKTDCRQLSWIDTIIFYSGVVNYDTDNFNKEILLDRSAGQNGAFKIKL
DYVKKPIPESVFVQILEKLYEEDIGAGMYALYPYGGIMDEISESAIPFPHRAGILYELWYICSW
EKQEDNEKHLNWIRNIYNFMTPYVSKNPRLAYLNYRDLDIGINDPKNPNNYTQARIWGEKYFGK
NFDRLVKVKTLVDPNNFFRNEQSIPPLPRHRH
DNA
Trichome-targeted UDP glycosyltransferase 76G1
Stevia rebaudiana
SEQ ID NO. 19
ATGAAGTGCTCAACATTCTCCTTTTGGTTTGTTTGCAAGATAATATTTTTCTTTTTCTCATTCA
ATATCCAAACTTCCATTGCTAATCCTCGAGAAAATAAAACTGAAACTACTGTTAGAAGAAGAAG
AAGAATTATTTTGTTTCCTGTTCCTTTTCAAGGACATATTAATCCTATTTTGCAATTGGCTAAT
GTTTTGTATTCAAAAGGATTTTCAATTACTATTTTTCATACTAATTTTAATAAACCTAAAACTT
CAAATTATCCTCATTTTACTTTTAGATTTATTTTGGATAATGATCCTCAAGATGAAAGAATTTC
AAATTTGCCTACTCATGGACCTTTGGCTGGAATGAGAATTCCTATTATTAATGAACATGGAGCT
GATGAATTGAGAAGAGAATTGGAATTGTTGATGTTGGCTTCAGAAGAAGATGAAGAAGTTTCAT
GCTTGATTACTGATGCTTTGTGGTATTTTGCTCAATCAGTTGCTGATTCATTGAATTTGAGAAG
ATTGGTTTTGATGACTTCATCATTGTTTAATTTTCATGCTCATGTTTCATTGCCTCAATTTGAT
GAATTGGGATATTTGGATCCTGATGATAAAACTAGATTGGAAGAACAAGCTTCAGGATTTCCTA
TGTTGAAAGTTAAAGATATTAAATCAGCTTATTCAAATTGGCAAATTTTGAAAGAAATTTTGGG
AAAAATGATTAAACAAACTAGAGCTTCATCAGGAGTTATTTGGAATTCATTTAAAGAATTGGAA
GAATCAGAATTGGAAACTGTTATTAGAGAAATTCCTGCTCCTTCATTTTTGATTCCTTTGCCTA
AACATTTGACTGCTTCATCATCATCATTGTTGGATCATGATAGAACTGTTTTTCAATGGTTGGA
TCAACAACCTCCTTCATCAGTTTTGTATGTTTCATTTGGATCAACTTCAGAAGTTGATGAAAAA
GATTTTTTGGAAATTGCTAGAGGATTGGTTGATTCAAAACAATCATTTTTGTGGGTTGTTAGAC
CTGGATTTGTTAAAGGATCAACTTGGGTTGAACCTTTGCCTGATGGATTTTTGGGAGAAAGAGG
AAGAATTGTTAAATGGGTTCCTCAACAAGAAGTTTTGGCTCATGGAGCTATTGGAGCTTTTTGG
ACTCATTCAGGATGGAATTCAACTTTGGAATCAGTTTGCGAAGGAGTTCCTATGATTTTTTCAG
ATTTTGGATTGGATCAACCTTTGAATGCTAGATATATGTCAGATGTTTTGAAAGTTGGAGTTTA
TTTGGAAAATGGATGGGAAAGAGGAGAAATTGCTAATGCTATTAGAAGAGTTATGGTTGATGAA
GAAGGAGAATATATTAGACAAAATGCTAGAGTTTTGAAACAAAAAGCTGATGTTTCATTGATGA
AAGGAGGATCATCATATGAATCATTGGAATCATTGGTTTCATATATTTCATCATTGTAA
Amino Acid
Trichome-targeted UDP glycosyltransferase 76G1
Stevia rebaudiana
SEQ ID NO. 20
MKCSTFSFWFVCKIIFFFFSFNIQTSIANPRENKTETTVRRRRRIILFPVPFQGHINPILQLAN
VLYSKGFSIIIFHTNFNKPKTSNYPHFTFRFILDNDPQDERISNLPTHGPLAGMRIPIINEHGA
DELRRELELLMLASEEDEEVSCLITDALWYFAQSVADSLNLRRLVLMTSSLFNFHAHVSLPQFD
ELGYLDPDDKTRLEEQASGFPMLKVKDIKSAYSNWQILKEILGKMIKQTRASSGVIWNSFKELE
ESELETVIREIPAPSFLIPLPKHLTASSSSLLDHDRTVFQWLDQQPPSSVLYVSFGSTSEVDEK
DFLEIARGLVDSKQSFLWVVRPGFVKGSTWVEPLPDGFLGERGRIVKWVPQQEVLAHGAIGAFW
THSGWNSTLESVCEGVPMIFSDFGLDQPLNARYMSDVLKVGVYLENGWERGEIANAIRRVMVDE
EGEYIRQNARVLKQKADVSLMKGGSSYESLESLVSYISSL
DNA
PM-UTR1
Arabidopsis thaliana
SEQ ID NO. 21
ATGGAGGTCCATGGCTCCGGATTCCGTCGAATTCTGTTGTTGGCGTTGTGTATCTCCGGGATCT
GGTCCGCCTACATCTACCAAGGCGTTCTTCAAGAGACTCTGTCCACGAAGAGATTTGGTCCAGA
TGAGAAGAGGTTCGAGCATCTTGCATTCTTGAACTTAGCTCAAAGTGTAGTCTGCTTGATCTGG
TCTTATATAATGATCAAGCTCTGGTCAAATGCTGGTAACGGTGGAGCACCATGGTGGACGTATT
GGAGTGCAGGCATTACTAATACAATTGGTCCTGCCATGGGAATTGAAGCCTTGAAGTATATCAG
TTATCCAGCTCAGGTTTTGGCAAAATCGTCAAAAATGATTCCAGTTATGCTAATGGGAACTTTA
GTTTACGGAATAAGATACACTTTCCCTGAATACATGTGCACCTTTCTTGTCGCTGGAGGAGTAT
CCATCTTTGCTCTTCTTAAGACAAGCTCTAAGACAATTAGCAAGCTAGCACATCCAAATGCTCC
CCTCGGTTACGCACTTTGTTCCTTAAACCTCGCCTTTGACGGATTCACAAATGCCACACAAGAC
TCCATTGCCTCAAGGTACCCAAAAACCGAAGCGTGGGACATAATGCTGGGAATGAACTTATGGG
GCACAATATACAACATTATCTACATGTTTGGCTTGCCACAAGGGATGGATTCGAAGCAATTCAG
TTCTGTAAGCTACACCCGGAAGCGGCATGGGACATTCTAAAGTATTGTATATGCGGTGCCGTGG
GACAAAACTTCATCTTCATGACAATAAGTAACTTCGGGTCACTAGCTAACACGACCATAACCAC
GACCAGGAAGTTTGTTAGCATTGTTGTATCATCAGTAATGAGCGGAAATCCATTGTCGTTGAAG
CAATGGGGATGTGTTTCGATGGTCTTTGGTGGTTTGGCATATCAAATTTATCTTAAATGGAAGA
AATTGCAGAGAGTGGAGTGCTCCATAATGAACTTAATGTGTGGGTCTACCTGCGCCGCTTGA
DNA
Cytostolic CBDA synthase (cytCBDAs)
Cannabis sativa
SEQ ID NO. 22
ATGAATCCTCGAGAAAACTTCCTTAAATGCTTCTCGCAATATATTCCCAATAATGCAACAAATC
TAAAACTCGTATACACTCAAAACAACCCATTGTATATGTCTGTCCTAAATTCGACAATACACAA
TCTTAGATTCACCTCTGACACAACCCCAAAACCACTTGTTATCGTCACTCCTTCACATGTCTCT
CATATCCAAGGCACTATTCTATGCTCCAAGAAAGTTGGCTTGCAGATTCGAACTCGAAGTGGTG
GTCATGATTCTGAGGGCATGTCCTACATATCTCAAGTCCCATTTGTTATAGTAGACTTGAGAAA
CATGCGTTCAATCAAAATAGATGTTCATAGCCAAACTGCATGGGTTGAAGCCGGAGCTACCCTT
GGAGAAGTTTATTATTGGGTTAATGAGAAAAATGAGAATCTTAGTTTGGCGGCTGGGTATTGCC
CTACTGTTTGCGCAGGTGGACACTTTGGTGGAGGAGGCTATGGACCATTGATGAGAAACTATGG
CCTCGCGGCTGATAATATCATTGATGCACACTTAGTCAACGTTCATGGAAAAGTGCTAGATCGA
AAATCTATGGGGGAAGATCTCTTTTGGGCTTTACGTGGTGGTGGAGCAGAAAGCTTCGGAATCA
TTGTAGCATGGAAAATTAGACTGGTTGCTGTCCCAAAGTCTACTATGTTTAGTGTTAAAAAGAT
CATGGAGATACATGAGCTTGTCAAGTTAGTTAACAAATGGCAAAATATTGCTTACAAGTATGAC
AAAGATTTATTACTCATGACTCACTTCATAACTAGGAACATTACAGATAATCAAGGGAAGAATA
AGACAGCAATACACACTTACTTCTCTTCAGTTTTCCTTGGTGGAGTGGATAGTCTAGTCGACTT
GATGAACAAGAGTTTTCCTGAGTTGGGTATTAAAAAAACGGATTGCAGACAATTGAGCTGGATT
GATACTATCATCTTCTATAGTGGTGTTGTAAATTACGACACTGATAATTTTAACAAGGAAATTT
TGCTTGATAGATCCGCTGGGCAGAACGGTGCTTTCAAGATTAAGTTAGACTACGTTAAGAAACC
AATTCCAGAATCTGTATTTGTCCAAATTTTGGAAAAATTATATGAAGAAGATATAGGAGCTGGG
ATGTATGCGTTGTACCCTTACGGTGGTATAATGGATGAGATTTCAGAATCAGCAATTCCATTCC
CTCATCGAGCTGGAATCTTGTATGAGTTATGGTACATATGTAGTTGGGAGAAGCAAGAAGATAA
CGAAAAGCATCTAAACTGGATTAGAAATATTTATAACTTCATGACTCCTTATGTGTCCAAAAAT
CCAAGATTGGCATATCTCAATTATAGAGACCTTGATATAGGAATAAATGATCCCAAGAATCCAA
ATAATTACACACAAGCACGTATTTGGGGTGAGAAGTATTTTGGTAAAAATTTTGACAGGCTAGT
AAAAGTGAAAACCCTGGTTGATCCCAATAACTTTTTTAGAAACGAACAAAGCATCCCACCTCTA
CCACGGCATCGTCATTAA
Amino Acid
Cytostolic CBDA synthase (cytCBDAs)
Cannabis sativa
SEQ ID NO. 23
MNPRENFLKCFSQYIPNNATNLKLVYTQNNPLYMSVLNSTIHNLRFTSDTTPKPLVIVTPSHVS
HIQGTILCSKKVGLQIRTRSGGHDSEGMSYISQVPFVIVDLRNMRSIKIDVHSQTAWVEAGATL
GEVYYWVNEKNENLSLAAGYCPTVCAGGHFGGGGYGPLMRNYGLAADNIIDAHLVNVHGKVLDR
KSMGEDLFWALRGGGAESFGIIVAWKIRLVAVPKSTMFSVKKIMEIHELVKLVNKWQNIAYKYD
KDLLLMTHFITRNITDNQGKNKTAIHTYFSSVFLGGVDSLVDLMNKSFPELGIKKTDCRQLSWI
DTIIFYSGVVNYDTDNFNKEILLDRSAGQNGAFKIKLDYVKKPIPESVFVQILEKLYEEDIGAG
MYALYPYGGIMDEISESAIPFPHRAGILYELWYICSWEKQEDNEKHLNWIRNIYNFMTPYVSKN
PRLAYLNYRDLDIGINDPKNPNNYTQARIWGEKYFGKNFDRLVKVKTLVDPNNFFRNEQSIPPL
PRHRH
DNA
Cytostolic-targeted UDP glycosyltransferase 76G1 (cytUTG)
Stevia rebaudiana
SEQ ID NO. 24
ATGGAAAATAAAACCGAAACCACCGTCCGCCGTCGTCGCCGTATCATTCTGTTCCCGGTCCCGT
TCCAGGGCCACATCAACCCGATTCTGCAACTGGCGAACGTGCTGTATTCGAAAGGTTTCAGCAT
CACCATCTTCCATACGAACTTCAACAAGCCGAAGACCAGCAATTACCCGCACTTTACGTTCCGT
TTTATTCTGGATAACGACCCGCAGGATGAACGCATCTCTAATCTGCCGACCCACGGCCCGCTGG
CGGGTATGCGTATTCCGATTATCAACGAACACGGCGCAGATGAACTGCGTCGCGAACTGGAACT
GCTGATGCTGGCCAGCGAAGAAGATGAAGAAGTTTCTTGCCTGATCACCGACGCACTGTGGTAT
TTTGCCCAGTCTGTTGCAGATAGTCTGAACCTGCGTCGCCTGGTCCTGATGACCAGCAGCCTGT
TCAATTTTCATGCCCACGTTAGTCTGCCGCAGTTCGATGAACTGGGTTATCTGGACCCGGATGA
CAAAACCCGCCTGGAAGAACAGGCGAGCGGCTTTCCGATGCTGAAAGTCAAGGATATTAAGTCA
GCGTACTCGAACTGGCAGATTCTGAAAGAAATCCTGGGTAAAATGATTAAGCAAACCAAAGCAA
GTTCCGGCGTCATCTGGAATAGTTTCAAAGAACTGGAAGAATCCGAACTGGAAACGGTGATTCG
TGAAATCCCGGCTCCGAGTTTTCTGATTCCGCTGCCGAAGCATCTGACCGCGAGCAGCAGCAGC
CTGCTGGATCACGACCGCACGGTGTTTCAGTGGCTGGATCAGCAACCGCCGAGTTCCGTGCTGT
ATGTTAGCTTCGGTAGTACCTCGGAAGTGGATGAAAAGGACTTTCTGGAAATCGCTCGTGGCCT
GGTTGATAGCAAACAATCTTTCCTGTGGGTGGTTCGCCCGGGTTTTGTGAAGGGCTCTACGTGG
GTTGAACCGCTGCCGGACGGCTTCCTGGGTGAACGTGGCCGCATTGTCAAATGGGTGCCGCAGC
AAGAAGTGCTGGCGCATGGCGCGATTGGCGCGTTTTGGACCCACTCCGGTTGGAACTCAACGCT
GGAATCGGTTTGTGAAGGTGTCCCGATGATTTTCTCAGATTTTGGCCTGGACCAGCCGCTGAAT
GCACGTTATATGTCGGATGTTCTGAAAGTCGGTGTGTACCTGGAAAACGGTTGGGAACGCGGCG
AAATTGCGAATGCCATCCGTCGCGTTATGGTCGATGAAGAAGGCGAATACATTCGTCAGAATGC
TCGCGTCCTGAAACAAAAGGCGGACGTGAGCCTGATGAAAGGCGGTTCATCGTATGAAAGTCTG
GAATCCCTGGTTTCATACATCAGCTCTCTGTAA
Amino Acid
Cytostolic-targeted UDP glycosyltransferase 76G1 (cytUTG)
Stevia rebaudiana
SEQ ID NO. 25
MENKTETTVRRRRRIILFPVPFQGHINPILQLANVLYSKGFSITIFHTNFNKPKTSNYPHFTFR
FILDNDPQDERISNLPTHGPLAGMRIPIINEHGADELRRELELLMLASEEDEEVSCLITDALWY
FAQSVADSLNLRRLVLMTSSLFNFHAHVSLPQFDELGYLDPDDKTRLEEQASGFPMLKVKDIKS
AYSNWQILKEILGKMIKQTKASSGVIWNSFKELEESELETVIREIPAPSFLIPLPKHLTASSSS
LLDHDRTVFQWLDQQPPSSVLYVSFGSTSEVDEKDFLEIARGLVDSKQSFLWVVRPGFVKGSTW
VEPLPDGFLGERGRIVKWVPQQEVLAHGAIGAFWTHSGWNSTLESVCEGVPMIFSDFGLDQPLN
ARYMSDVLKVGVYLENGWERGEIANAIRRVMVDEEGEYIRQNARVLKQKADVSLMKGGSSYESL
ESLVSYISSL
Amino Acid
Glycosyltransferase (NtGT5a)
Nicotiana tabacum
SEQ ID NO. 26
MGSIGAELTKPHAVCIPYPAQGHINPMLKLAKILHHKGFHITFVNTEFNHRRLLKSRGPDSLKG
LSSFRFETIPDGLPPCEADATQDIPSLCESTTNTCLAPFRDLLAKLNDTNTSNVPPVSCIVSDG
VMSFTLAAAQELGVPEVLFWTTSACGFLGYMHYCKVIEKGYAPLKDASDLTNGYLETTLDFIPG
MKDVRLRDLPSFLRTTNPDEFMIKFVLQETERARKASAIILNTFETLEAEVLESLRNLLPPVYP
IGPLHFLVKHVDDENLKGLRSSLWKEEPECIQWLDTKEPNSVVYVNFGSITVMTPNQLIEFAWG
LANSQQTFLWIIRPDIVSGDASILPPEFVEETKNRGMLASWCSQEEVLSHPAIVGFLTHSGWNS
TLESISSGVPMICWPFFAEQQTNCWFSVTKWDVGMEIDSDVKRDEVESLVRELMVGGKGKKMKK
KAMEWKELAEASAKEHSGSSYVNIEKLVNDILLSSKH
DNA
Glycosyltransferase (NtGT5a)
Nicotiana tabacum
SEQ ID NO. 27
ATGGGTTCCATTGGTGCTGAATTAACAAAGCCACATGCAGTTTGCATACCATATCCCGCCCAAG
GCCATATTAACCCCATGTTAAAGCTAGCCAAAATCCTTCATCACAAAGGCTTTCACATCACTTT
TGTCAATACTGAATTTAACCACCGACGTCTCCTTAAATCTCGTGGCCCTGATTCTCTCAAGGGT
CTTTCTTCTTTCCGTTTTGAGACCATTCCTGATGGACTTCCGCCATGTGAGGCAGATGCCACAC
AAGATATACCTTCTTTGTGTGAATCTACAACCAATACTTGCTTGGCTCCTTTTAGGGATCTTCT
TGCGAAACTCAATGATACTAACACATCTAACGTGCCACCCGTTTCGTGCATCGTCTCGGATGGT
GTCATGAGCTTCACCTTAGCCGCTGCACAAGAATTGGGAGTCCCTGAAGTTCTGTTTTGGACCA
CTAGTGCTTGTGGTTTCTTAGGTTACATGCATTACTGCAAGGTTATTGAAAAAGGATATGCTCC
ACTTAAAGATGCGAGTGACTTGACAAATGGATACCTAGAGACAACATTGGATTTTATACCAGGC
ATGAAAGACGTACGTTTAAGGGATCTTCCAAGTTTCTTGAGAACTACAAATCCAGATGAATTCA
TGATCAAATTTGTCCTCCAAGAAACAGAGAGAGCAAGAAAGGCTTCTGCAATTATCCTCAACAC
ATTTGAAACACTAGAGGCTGAAGTTCTTGAATCGCTCCGAAATCTTCTTCCTCCAGTCTACCCC
ATAGGGCCCTTGCATTTTCTAGTGAAACATGTTGATGATGAGAATTTGAAGGGACTTAGATCCA
GCCTTTGGAAAGAGGAACCAGAGTGTATACAATGGCTTGATACCAAAGAACCAAATTCTGTTGT
TTATGTTAACTTTGGAAGCATTACTGTTATGACTCCTAATCAGCTTATTGAGTTTGCTTGGGGA
CTTGCAAACAGCCAGCAAACATTCTTATGGATCATAAGACCTGATATTGTTTCAGGTGATGCAT
CGATTCTTCCACCCGAATTCGTGGAAGAAACGAAGAACAGAGGTATGCTTGCTAGTTGGTGTTC
ACAAGAAGAAGTACTTAGTCACCCTGCAATAGTAGGATTCTTGACTCACAGTGGATGGAATTCG
ACACTCGAAAGTATAAGCAGTGGGGTGCCTATGATTTGCTGGCCATTTTTCGCTGAACAGCAAA
CAAATTGTTGGTTTTCCGTCACTAAATGGGATGTTGGAATGGAGATTGACAGTGATGTGAAGAG
AGATGAAGTGGAAAGCCTTGTAAGGGAATTGATGGTTGGGGGAAAAGGCAAAAAGATGAAGAAA
AAGGCAATGGAATGGAAGGAATTGGCTGAAGCATCTGCTAAAGAACATTCAGGGTCATCTTATG
TGAACATTGAAAAGTTGGTCAATGATATTCTTCTTTCATCCAAACATTAA
Amino Acid
Glycosyltransferase (NtGT5b)
Nicotiana tabacum
SEQ ID NO. 28
MGSIGAEFTKPHAVCIPYPAQGHINPMLKLAKILHHKGFHITFVNTEFNHRRLLKSRGPDSLKG
LSSFRFETIPDGLPPCDADATQDIPSLCESTTNTCLGPFRDLLAKLNDTNTSNVPPVSCIISDG
VMSFTLAAAQELGVPEVLFWTTSACGFLGYMHYYKVIEKGYAPLKDASDLTNGYLETTLDFIPC
MKDVRLRDLPSFLRTTNPDEFMIKFVLQETERARKASAIILNTYETLEAEVLESLRNLLPPVYP
IGPLHFLVKHVDDENLKGLRSSLWKEEPECIQWLDTKEPNSVVYVNFGSITVMTPNQLIEFAWG
LANSQQSFLWIIRPDIVSGDASILPPEFVEETKKRGMLASWCSQEEVLSHPAIGGFLTHSGWNS
TLESISSGVPMICWPFFAEQQTNCWFSVTKWDVGMEIDCDVKRDEVESLVRELMVGGKGKKMKK
KAMEWKELAEASAKEHSGSSYVNIEKVVNDILLSSKH
DNA
Glycosyltransferase (NtGT5b)
Nicotiana tabacum
SEQ ID NO. 29
ATGGGTTCCATTGGTGCTGAATTTACAAAGCCACATGCAGTTTGCATACCATATCCCGCCCAAG
GCCATATTAACCCCATGTTAAAGCTAGCCAAAATCCTTCATCACAAAGGCTTTCACATCACTTT
TGTCAATACTGAATTTAACCACAGACGTCTGCTTAAATCTCGTGGCCCTGATTCTCTCAAGGGT
CTTTCTTCTTTCCGTTTTGAGACAATTCCTGATGGACTTCCGCCATGTGATGCAGATGCCACAC
AAGATATACCTTCTTTGTGTGAATCTACAACCAATACTTGCTTGGGTCCTTTTAGGGATCTTCT
TGCGAAACTCAATGATACTAACACATCTAACGTGCCACCCGTTTCGTGCATCATCTCAGATGGT
GTCATGAGCTTCACCTTAGCCGCTGCACAAGAATTGGGAGTCCCTGAAGTTCTGTTTTGGACCA
CTAGTGCTTGTGGTTTCTTAGGTTACATGCATTATTACAAGGTTATTGAAAAAGGATACGCTCC
ACTTAAAGATGCGAGTGACTTGACAAATGGATACCTAGAGACAACATTGGATTTTATACCATGC
ATGAAAGACGTACGTTTAAGGGATCTTCCAAGTTTCTTGAGAACTACAAATCCAGATGAATTCA
TGATCAAATTTGTCCTCCAAGAAACAGAGAGAGCAAGAAAGGCTTCTGCAATTATCCTCAACAC
ATATGAAACACTAGAGGCTGAAGTTCTTGAATCGCTCCGAAATCTTCTTCCTCCAGTCTACCCC
ATTGGGCCCTTGCATTTTCTAGTGAAACATGTTGATGATGAGAATTTGAAGGGACTTAGATCCA
GCCTTTGGAAAGAGGAACCAGAGTGTATACAATGGCTTGATACCAAAGAACCAAATTCTGTTGT
TTATGTTAACTTTGGAAGCATTACTGTTATGACTCCTAATCAACTTATTGAATTTGCTTGGGGA
CTTGCAAACAGCCAACAATCATTCTTATGGATCATAAGACCTGATATTGTTTCAGGTGATGCAT
CGATTCTTCCCCCCGAATTCGTGGAAGAAACGAAGAAGAGAGGTATGCTTGCTAGTTGGTGTTC
ACAAGAAGAAGTACTTAGTCACCCTGCAATAGGAGGATTCTTGACTCACAGTGGATGGAATTCG
ACACTCGAAAGTATAAGCAGTGGGGTGCCTATGATTTGCTGGCCATTTTTCGCTGAACAGCAAA
CAAATTGTTGGTTTTCCGTCACTAAATGGGATGTTGGAATGGAGATTGACTGTGATGTGAAGAG
GGATGAAGTGGAAAGCCTTGTAAGGGAATTGATGGTTGGGGGAAAAGGCAAAAAGATGAAGAAA
AAGGCAATGGAATGGAAGGAATTGGCTGAAGCATCTGCTAAAGAACATTCAGGGTCATCTTATG
TGAACATTGAGAAGGTGGTCAATGATATTCTTCTTTCGTCCAAACATTAA
Amino Acid
UDP-glycosyltransferase 73C3 (NtGT4)
Nicotiana tabacum
SEQ ID NO. 30
MATQVHKLHFILFPLMAPGHMIPMIDIAKLLANRGVITTIITTPVNANRFSSTITRAIKSGLRI
QILTLKFPSVEVGLPEGCENIDMLPSLDLASKFFAAISMLKQQVENLLEGINPSPSCVISDMGF
PWTTQIAQNFNIPRIVFHGTCCFSLLCSYKILSSNILENITSDSEYFVVPDLPDRVELTKAQVS
GSTKNTTSVSSSVLKEVTEQIRLAEESSYGVIVNSFEELEQVYEKEYRKARGKKVWCVGPVSLC
NKEIEDLVTRGNKTAIDNQDCLKWLDNFETESVVYASLGSLSRLTLLQMVELGLGLEESNRPFV
WVLGGGDKLNDLEKWILENGFEQRIKERGVLIRGWAPQVLILSHPAIGGVLTHCGWNSTLEGIS
AGLPMVTWPLFAEQFCNEKLVVQVLKIGVSLGVKVPVKWGDEENVGVLVKKDDVKKALDKLMDE
GEEGQVRRTKAKELGELAKKAFGEGGSSYVNLTSLIEDIIEQQNHKEK
DNA
UDP-glycosyltransferase 73C3 (NtGT4)
Nicotiana tabacum
SEQ ID NO. 31
ATGGCAACTCAAGTGCACAAACTTCATTTCATACTATTCCCTTTAATGGCTCCAGGCCACATGA
TTCCTATGATAGACATAGCTAAACTTCTAGCAAATCGCGGTGTCATTACCACTATCATCACCAC
TCCAGTAAACGCCAATCGTTTCAGTTCAACAATTACTCGTGCCATAAAATCCGGTCTAAGAATC
CAAATTCTTACACTCAAATTTCCAAGTGTAGAAGTAGGATTACCAGAAGGTTGCGAAAATATTG
ACATGCTTCCTTCTCTTGACTTGGCTTCAAAGTTTTTTGCTGCAATTAGTATGCTGAAACAACA
AGTTGAAAATCTCTTAGAAGGAATAAATCCAAGTCCAAGTTGTGTTATTTCAGATATGGGATTT
CCTTGGACTACTCAAATTGCACAAAATTTTAATATCCCAAGAATTGTTTTTCATGGTACTTGTT
GTTTCTCACTTTTATGTTCCTATAAAATACTTTCCTCCAACATTCTTGAAAATATAACCTCAGA
TTCAGAGTATTTTGTTGTTCCTGATTTACCCGATAGAGTTGAACTAACGAAAGCTCAGGTTTCA
GGATCGACGAAAAATACTACTTCTGTTAGTTCTTCTGTATTGAAAGAAGTTACTGAGCAAATCA
GATTAGCCGAGGAATCATCATATGGTGTAATTGTTAATAGTTTTGAGGAGTTGGAGCAAGTGTA
TGAGAAAGAATATAGGAAAGCTAGAGGGAAAAAAGTTTGGTGTGTTGGTCCTGTTTCTTTGTGT
AATAAGGAAATTGAAGATTTGGTTACAAGGGGTAATAAAACTGCAATTGATAATCAAGATTGCT
TGAAATGGTTAGATAATTTTGAAACAGAATCTGTGGTTTATGCAAGTCTTGGAAGTTTATCTCG
TTTGACATTATTGCAAATGGTGGAACTTGGTCTTGGTTTAGAAGAGTCAAATAGGCCTTTTGTA
TGGGTATTAGGAGGAGGTGATAAATTAAATGATTTAGAGAAATGGATTCTTGAGAATGGATTTG
AGCAAAGAATTAAAGAAAGAGGAGTTTTGATTAGAGGATGGGCTCCTCAAGTGCTTATACTTTC
ACACCCTGCAATTGGTGGAGTATTGACTCATTGCGGATGGAATTCTACATTGGAAGGTATTTCA
GCAGGATTACCAATGGTAACATGGCCACTATTTGCTGAGCAATTTTGCAATGAGAAGTTAGTAG
TCCAAGTGCTAAAAATTGGAGTGAGCCTAGGTGTGAAGGTGCCTGTCAAATGGGGAGATGAGGA
AAATGTTGGAGTTTTGGTAAAAAAGGATGATGTTAAGAAAGCATTAGACAAACTAATGGATGAA
GGAGAAGAAGGACAAGTAAGAAGAACAAAAGCAAAAGAGTTAGGAGAATTGGCTAAAAAGGCAT
TTGGAGAAGGTGGTTCTTCTTATGTTAACTTAACATCTCTGATTGAAGACATCATTGAGCAACA
AAATCACAAGGAAAAATAG
Amino Acid
Glycosyltransferase (NtGT1b)
Nicotiana tabacum
SEQ ID NO. 32
MKTAELVFIPAPGMGHLVPTVEVAKQLVDRHEQLSITVLIMTIPLETNIPSYTKSLSSDYSSRI
TLLPLSQPETSVTMSSFNAINFFEYISSYKGRVKDAVSETSFSSSNSVKLAGFVIDMFCTAMID
VANEFGIPSYVFYTSSAAMLGLQLHFQSLSIECSPKVHNYVEPESEVLISTYMNPVPVKCLPGI
ILVNDESSTMFVNHARRFRETKGIMVNTFTELESHALKALSDDEKIPPIYPVGPILNLENGNED
HNQEYDAIMKWLDEKPNSSVVFLCFGSKGSFEEDQVKEIANALESSGYHFLWSLRRPPPKDKLQ
FPSEFENPEEVLPEGFFQRTKGRGKVIGWAPQLAILSHPSVGGFVSHCGWNSTLESVRSGVPIA
TWPLYAEQQSNAFQLVKDLGMAVEIKMDYREDFNTRNPPLVKAEEIEDGIRKLMDSENKIRAKV
TEMKDKSRAALLEGGSSYVALGHFVETVMKN
DNA
Glycosyltransferase (NtGT1b)
Nicotiana tabacum
SEQ ID NO. 33
ATGAAGACAGCAGAGTTAGTATTCATTCCTGCTCCTGGGATGGGTCACCTTGTACCAACTGTGG
AGGTGGCAAAGCAACTAGTCGACAGACACGAGCAGCTTTCGATCACAGTTCTAATCATGACAAT
TCCTTTGGAAACAAATATTCCATCATATACTAAATCACTGTCCTCAGACTACAGTTCTCGTATA
ACGCTGCTTCCACTCTCTCAACCTGAGACCTCTGTTACTATGAGCAGTTTTAATGCCATCAATT
TTTTTGAGTACATCTCCAGCTACAAGGGTCGTGTCAAAGATGCTGTTAGTGAAACCTCCTTTAG
TTCGTCAAATTCTGTGAAACTTGCAGGATTTGTAATAGACATGTTCTGCACTGCGATGATTGAT
GTAGCGAACGAGTTTGGAATCCCAAGTTATGTGTTCTACACTTCTAGTGCAGCTATGCTTGGAC
TACAACTGCATTTTCAAAGTCTTAGCATTGAATGCAGTCCGAAAGTTCATAACTACGTTGAACC
TGAATCAGAAGTTCTGATCTCAACTTACATGAATCCGGTTCCAGTCAAATGTTTGCCCGGAATT
ATACTAGTAAATGATGAAAGTAGCACCATGTTTGTCAATCATGCACGAAGATTCAGGGAGACGA
AAGGAATTATGGTGAACACGTTCACTGAGCTTGAATCACACGCTTTGAAAGCCCTTTCCGATGA
TGAAAAAATCCCACCAATCTACCCAGTTGGACCTATACTTAACCTTGAAAATGGGAATGAAGAT
CACAATCAAGAATATGATGCGATTATGAAGTGGCTTGACGAGAAGCCTAATTCATCAGTGGTGT
TCTTATGCTTTGGAAGCAAGGGGTCTTTCGAAGAAGATCAGGTGAAGGAAATAGCAAATGCTCT
AGAGAGCAGTGGCTACCACTTCTTGTGGTCGCTAAGGCGACCGCCACCAAAAGACAAGCTACAA
TTCCCAAGCGAATTCGAGAATCCAGAGGAAGTCTTACCAGAGGGATTCTTTCAAAGGACTAAAG
GAAGAGGAAAGGTGATAGGATGGGCACCCCAGTTGGCTATTTTGTCTCATCCTTCAGTAGGAGG
ATTCGTGTCGCATTGTGGGTGGAATTCAACTCTGGAGAGCGTTCGAAGTGGAGTGCCGATAGCA
ACATGGCCATTGTATGCAGAGCAACAGAGCAATGCATTTCAACTGGTGAAGGATTTGGGTATGG
CAGTAGAGATTAAGATGGATTACAGGGAAGATTTTAATACGAGAAATCCACCACTGGTTAAAGC
TGAGGAGATAGAAGATGGAATTAGGAAGCTGATGGATTCAGAGAATAAAATCAGGGCTAAGGTG
ACGGAGATGAAGGACAAAAGTAGAGCAGCACTGCTGGAGGGCGGATCATCATATGTAGCTCTTG
GGCATTTTGTTGAGACTGTCATGAAAAACTAG
Amino Acid
Glycosyltransferase (NtGT1a)
Nicotiana tabacum
SEQ ID NO. 34
MKTTELVFIPAPGMGHLVPTVEVAKQLVDRDEQLSITVLIMTLPLETNIPSYTKSLSSDYSSRI
TLLQLSQPETSVSMSSFNAINFFEYISSYKDRVKDAVNETFSSSSSVKLKGFVIDMFCTAMIDV
ANEFGIPSYVFYTSNAAMLGLQLHFQSLSIEYSPKVHNYLDPESEVAISTYINPIPVKCLPGII
LDNDKSGTMFVNHARRFRETKGIMVNTFAELESHALKALSDDEKIPPIYPVGPILNLGDGNEDH
NQEYDMIMKWLDEQPHSSVVFLCFGSKGSFEEDQVKEIANALERSGNRFLWSLRRPPPKDTLQF
PSEFENPEEVLPVGFFQRTKGRGKVIGWAPQLAILSHPAVGGFVSHCGWNSTLESVRSGVPIAT
WPLYAEQQSNAFQLVKDLGMAVEIKMDYREDFNKTNPPLVKAEEIEDGIRKLMDSENKIRAKVM
EMKDKSRAALLEGGSSYVALGHFVETVMKN
DNA
Glycosyltransferase (NtGT1a)
Nicotiana tabacum
SEQ ID NO. 35
ATGAAGACAACAGAGTTAGTATTCATTCCTGCTCCTGGCATGGGTCACCTTGTACCCACTGTGG
AGGTGGCAAAGCAACTAGTCGACAGAGACGAACAGCTTTCAATCACAGTTCTCATCATGACGCT
TCCTTTGGAAACAAATATTCCATCATATACTAAATCACTGTCCTCAGACTACAGTTCTCGTATA
ACGCTGCTTCAACTTTCTCAACCTGAGACCTCTGTTAGTATGAGCAGTTTTAATGCCATCAATT
TTTTTGAGTACATCTCCAGCTACAAGGATCGTGTCAAAGATGCTGTTAATGAAACCTTTAGTTC
GTCAAGTTCTGTGAAACTCAAAGGATTTGTAATAGACATGTTCTGCACTGCGATGATTGATGTG
GCGAACGAGTTTGGAATCCCAAGTTATGTCTTCTACACTTCTAATGCAGCTATGCTTGGACTCC
AACTCCATTTTCAAAGTCTTAGTATTGAATACAGTCCGAAAGTTCATAATTACCTAGACCCTGA
ATCAGAAGTAGCGATCTCAACTTACATTAATCCGATTCCAGTCAAATGTTTGCCCGGGATTATA
CTAGACAATGATAAAAGTGGCACCATGTTCGTCAATCATGCACGAAGATTCAGG
GAGACGAAAGGAATTATGGTGAACACATTCGCTGAGCTTGAATCACACGCTTTGAAAGCCCTTT
CCGATGATGAGAAAATCCCACCAATCTACCCAGTTGGGCCTATACTTAACCTTGGAGATGGGAA
TGAAGATCACAATCAAGAATATGATATGATTATGAAGTGGCTCGACGAGCAGCCTCATTCATCA
GTGGTGTTCCTATGCTTTGGAAGCAAGGGATCTTTCGAAGAAGATCAAGTGAAGGAAATAGCAA
ATGCTCTAGAGAGAAGTGGTAACCGGTTCTTGTGGTCGCTAAGACGACCGCCACCAAAAGACAC
GCTACAATTCCCAAGCGAATTCGAGAATCCAGAGGAAGTCTTGCCGGTGGGATTCTTTCAAAGG
ACTAAAGGAAGAGGAAAGGTGATAGGATGGGCACCCCAGTTGGCTATTTTGTCTCATCCTGCAG
TAGGAGGATTCGTGTCGCATTGTGGGTGGAATTCAACTTTGGAGAGTGTTCGTAGTGGAGTACC
GATAGCAACATGGCCATTGTATGCAGAGCAACAGAGCAATGCATTTCAACTGGTGAAGGATTTG
GGGATGGCAGTGGAGATTAAGATGGATTACAGGGAAGATTTTAATAAGACAAATCCACCACTGG
TTAAAGCTGAGGAGATAGAAGATGGAATTAGGAAGCTGATGGATTCAGAGAATAAAATCAGGGC
TAAGGTGATGGAGATGAAGGACAAAAGTAGAGCAGCGTTATTAGAAGGCGGATCATCATATGTA
GCTCTCGGGCATTTTGTTGAGACTGTCATGAAAAACTAA
Amino Acid
Glycosyltransferase (NtGT3)
Nicotiana tabacum
SEQ ID NO. 36
MKETKKIELVFIPSPGIGHLVSTVEMAKLLIAREEQLSITVLIIQWPNDKKLDSYIQSVANFSS
RLKFIRLPQDDSIMQLLKSNIFTTFIASHKPAVRDAVADILKSESNNTLAGIVIDLFCTSMIDV
ANEFELPTYVFYTSGAATLGLHYHIQNLRDEFNKDITKYKDEPEEKLSIATYLNPFPAKCLPSV
ALDKEGGSTMFLDLAKRFRETKGIMINTFLELESYALNSLSRDKNLPPIYPVGPVLNLNNVEGD
NLGSSDQNTMKWLDDQPASSVVFLCFGSGGSFEKHQVKEIAYALESSGCRFLWSLRRPPTEDAR
FPSNYENLEEILPEGFLERTKGIGKVIGWAPQLAILSHKSTGGFVSHCGWNSTLESTYFGVPIA
TWPMYAEQQANAFQLVKDLRMGVEIKMDYRKDMKVMGKEVIVKAEEIEKAIREIMDSESEIRVK
VKEMKEKSRAAQMEGGSSYTSIGGFIQIIMENSQ
DNA
Glycosyltransferase (NtGT3)
Nicotiana tabacum
SEQ ID NO. 37
ATGAAAGAAACCAAGAAAATAGAGTTAGTCTTCATTCCTTCACCAGGAATTGGCCATTTAGTAT
CCACAGTTGAAATGGCAAAGCTTCTTATAGCTAGAGAAGAGCAGCTATCTATCACAGTCCTCAT
CATCCAATGGCCTAACGACAAGAAGCTCGATTCTTATATCCAATCAGTCGCCAATTTCAGCTCG
CGTTTGAAATTCATTCGACTCCCTCAGGATGATTCCATTATGCAGCTACTCAAAAGCAACATTT
TCACCACGTTTATTGCCAGTCATAAGCCTGCAGTTAGAGATGCTGTTGCTGATATTCTCAAGTC
AGAATCAAATAATACGCTAGCAGGTATTGTTATCGACTTGTTCTGCACCTCAATGATAGACGTG
GCCAATGAGTTCGAGCTACCAACCTATGTTTTCTACACGTCTGGTGCAGCAACCCTTGGTCTTC
ATTATCATATACAGAATCTCAGGGATGAATTTAACAAAGATATTACCAAGTACAAAGACGAACC
TGAAGAAAAACTCTCTATAGCAACATATCTCAATCCATTTCCAGCAAAATGTTTGCCGTCTGTA
GCCTTAGACAAAGAAGGTGGTTCAACAATGTTTCTTGATCTCGCAAAAAGGTTTCGAGAAACCA
AAGGTATTATGATAAACACATTTCTAGAGCTCGAATCCTATGCATTAAACTCGCTCTCACGAGA
CAAGAATCTTCCACCTATATACCCTGTCGGACCAGTATTGAACCTTAACAATGTTGAAGGTGAC
AACTTAGGTTCATCTGACCAGAATACTATGAAATGGTTAGATGATCAGCCCGCTTCATCTGTAG
TGTTCCTTTGTTTTGGTAGTGGTGGAAGCTTTGAAAAACATCAAGTTAAGGAAATAGCCTATGC
TCTGGAGAGCAGTGGGTGTCGGTTTTTGTGGTCGTTAAGGCGACCACCAACCGAAGATGCAAGA
TTTCCAAGCAACTATGAAAATCTTGAAGAAATTTTGCCAGAAGGATTCTTGGAAAGAACAAAAG
GGATTGGAAAAGTGATAGGATGGGCACCTCAGTTGGCGATTTTGTCACATAAATCGACGGGGGG
ATTTGTGTCGCACTGTGGATGGAATTCGACTTTGGAAAGTACATATTTTGGAGTGCCAATAGCA
ACCTGGCCAATGTACGCGGAGCAACAAGCGAATGCATTTCAATTGGTTAAGGATTTGAGAATGG
GAGTTGAGATTAAGATGGATTATAGGAAGGATATGAAAGTGATGGGCAAAGAAGTTATAGTGAA
AGCTGAGGAGATTGAGAAAGCAATAAGAGAAATTATGGATTCCGAGAGTGAAATTCGGGTGAAG
GTGAAAGAGATGAAGGAGAAGAGCAGAGCAGCACAAATGGAAGGTGGCTCTTCTTACACTTCTA
TTGGAGGTTTCATCCAAATTATCATGGAGAATTCTCAATAA
Amino Acid
Glycosyltransferase (NtGT2)
Nicotiana tabacum
SEQ ID NO. 38
MVQPHVLLVTFPAQGHINPCLQFAKRLIRMGIEVTFATSVFAHRRMAKTTTSTLSKGLNFAAFS
DGYDDGFKADEHDSQHYMSEIKSRGSKTLKDIILKSSDEGRPVTSLVYSLLLPWAAKVAREFHI
PCALLWIQPATVLDIYYYYFNGYEDAIKGSTNDPNWCIQLPRLPLLKSQDLPSFLLSSSNEEKY
SFALPTFKEQLDTLDVEENPKVLVNTFDALEPKELKAIEKYNLIGIGPLIPSTFLDGKDPLDSS
FGGDLFQKSNDYIEWLNSKANSSVVYISFGSLLNLSKNQKEEIAKGLIEIKKPFLWVIRDQENG
KGDEKEEKLSCMMELEKQGKIVPWCSQLEVLTHPSIGCFVSHCGWNSTLESLSSGVSVVAFPHW
TDQGTNAKLIEDVWKTGVRLKKNEDGVVESEEIKRCIEMVMDGGEKGEEMRRNAQKWKELAREA
VKEGGSSEMNLKAFVQEVGKGC
DNA
Glycosyltransferase (NtGT2)
Nicotiana tabacum
SEQ ID NO. 39
ATGGTGCAACCCCATGTCCTCTTGGTGACTTTTCCAGCACAAGGCCATATTAATCCATGTCTCC
AATTTGCCAAGAGGCTAATTAGAATGGGCATTGAGGTAACTTTTGCCACGAGCGTTTTCGCCCA
TCGTCGTATGGCAAAAACTACGACTTCCACTCTATCCAAGGGCTTAAATTTTGCGGCATTCTCT
GATGGGTACGACGATGGTTTCAAGGCCGATGAGCATGATTCTCAACATTACATGTCGGAGATAA
AAAGTCGCGGTTCTAAAACCCTAAAAGATATCATTTTGAAGAGCTCAGACGAGGGACGTCCTGT
GACATCCCTCGTCTATTCTCTTTTGCTTCCATGGGCTGCAAAGGTAGCGCGTGAATTTCACATA
CCGTGCGCGTTACTATGGATTCAACCAGCAACTGTGCTAGACATATATTATTATTACTTCAATG
GCTATGAGGATGCCATAAAAGGTAGCACCAATGATCCAAATTGGTGTATTCAATTGCCTAGGCT
TCCACTACTAAAAAGCCAAGATCTTCCTTCTTTTTTACTTTCTTCTAGTAATGAAGAAAAATAT
AGCTTTGCTCTACCAACATTTAAAGAGCAACTTGACACATTAGATGTTGAAGAAAATCCTAAAG
TACTTGTGAACACATTTGATGCATTAGAGCCAAAGGAACTCAAAGCTATTGAAAAGTACAATTT
AATTGGGATTGGACCATTGATTCCTTCAACATTTTTGGACGGAAAAGACCCTTTGGATTCTTCC
TTTGGTGGTGATCTTTTTCAAAAGTCTAATGACTATATTGAATGGTTGAACTCAAAGGCTAACT
CATCTGTGGTTTATATCTCATTTGGGAGTCTCTTGAATTTGTCAAAAAATCAAAAGGAGGAGAT
TGCAAAAGGGTTGATAGAGATTAAAAAGCCATTCTTGTGGGTAATAAGAGATCAAGAAAATGGT
AAGGGAGATGAAAAAGAAGAGAAATTAAGTTGTATGATGGAGTTGGAAAAGCAAGGGAAAATAG
TACCATGGTGTTCACAACTTGAAGTCTTAACACATCCATCTATAGGATGTTTCGTGTCACATTG
TGGATGGAATTCGACTCTGGAAAGTTTATCGTCAGGCGTGTCAGTAGTGGCATTTCCTCATTGG
ACGGATCAAGGGACAAATGCTAAACTAATTGAAGATGTTTGGAAGACAGGTGTAAGGTTGAAAA
AGAATGAAGATGGTGTGGTTGAGAGTGAAGAGATAAAAAGGTGCATAGAAATGGTAATGGATGG
TGGAGAGAAAGGAGAAGAAATGAGAAGAAATGCTCAAAAATGGAAAGAATTGGCAAGGGAAGCT
GTAAAAGAAGGCGGATCTTCGGAAATGAATCTAAAAGCTTTTGTTCAAGAAGTTGGCAAAGGTT
GCTGA
Amino Acid
THCA Synthase Trichome targeting domain
Cannabis
SEQ ID NO. 40
MNCSAFSFWFVCKIIFFFLSFHIQISIA
Amino Acid
CBDA Synthase Trichome targeting domain
Cannabis
SEQ ID NO. 41
MKCSTFSFWFVCKIIFFFFSFNIQTSIA
Amino Acid
THCA Synthase
Cannabis
SEQ ID NO. 42
MNCSAFSFWFVCKIIFFFLSFHIQISIANPRENFLKCFSKHIPNNVANPKLVYTQHDQLYMSIL
NSTIQNLRFISDTTPKPLVIVTPSNNSHIQATILCSKKVGLQIRTRSGGHDAEGMSYISQVPFV
VVDLRNMHSIKIDVHSQTAWVEAGATLGEVYYWINEKNENLSFPGGYCPTVGVGGHFSGGGYGA
LMRNYGLAADNIIDAHLVNVDGKVLDRKSMGEDLFWAIRGGGGENFGIIAAWKIKLVDVPSKST
IFSVKKNMEIHGLVKLFNKWQNIAYKYDKDLVLMTHFITKNITDNHGKNKTTVHGYFSSIFHGG
VDSLVDLMNKSFPELGIKKTDCKEFSWIDTTIFYSGVVNFNTANFKKEILLDRSAGKKTAFSIK
LDYVKKPIPETAMVKILEKLYEEDVGAGMYVLYPYGGIMEEISESAIPFPHRAGIMYELWYTAS
WEKQEDNEKHINWVRSVYNFTTPYVSQNPRLAYLNYRDLDLGKTNHASPNNYTQARIWGEKYFG
KNFNRLVKVKTKVDPNNFFRNEQSIPPLPPHHH
Amino Acid
MYB8-orthologue for CAN738
Humulus lupulus
SEQ ID NO. 43
MGRAPCCEKVGLKKGRWTSEEDEILTKYIQSNGEGCWRSLPKNAGLLRCGKSCRLRWINYLRAD
LKRGNISSEEEDIIIKLHSTLGNRWSLIASHLPGRTDNEIKNYWNSHLSRKIHTFRRCNNTTTH
HHHLPNLVTVTKVNLPIPKRKGGRTSRLAMKKNKSSTSNQNSSVIKNDVGSSSSTTTTSVHQRT
TITTPTMDDQQKRQLSRCRLEEKEDQDGASTGTVVMMLGQAAAVGSSCDEDMLGHDQLSFLCCS
EEKTTENSMTNLKENGDHEVSGPYDYDHRYEKETSVDEGMLLCFNDIIDSNLLNPNEVLTLSEE
SLNLGGALMDTTTSTTTNNNNYSLSYNNNGDCVISDDHDQYWLDDVVGVDFWSWESSTTVTQEQ
EQEQEQEQEQEQEQEQEQEHHHQQDQKKNTWDNEKEKMLALLWDSDNSNWELQDNNNYHKCQEI
TSDKENAMVAWLLS
Amino Acid
atMYB12-orthologue for CAN739
Arabidopsis thaliana
SEQ ID NO. 44
MGRAPCCEKVGIKRGRWTAEEDQILSNYIQSNGEGSWRSLPKNAGLKRCGKSCRLRWINYLRSD
LKRGNITPEEEELVVKLHSTLGNRWSLIAGHLPGRTDNEIKNYWNSHLSRKLHNFIRKPSISQD
VSAVIMTNASSAPPPPQAKRRLGRTSRSAMKPKIHRTKTRKTKKTSAPPEPNADVAGADKEALM
VESSGAEAELGRPCDYYGDDCNKNLMSINGDNGVLTFDDDIIDLLLDESDPGHLYTNTTCGGDG
ELHNIRDSEGARGFSDTWNQGNLDCLLQSCPSVESFLNYDHQVNDASTDEFIDWDCVWQEGSDN
NLWHEKENPDSMVSWLLDGDDEATIGNSNCENFGEPLDHDDESALVAWLLS
Amino Acid
MYB112-orthologue for CAN833
Arabidopsis thaliana
SEQ ID NO. 45
MNISRTEFANCKTLINHKEEVEEVEKKMEIEIRRGPWTVEEDMKLVSYISLHGEGRWNSLSRSA
GLNRTGKSCRLRWLNYLRPDIRRGDISLQEQFIILELHSRWGNRWSKIAQHLPGRTDNEIKNYW
RTRVQKHAKLLKCDVNSKQFKDTIKHLWMPRLIERIAATQSVQFTSNHYSPENSSVATATSSTS
SSEAVRSSFYGGDQVEFGTLDHMTNGGYWFNGGDTFETLCSFDELNKWLIQ
Amino Acid
Cytosolic targeted THCA Synthase (ctTHCAs)
Cannabis
SEQ ID NO. 46
NPRENFLKCFSKHIPNNVANPKLVYTQHDQLYMSILNSTIQNLRFISDTTPKPLVIVTPSNNSH
IQATILCSKKVGLQIRTRSGGHDAEGMSYISQVPFVVVDLRNMHSIKIDVHSQTAWVEAGATLG
EVYYWINEKNENLSFPGGYCPTVGVGGHFSGGGYGALMRNYGLAADNIIDAHLVNVDGKVLDRK
SMGEDLFWAIRGGGGENFGIIAAWKIKLVDVPSKSTIFSVKKNMEIHGLVKLFNKWQNIAYKYD
KDLVLMTHFITKNITDNHGKNKTTVHGYFSSIFHGGVDSLVDLMNKSFPELGIKKTDCKEFSWI
DTTIFYSGVVNFNTANFKKEILLDRSAGKKTAFSIKLDYVKKPIPETAMVKILEKLYEEDVGAG
MYVLYPYGGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFRNEQSIPPL
PPHHH
Amino Acid
Trichome targeted Catalase with THCA Synthase Trichome targeting domain
Arabidopsis thaliana
SEQ ID NO. 47
MNCSAFSFWFVCKIIFFFLSFHIQISIAMDPYKYRPASSYNSPFFTTNSGAPVWNNNSSMTVGP
RGLILLEDYHLVEKLANFDRERIPERVVHARGASAKGFFEVTHDISNLTCADFLRAPGVQTPVI
VRFSTVIHARGSPETLRDPRGFAVKFYTREGNFDLVGNNFPVFFIRDGMKFPDIVHALKPNPKS
HIQENWRILDFFSHHPESLNMFTFLFDDIGIPQDYRHMDGSGVNTYMLINKAGKAHYVKFHWKP
TCGVKSLLEEDAIRLGGTNHSHATQDLYDSIAAGNYPEWKLFIQIIDPADEDKFDFDPLDVTKT
WPEDILPLQPVGRMVLNKNIDNFFAENEQLAFCPAIIVPGIHYSDDKLLQTRVFSYADTQRHRL
GPNYLQLPVNAPKCAHHNNHHEGFMNFMHRDEEVNYFPSRYDQVRHAEKYPTPPAVCSGKRERC
IIEKENNFKEPGERYRTFTPERQERFIQRWIDALSDPRITHEIRSIWISYWSQADKSLGQKLAS
RLNVRPSI
Amino Acid
Trichome targeted Catalase with CBDA Synthase Trichome targeting domain
Arabidopsis thaliana
SEQ ID NO. 48
MKCSTFSFWFVCKIIFFFFSFNIQTSIAMDPYKYRPASSYNSPFFTTNSGAPVWNNNSSMTVGP
RGLILLEDYHLVEKLANFDRERIPERVVHARGASAKGFFEVTHDISNLTCADFLRAPGVQTPVI
VRFSTVIHARGSPETLRDPRGFAVKFYTREGNFDLVGNNFPVFFIRDGMKFPDIVHALKPNPKS
HIQENWRILDFFSHHPESLNMFTFLFDDIGIPQDYRHMDGSGVNTYMLINKAGKAHYVKFHWKP
TCGVKSLLEEDAIRLGGTNHSHATQDLYDSIAAGNYPEWKLFIQIIDPADEDKFDFDPLDVTKT
WPEDILPLQPVGRMVLNKNIDNFFAENEQLAFCPAIIVPGIHYSDDKLLQTRVFSYADTQRHRL
GPNYLQLPVNAPKCAHHNNHHEGFMNFMHRDEEVNYFPSRYDQVRHAEKYPTPPAVCSGKRERC
IIEKENNFKEPGERYRTFTPERQERFIQRWIDALSDPRITHEIRSIWISYWSQADKSLGQKLAS
RLNVRPSI
Amino Acid
Catalase HPII (KatE) with THCA Synthase Trichome targeting domain
Escherichia coli
SEQ ID NO. 49
MNCSAFSFWFVCKIIFFFLSFHIQTSIAMSQHNEKNPHQHQSPLHDSSEAKPGMDSLAPEDGSH
RPAAEPTPPGAQPTAPGSLKAPDTRNEKLNSLEDVRKGSENYALTTNQGVRIADDQNSLRAGSR
GPTLLEDFILREKITHFDHERIPERIVHARGSAAHGYFQPYKSLSDITKADFLSDPNKITPVFV
RFSTVQGGAGSADTVRDIRGFATKFYTEEGIFDLVGNNTPIFFIQDAHKFPDFVHAVKPEPHWA
IPQGQSAHDTFWDYVSLQPETLHNVMWAMSDRGIPRSYRTMEGFGIHTFRLINAEGKATFVRFH
WKPLAGKASLVWDEAQKLTGRDPDFHRRELWEAIEAGDFPEYELGFQLIPEEDEFKFDFDLLDP
TKLIPEELVPVQRVGKMVLNRNPDNFFAENEQAAFHPGHIVPGLDFTNDPLLQGRLFSYTDTQI
SRLGGPNFHEIPINRPTCPYHNFQRDGMHRMGIDTNPANYEPNSINDNWPRETPPGPKRGGFES
YQERVEGNKVRERSPSFGEYYSHPRLFWLSQTPFEQRHIVDGFSFELSKVVRPYIRERVVDQLA
HIDLTLAQAVAKNLGIELTDDQLNITPPPDVNGLKKDPSLSLYAIPDGDVKGRVVAILLNDEVR
SADLLAILKALKAKGVHAKLLYSRMGEVTADDGTVLPIAATFAGAPSLTVDAVIVPCGNIADIA
DNGDANYYLMEAYKHLKPIALAGDARKFKATIKIADQGEEGIVEADSADGSFMDELLTLMAAHR
VWSRIPKIDKIPA
Amino Acid
Catalase HPII (KatE) with CBDA Synthase Trichome targeting domain
Escherichia coli
SEQ ID NO. 50
MKCSTFSFWFVCKIIFFFFSFNIQTSIAMSQHNEKNPHQHQSPLHDSSEAKPGMDSLAPEDGSH
RPAAEPTPPGAQPTAPGSLKAPDTRNEKLNSLEDVRKGSENYALTTNQGVRIADDQNSLRAGSR
GPTLLEDFILREKITHFDHERIPERIVHARGSAAHGYFQPYKSLSDITKADFLSDPNKITPVFV
RFSTVQGGAGSADTVRDIRGFATKFYTEEGIFDLVGNNTPIFFIQDAHKFPDFVHAVKPEPHWA
IPQGQSAHDTFWDYVSLQPETLHNVMWAMSDRGIPRSYRTMEGFGIHTFRLINAEGKATFVRFH
WKPLAGKASLVWDEAQKLTGRDPDFHRRELWEAIEAGDFPEYELGFQLIPEEDEFKFDFDLLDP
TKLIPEELVPVQRVGKMVLNRNPDNFFAENEQAAFHPGHIVPGLDFTNDPLLQGRLFSYTDTQI
SRLGGPNFHEIPINRPTCPYHNFQRDGMHRMGIDTNPANYEPNSINDNWPRETPPGPKRGGFES
YQERVEGNKVRERSPSFGEYYSHPRLFWLSQTPFEQRHIVDGFSFELSKVVRPYIRERVVDQLA
HIDLTLAQAVAKNLGIELTDDQLNITPPPDVNGLKKDPSLSLYAIPDGDVKGRVVAILLNDEVR
SADLLAILKALKAKGVHAKLLYSRMGEVTADDGTVLPIAATFAGAPSLTVDAVIVPCGNIADIA
DNGDANYYLMEAYKHLKPIALAGDARKFKATIKIADQGEEGIVEADSADGSFMDELLTLMAAHR
VWSRIPKIDKIPA
DNA
Glycosyltransferase (NtGT1b-codon optimized for yeast expression)
Nicotiana tabacum
SEQ ID NO. 51
ATGAAAACAACAGAACTTGTCTTCATACCCGCCCCCGGTATGGGTCACCTTGTACCCACAGTCG
AAGTCGCCAAACAACTAGTTGATAGAGACGAACAGTTGTCTATTACCGTCTTGATAATGACGTT
ACCCCTGGAGACTAATATCCCAAGTTACACCAAGAGTTTGTCCTCTGACTATTCATCCCGTATC
ACGTTGTTACAACTAAGTCAACCTGAGACGAGTGTCTCAATGAGTAGTTTTAACGCCATAAACT
TCTTCGAATACATTAGTTCCTATAAGGATCGTGTTAAAGATGCCGTAAACGAGACATTCTCCTC
TTCATCCTCCGTCAAACTTAAAGGATTTGTAATCGACATGTTTTGCACGGCAATGATAGACGTG
GCCAACGAGTTCGGTATTCCATCTTATGTATTCTACACGTCCAACGCTGCCATGCTAGGCCTAC
AACTTCACTTCCAATCCTTGTCCATCGAATATTCACCTAAGGTTCATAATTATTTAGACCCTGA
ATCTGAGGTAGCTATATCAACGTACATTAACCCAATACCAGTAAAATGCTTACCCGGTATAATT
CTTGACAATGATAAGAGTGGCACTATGTTCGTAAACCATGCCAGGAGATTCCGTGAAACAAAGG
GTATAATGGTAAATACTTTTGCAGAATTAGAAAGTCACGCCCTAAAGGCACTTAGTGACGATGA
GAAAATTCCTCCAATCTATCCCGTCGGACCCATTCTAAACTTGGGTGATGGTAATGAGGATCAT
AACCAAGAGTACGACATGATAATGAAATGGCTGGATGAACAACCACACAGTTCAGTGGTTTTCC
TGTGCTTCGGTTCCAAAGGTTCATTTGAAGAAGACCAGGTTAAAGAGATAGCAAATGCTTTAGA
GAGGAAAAGTCATCGGATGGGCACCACAGCTTGCAATTCTATCTCACCCTGCCGTCGGTGGATT
CCTTCCGAATTTGAAAACCCCGAGGAAGTGCTACCTGTAGGATTTTTTCAAAGAACCAAAGGCA
GAGGAAAAGTCATCGGATGGGCACCACAGCTTGCAATTCTATCTCACCCTGCCGTCGGTGGATT
CGTTTCCCACTGCGGCTGGAATAGTACTTTGGAATCAGTTAGATCAGGTGTACCCATAGCAACA
TGGCCTCTTTATGCAGAGCAGCAGTCCAATGCATTTCAATTGGTCAAGGATCTAGGTATGGCCG
TCGAAATTAAAATGGATTACCGTGAGGACTTTAACAAGACTAATCCTCCATTGGTAAAGGCAGA
GGAAATAGAAGACGGCATTAGGAAGTTGATGGACTCCGAGAATAAGATTAGGGCAAAGGTGATG
GAAATGAAAGATAAGTCCAGAGCTGCATTACTGGAAGGAGGATCCTCCTATGTTGCACTGGGTC
ACTTCGTGGAGACCGTAATGAAGAACTAA
Amino Acid
Glycosyltransferase (NtGT1b-generated from codon optimized sequence for yeast
expression)
Nicotiana tabacum
SEQ ID NO. 52
MKTTELVFIPAPGMGHLVPTVEVAKQLVDRDEQLSITVLIMTLPLETNIPSYTKSLSSDYSSRI
TLLQLSQPETSVSMSSFNAINFFEYISSYKDRVKDAVNETFSSSSSVKLKGFVIDMFCTAMIDV
ANEFGIPSYVFYTSNAAMLGLQLHFQSLSIEYSPKVHNYLDPESEVAISTYINPIPVKCLPGII
LDNDKSGTMFVNHARRFRETKGIMVNTFAELESHALKALSDDEKIPPIYPVGPILNLGDGNEDH
NQEYDMIMKWLDEQPHSSVVFLCFGSKGSFEEDQVKEIANALERSGNRFLWSLRRPPPKDTLQF
PSEFENPEEVLPVGFFQRTKGRGKVIGWAPQLAILSHPAVGGFVSHCGWNSTLESVRSGVPIAT
WPLYAEQQSNAFQLVKDLGMAVEIKMDYREDFNKTNPPLVKAEEIEDGIRKLMDSENKIRAKVM
EMKDKSRAALLEGGSSYVALGHFVETVMKN
DNA
Glycosyltransferase (NtGT2-codon optimized for yeast expression)
Nicotiana tabacum
SEQ ID NO. 53
ATGGTTCAACCACACGTCTTACTGGTTACTTTTCCAGCACAAGGCCATATCAACCCTTGCCTAC
AATTCGCCAAAAGACTAATAAGGATGGGCATCGAAGTAACTTTTGCCACGAGTGTATTCGCACA
TAGGCGTATGGCTAAAACTACGACATCAACTTTGTCCAAAGGACTAAACTTCGCCGCCTTCAGT
GATGGCTATGACGATGGATTCAAAGCCGACGAACATGACAGTCAACACTACATGAGTGAAATAA
AGTCCCGTGGATCTAAAACACTTAAGGATATTATACTTAAATCCTCCGATGAGGGAAGACCCGT
TACCTCTTTAGTTTATTCACTGTTACTGCCCTGGGCTGCAAAAGTCGCCAGAGAGTTTCATATT
CCTTGCGCTTTATTGTGGATCCAACCAGCTACGGTATTAGACATCTACTATTACTACTTCAATG
GATACGAGGATGCAATAAAGGGATCAACAAACGACCCCAACTGGTGTATTCAACTGCCTAGACT
TCCTCTATTAAAAAGTCAGGACTTACCTAGTTTTTTACTGTCATCCAGTAACGAAGAAAAATAT
TCATTCGCTTTACCCACCTTCAAAGAGCAGCTTGACACTTTGGATGTTGAAGAGAACCCCAAGG
TTTTGGTCAATACTTTTGACGCTTTGGAGCCAAAAGAGCTAAAGGCTATTGAAAAATATAACCT
TATCGGCATAGGACCTTTAATCCCCTCTACTTTCTTAGATGGCAAAGACCCTCTAGATTCAAGT
TTCGGAGGTGATTTGTTTCAAAAGAGTAACGATTATATCGAGTGGCTAAATAGTAAAGCCAACT
CCAGTGTGGTCTACATTTCTTTCGGAAGTCTTCTGAATTTATCAAAAAACCAAAAGGAAGAGAT
CGCAAAAGGACTGATAGAGATAAAAAAACCTTTCTTATGGGTGATCAGAGACCAGGAAAACGGT
AAAGGCGATGAGAAGGAGGAAAAACTGTCCTGTATGATGGAGCTAGAGAAACAAGGAAAAATCG
TTCCCTGGTGTTCACAGTTAGAAGTGTTAACCCATCCATCCATAGGTTGCTTCGTATCACATTG
TGGTTGGAATAGTACACTTGAAAGTCTTTCATCAGGCGTCTCTGTCGTCGCATTCCCCCACTGG
ACGGACCAGGGCACAAACGCCAAACTGATCGAAGATGTATGGAAGACGGGCGTCAGGCTAAAAA
AAAATGAGGATGGCGTGGTAGAGAGTGAAGAGATAAAGCGTTGCATAGAAATGGTCATGGATGG
CGGTGAAAAGGGAGAGGAAATGAGGCGTAACGCACAAAAGTGGAAGGAACTAGCCCGTGAAGCA
GTGAAAGAAGGAGGTTCTAGTGAGATGAATTTAAAAGCTTTCGTGCAGGAAGTTGGAAAAGGCT
GCTGA
Amino Acid
Glycosyltransferase (NtGT2-generated from codon optimized sequence for yeast
expression)
Nicotiana tabacum
SEQ ID NO. 54
MVQPHVLLVTFPAQGHINPCLQFAKRLIRMGIEVTFATSVFAHRRMAKTTTSTLSKGLNFAAFS
DGYDDGFKADEHDSQHYMSEIKSRGSKTLKDIILKSSDEGRPVTSLVYSLLLPWAAKVAREFHI
PCALLWIQPATVLDIYYYYFNGYEDAIKGSTNDPNWCIQLPRLPLLKSQDLPSFLLSSSNEEKY
SFALPTFKEQLDTLDVEENPKVLVNTFDALEPKELKAIEKYNLIGIGPLIPSTFLDGKDPLDSS
FGGDLFQKSNDYIEWLNSKANSSVVYISFGSLLNLSKNQKEEIAKGLIEIKKPFLWVIRDQENG
KGDEKEEKLSCMMELEKQGKIVPWCSQLEVLTHPSIGCFVSHCGWNSTLESLSSGVSVVAFPHW
TDQGTNAKLIEDVWKTGVRLKKNEDGVVESEEIKRCIEMVMDGGEKGEEMRRNAQKWKELAREA
VKEGGSSEMNLKAFVQEVGKGC
DNA
Glycosyltransferase (NtGT3-codon optimized for yeast expression)
Nicotiana tabacum
SEQ ID NO. 55
ATGAAAGAGACTAAAAAAATTGAGTTAGTTTTTATCCCCAGTCCTGGTATAGGACACTTAGTCT
CAACTGTGGAGATGGCCAAACTGTTGATAGCCCGTGAAGAGCAACTTTCTATTACTGTCCTGAT
TATACAATGGCCTAATGATAAAAAGCTAGACAGTTATATCCAGTCCGTCGCAAACTTTAGTTCT
AGACTGAAGTTTATACGTCTGCCCCAAGATGACTCAATCATGCAACTTTTGAAATCAAACATTT
TCACGACATTCATCGCCTCTCACAAGCCAGCTGTAAGAGACGCCGTTGCTGACATACTAAAGAG
TGAAAGTAATAACACATTGGCAGGCATTGTAATCGATCTTTTCTGCACATCCATGATCGATGTA
GCCAATGAGTTTGAGCTGCCTACTTATGTGTTTTACACTAGTGGCGCAGCCACGTTGGGTCTGC
ACTACCATATTTCTCAAAAGCGTGATGAGTTTAATAAAGACATTACCAAATATAAGGATGAGCC
AGAAGAAAAATTAAGTATAGCCACGTACCTTAACCCATTCCCTGCTAAGTGTCTACCCTCCGTG
GCATTGGATAAGGAAGGAGGATCAACGATGTTCCTAGACTTAGCTAAGAGGTTCAGGGAGACCA
AAGGCATAATGATTAACACTTTTCTTGAGCTGGAATCATACGCTCTAAACTCATTGTCTAGAGA
TAAAAACTTGCCCCCTATATACCCTGTAGGCCCTGTTTTGAACTTGAACAACGTTGAGGGTGAT
AACTTGGGCTCTAGTGATCAAAATACCATGAAATGGCTGGACGACCAGCCAGCTTCTTCCGTTG
TGTTCCTATGTTTTGGCTCAGGAGGAAGTTTCGAAAAACACCAAGTCAAAGAAATAGCTTATGC
CTTAGAATCTTCCGGATGCAGGTTCTTGTGGAGTTTGCGTAGACCCCCCACGGAAGATGCTAGG
TTCCCTTCTAATTACGAAAACTTAGAGGAAATTTTACCAGAGGGATTTCTGGAAAGAACGAAAG
GCATTGGTAAGGTCATTGGATGGGCCCCACAGTTAGCAATCTTGTCTCACAAGTCCACAGGAGG
ATTCGTGTCTCATTGCGGATGGAACTCTACCCTTGAAAGTACCTATTTCGGCGTTCCTATTGCT
ACTTGGCCAATGTATGCTGAACAACAGGCCAACGCTTTTCAACTTGTTAAAGATTTGAGGATGG
GTGTTGAGATCAAAATGGATTATAGGAAGGATATGAAGGTAATGGGCAAGGAGGTTATCGTTAA
GGCAGAAGAAATTGAAAAGGCCATAAGGGAAATCATGGACTCAGAATCAGAAATCAGGGTCAAG
GTCAAAGAGATGAAGGAGAAAAGTCGTGCAGCCCAAATGGAAGGAGGATCATCATATACCTCTA
TCGGCGGCTTCATTCAAATAATCATGGAGAACTCACAGTAA
Amino Acid
Glycosyltransferase (NtGT3-generated from codon optimized sequence for yeast
expression)
Nicotiana tabacum
SEQ ID NO. 56
MKETKKIELVFIPSPGIGHLVSTVEMAKLLIAREEQLSITVLIIQWPNDKKLDSYIQSVANFSS
RLKFIRLPQDDSIMQLLKSNIFTTFIASHKPAVRDAVADILKSESNNTLAGIVIDLFCTSMIDV
ANEFELPTYVFYTSGAATLGLHYHIQNLRDEFNKDITKYKDEPEEKLSIATYLNPFPAKCLPSV
ALDKEGGSTMFLDLAKRFRETKGIMINTFLELESYALNSLSRDKNLPPIYPVGPVLNLNNVEGD
NLGSSDQNTMKWLDDQPASSVVFLCFGSGGSFEKHQVKEIAYALESSGCRFLWSLRRPPTEDAR
FPSNYENLEEILPEGFLERTKGIGKVIGWAPQLAILSHKSTGGFVSHCGWNSTLESTYFGVPIA
TWPMYAEQQANAFQLVKDLRMGVEIKMDYRKDMKVMGKEVIVKAEEIEKAIREIMDSESEIRVK
VKEMKEKSRAAQMEGGSSYTSIGGFIQIIMENSQ
DNA
UDP-glycosyltransferase 73C3 (NtGT4-codon optimized for yeast expression)
Nicotiana tabacum
SEQ ID NO. 57
ATGGCTACTCAGGTGCATAAATTGCATTTCATTCTGTTCCCACTGATGGCTCCCGGTCACATGA
TCCCTATGATAGACATCGCAAAACTATTGGCTAACCGTGGCGTGATAACTACCATAATAACTAC
GCCCGTTAACGCCAATCGTTTTTCCTCTACGATCACTAGGGCCATTAAATCAGGCCTAAGAATC
CAGATTTTAACCTTAAAATTCCCATCAGTTGAGGTAGGCCTGCCTGAAGGATGTGAAAACATCG
ACATGTTGCCATCTTTGGACTTAGCCTCTAAATTCTTTGCTGCTATTTCTATGCTTAAACAACA
AGTGGAGAACTTGCTAGAGGGTATTAACCCTAGTCCCTCATGCGTTATTTCTGACATGGGCTTC
CCATGGACGACACAGATCGCTCAAAATTTCAATATTCCTCGTATCGTATTTCATGGCACGTGTT
GCTTTTCTCTTCTTTGTTCTTACAAAATCCTGTCATCCAATATCTTAGAGAACATTACTAGTGA
CTCAGAGTATTTTGTCGTGCCAGATCTGCCAGACCGTGTCGAGCTAACTAAGGCCCAAGTCTCT
GGATCTACAAAGAATACTACATCAGTAAGTAGTTCAGTACTGAAGGAGGTTACAGAGCAGATCA
GGCTTGCAGAGGAATCATCCTACGGTGTGATAGTTAATTCCTTCGAAGAACTGGAACAGGTGTA
TGAAAAAGAGTACAGAAAAGCCAGGGGCAAAAAGGTCTGGTGCGTGGGTCCTGTCTCTTTGTGC
AACAAGGAGATTGAAGATCTTGTTACTAGAGGAAACAAAACCGCTATAGACAATCAGGATTGTC
TTAAGTGGTTAGACAACTTCGAGACTGAATCCGTCGTCTATGCAAGTTTAGGCTCACTAAGTAG
GCTTACGTTACTGCAAATGGTTGAGCTGGGATTGGGACTGGAGGAGAGTAATAGGCCATTTGTA
TGGGTTCTGGGAGGAGGAGACAAACTAAATGATCTTGAGAAATGGATATTGGAGAATGGCTTTG
AACAGCGTATAAAGGAGAGAGGTGTCCTGATACGTGGCTGGGCACCTCAAGTATTGATTTTAAG
TCACCCCGCAATTGGAGGAGTTTTAACGCATTGTGGATGGAACTCTACATTAGAGGGCATTTCA
GCCGGACTACCCATGGTCACCTGGCCACTATTTGCCGAACAGTTCTGTAACGAAAAATTAGTAG
TGCAGGTTCTTAAAATCGGTGTCTCACTTGGAGTGAAGGTCCCTGTTAAGTGGGGTGACGAAGA
GAACGTAGGTGTCTTAGTGAAAAAGGATGACGTTAAAAAAGCACTGGATAAGCTAATGGATGAG
GGTGAGGAGGGCCAGGTTAGGAGGACCAAAGCCAAAGAGCTTGGTGAGTTAGCTAAAAAAGCCT
TTGGAGAGGGCGGATCATCCTACGTGAACCTAACGTCCCTAATTGAAGATATAATCGAGCAGCA
GAACCATAAGGAGAAGTAG
Amino Acid
UDP-glycosyltransferase 73C3 (NtGT4-generated from codon optimized sequence for
yeast expression)
Nicotiana tabacum
SEQ ID NO. 58
MATQVHKLHFILFPLMAPGHMIPMIDIAKLLANRGVITTIITTPVNANRFSSTITRAIKSGLRI
QILTLKFPSVEVGLPEGCENIDMLPSLDLASKFFAAISMLKQQVENLLEGINPSPSCVISDMGF
PWTTQIAQNFNIPRIVFHGTCCFSLLCSYKILSSNILENITSDSEYFVVPDLPDRVELTKAQVS
GSTKNTTSVSSSVLKEVTEQIRLAEESSYGVIVNSFEELEQVYEKEYRKARGKKVWCVGPVSLC
NKEIEDLVTRGNKTAIDNQDCLKWLDNFETESVVYASLGSLSRLTLLQMVELGLGLEESNRPFV
WVLGGGDKLNDLEKWILENGFEQRIKERGVLIRGWAPQVLILSHPAIGGVLTHCGWNSTLEGIS
AGLPMVTWPLFAEQFCNEKLVVQVLKIGVSLGVKVPVKWGDEENVGVLVKKDDVKKALDKLMDE
GEEGQVRRTKAKELGELAKKAFGEGGSSYVNLTSLIEDIIEQQNHKEK
DNA
Glycosyltransferase (NtGT5-codon optimized for yeast expression)
Nicotiana tabacum
SEQ ID NO. 59
ATGGGCTCTATCGGTGCAGAACTAACCAAGCCACACGCCGTATGCATTCCCTATCCCGCCCAGG
GACACATAAATCCTATGCTGAAGTTAGCTAAGATACTGCATCACAAGGGCTTCCATATAACCTT
CGTAAATACGGAATTTAATCACAGGCGTCTGCTGAAGTCCAGAGGTCCTGACTCCCTGAAAGGT
CTTTCAAGTTTCAGGTTCGAGACGATACCTGACGGACTGCCCCCATGCGAAGCTGACGCTACAC
AGGACATTCCTTCACTGTGTGAATCCACGACTAATACATGTCTAGCTCCTTTTAGAGACCTACT
TGCTAAGCTAAATGATACGAATACTTCTAACGTCCCTCCCGTAAGTTGTATTGTCAGTGACGGA
GTGATGTCATTTACCCTTGCAGCTGCACAGGAACTGGGTGTCCCAGAGGTTTTATTTTGGACTA
CATCTGCTTGTGGATTCTTAGGTTACATGCACTATTGCAAAGTCATTGAAAAAGGATATGCTCC
ATTAAAAGACGCATCAGACCTGACGAATGGCTATCTTGAGACAACCTTGGACTTCATCCCCGGC
ATGAAGGACGTCAGGCTGAGAGACTTACCTTCCTTTCTTAGGACCACCAATCCAGACGAATTTA
TGATTAAGTTTGTACTACAGGAAACTGAGCGTGCTCGTAAGGCCAGTGCCATAATACTTAATAC
CTTTGAAACCTTAGAGGCAGAGGTATTAGAATCATTAAGGAACCTTCTACCCCCCGTCTATCCA
ATCGGCCCCTTGCATTTCCTTGTCAAACACGTAGACGATGAGAACCTAAAAGGTCTACGTTCCT
CACTTTGGAAGGAGGAACCTGAATGTATTCAATGGTTAGACACCAAAGAACCTAACTCTGTCGT
GTACGTGAATTTCGGATCCATTACTGTGATGACTCCCAATCAATTAATAGAGTTCGCTTGGGGA
CTGGCAAACTCTCAACAGACCTTCCTTTGGATCATAAGGCCTGACATCGTAAGTGGTGATGCTT
CCATATTACCTCCCGAGTTTGTTGAGGAGACTAAGAACAGAGGCATGCTTGCCTCCTGGTGCTC
TCAGGAGGAGGTACTATCCCATCCCGCAATAGTGGGATTTTTGACGCACTCTGGTTGGAACTCA
ACTTTAGAATCAATTTCTAGTGGCGTCCCCATGATCTGTTGGCCTTTCTTTGCTGAGCAGCAAA
CGAACTGCTGGTTTTCAGTGACGAAGTGGGACGTTGGAATGGAAATTGATTCAGATGTGAAGAG
AGATGAAGTAGAGAGTTTAGTAAGAGAGTTAATGGTGGGTGGTAAAGGCAAGAAGATGAAGAAG
AAGGCAATGGAGTGGAAGGAACTGGCCGAGGCTTCAGCAAAAGAACACTCTGGCTCCTCTTACG
TCAATATCGAGAAGTTGGTTAACGATATATTACTATCTAGTAAGCACTAA
Amino Acid
Glycosyltransferase (NtGT5-generated from codon optimized sequence for yeast
expression)
Nicotiana tabacum
SEQ ID NO. 60
MGSIGAELTKPHAVCIPYPAQGHINPMLKLAKILHHKGFHITFVNTEFNHRRLLKSRGPDSLKG
LSSFRFETIPDGLPPCEADATQDIPSLCESTTNTCLAPFRDLLAKLNDTNTSNVPPVSCIVSDG
VMSFTLAAAQELGVPEVLFWTTSACGFLGYMHYCKVIEKGYAPLKDASDLTNGYLETTLDFIPG
MKDVRLRDLPSFLRTTNPDEFMIKFVLQETERARKASAIILNTFETLEAEVLESLRNLLPPVYP
IGPLHFLVKHVDDENLKGLRSSLWKEEPECIQWLDTKEPNSVVYVNFGSITVMTPNQLIEFAWG
LANSQQTFLWIIRPDIVSGDASILPPEFVEETKNRGMLASWCSQEEVLSHPAIVGFLTHSGWNS
TLESISSGVPMICWPFFAEQQTNCWFSVTKWDVGMEIDSDVKRDEVESLVRELMVGGKGKKMKK
KAMEWKELAEASAKEHSGSSYVNIEKLVNDILLSSKH
DNA
UDP glycosyltransferase 76G1 (UGT76G1-codon optimized for yeast expression)
Stevia rebaudiana
SEQ ID NO. 61
ATGGAGAACAAAACCGAGACAACCGTTAGGCGTAGACGTAGGATAATATTGTTTCCCGTGCCCT
TTCAAGGCCATATAAACCCAATCCTGCAGCTAGCCAACGTATTGTACTCAAAGGGCTTCAGTAT
AACGATCTTCCACACCAACTTTAATAAGCCAAAAACGTCTAATTATCCACACTTCACATTTAGA
TTTATACTTGATAACGACCCACAGGATGAAAGAATATCAAACTTGCCCACGCACGGCCCACTAG
CCGGAATGAGAATACCAATAATCAATGAGCATGGCGCCGACGAGTTGCGTAGAGAGCTGGAATT
GTTGATGCTAGCCAGTGAGGAAGACGAAGAGGTGTCCTGCTTAATAACGGATGCACTTTGGTAT
TTTGCTCAATCTGTGGCCGACTCCCTTAACCTGAGGCGTCTTGTCCTTATGACCTCCAGTCTAT
TCAACTTTCATGCCCATGTCTCATTGCCCCAATTTGATGAGCTTGGCTATTTGGATCCTGATGA
CAAAACTAGGCTGGAGGAACAGGCTTCCGGTTTTCCCATGCTAAAGGTTAAGGACATCAAATCC
GCCTACTCAAACTGGCAGATCCTTAAGGAAATTCTTGGCAAAATGATCAAACAGACGAGGGCAT
CCAGTGGCGTCATCTGGAACTCCTTTAAGGAACTTGAAGAATCAGAACTTGAAACAGTAATCAG
AGAAATACCTGCCCCAAGTTTCTTGATCCCTCTACCTAAGCACCTTACGGCTTCTAGTTCTTCT
TTGTTGGACCACGATCGTACTGTCTTTCAATGGTTAGATCAGCAACCCCCCTCATCAGTGCTAT
ATGTGTCATTCGGTAGTACATCAGAAGTGGACGAAAAGGATTTCCTTGAGATAGCCCGTGGATT
GGTGGACTCTAAACAGTCCTTTTTATGGGTTGTGAGACCTGGATTTGTAAAGGGATCCACGTGG
GTCGAACCCTTGCCCGATGGTTTCCTGGGTGAAAGAGGAAGGATAGTGAAGTGGGTCCCTCAGC
AAGAGGTACTGGCCCATGGTGCTATAGGTGCTTTCTGGACCCACTCCGGCTGGAATAGTACACT
AGAATCCGTTTGCGAGGGTGTCCCTATGATTTTTTCTGATTTTGGTTTAGATCAACCCCTGAAT
GCTAGGTACATGTCAGACGTCCTTAAAGTCGGCGTCTACCTAGAAAATGGCTGGGAGAGGGGTG
AGATAGCAAACGCTATCAGACGTGTTATGGTAGACGAAGAGGGAGAGTACATAAGGCAAAACGC
CAGGGTCCTGAAACAAAAAGCCGATGTGTCCTTGATGAAGGGCGGCTCTTCATACGAAAGTCTA
GAAAGTCTTGTTTCTTATATTTCCTCACTATAA
Amino Acid
UDP glycosyltransferase 76G1 (UGT76G1-generated from codon optimized sequence for
yeast expression)
Stevia rebaudiana
SEQ ID NO. 62
MENKTETTVRRRRRIILFPVPFQGHINPILQLANVLYSKGFSITIFHTNFNKPKTSNYPHFTFR
FILDNDPQDERISNLPTHGPLAGMRIPIINEHGADELRRELELLMLASEEDEEVSCLITDALWY
FAQSVADSLNLRRLVLMTSSLFNFHAHVSLPQFDELGYLDPDDKTRLEEQASGFPMLKVKDIKS
AYSNWQILKEILGKMIKQTRASSGVIWNSFKELEESELETVIREIPAPSFLIPLPKHLTASSSS
LLDHDRTVFQWLDQQPPSSVLYVSFGSTSEVDEKDFLEIARGLVDSKQSFLWVVRPGFVKGSTW
VEPLPDGFLGERGRIVKWVPQQEVLAHGAIGAFWTHSGWNSTLESVCEGVPMIFSDFGLDQPLN
ARYMSDVLKVGVYLENGWERGEIANAIRRVMVDEEGEYIRQNARVLKQKADVSLMKGGSSYESL
ESLVSYISSL
DNA
glycosyltransferase (UGT73A10)
Lycium barbarum
SEQ ID NO. 63
ATGGGTCAATTGCATTTTTTTTTGTTTCCAATGATGGCTCAAGGTCATATGATTCCAACTTTGG
ATATGGCTAAGTTGATTGCTTCTAGAGGTGTTAAGGCTACTATTATTACTACTCCATTGAACGA
ATCTGTTTTTTCTAAGGCTATTCAAAGAAACAAGCAATTGGGTATTGAAATTGAAATTGAAATT
AGATTGATTAAGTTTCCAGCTTTGGAAAACGATTTGCCAGAAGATTGTGAAAGATTGGATTTGA
TTCCAACTGAAGCTCATTTGCCAAACTTTTTTAAGGCTGCTGCTATGATGCAAGAACCATTGGA
ACAATTGATTCAAGAATGTAGACCAGATTGTTTGGTTTCTGATATGTTTTTGCCATGGACTACT
GATACTGCTGCTAAGTTTAACATTCCAAGAATTGTTTTTCATGGTACTAACTACTTTGCTTTGT
GTGTTGGTGATTCTATGAGAAGAAACAAGCCATTTAAGAACGTTTCTTCTGATTCTGAAACTTT
TGTTGTTCCAAACTTGCCACATGAAATTAAGTTGACTAGAACTCAAGTTTCTCCATTTGAACAA
TCTGATGAAGAATCTGTTATGTCTAGAGTTTTGAAGGAAGTTAGAGAATCTGATTTGAAGTCTT
ACGGTGTTATTTTTAACTCTTTTTACGAATTGGAACCAGATTACGTTGAACATTACACTAAGGT
TATGGGTAGAAAGTCTTGGGCTATTGGTCCATTGTCTTTGTGTAACAGAGATGTTGAAGATAAG
GCTGAAAGAGGTAAGAAGTCTTCTATTGATAAGCATGAATGTTTGGAATGGTTGGATTCTAAGA
AGCCATCTTCTATTGTTTACGTTTGTTTTGGTTCTGTTGCTAACTTTACTGTTACTCAAATGAG
AGAATTGGCTTTGGGTTTGGAAGCTTCTGGTTTGGATTTTATTTGGGCTGTTAGAGCTGATAAC
GAAGATTGGTTGCCAGAAGGTTTTGAAGAAAGAACTAAGGAAAAGGGTTTGATTATTAGAGGTT
GGGCTCCACAAGTTTTGATTTTGGATCATGAATCTGTTGGTGCTTTTGTTACTCATTGTGGTTG
GAACTCTACTTTGGAAGGTATTTCTGCTGGTGTTCCAATGGTTACTTGGCCAGTTTTTGCTGAA
CAATTTTTTAACGAAAAGTTGGTTACTCAAGTTATGAGAACTGGTGCTGGTGTTGGTTCTGTTC
AATGGAAGAGATCTGCTTCTGAAGGTGTTGAAAAGGAAGCTATTGCTAAGGCTATTAAGAGAGT
TATGGTTTCTGAAGAAGCTGAAGGTTTTAGAAACAGAGCTAGAGCTTACAAGGAAATGGCTAGA
CAAGCTATTGAAGAAGGTGGTTCTTCTTACACTGGTTTGACTACTTTGTTGGAAGATATTTCTT
CTTACGAATCTTTGTCTTCTGATTAA
Amino Acid
Glycosyltransferase (UGT73A10)
Lycium barbarum
SEQ ID NO. 64
MGQLHFFLFPMMAQGHMIPTLDMAKLIASRGVKATIITTPLNESVFSKAIQRNKQLGIEIEIEI
RLIKFPALENDLPEDCERLDLIPTEAHLPNFFKAAAMMQEPLEQLIQECRPDCLVSDMFLPWTT
DTAAKFNIPRIVFHGTNYFALCVGDSMRRNKPFKNVSSDSETFVVPNLPHEIKLTRTQVSPFEQ
SDEESVMSRVLKEVRESDLKSYGVIFNSFYELEPDYVEHYTKVMGRKSWAIGPLSLCNRDVEDK
AERGKKSSIDKHECLEWLDSKKPSSIVYVCFGSVANFTVTQMRELALGLEASGLDFIWAVRADN
EDWLPEGFEERTKEKGLIIRGWAPQVLILDHESVGAFVTHCGWNSTLEGISAGVPMVTWPVFAE
QFFNEKLVTQVMRTGAGVGSVQWKRSASEGVEKEAIAKAIKRVMVSEEAEGFRNRARAYKEMAR
QAIEEGGSSYTGLTTLLEDISSYESLSSD
DNA
Catalase HPII (KatE-codon optimized for yeast expression)
Escherichia coli
SEQ ID NO. 65
ATGTCTCAACATAACGAGAAAAACCCACATCAGCATCAATCACCACTACATGACTCCTCTGAAG
CAAAGCCAGGAATGGACTCCCTGGCTCCTGAAGATGGCTCTCACCGTCCCGCTGCCGAACCTAC
GCCACCCGGCGCACAGCCAACTGCCCCCGGTTCCCTAAAGGCCCCTGACACAAGAAATGAAAAG
TTAAATTCTCTTGAAGACGTGCGTAAAGGCAGTGAAAATTACGCTCTTACCACTAATCAAGGCG
TAAGGATAGCTGACGACCAAAACTCCCTGCGTGCTGGCTCTAGAGGCCCTACCCTTCTTGAGGA
TTTTATCCTTCGTGAAAAGATTACTCACTTCGATCACGAAAGGATTCCTGAGAGGATCGTCCAT
GCTAGAGGTTCTGCTGCTCACGGTTATTTTCAGCCCTATAAATCCCTTTCCGACATAACGAAGG
CAGATTTTTTGAGTGATCCTAATAAAATAACGCCTGTATTTGTTAGATTTTCTACTGTCCAAGG
TGGTGCTGGATCAGCTGACACTGTTAGAGACATCAGGGGATTTGCTACGAAGTTTTACACTGAA
GAGGGCATCTTCGACTTGGTTGGTAATAATACACCAATATTCTTTATCCAAGACGCACACAAAT
TCCCAGACTTTGTGCATGCTGTCAAACCCGAGCCACATTGGGCTATTCCACAGGGCCAGTCTGC
CCATGACACGTTCTGGGATTACGTTTCTCTGCAACCTGAGACGCTGCACAACGTTATGTGGGCA
ATGTCAGATCGTGGAATACCTAGATCTTACAGGACAATGGAAGGCTTTGGCATACATACTTTCA
GGTTAATAAATGCCGAAGGAAAGGCCACATTCGTCAGGTTTCATTGGAAGCCCTTAGCAGGTAA
GGCCTCTCTAGTATGGGACGAAGCTCAAAAACTTACTGGTAGAGATCCAGACTTTCATAGGCGT
GAATTGTGGGAAGCAATCGAAGCCGGCGACTTTCCTGAGTATGAGCTGGGCTTCCAGTTGATCC
CAGAAGAGGACGAATTTAAATTTGATTTCGACTTACTTGATCCAACGAAACTGATTCCCGAGGA
GTTGGTCCCTGTCCAACGTGTCGGTAAAATGGTGTTGAACAGGAACCCTGACAATTTCTTTGCA
GAAAACGAACAAGCCGCCTTCCATCCAGGCCATATAGTACCAGGCTTAGACTTCACTAATGACC
CACTGCTGCAAGGTAGACTGTTTAGTTACACTGATACACAGATATCCAGACTAGGTGGTCCAAA
CTTCCATGAAATCCCCATCAACAGGCCCACGTGCCCCTATCACAATTTCCAGCGTGATGGCATG
CATAGAATGGGTATTGACACGAATCCCGCTAATTATGAGCCAAACTCTATAAACGATAACTGGC
CTAGAGAGACGCCACCAGGCCCTAAGCGTGGTGGTTTTGAATCCTATCAAGAGCGTGTCGAAGG
TAATAAAGTAAGGGAGAGATCACCCTCTTTCGGCGAATATTATAGTCATCCCCGTTTGTTTTGG
TTATCACAGACGCCTTTCGAACAACGTCACATAGTTGATGGATTCTCTTTTGAGCTTTCAAAAG
TGGTTCGTCCCTATATCAGGGAAAGGGTTGTCGACCAGCTTGCCCATATTGATTTAACACTTGC
ACAAGCTGTTGCCAAAAACCTAGGAATAGAGCTGACAGACGATCAACTAAATATCACCCCACCT
CCTGATGTCAACGGCTTAAAGAAGGATCCATCTTTAAGTCTATACGCAATTCCCGACGGTGATG
TTAAAGGTAGAGTGGTAGCAATTTTGCTAAACGATGAAGTGCGTAGTGCTGACCTACTAGCCAT
CTTAAAGGCCTTGAAAGCAAAGGGAGTGCACGCAAAGTTACTGTACAGTCGTATGGGAGAGGTT
ACTGCTGACGACGGTACGGTACTACCTATCGCCGCAACATTTGCCGGAGCCCCAAGTTTGACAG
TCGATGCCGTTATCGTACCTTGTGGTAATATCGCCGATATTGCCGACAACGGAGACGCTAATTA
CTACTTAATGGAGGCCTATAAGCACTTGAAGCCCATAGCACTGGCTGGAGACGCTCGTAAATTT
AAGGCTACTATCAAGATTGCAGATCAGGGCGAGGAGGGTATTGTTGAGGCAGACAGTGCAGATG
GATCTTTCATGGATGAGCTTCTAACACTAATGGCAGCACATAGAGTATGGTCTCGTATCCCCAA
GATCGACAAAATCCCTGCGTAA
Amino Acid
Catalase HPII (KatE-generated from codon optimized sequence for yeast expression)
Escherichia coli
SEQ ID NO. 66
MSQHNEKNPHQHQSPLHDSSEAKPGMDSLAPEDGSHRPAAEPTPPGAQPTAPGSLKAPDTRNEK
LNSLEDVRKGSENYALTTNQGVRIADDQNSLRAGSRGPTLLEDFILREKITHFDHERIPERIVH
ARGSAAHGYFQPYKSLSDITKADFLSDPNKITPVFVRFSTVQGGAGSADTVRDIRGFATKFYTE
EGIFDLVGNNTPIFFIQDAHKFPDFVHAVKPEPHWAIPQGQSAHDTFWDYVSLQPETLHNVMWA
MSDRGIPRSYRTMEGFGIHTFRLINAEGKATFVRFHWKPLAGKASLVWDEAQKLTGRDPDFHRR
ELWEAIEAGDFPEYELGFQLIPEEDEFKFDFDLLDPTKLIPEELVPVQRVGKMVLNRNPDNFFA
ENEQAAFHPGHIVPGLDFTNDPLLQGRLFSYTDTQISRLGGPNFHEIPINRPTCPYHNFQRDGM
HRMGIDTNPANYEPNSINDNWPRETPPGPKRGGFESYQERVEGNKVRERSPSFGEYYSHPRLFW
LSQTPFEQRHIVDGFSFELSKVVRPYIRERVVDQLAHIDLTLAQAVAKNLGIELTDDQLNITPP
PDVNGLKKDPSLSLYAIPDGDVKGRVVAILLNDEVRSADLLAILKALKAKGVHAKLLYSRMGEV
TADDGTVLPIAATFAGAPSLTVDAVIVPCGNIADIADNGDANYYLMEAYKHLKPIALAGDARKF
KATIKIADQGEEGIVEADSADGSFMDELLTLMAAHRVWSRIPKIDKIPA
DNA
ABC transporter ABCG2
Mus musculus
SEQ ID NO. 67
ATGTCTTCTTCTAACGATCATGTTTTGGTTCCAATGTCTCAAAGAAACAACAACGGTTTGCCAA
GAATGAACTCTAGAGCTGTTAGAACTTTGGCTGAAGGTGATGTTTTGTCTTTTCATCATATTAC
TTACAGAGTTAAGGTTAAGTCTGGTTTTTTGGTTAGAAAGACTGTTGAAAAGGAAATTTTGTCT
GATATTAACGGTATTATGAAGCCAGGTTTGAACGCTATTTTGGGTCCAACTGGTGGTGGTAAGT
CTTCTTTGTTGGATGTTTTGGCTGCTAGAAAGGATCCAAAGGGTTTGTCTGGTGATGTTTTGAT
TAACGGTGCTCCACAACCAGCTCATTTTAAGTGTTGTTCTGGTTACGTTGTTCAAGATGATGTT
GTTATGGGTACTTTGACTGTTAGAGAAAACTTGCAATTTTCTGCTGCTTTGAGATTGCCAACTA
CTATGAAGAACCATGAAAAGAACGAAAGAATTAACACTATTATTAAGGAATTGGGTTTGGAAAA
GGTTGCTGATTCTAAGGTTGGTACTCAATTTATTAGAGGTATTTCTGGTGGTGAAAGAAAGAGA
ACTTCTATTGGTATGGAATTGATTACTGATCCATCTATTTTGTTTTTGGATGAACCAACTACTG
GTTTGGATTCTTCTACTGCTAACGCTGTTTTGTTGTTGTTGAAGAGAATGTCTAAGCAAGGTAG
AACTATTATTTTTTCTATTCATCAACCAAGATACTCTATTTTTAAGTTGTTTGATTCTTTGACT
TTGTTGGCTTCTGGTAAGTTGGTTTTTCATGGTCCAGCTCAAAAGGCTTTGGAATACTTTGCTT
CTGCTGGTTACCATTGTGAACCATACAACAACCCAGCTGATTTTTTTTTGGATGTTATTAACGG
TGATTCTTCTGCTGTTATGTTGAACAGAGAAGAACAAGATAACGAAGCTAACAAGACTGAAGAA
CCATCTAAGGGTGAAAAGCCAGTTATTGAAAACTTGTCTGAATTTTACATTAACTCTGCTATTT
ACGGTGAAACTAAGGCTGAATTGGATCAATTGCCAGGTGCTCAAGAAAAGAAGGGTACTTCTGC
TTTTAAGGAACCAGTTTACGTTACTTCTTTTTGTCATCAATTGAGATGGATTGCTAGAAGATCT
TTTAAGAACTTGTTGGGTAACCCACAAGCTTCTGTTGCTCAATTGATTGTTACTGTTATTTTGG
GTTTGATTATTGGTGCTATTTACTTTGATTTGAAGTACGATGCTGCTGGTATGCAAAACAGAGC
TGGTGTTTTGTTTTTTTTGACTACTAACCAATGTTTTTCTTCTGTTTCTGCTGTTGAATTGTTT
GTTGTTGAAAAGAAGTTGTTTATTCATGAATACATTTCTGGTTACTACAGAGTTTCTTCTTACT
TTTTTGGTAAGGTTATGTCTGATTTGTTGCCAATGAGATTTTTGCCATCTGTTATTTTTACTTG
TATTTTGTACTTTATGTTGGGTTTGAAGAAGACTGTTGATGCTTTTTTTATTATGATGTTTACT
TTGATTATGGTTGCTTACACTGCTTCTTCTATGGCTTTGGCTATTGCTACTGGTCAATCTGTTG
TTTCTGTTGCTACTTTGTTGATGACTATTGCTTTTGTTTTTATGATGTTGTTTTCTGGTTTGTT
GGTTAACTTGAGAACTATTGGTCCATGGTTGTCTTGGTTGCAATACTTTTCTATTCCAAGATAC
GGTTTTACTGCTTTGCAATACAACGAATTTTTGGGTCAAGAATTTTGTCCAGGTTTTAACGTTA
CTGATAACTCTACTTGTGTTAACTCTTACGCTATTTGTACTGGTAACGAATACTTGATTAACCA
AGGTATTGAATTGTCTCCATGGGGTTTGTGGAAGAACCATGTTGCTTTGGCTTGTATGATTATT
ATTTTTTTGACTATTGCTTACTTGAAGTTGTTGTTTTTGAAGAAGTACTCTTAA
Amino Acid
ABC transporter ABCG2
Mus musculus
SEQ ID NO. 68
MSSSNDHVLVPMSQRNNNGLPRMNSRAVRTLAEGDVLSFHHITYRVKVKSGFLVRKTVEKEILS
DINGIMKPGLNAILGPTGGGKSSLLDVLAARKDPKGLSGDVLINGAPQPAHFKCCSGYVVQDDV
VMGTLIVRENLQFSAALRLPTTMKNHEKNERINTIIKELGLEKVADSKVGTQFIRGISGGERKR
TSIGMELITDPSILFLDEPTTGLDSSTANAVLLLLKRMSKQGRTIIFSIHQPRYSIFKLFDSLT
LLASGKLVFHGPAQKALEYFASAGYHCEPYNNPADFFLDVINGDSSAVMLNREEQDNEANKTEE
PSKGEKPVIENLSEFYINSAIYGETKAELDQLPGAQEKKGTSAFKEPVYVTSFCHQLRWIARRS
FKNLLGNPQASVAQLIVTVILGLIIGAIYFDLKYDAAGMQNRAGVLFFLTTNQCFSSVSAVELF
VVEKKLFIHEYISGYYRVSSYFFGKVMSDLLPMRFLPSVIFTCILYFMLGLKKTVDAFFIMMFT
LIMVAYTASSMALAIATGQSVVSVATLLMTIAFVFMMLFSGLLVNLRTIGPWLSWLQYFSIPRY
GFTALQYNEFLGQEFCPGFNVTDNSTCVNSYAICTGNEYLINQGIELSPWGLWKNHVALACMII
IFLTIAYLKLLFLKKYS
DNA
Cytochrome P450 (CYP3A4)
Mus musculus
SEQ ID NO. 69
ATGAACTTGTTTTCTGCTTTGTCTTTGGATACTTTGGTTTTGTTGGCTATTATTTTGGTTTTGT
TGTACAGATACGGTACTAGAACTCATGGTTTGTTTAAGAAGCAAGGTATTCCAGGTCCAAAGCC
ATTGCCATTTTTGGGTACTGTTTTGAACTACTACACTGGTATTTGGAAGTTTGATATGGAATGT
TACGAAAAGTACGGTAAGACTTGGGGTTTGTTTGATGGTCAAACTCCATTGTTGGTTATTACTG
ATCCAGAAACTATTAAGAACGTTTTGGTTAAGGATTGTTTGTCTGTTTTTACTAACAGAAGAGA
ATTTGGTCCAGTTGGTATTATGTCTAAGGCTATTTCTATTTCTAAGGATGAAGAATGGAAGAGA
TACAGAGCTTTGTTGTCTCCAACTTTTACTTCTGGTAGATTGAAGGAAATGTTTCCAGTTATTG
AACAATACGGTGATATTTTGGTTAAGTACTTGAGACAAGAAGCTGAAAAGGGTATGCCAGTTGC
TATGAAGGATGTTTTGGGTGCTTACTCTATGGATGTTATTACTTCTACTTCTTTTGGTGTTAAC
GTTGATTCTTTGAACAACCCAGAAGATCCATTTGTTGAAGAAGCTAAGAAGTTTTTGAGAGTTG
ATTTTTTTGATCCATTGTTGTTTTCTGTTGTTTTGTTTCCATTGTTGACTCCAGTTTACGAAAT
GTTGAACATTTGTATGTTTCCAAACGATTCTATTGAATTTTTTAAGAAGTTTGTTGATAGAATG
CAAGAATCTAGATTGGATTCTAACCAAAAGCATAGAGTTGATTTTTTGCAATTGATGATGAACT
CTCATAACAACTCTAAGGATAAGGATTCTCATAAGGCTTTTTCTAACATGGAAATTACTGTTCA
ATCTATTATTTTTATTTCTGCTGGTTACGAAACTACTTCTTCTACTTTGTCTTTTACTTTGTAC
TGTTTGGCTACTCATCCAGATATTCAAAAGAAGTTGCAAGCTGAAATTGATAAGGCTTTGCCAA
ACAAGGCTACTCCAACTTGTGATACTGTTATGGAAATGGAATACTTGGATATGGTTTTGAACGA
AACTTTGAGATTGTACCCAATTGTTACTAGATTGGAAAGAGTTTGTAAGAAGGATGTTGAATTG
AACGGTGTTTACATTCCAAAGGGTTCTATGGTTATGATTCCATCTTACGCTTTGCATCATGATC
CACAACATTGGCCAGATCCAGAAGAATTTCAACCAGAAAGATTTTCTAAGGAAAACAAGGGTTC
TATTGATCCATACGTTTACTTGCCATTTGGTATTGGTCCAAGAAACTGTATTGGTATGAGATTT
GCTTTGATGAACATGAAGTTGGCTGTTACTAAGGTTTTGCAAAACTTTTCTTTTCAACCATGTC
AAGAAACTCAAATTCCATTGAAGTTGTCTAGACAAGGTATTTTGCAACCAGAAAAGCCAATTGT
TTTGAAGGTTGTTCCAAGAGATGCTGTTATTACTGGTGCTTAA
Amino Acid
Cytochrome P450 (CYP3A4)
Mus musculus
SEQ ID NO. 70
MNLFSALSLDTLVLLAIILVLLYRYGTRTHGLFKKQGIPGPKPLPFLGTVLNYYTGIWKFDMEC
YEKYGKTWGLFDGQTPLLVITDPETIKNVLVKDCLSVFTNRREFGPVGIMSKAISISKDEEWKR
YRALLSPTFTSGRLKEMFPVIEQYGDILVKYLRQEAEKGMPVAMKDVLGAYSMDVIISISFGVN
VDSLNNPEDPFVEEAKKFLRVDFFDPLLFSVVLFPLLTPVYEMLNICMFPNDSIEFFKKFVDRM
QESRLDSNQKHRVDFLQLMMNSHNNSKDKDSHKAFSNMEITVQSIIFISAGYETTSSTLSFTLY
CLATHPDIQKKLQAEIDKALPNKATPTCDTVMEMEYLDMVLNETLRLYPIVTRLERVCKKDVEL
NGVYIPKGSMVMIPSYALHHDPQHWPDPEEFQPERFSKENKGSIDPYVYLPFGIGPRNCIGMRF
ALMNMKLAVTKVLQNFSFQPCQETQIPLKLSRQGILQPEKPIVLKVVPRDAVITGA
DNA
P450 oxidoreductase gene (CYP oxidoreductase)
Mus musculus
SEQ ID NO. 71
ATGGGTGATTCTCATGAAGATACTTCTGCTACTGTTCCAGAAGCTGTTGCTGAAGAAGTTTCTT
TGTTTTCTACTACTGATATTGTTTTGTTTTCTTTGATTGTTGGTGTTTTGACTTACTGGTTTAT
TTTTAAGAAGAAGAAGGAAGAAATTCCAGAATTTTCTAAGATTCAAACTACTGCTCCACCAGTT
AAGGAATCTTCTTTTGTTGAAAAGATGAAGAAGACTGGTAGAAACATTATTGTTTTTTACGGTT
CTCAAACTGGTACTGCTGAAGAATTTGCTAACAGATTGTCTAAGGATGCTCATAGATACGGTAT
GAGAGGTATGTCTGCTGATCCAGAAGAATACGATTTGGCTGATTTGTCTTCTTTGCCAGAAATT
GATAAGTCTTTGGTTGTTTTTTGTATGGCTACTTACGGTGAAGGTGATCCAACTGATAACGCTC
AAGATTTTTACGATTGGTTGCAAGAAACTGATGTTGATTTGACTGGTGTTAAGTTTGCTGTTTT
TGGTTTGGGTAACAAGACTTACGAACATTTTAACGCTATGGGTAAGTACGTTGATCAAAGATTG
GAACAATTGGGTGCTCAAAGAATTTTTGAATTGGGTTTGGGTGATGATGATGGTAACTTGGAAG
AAGATTTTATTACTTGGAGAGAACAATTTTGGCCAGCTGTTTGTGAATTTTTTGGTGTTGAAGC
TACTGGTGAAGAATCTTCTATTAGACAATACGAATTGGTTGTTCATGAAGATATGGATACTGCT
AAGGTTTACACTGGTGAAATGGGTAGATTGAAGTCTTACGAAAACCAAAAGCCACCATTTGATG
CTAAGAACCCATTTTTGGCTGCTGTTACTACTAACAGAAAGTTGAACCAAGGTACTGAAAGACA
TTTGATGCATTTGGAATTGGATATTTCTGATTCTAAGATTAGATACGAATCTGGTGATCATGTT
GCTGTTTACCCAGCTAACGATTCTACTTTGGTTAACCAAATTGGTGAAATTTTGGGTGCTGATT
TGGATGTTATTATGTCTTTGAACAACTTGGATGAAGAATCTAACAAGAAGCATCCATTTCCATG
TCCAACTACTTACAGAACTGCTTTGACTTACTACTTGGATATTACTAACCCACCAAGAACTAAC
GTTTTGTACGAATTGGCTCAATACGCTTCTGAACCATCTGAACAAGAACATTTGCATAAGATGG
CTTCTTCTTCTGGTGAAGGTAAGGAATTGTACTTGTCTTGGGTTGTTGAAGCTAGAAGACATAT
TTTGGCTATTTTGCAAGATTACCCATCTTTGAGACCACCAATTGATCATTTGTGTGAATTGTTG
CCAAGATTGCAAGCTAGATACTACTCTATTGCTTCTTCTTCTAAGGTTCATCCAAACTCTGTTC
ATATTTGTGCTGTTGCTGTTGAATACGAAGCTAAGTCTGGTAGAGTTAACAAGGGTGTTGCTAC
TTCTTGGTTGAGAACTAAGGAACCAGCTGGTGAAAACGGTAGAAGAGCTTTGGTTCCAATGTTT
GTTAGAAAGTCTCAATTTAGATTGCCATTTAAGCCAACTACTCCAGTTATTATGGTTGGTCCAG
GTACTGGTGTTGCTCCATTTATGGGTTTTATTCAAGAAAGAGCTTGGTTGAGAGAACAAGGTAA
GGAAGTTGGTGAAACTTTGTTGTACTACGGTTGTAGAAGATCTGATGAAGATTACTTGTACAGA
GAAGAATTGGCTAGATTTCATAAGGATGGTGCTTTGACTCAATTGAACGTTGCTTTTTCTAGAG
AACAAGCTCATAAGGTTTACGTTCAACATTTGTTGAAGAGAGATAAGGAACATTTGTGGAAGTT
GATTCATGAAGGTGGTGCTCATATTTACGTTTGTGGTGATGCTAGAAACATGGCTAAGGATGTT
CAAAACACTTTTTACGATATTGTTGCTGAATTTGGTCCAATGGAACATACTCAAGCTGTTGATT
ACGTTAAGAAGTTGATGACTAAGGGTAGATACTCTTTGGATGTTTGGTCTTAA
Amino Acid
P450 oxidoreductase (CYP oxidoreductase)
Mus musculus
SEQ ID NO. 72
MGDSHEDTSATVPEAVAEEVSLFSTTDIVLFSLIVGVLTYWFIFKKKKEEIPEFSKIQTTAPPV
KESSFVEKMKKTGRNIIVFYGSQTGTAEEFANRLSKDAHRYGMRGMSADPEEYDLADLSSLPEI
DKSLVVFCMATYGEGDPTDNAQDFYDWLQETDVDLTGVKFAVFGLGNKTYEHFNAMGKYVDQRL
EQLGAQRIFELGLGDDDGNLEEDFITWREQFWPAVCEFFGVEATGEESSIRQYELVVHEDMDTA
KVYTGEMGRLKSYENQKPPFDAKNPFLAAVTTNRKLNQGTERHLMHLELDISDSKIRYESGDHV
AVYPANDSTLVNQIGEILGADLDVIMSLNNLDEESNKKHPFPCPTTYRTALTYYLDITNPPRTN
VLYELAQYASEPSEQEHLHKMASSSGEGKELYLSWVVEARRHILAILQDYPSLRPPIDHLCELL
PRLQARYYSIASSSKVHPNSVHICAVAVEYEAKSGRVNKGVATSWLRTKEPAGENGRRALVPMF
VRKSQFRLPFKPTTPVIMVGPGTGVAPFMGFIQERAWLREQGKEVGETLLYYGCRRSDEDYLYR
EELARFHKDGALTQLNVAFSREQAHKVYVQHLLKRDKEHLWKLIHEGGAHIYVCGDARNMAKDV
QNTFYDIVAEFGPMEHTQAVDYVKKLMTKGRYSLDVWS
Amino Acid
Catalase 1
Arabidopsis thaliana
SEQ ID NO. 73
MDPYRVRPSSAHDSPFFTTNSGAPVWNNNSSLTVGTRGPILLEDYHLLEKLANFDRERIP
ERVVHARGASAKGFFEVTHDITQLTSADFLRGPGVQTPVIVRFSTVIHERGSPETLRDPR
GFAVKFYTREGNFDLVGNNFPVFFVRDGMKFPDMVHALKPNPKSHIQENWRILDFFSHHP
ESLHMFSFLFDDLGIPQDYRHMEGAGVNTYMLINKAGKAHYVKFHWKPTCGIKCLSDEEA
IRVGGANHSHATKDLYDSIAAGNYPQWNLFVQVMDPAHEDKFDFDPLDVTKIWPEDILPL
QPVGRLVLNKNIDNFFNENEQIAFCPALVVPGIHYSDDKLLQTRIFSYADSQRHRLGPNY
LQLPVNAPKCAHHNNHHDGFMNFMHRDEEVNYFPSRLDPVRHAEKYPTTPIVCSGNREKC
FIGKENNFKQPGERYRSWDSDRQERFVKRFVEALSEPRVTHEIRSIWISYWSQADKSLGQ
KLATRLNVRPNF
Amino Acid
Catalase 2
Arabidopsis thaliana
SEQ ID NO. 74
MDPYKYRPASSYNSPFFTTNSGAPVWNNNSSMTVGPRGPILLEDYHLVEKLANFDRERIP
ERVVHARGASAKGFFEVTHDISNLTCADFLRAPGVQTPVIVRFSTVIHERGSPETLRDPR
GFAVKFYTREGNFDLVGNNFPVFFIRDGMKFPDMVHALKPNPKSHIQENWRILDFFSHHP
ESLNMFTFLFDDIGIPQDYRHMDGSGVNTYMLINKAGKAHYVKFHWKPTCGVKSLLEEDA
IRVGGTNHSHATQDLYDSIAAGNYPEWKLFIQIIDPADEDKFDFDPLDVTKTWPEDILPL
QPVGRMVLNKNIDNFFAENEQLAFCPAIIVPGIHYSDDKLLQTRVFSYADTQRHRLGPNY
LQLPVNAPKCAHHNNHHEGFMNFMHRDEEVNYFPSRYDQVRHAEKYPTPPAVCSGKRERC
IIEKENNFKEPGERYRTFTPERQERFIQRWIDALSDPRITHEIRSIWISYWSQADKSLGQ
KLASRLNVRPSI
Amino Acid
Catalase 3
Arabidopsis thaliana
SEQ ID NO. 75
MDPYKYRPSSAYNAPFYTTNGGAPVSNNISSLTIGERGPVLLEDYHLIEKVANFTRERIP
ERVVHARGISAKGFFEVTHDISNLTCADFLRAPGVQTPVIVRFSTVVHERASPETMRDIR
GFAVKFYTREGNFDLVGNNTPVFFIRDGIQFPDVVHALKPNPKTNIQEYWRILDYMSHLP
ESLLTWCWMFDDVGIPQDYRHMEGFGVHTYTLIAKSGKVLFVKFHWKPTCGIKNLTDEEA
KVVGGANHSHATKDLHDAIASGNYPEWKLFIQTMDPADEDKFDFDPLDVTKIWPEDILPL
QPVGRLVLNRTIDNFFNETEQLAFNPGLVVPGIYYSDDKLLQCRIFAYGDTQRHRLGPNY
LQLPVNAPKCAHHNNHHEGFMNFMHRDEEINYYPSKFDPVRCAEKVPTPTNSYTGIRTKC
VIKKENNFKQAGDRYRSWAPDRQDRFVKRWVEILSEPRLTHEIRGIWISYWSQADRSLGQ
KLASRLNVRPSI
DNA
forward primer of CYP3A4
Artificial
SEQ ID NO. 76
TGCCTAATAAAGCTCCTCCTACT
DNA
reverse primer of CYP3A4
Artificial
SEQ ID NO. 77
GCTCCTGAAACAGTTCCATCTC
DNA
forward primer of P450 oxidoreductase
Artificial
SEQ ID NO. 78
GGAAGAGCTTTGGTTCCTATGT
DNA
reverse primer of P450 oxidoreductase
Artificial
SEQ ID NO. 79
GCTCCCAATTCAGCAACAATATC
DNA
forward primer of CBDA synthase
Artificial
SEQ ID NO. 80
ACATCACAATCACACAAAACTAACAAAAG
DNA
reverse primer of CBDA synthase
Artificial
SEQ ID NO. 81
GGCCATAGTTTCTCATCAATGG
DNA
forward primer of UGT76G1
Artificial
SEQ ID NO. 82
GATTGGAAGAACAAGCTTCAGGATTTCC
DNA
reverse primer of UGT76G1
Artificial
SEQ ID NO. 83
CCATCCTGAATGAGTCCAAAAAGCTC
DNA
forward primer of ABCG2
Artificial
SEQ ID NO. 84
CCTTCAGGATTGTCAGGAGATG
DNA
reverse primer of ABCG2
Artificial
SEQ ID NO. 85
GCAGGTCCATGAAACATCAATC
DNA
forward primer of Trichome-targeted CBDAs
Artificial
SEQ ID NO. 86
AAAGATCAAAAGCAAGTTCTTCACTGT
DNA
reverse primer of Trichome-targeted CBDAs
Artificial
SEQ ID NO. 87
CCATGCAGTTTGGCTATGAACATCT
DNA
forward primer of Trichome-targeted UGT
Artificial
SEQ ID NO. 88
AGTGCTCAACATTCTCCTTTTGGTT
DNA
reverse primer of Trichome-targeted UGT
Artificial
SEQ ID NO. 89
TCTGAAGCCAACATCAACAATTCCA
DNA
forward primer of Plasma membrane-targeted UTRI
Artificial
SEQ ID NO. 90
TTGTTCCTTAAACCTCGCCTTTGAC
DNA
reverse primer of Plasma membrane-targeted UTRI
Artificial
SEQ ID NO. 91
TCATTATGGAGCACTCCACTCTCTG
DNA
forward primer of Cytosolic-targeted CBDA synthase
Artificial
SEQ ID NO. 92
AAAGATCAAAAGCAAGTTCTTCACTGT
DNA
reverse primer of Cytosolic-targeted CBDA synthase
Artificial
SEQ ID NO. 93
ATAAACTTCTCCAAGGGTAGCTCCG
DNA
forward primer of Cytosolic-targeted UGT
Artificial
SEQ ID NO. 94
AGAACTGGAAGAATCCGAACTGGAA
DNA
reverse primer of Cytosolic-targeted UGT
Artificial
SEQ ID NO. 95
AAATCATCGGGACACCTTCACAAAC
DNA
Forward primer of NtGT1
Artificial
SEQ. ID NO. 96
ATGAAAACAACAGAACTTGTCTTCA
DNA
Reverse primer of NtGT1
Artificial
SEQ. ID NO. 97
TGAAGTTGTAGGCCTAGCATGG
DNA
Forward primer of NtGT2
Artificial
SEQ. ID NO. 98
ATGGTTCAACCACACGTCTTACTGG
DNA
Reverse primer of NtGT2
Artificial
SEQ. ID NO. 99
TTGAATACACCAGTTGGGGTCG
DNA
Forward primer of NtGT3
Artificial
SEQ. ID NO. 100
ATGAAAGAGACTAAAAAAATTGAGT
DNA
Reverse primer of NtGT3
Artificial
SEQ. ID NO. 101
CATCACGCAGATTTTGAATATGG
DNA
Forward primer of NtGT4
Artificial
SEQ. ID NO. 102
ATGGCTACTCAGGTGCATAAATTGC
DNA
Reverse primer of NtGT4
Artificial
SEQ. ID NO. 103
GGCCTTAGTTAGCTCGACACGG
DNA
Forward primer of NtGT5
Artificial
SEQ. ID NO. 104
ATGGGCTCTATCGGTGCAGAACTAA
DNA
Reverse primer of NtGT5
Artificial
SEQ. ID NO. 105
CGGGGATGAAGTCCAAGGTTGT
DNA
Forward primer of Kat-E
Artificial
SEQ. ID NO. 106
ATGTCTCAACATAACGAGAAAAACC
DNA
Reverse primer of Kat-E
Artificial
SEQ. ID NO. 107
CGTAGCAAATCCCCTGATGTCT
DNA
Forward primer of UGT76G1
Artificial
SEQ. ID NO. 108
ATGGAGAACAAAACCGAGACAACCG
DNA
Reverse primer of UGT76G1
Artificial
SEQ. ID NO. 109
CCTTTAGCATGGGAAAACCGGA
DNA
Forward primer of UGT76G1 (for tobacco BY2 cells)
Artificial
SEQ. ID NO. 110
GATTGGAAGAACAAGCTTCAGGATTTCC
DNA
Reverse primer of UGT76G1 (for tobacco BY2 cells)
Artificial
SEQ. ID NO. 111
CCATCCTGATGAGTCCAAAAAGCTC
DNA
Forward primer of ABCG2 (for tobacco BY2 cells)
Artificial
SEQ. ID NO. 112
CCTTCAGGATTGTCAGGAGATG
DNA
Reverse primer of ABCG2 (for tobacco BY2 cells)
Artificial
SEQ. ID NO. 113
GCAGGTCCATGAAACATCAATC

What is claimed is:

1. A chemically modified cannabinoid food and beverage additive having enhanced water solubility comprising:
an aqueous solution;
at least one chemically modified cannabinoid dissolved in said aqueous solution, wherein said chemically modified cannabinoid comprises an water soluble acetylated cannabinoid glycoside; and
wherein said consumable fluid composition may be introduced to a food or beverage, and wherein said water soluble acetylated cannabinoid glycoside exhibits an enhanced ability to stay in solution compared to a cannabinoid glycoside, and an unmodified cannabinoid.

2. The consumable fluid composition of claim 1, wherein said one or more water soluble acetylated cannabinoid glycosides comprises a water soluble O-acetyl cannabinoid glycoside.

3. The consumable fluid composition of claim 1, wherein said water soluble acetylated cannabinoid glycoside comprises a water soluble acetylated cannabinoid glycoside generated in vivo through incubation of a cannabinoid in a tobacco cell suspension culture.

4. The consumable fluid composition of claim 1, wherein said water soluble acetylated cannabinoid glycoside comprises a water soluble acetylated cannabinoid glycoside generated in vitro through incubation with a glycosyltransferase protein having glycosylation activity towards one or more cannabinoids and acetyltransferase protein having acetylation activity toward one or more cannabinoids.

5. The consumable fluid composition of claim 1, wherein said glycosyltransferase protein having glycosylation activity towards one or more cannabinoids comprises a glycosyltransferase protein from tobacco selected from the group consisting of: NtGT3; NtGT4; and NtGT5, and a homolog or ortholog of NtGT3, NtGT4, and NtGT5 having glycosylation activity towards one or more cannabinoids.

6. The consumable fluid composition of claim 1, wherein said aqueous solution comprises an aqueous solution selected from the group consisting of: water, saline, purified water, propylene glycol, deionized water, or combinations thereof.

7. The consumable fluid composition of claim 1, wherein said composition is added to a quantity of an alcohol beverage, wherein said quantity of alcohol is selected from the group consisting of: a quantity of ethanol, a quantity of beer, a quantity of wine, and a quantity of distilled spirits.

8. The consumable fluid composition of claim 1, and further comprising at least one buffer.

9. The consumable fluid composition of claim 8, wherein said at least one buffer maintains said aqueous solution at a pH at or below 7.4.

10. The consumable fluid composition of claim 1, wherein the cannabinoid to be chemically modified into said water soluble acetylated cannabinoid glycoside comprises a cannabinoid from hemp.

11. The consumable fluid composition of claim 10, wherein said cannabinoid from hemp comprises is a non-psychoactive cannabinoid.

12. A chemically modified cannabinoid food additive comprising:
a consumable food additive composition of claim comprising at least one chemically modified cannabinoid provided in a dehydrated powder or liquid form, and wherein said chemically modified cannabinoid comprises a cannabinoid glycoside, an acetylated cannabinoid glycoside, or a mixture of both; and
wherein said consumable food additive composition may be introduced to a food or beverage, and wherein said cannabinoid glycoside, and said acetylated cannabinoid glycoside exhibit enhanced solubility compared to unmodified cannabinoids.

13. The consumable food additive composition of claim 12, wherein said one or more water soluble acetylated cannabinoid glycosides comprises a water soluble O-acetyl cannabinoid glycoside.

14. The consumable food additive composition of claim 12, wherein said cannabinoid glycoside comprises a cannabinoid glycoside having one, two or three sugar moieties.

15. The consumable food additive composition of claim 12, wherein said water soluble acetylated cannabinoid glycoside comprises a water soluble acetylated cannabinoid glycoside generated in vivo through incubation of a cannabinoid in a tobacco cell suspension culture.

16. The consumable food additive composition of claim 12, wherein said water soluble acetylated cannabinoid glycoside comprises a water soluble acetylated cannabinoid glycoside generated in vitro through incubation with a glycosyltransferase protein having glycosylation activity towards one or more cannabinoids and acetyltransferase protein having acetylation activity toward one or more cannabinoids.

17. The consumable food additive composition of claim 12, wherein said glycosyltransferase protein having glycosylation activity towards one or more cannabinoids comprises a glycosyltransferase protein from tobacco selected from the group consisting of: NtGT3; NtGT4; and NtGT5, and a homolog or ortholog of NtGT3, NtGT4, and NtGT5 having glycosylation activity towards one or more cannabinoids.

18. A consumable food additive composition of claim 12, and further comprising a food additive polysaccharide.

19. A consumable food additive composition of claim 18, wherein said food additive polysaccharide comprises dextrin, and maltodextrin.

20. A consumable food additive composition of claim 12, and further comprising an emulsifier.

21. A consumable food additive composition of claim 20, wherein said emulsifier is selected from the group consisting of: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.

22. A consumable food additive composition of claim 12, and further comprising a flavoring agent.

23. A consumable food additive composition of claim 22, wherein said flavoring agent comprises a flavoring agent selected from the group consisting of: Sucrose (sugar), glucose, fructose, sorbitol, mannitol, corn syrup, high fructose corn syrup, saccharin, aspartame, sucralose, acesulfame potassium (acesulfame-K), and neotame.

24. A consumable food additive composition of claim 12, and further comprising a coloring agent, and wherein said coloring agent comprises a coloring agent selected from the group consisting of: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate, and inosinate, or combinations thereof.

25. A consumable food additive composition of claim 12, and further comprising a surfactant.

26. A consumable food additive composition of claim 25, wherein said surfactant comprises a surfactant selected from the group consisting of glycerol monostearate and polysorbate 80.

27. A consumable food additive composition of claim 12, and further comprising a preservative, and wherein said preservative comprises a preservative selected from the group consisting of: ascorbic acid, citric acid, sodium benzoate, calcium propionate, sodium erythorbate, sodium nitrite, calcium sorbate, potassium sorbate, BHA, BHT, EDTA, tocopherols.

28. The consumable fluid composition of claim 12, wherein the cannabinoid to be chemically modified cannabinoid comprises a cannabinoid from hemp.

29. A consumable food additive composition of claim 12, wherein said chemically modified cannabinoid food additive is added to an alcohol beverage, and wherein said alcohol beverage is selected from the group consisting of: ethanol, beer, wine, and distilled spirits.

30. A consumable beverage composition comprising:
an alcohol continuing consumable beverage containing a chemically modified water soluble cannabinoid, wherein said chemically modified water soluble cannabinoid comprises a glycosylated cannabinoid glycoside, an acetylated cannabinoid glycoside, or a mixture of both.
